(19) United States
(12) Patent Application Publication
(10) Pub. No.: US 2014/0235486 A1
(43) Pub. Date: Aug. 21, 2014

(54) Methods and Compositions for the Treatment and Diagnosis of Breast Cancer

(71) Applicants: Karen Chapman, Mill Valley, CA (US); Joseph Wagner, San Ramon, CA (US); Michael West, Mill Valley, CA (US); Jennifer Lorrie Kidd, Alameda, CA (US); Maria Prendes, Santa Cruz, CA (US)

(72) Inventors: Karen Chapman, Mill Valley, CA (US); Joseph Wagner, San Ramon, CA (US); Michael West, Mill Valley, CA (US); Jennifer Lorrie Kidd, Alameda, CA (US); Maria Prendes, Santa Cruz, CA (US)

(73) Assignee: ONCOCYTE CORPORATION, Alameda, CA (US)

(21) Appl. No.: 14/238,726

(22) PCT Filed: Aug. 16, 2012
(86) PCT No.: PCT/US12/51235

§ 371 Date: Feb. 12, 2014

Related U.S. Application Data
(60) Provisional application No. 61/524,170, filed on Aug. 16, 2011.
(60) Provisional application No. 61/553,706, filed on Oct. 31, 2011.

(51) Int. Cl. C12Q 1/68; G01N 33/574

(52) U.S. Cl. CPC C12Q 1/6886; G01N 33/57415
USPC 506/9; 435/7.1; 435/6.12

(57) ABSTRACT

The invention provides for methods of diagnosis, prognosis and treatment of cancer including, but not limited to, breast cancer.

This application claims priority to U.S. Provisional Application No. 61/524,170 filed on Aug. 16, 2011 and U.S. Provisional Application No. 61/553,706 filed on Oct. 31, 2011, both of which are incorporated by reference in their entirety.

### FIELD OF THE INVENTION

The field of the invention relates to cancer and the diagnosis and treatment of cancer.

### BACKGROUND

Early detection of cancer can impact treatment outcomes and disease progression. Typically, cancer detection relies on diagnostic information obtained from biopsy, x-rays, CAT scans, NMR and the like. These procedures may be invasive, time consuming and expensive. Moreover, they have limitations with regard to sensitivity and specificity. There is a need in the field of cancer diagnostics for a highly specific, highly sensitive, rapid, inexpensive, and relatively non-invasive method of diagnosing cancer. Various embodiments of the invention described below meet this need as well as other needs in the field of diagnosing and treating cancer.

### SUMMARY OF THE INVENTION

Embodiments of the disclosure provide methods of diagnosis, prognosis and treatment of cancer, such as breast cancer. Other embodiments provide compositions relating to the diagnosis, prognosis and treatment of cancer such as breast cancer.

In certain embodiments the invention provides a method of detecting breast cancer in a subject comprising a) obtaining a sample from a subject; b) contacting the sample obtained from the subject with one or more agents that detect one or more markers expressed by a breast cancer cell c) contacting a non-cancerous cell with the one or more agents from b); and d) comparing the expression level of the marker in the sample obtained from the subject with the expression level in the non-cancerous cell, wherein a higher level of expression of the marker in the sample compared to the non-cancerous cell indicates that the subject has breast cancer.

In certain embodiments the invention provides a method of detecting breast cancer in a subject comprising a) obtaining a sample from a subject; b) contacting the sample obtained from the subject with one or more agents that detect expression of at least one of the markers listed in Table 1; c) contacting a non-cancerous cell, e.g. a non-cancerous cell from breast tissue, with the one or more agents from b); and d) comparing the expression level of one or more of the markers listed in Table 1 in the sample obtained from the subject with the expression level of one or more of the markers listed in Table 1 in the non-cancerous cell, wherein a higher level of expression of one or more of the markers listed in Table 1 in the sample compared to the non-cancerous cell indicates that the subject has breast cancer.

In other embodiments the invention provides a method of detecting breast cancer in a subject comprising a) obtaining a sample from a subject; b) contacting the sample obtained from the subject with one or more agents that detect expression of at least one of the markers encoded by SEQ ID NOS: 1-70 or a complement thereof; c) contacting a non-cancerous cell, e.g. a non-cancerous cell from breast tissue, with the one or more agents from b); and d) comparing the expression level of one or more of the markers encoded by SEQ ID NOS: 1-70 or a complement thereof in the sample obtained from the subject with the expression level of one or more of the markers encoded by SEQ ID NOS: 1-70 or a complement thereof in the non-cancerous cell, wherein a higher level of expression of one or more of the markers listed in Table 1 in the sample compared to the non-cancerous cell indicates that the subject has breast cancer.

In some embodiments the invention provides a method of detecting breast cancer in a subject comprising a) obtaining a sample from a subject b) contacting the sample obtained from the subject with one or more agents that detect expression of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof; c) contacting a non-cancerous cell, e.g. a non-cancerous cell from breast tissue, with the one or more agents from b); and d) comparing the expression level of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof in the sample obtained from the subject with the expression level of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof in the non-cancerous cell, wherein a higher level of expression of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10AP, NMU or a complement thereof in the sample compared to the non-cancerous cell indicates that the subject has breast cancer.

In further embodiments the invention provides a method of detecting breast cancer cells in a sample comprising a) obtaining a sample b) contacting the sample obtained in a) with one or more agents that detect expression of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof; c) contacting a non-cancerous cell, e.g. a non-cancerous cell from breast tissue, with the one or more agents from b); and d) comparing the expression level of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof in the sample obtained in a) with the expression level of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof in the non-cancerous cell, wherein a higher level of expression of one or more of the markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof in the sample compared to the non-cancerous cell indicates that the sample contains breast cancer cells. The sample may be an in vitro sample or an in vivo sample, or derived from an in vivo sample.

With regard to the embodiments described in the preceding paragraphs, the sample may be any sample as described infra, for example, a bodily fluid, such as blood, serum or urine. The sample may be a cellular sample or the extract of a cellular sample. The agent may be one or more molecules that bind specifically to one or more proteins expressed by the cancer cell or one or more nucleic acids expressed by the cell. For example, the agent may be a ptoein such as an antibody that binds specifically to the protein expressed by one of the marker genes identified infra. The agent may be one or more nucleic acids that hybridize to a nucleic acid expressed by the cancer cell. The nucleic acid expressed by the cancer cell may be an RNA molecule, e.g. an mRNA molecule. The nucleic acid molecule that hybridizes to the nucleic acid expressed by the cancer cell may be a DNA molecule, such as a DNA probe.

In still other embodiments the invention provides a composition of matter useful in distinguishing a breast cancer cell from a non-cancerous cell comprising one or more molecules that specifically bind to a molecule expressed at higher levels on a breast cancer cell compared to a non-cancer cell. As an example, the composition may comprise a protein, that binds to one or more molecules expressed by the cancer cell at higher levels compared to the non-cancer cell. As another example, the composition may comprise a nucleic acid that binds to one or more molecules expressed by the breast cancer cell at higher levels compared to the non-cancer cell.

In some embodiments the invention provides a composition of matter comprising a protein, such as an antibody, that specifically binds to a molecule expressed by a breast cancer cell chosen from the markers encoded by the sequences listed in Table 1. The molecule expressed by the breast cancer cell may be expressed by the breast cancer cell at level that is higher than the level expressed by a non-cancerous cell such as a non-cancerous breast tissue cell.

In other embodiments the invention provides a composition of matter comprising a protein, such as an antibody, that specifically binds to a molecule expressed by a breast cancer cell chosen from the markers encoded by SEQ ID NOS: 1-70. The molecule expressed by the breast cancer cell may be expressed by the breast cancer cell at level that is higher than the level expressed by a non-cancerous cell such as a non-cancerous breast tissue cell.

In certain embodiments the invention provides a composition of matter comprising a protein, such as an antibody, that specifically binds to a molecule expressed by a breast cancer cell chosen from a molecule encoded by one or more of the genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof. The molecule expressed by the breast cancer cell may be expressed by the breast cancer cell at level that is higher than the level expressed by a non-cancerous cell such as a non-cancerous breast tissue cell.

In other embodiments the invention provides a composition of matter comprising a nucleic acid that specifically binds to a molecule, such as an mRNA molecule, expressed by a breast cancer cell wherein the molecule is chosen from a marker encoded for by the genes listed in Table 1. The molecule expressed by the breast cancer cell may be expressed by the breast cancer cell at level that is higher than the level expressed by a non-cancerous cell such as a non-cancerous breast tissue cell.

In further embodiments the invention provides a composition of matter comprising a nucleic acid that specifically binds to a molecule, such as an mRNA molecule, expressed by a breast cancer cell wherein the mRNA molecule is chosen from an mRNA encoded for by SEQ ID NOS: 1-70. The molecule expressed by the breast cancer cell may be expressed by the breast cancer cell at level that is higher than the level expressed by a non-cancerous cell such as a non-cancerous breast tissue cell.

In other embodiments the invention provides a composition of matter comprising a nucleic acid that specifically binds to a molecule, such as an mRNA molecule, expressed by a breast cancer cell wherein the molecule is encoded for by a gene chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HITST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL 10A1, NMU or a complement thereof. The molecule expressed by the breast cancer cell may be expressed by the breast cancer cell at level that is higher than the level expressed by a non-cancerous cell such as a non-cancerous breast tissue cell.

In still further embodiments the invention provides a method of determining if a cancer in a subject is advancing comprising a) measuring the expression level of one or more markers associated with cancer at a first time point; b) measuring the expression level of the one or more markers measured in a) at a second time point, wherein the second time point is subsequent to the first time point; and c) comparing the expression level measured in a) and b), wherein an increase in the expression level of the one or more markers in b) compared to a) indicates that the subject's cancer is advancing.

In some embodiments the invention provides a method of determining if a breast cancer in a subject is advancing comprising a) measuring the expression level of one or more markers listed in Table 1 at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's breast cancer is advancing.

In further embodiments the invention provides a method of determining if a breast cancer in a subject is advancing comprising a) measuring the expression level of one or more markers encoded for by SEQ ID NOS: 1-70 at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's breast cancer is advancing.

In other embodiments the invention provides a method of determining if a breast cancer in a subject is advancing comprising a) measuring the expression level of one or more markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a complement thereof at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's breast cancer is advancing.

In some embodiments the invention provides antigens (i.e. cancer-associated polypeptides) associated with breast cancer as targets for diagnostic and/or therapeutic antibodies. In some embodiments, the antigen may be chosen from a protein encoded by, a gene listed in Table 1, a fragment thereof, or a combination of proteins encoded by a gene listed in Table 1.

In other embodiments the invention provides antigens (i.e. cancer-associated polypeptides) associated with breast cancer as targets for diagnostic and/or therapeutic antibodies. In some embodiments, the antigen may be chosen from a protein encoded by, a sequence chosen from SEQ ID NOS: 1-70, a fragment thereof, or a combination of proteins encoded by a sequence chosen from SEQ ID NOS: 1-70.

In some embodiments the invention provides antigens (i.e. cancer-associated polypeptides) associated with breast cancer as targets for diagnostic and/or therapeutic antibodies. In some embodiments, the antigen may be chosen from a protein encoded by, a gene chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, a fragment thereof, or a combination of proteins encoded by a gene (or fragments thereof) chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU.

In yet other embodiments the invention provides a method of eliciting an immune response to a breast cancer cell comprising contacting a subject with a protein or protein fragment that is expressed by a breast cancer cell thereby eliciting an immune response to the cancer cell. As an example the subject may be contacted intravenously or intramuscularly.

In further embodiments the invention provides a method of eliciting an immune response to a breast cancer cell comprising contacting a subject with one or more proteins or protein fragments that is encoded by a gene chosen from the genes listed in Table 1, thereby eliciting an immune response to a breast cancer cell. As an example the subject may be contacted intravenously or intramuscularly.

In yet other embodiments the invention provides a method of eliciting an immune response to a breast cancer cell comprising contacting a subject with one or more proteins or protein fragments that is encoded by a sequence listed in SEQ ID NOS: 1-70, thereby eliciting an immune response to a breast cancer cell. As an example the subject may be contacted intravenously or intramuscularly.

In still other embodiments the invention provides a method of eliciting an immune response to a breast cancer cell comprising contacting a subject with one or more proteins or protein fragments that is encoded by a gene chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU thereby eliciting an immune response to a breast cancer cell. As an example the subject may be contacted intravenously or intramuscularly.

In other embodiments the invention provides a kit for detection of cancer in a sample obtained from a subject. The kit may comprise one or more agents that bind specifically to a molecule expressed specifically by a breast cancer cell. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The kit may further contain a positive control (e.g. one or more cancerous breast cells; or specific known quantities of the molecule expressed by the cancer cell) and a negative control (e.g. a tissue or cell sample that is non-cancerous).

In some embodiments the invention provides a kit for the detection of breast cancer comprising one or more agents that specifically bind one or more markers encoded by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC72794 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU. The agent may be a protein, such as an antibody. Alternatively, the agent may be a nucleic such as a DNA molecule or an RNA molecule. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The kit may further contain a positive control (e.g. one or more cancerous cells; or specific known quantities of the molecule expressed by the cancer cell) and a negative control (e.g. a tissue or cell sample that is non-cancerous). As an example the kit may take the form of an ELISA or a DNA microarray.

Some embodiments are directed to a method of treating breast cancer in a subject, the method comprising administering to a subject in need thereof a therapeutic agent modulating the activity of a cancer associated protein, wherein the cancer associated protein is encoded by gene listed in Table 1, homologs thereof, combinations thereof, or a fragment thereof. In some embodiments, the therapeutic agent binds to the breast cancer associated protein. In some embodiments, the therapeutic agent is an antibody. In some embodiments, the antibody may be a monoclonal antibody or a polyclonal antibody. In some embodiments, the antibody is a humanized or human antibody.

Other embodiments are directed to a method of treating breast cancer in a subject, the method comprising administering to a subject in need thereof a therapeutic agent modulating the activity of a cancer associated protein, wherein the cancer associated protein is encoded by one or more sequences chosen from SEQ ID NOS: 1-70, homologs thereof, combinations thereof, or a fragment thereof. In some embodiments, the therapeutic agent binds to the breast cancer associated protein. In some embodiments, the therapeutic agent is an antibody. In some embodiments, the antibody may be a monoclonal antibody or a polyclonal antibody. In some embodiments, the antibody is a humanized or human antibody. In some embodiments, the antibody is a humanized or human antibody.

Some embodiments herein are directed to a method of treating breast cancer in a subject, the method comprising administering to a subject in need thereof a therapeutic agent modulating the activity of a cancer associated protein, wherein the cancer associated protein is encoded by gene chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU homologs thereof, combinations thereof, or a fragment thereof. In some embodiments, the therapeutic agent binds to the breast cancer associated protein. In some embodiments, the therapeutic agent is an antibody. In some embodiments, the antibody may be a monoclonal antibody or a polyclonal antibody. In some embodiments, the antibody is a humanized or human antibody.

In some embodiments, a method of treating breast cancer in a subject may comprise administering to a subject in need thereof a therapeutic agent that modulates the expression of one or more genes chosen from those listed in Table 1, fragments thereof, homologs thereof, and/or complements thereof.

In some embodiments, a method of treating breast cancer in a subject may comprise administering to a subject in need thereof a therapeutic agent that modulates the expression of one or more sequences chosen from SEQ ID NOS: 1-70, fragments thereof, homologs thereof, and/or complements thereof.

In some embodiments, a method of treating breast cancer in a subject may comprise administering to a subject in need thereof a therapeutic agent that modulates the expression of one or more genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU fragments thereof, homologs thereof, and or compliments thereof.

In further embodiments, the invention provides a method of treating breast cancer may comprising a gene knockdown of one or more genes listed in Table 1 fragments thereof, homologs thereof, and or compliments thereof. In some embodiments, a method of treating breast cancer may comprise treating cells to knockdown or inhibit expression of a gene encoding an mRNA of one or more genes chosen from those listed a Table 1, fragments thereof, homologs thereof, and or compliments thereof.

In other embodiments, a method of treating breast cancer may comprise gene knockdown of one or more genes selected from C1orf64, LOC338579, LOC648879, HIST1H4H, ASCL1, COL10A1, MMP11, DSCR6, CYP4Z1, HIST2H41B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU. In some embodiments, a method of treating breast cancer may comprise treating cells to knockdown or inhibit expression of a gene encoding an mRNA of one or more genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL 11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU.

In still other embodiments, the present invention provides methods of screening a drug candidate for activity against breast cancer, the method comprising: (a) contacting a cell that expresses one or more cancer associated genes chosen from those listed in Table 1 with a drug candidate; (b) detecting an effect of the drug candidate on expression of the one or more breast cancer associated genes in the cell from a); and (c) comparing the level of expression of one or more of the genes recited in a) in the absence of the drug candidate to the level of expression of the one or more genes in the presence of the drug candidate; wherein a decrease in the expression of the breast cancer associated gene in the presence of the drug candidate indicates that the candidate has activity against breast cancer.

In yet other embodiments, the present invention provides methods of screening a drug candidate for activity against breast cancer, the method comprising: (a) contacting a cell that expresses one or more cancer associated genes chosen from those encoded for by SEQ ID NOS: 1-70 with a drug candidate; (b) detecting an effect of the drug candidate on expression of the one or more breast cancer associated genes in the cell from a); and (c) comparing the level of expression of one or more of the genes recited in a) in the absence of the drug candidate to the level of expression of the one or more genes in the presence of the drug candidate; wherein a decrease in the expression of the breast cancer associated gene in the presence of the drug candidate indicates that the candidate has activity against breast cancer.

In further embodiments, the present invention provides methods of screening a drug candidate for activity against breast cancer, the method comprising: (a) contacting a cell that expresses one or more breast cancer associated genes chosen from C orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23 152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU with a drug candidate; (b) detecting an effect of the drug candidate on an expression of the one or more breast cancer associated genes in the cell from a); and (c) comparing the level of expression of one or more of the genes recited in a) in the absence of the drug candidate to the level of expression in the presence of the drug candidate; wherein a decrease in the expression of the breast cancer associated gene in the presence of the drug candidate indicates that the candidate has activity against breast cancer.

In some embodiments, the present invention provides methods of visualizing a breast cancer tumor in a subject comprising a) targeting one or more breast cancer associated proteins with a labeled molecule that binds specifically to the breast cancer tumor, wherein the cancer associated protein is selected from a protein encoded for by one or more genes chosen from those listed in Table 1; and b) detecting the labeled molecule, wherein the labeled molecule visualizes the tumor in the subject. Visualization may be done in vivo, or in vitro.

In other embodiments, the present invention provides methods of visualizing a breast cancer tumor in a subject comprising a) targeting one or more breast cancer associated proteins with a labeled molecule that binds specifically to the breast cancer tumor, wherein the cancer associated protein is selected from a protein encoded for by one or more sequences chosen from SEQ ID NOS: 1-70; and b) detecting the labeled molecule, wherein the labeled molecule visualizes the tumor in the subject. Visualization may be done in vivo, or in vitro.

In still other embodiments, the present invention provides methods of visualizing a breast cancer tumor in a subject comprising a) targeting one or more breast cancer associated proteins with a labeled molecule that binds specifically to the breast cancer tumor, wherein the cancer associated protein is selected from a protein encoded for by one or more genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU; and b) detecting the labeled molecule, wherein the labeled molecule visualizes the tumor in the subject. Visualization may be done in vivo or in vitro.

### DESCRIPTION OF DRAWINGS

For a fuller understanding of the nature and advantages of the present invention, reference should be had to the following detailed description taken in connection with the accompanying drawings, in which:

FIG. 1 shows the expression of C1orf64 in breast tumors and normal tissues.

FIG. 2 shows the expression of LOC648879 in breast tumors and normal tissues.

FIG. 3 shows the expression of HIST1H4H in breast tumors and normal tissues.

FIG. 4 shows the expression of HIST2H4B in breast tumors and normal tissues.

FIG. 5 shows the expression of BX116033 in breast tumors and normal tissues.

FIG. 6 shows the expression of DSCR6 in breast tumors, malignant tumors of various types, and normal tissues.

FIG. 7 shows the expression of DSCR6 in metastatic tumors of diverse tissues of origin and normal tissues.

FIG. 8 shows the expression of POTEC in breast tumors v. normal tissues.

FIG. 9 shows the expression of FSIP1 in breast tumors v. normal tissues.

FIG. 10 shows the expression of GFRA1 in breast tumors v. normal tissues.

FIG. 11 shows the expression of POTEF, POTEE, and POTEK in breast tumors v. normal tissues.

FIG. 12 shows the expression of C2orf27A in breast tumors v. normal tissues.

FIG. 13 shows the expression of LOC727941 in breast tumors v. normal tissues.

FIG. 14 shows the expression of NBPF22P in breast tumors v, normal tissues.

FIG. 15 shows the expression of POTEG in breast tumors v. normal tissues.

FIG. 16 shows the expression of RET in breast tumors v. normal tissues.

FIG. 17 shows the expression of TMEM145 in breast tumors v. normal tissues.

FIG. 18 shows the expression of LOC727941 in breast tumors v. normal tissues.

FIG. 19 shows the expression of NAT1 in breast tumors v. normal tissues.

FIG. 20 shows the expression of NXPH1 in breast tumors v. normal tissues.

FIG. 21 shows the expression of SERHL2 in breast tumors v. normal tissues.

FIG. 22 shows the expression of SYCP2 in breast tumors v. normal tissues.

FIG. 23 shows the expression of D59687 in breast tumors v. normal tissues.

FIG. 24 shows the expression of CYP4Z1 in breast tumors v. normal tissues.

FIG. 25 shows the expression of LOC730024 in breast tumors v. normal tissues.

FIG. 26 shows the expression of NOS1AP in breast tumors v. normal tissues.

FIG. 27 shows the expression of UGT2B28 in breast tumors v. normal tissues.

FIG. 28 shows the expression of GRM4 in breast tumors v. normal tissues.

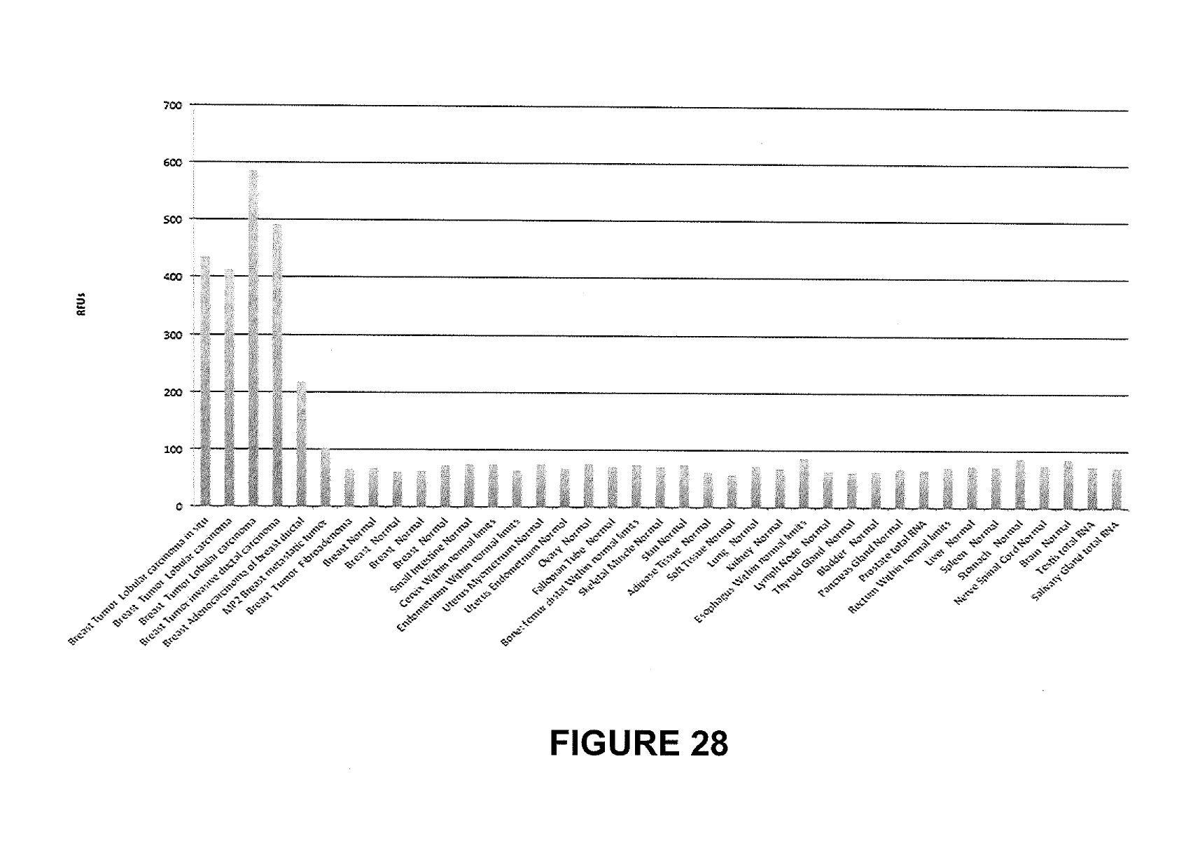

FIG. 29 shows the expression of FLJ30428 in breast tumors v. normal tissues.

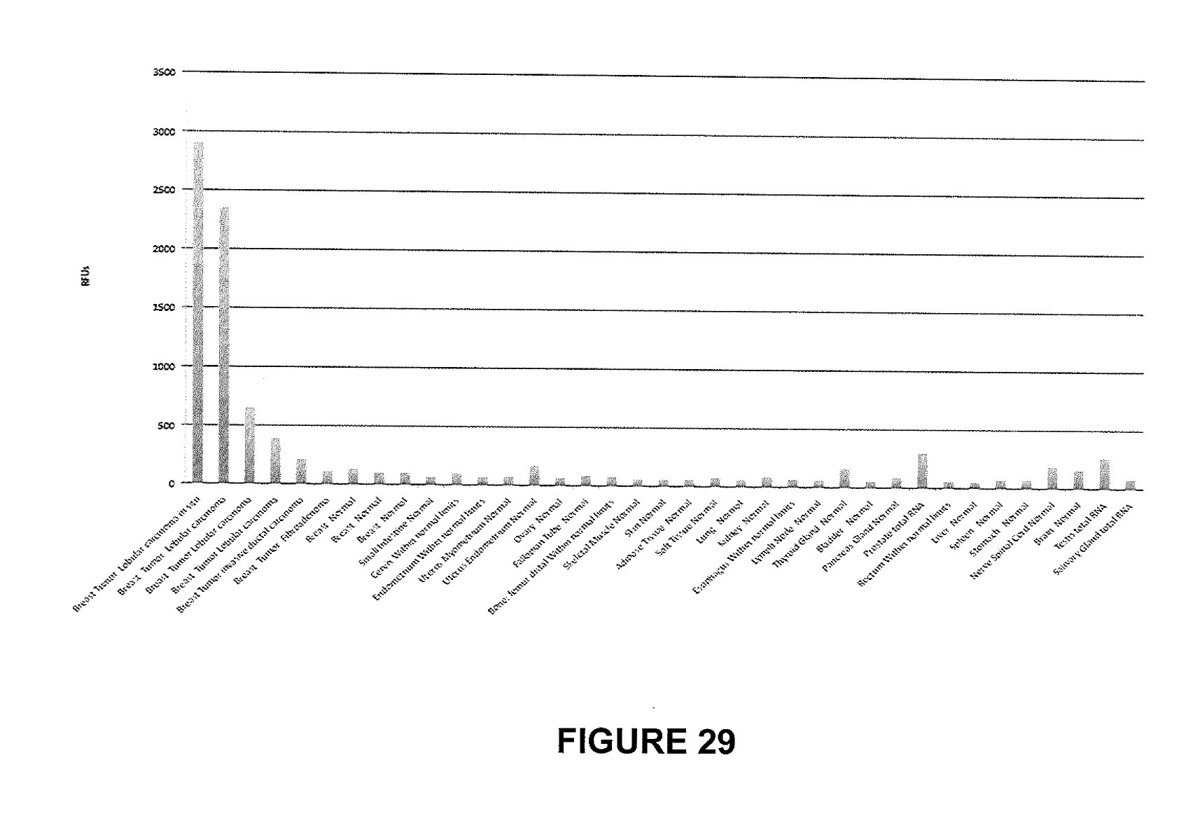

FIG. 30 shows the expression of LOC440905 in breast tumors v. normal tissues.

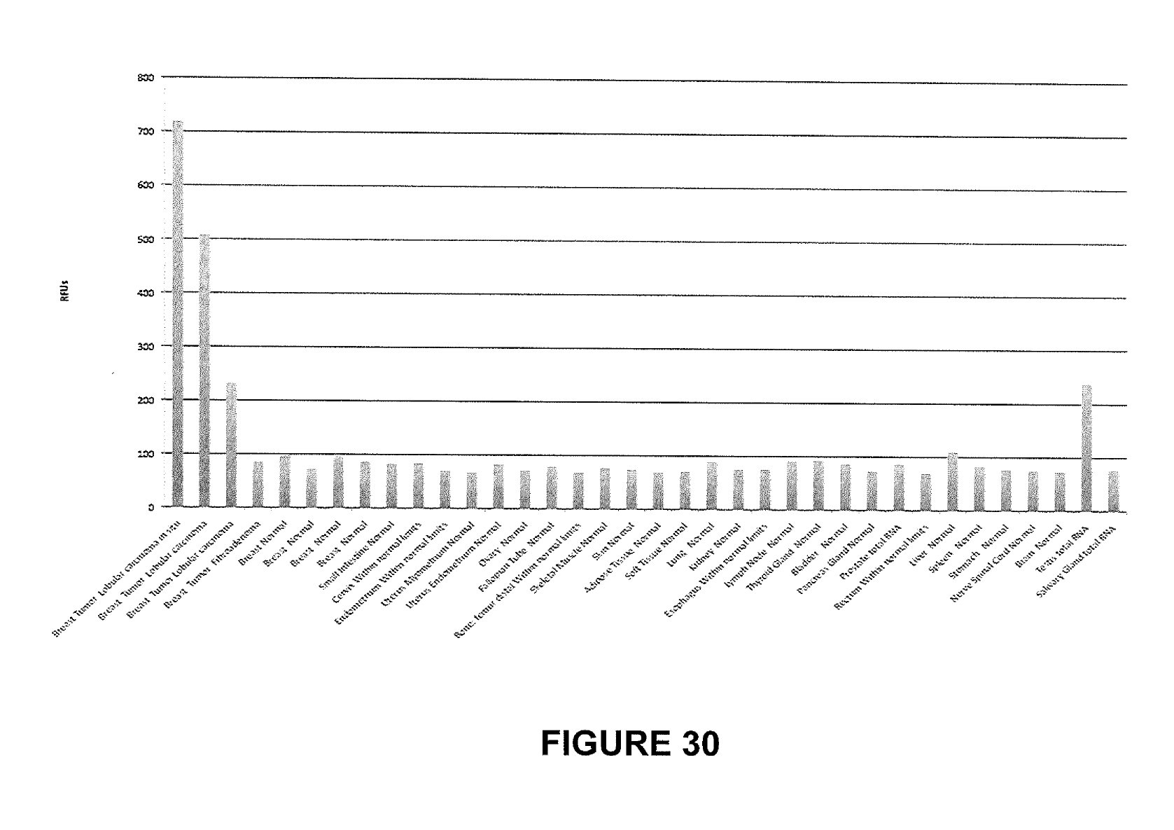

FIG. 31 shows the expression of LOC642460 in breast tumors v. normal tissues.

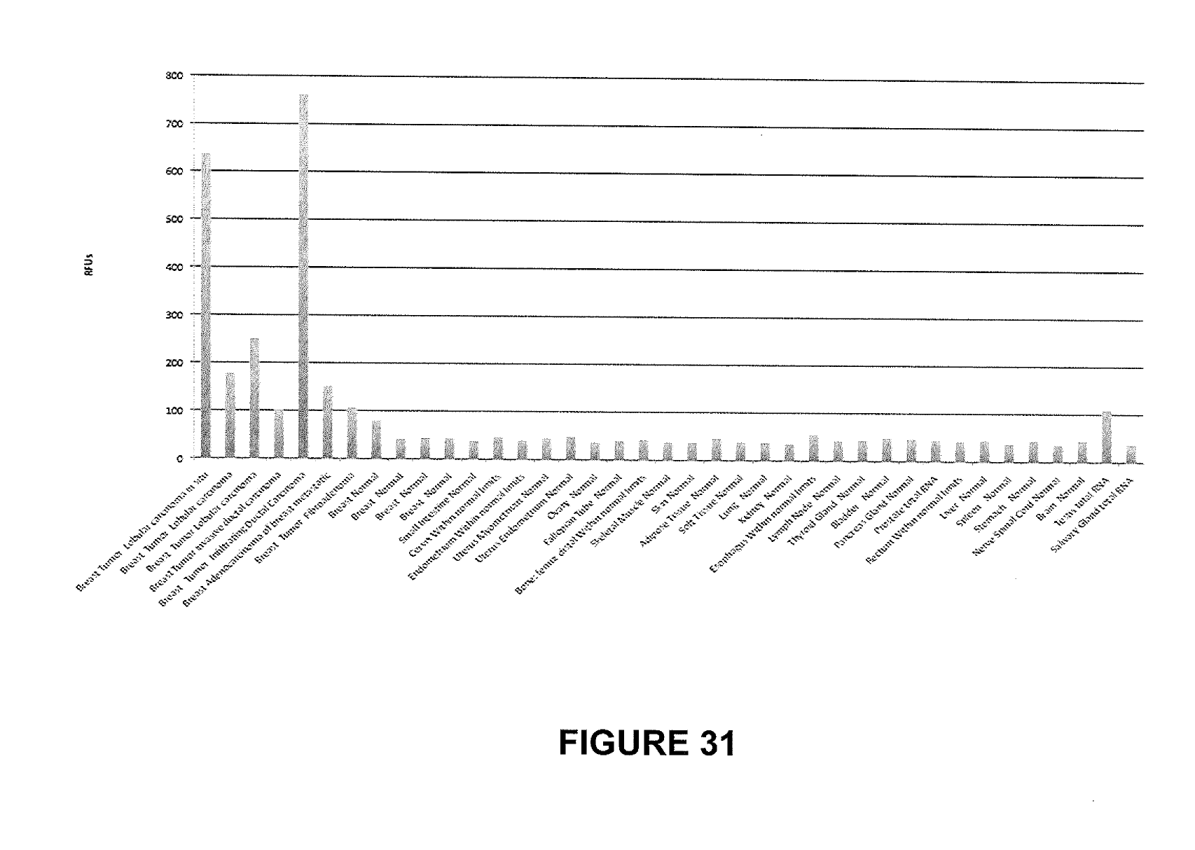

FIG. 32 shows the expression of MTL5 in breast tumors v. normal tissues.

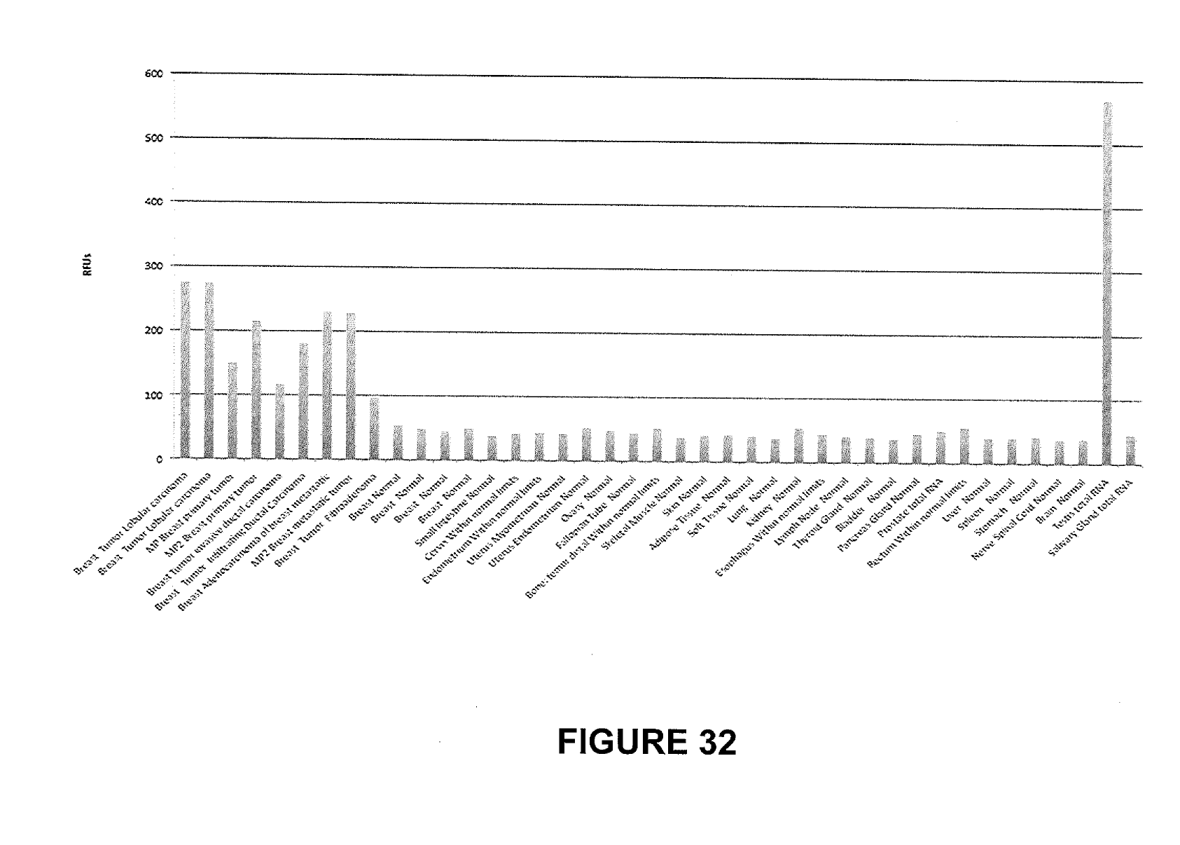

FIG. 33 shows the expression of GRPR in breast tumors v. normal tissues.

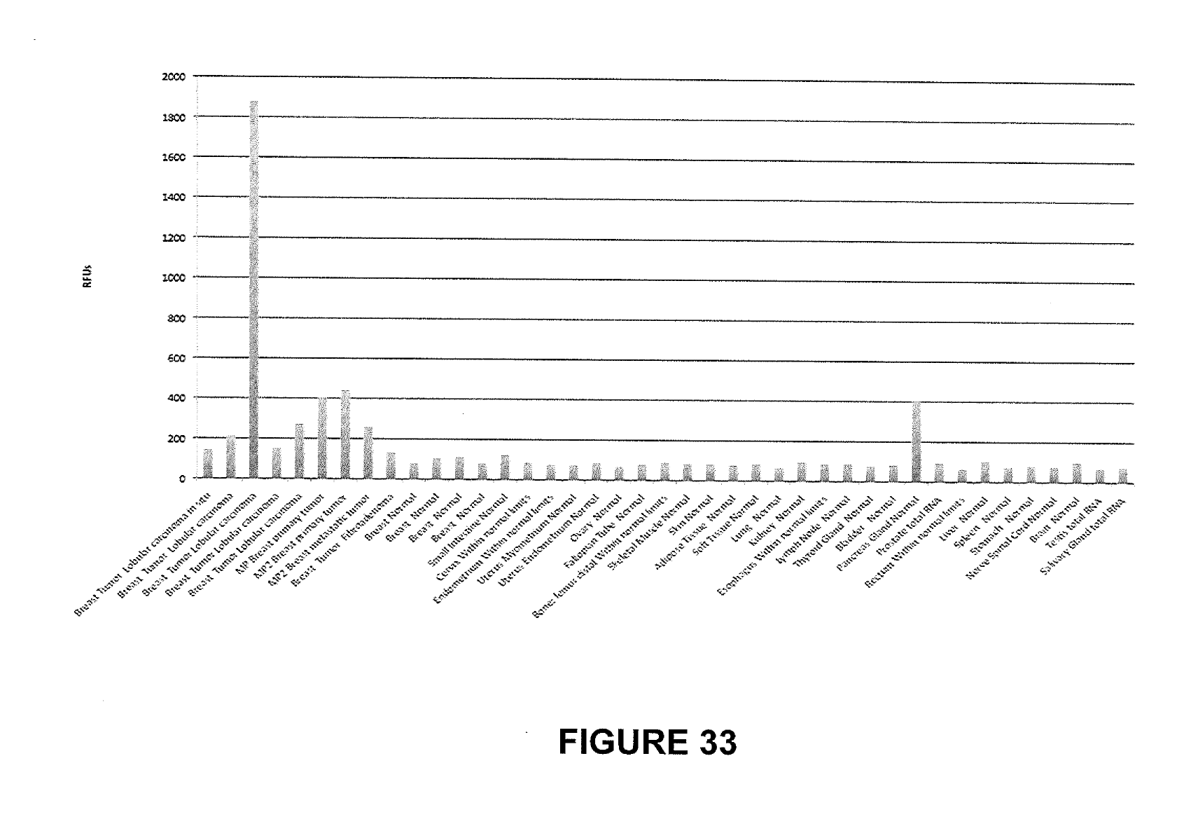

FIG. 34 shows the expression of COL10A1 in breast tumors v. normal tissues.

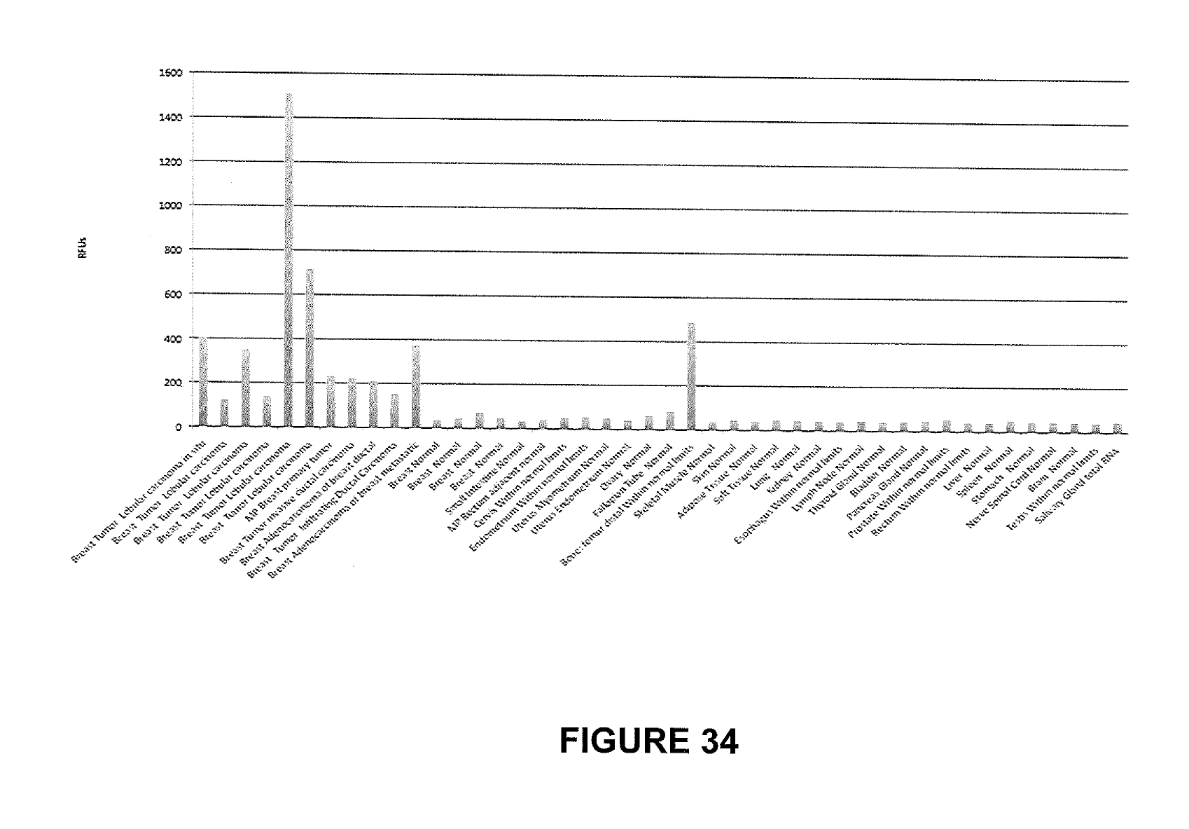

FIG. 35 shows the expression level of ASCL1 in breast tumors v. normal tissues.

FIG. 36 shows the expression level of BX116033 in breast tumors v. normal tissues.

FIG. 37 shows the expression level of C1orf64 in breast tumors v. normal tissues.

FIG. 38 shows the expression level of COL10A1 in breast tumors v. normal tissues.

FIG. 39 shows the expression level of DSCR6 in breast tumors v. normal tissues.

FIG. 40 shows the expression level of FLJ23152 in breast tumors v. normal tissues.

FIG. 41 shows the expression level of GRM4 in breast tumors v. normal tissues.

FIG. 42 shows the expression level of TMEM145 in breast tumors v. normal tissues.

FIG. 43 shows the expression level of POTEG in breast tumors v. normal tissues.

FIG. 44 shows the expression level of FSIP1 in breast tumors v. normal tissues.

FIG. 45 shows expression of collagen 10 (COL 10A1) in breast tumors.

FIG. 46 shows expression of MMP11 in breast tumors.

FIG. 47 shows expression levels of ANKRD30A in serum from breast cancer patients v. normal donor serum.

FIG. 48 shows expression levels of C1orf64 in serum from breast cancer patients v. normal donor serum.

FIG. 49 shows expression levels of COL10A1 in serum from breast cancer patients v. normal donor serum.

FIG. 50 shows expression levels of MMP11 in serum from breast cancer patients v. normal donor serum.

FIG. 51 shows expression levels of COL11A1 in serum from breast cancer patients v. normal donor serum.

FIG. 52 shows expression levels of POTEG in serum from breast cancer patients v. normal donor serum.

FIG. 53 shows expression of FSIP1 in breast tumors.

FIG. 54 shows expression levels of NMU in serum from breast cancer patients v. normal donor serum.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. Nothing herein is to be construed as an admission that the invention is not entitled to antedate such disclosure contained in a cited publication by virtue of prior invention.

### DEFINITIONS

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 45% to 55%.

“Administering,” when used in conjunction with a therapeutic, means to administer a therapeutic directly into or onto a target tissue or to administer a therapeutic to a patient whereby the therapeutic positively impacts the tissue to which it is targeted. Thus, as used herein, the term “administering,” when used in conjunction with a therapeutic, can include, but is not limited to, providing the therapeutic into or onto the target tissue; providing the therapeutic systemically to a patient by, e.g., intravenous injection whereby the therapeutic reaches the target tissue; providing the therapeutic in the form of the encoding sequence thereof to the target tissue (e.g., by so-called gene-therapy techniques). “Administering” a composition may be accomplished by oral administration, intravenous injection, intraperitoneal injection, intramuscular injection, subcutaneous injection, transdermal diffusion or electrophoresis, local injection, extended release delivery devices including locally implanted extended release devices such as bioerodible or reservoir-based implants, as protein therapeutics or as nucleic acid therapeutic via gene therapy vectors, topical administration, or by any of these methods in combination with other known techniques. Such combination techniques include, without limitation, heating, radiation and ultrasound.

The term “animal,” “patient” or “subject” as used herein includes, but is not limited to, humans and non-human vertebrates such as wild, domestic and farm animals. A subject can be for example any mammal, including humans, non-human primates, dogs, cats, rodents such as rats or mice, rabbits, guinea pigs, pigs, cows, sheep and the like. In some embodiments, the term “subject,” “patient” or “animal” refers to a male. In some embodiments, the term “subject,” “patient” or “animal” refers to a female.

The term “breast cancer” as used herein may include one or more of the following: ductal carcinoma in situ (DCIS), invasive ductal carcinoma (WC), medullary carcinoma, invasive lobular carcinoma (ILC), tubular carcinoma, mucinous carcinoma, inflammatory breast cancer (IBC), lobular carcinoma in situ (LCIS), male breast cancer, Paget's disease of the nipple, phyllodes tumors of the breast, recurrent and metastatic breast cancer.

The term “capture reagent” refers to a reagent, for example an antibody or antigen binding protein, capable of binding a target molecule or analyte to be detected in a sample.

The term “inhibiting” includes the administration of a compound of the present disclosure to prevent the onset of the symptoms, alleviating the symptoms, or eliminating the disease, condition or disorder.

The term “differentiated cells” when used in reference to cells made by methods of this invention from pluripotent stein cells refer to cells having reduced potential to differentiate when compared to the parent pluripotent stein cells. The differentiated cells of this invention comprise cells that could differentiate further (i.e., they may not be terminally differentiated).

The term “gene expression result” refers to a qualitative and/or quantitative result regarding the expression of a gene or gene product. The gene expression result can be an amount or copy number of the gene, the RNA encoded by the gene, the mRNA encoded by the gene, the protein product encoded by the gene, or any combination thereof. The gene expression result can also be normalized or compared to a standard. The gene expression result can be used, for example, to determine if a gene is expressed, overexpressed, or differentially expressed in two or more samples.

The term “homology,” as used herein, refers to a degree of complementarity. There may be partial homology or complete homology. The word “identity” may substitute for the word “homology,” A partially complementary nucleic acid sequence that at least partially inhibits an identical sequence from hybridizing to a target nucleic acid is referred to as “substantially homologous.” The inhibition of hybridization of the completely complementary nucleic acid sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The phrases “percent homology,” “% homology,” “percent identity,” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645). Identity between sequences can also be determined by other methods known in the art, e.g., by varying hybridization conditions.

The term “label” or “detectable substance” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by a device or method, such as, but not limited to, a spectroscopic, photochemical, biochemical, immunochemical, electrical, optical, chemical detection device or any other appropriate device. In some embodiments, the label may be detectable visually without the aid of a device. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

“Microarray” as used herein, refers to a linear or two-dimensional array of, for example, discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to identify, amplify, detect, or clone target polynucleotides. Since the position of each particular group of primers in the array is known, the identities of the target polynucleotides can be determined based on their binding to a particular position in the microarray.

As used herein, the term “naturally occurring” refers to sequences or structures that may be in a form normally found in nature. “Naturally occurring” may include sequences in a form normally found in any animal.

The term “nucleic acid,” “polynucleotide” or “oligonucleotide” or equivalents herein means at least two nucleotides covalently linked together. In some embodiments, an oligonucleotide is an oligomer of 6, 8, 10, 12, 20, 30, or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, PNA or a polymer of nucleotides linked by phosphodiester and/or any alternate bonds.

By “pharmaceutically acceptable”, it is meant the carrier, diluent or excipient must be compatible with the other ingredients of the formulation and not deleterious to the recipient thereof.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a cancer associated gene.

“Recombinant Protein,” as used herein, is a protein made using recombinant techniques, for example, but not limited to, through the expression of a recombinant nucleic acid as depicted above. A recombinant protein may be distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75%, about 80%, or about 90%. In some embodiments, a substantially pure protein comprises about 80-99%, 85-99%, 90-99%, 95-99%, or 97-99% by weight of the total protein. A recombinant protein can also include the production of a cancer associated protein from one organism (e.g. human) in a different organism (e.g. yeast, E. coli, or the like) or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed herein.

The terms “specific binding,” “specifically binds,” and the like, refer to instances where two or more molecules form a complex that is measurable under physiologic or assay conditions and is selective. An antibody or antigen binding protein or other molecule is said to “specifically bind” to a protein, antigen, or epitope if; under appropriately selected conditions, such binding is not substantially inhibited, while at the same time non-specific binding is inhibited. Specific binding is characterized by a high affinity and is selective for the compound, protein, epitope, or antigen. Nonspecific binding usually has a low affinity.

Specific Binding in IgG antibodies, for example, is generally characterized by an affinity of at least about 10⁻⁷ M or higher, such as at least about 10⁻⁸ M or higher, or at least about 10⁻⁹ M or higher, or at least about 10⁻¹⁰ or higher, or at least about 10⁻¹¹ M or higher, or at least about 10⁻¹² M or higher. The term is also applicable where, e.g., an antigen-binding domain is specific for a particular epitope that is not carried by numerous antigens, in which case the antibody or antigen binding protein carrying the antigen-binding domain will generally not bind other antigens.

As used herein, the term “sample” refers to composition that is being tested or treated with a reagent, such as but not limited to a therapeutic, drug, or candidate agent. Samples may be obtained from subjects. In some embodiments, the sample may be blood, plasma, serum, or any combination thereof. A sample may be derived from blood, plasma, serum, or any combination thereof. Other typical samples include, but are not limited to, any bodily fluid obtained from a mammalian subject, tissue biopsy, sputum, lymphatic fluid, blood cells (e.g., peripheral blood mononuclear cells), tissue or fine needle biopsy samples, urine, peritoneal fluid, colostrums, breast milk, fetal fluid, fecal material, tears, pleural fluid, or cells therefrom. The sample may processed in some manner before being used in a method described herein, for example a particular component to be analyzed or tested according to any of the methods described infra may be isolated from a sample.

The term “support” refers to conventional supports such as beads, particles, dipsticks, fibers, filters, membranes, and silane or silicate supports such as glass slides.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

As used herein, the term “therapeutic” or “therapeutic agent” means an agent that can be used to treat, combat, ameliorate, prevent or improve an unwanted condition or disease of a patient. In part, embodiments of the present disclosure are directed to the treatment of cancer or the decrease in proliferation of cells. In some embodiments, the term “therapeutic” or “therapeutic agent” may refer to any molecule that associates with or affects the target marker, its expression or its function. In various embodiments, such therapeutics may include molecules such as, for example, a therapeutic cell, a therapeutic peptide, a therapeutic gene, a therapeutic compound, or the like, that associates with or affects the target marker, its expression or its function.

A “therapeutically effective amount” or “effective amount” of a composition is a predetermined amount calculated to achieve the desired effect, i.e., to inhibit, block, or reverse the activation, migration, or proliferation of cells. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable excipient composition, it is sufficient to achieve an effective systemic concentration or local concentration in the targeted tissue.

The term “tissue” refers to any aggregation of similarly specialized cells that are united in the performance of a particular function.

The terms “treat,” “treated,” or “treating” as used herein can refer to both therapeutic treatment or prophylactic or preventative measures, wherein the object is to prevent or slow down (lessen) an undesired physiological condition, disorder or disease, or to obtain beneficial or desired clinical results. The terms can also refer to the amelioration of one or more symptoms associated with a disease or condition. In some embodiments, the term may refer to both treating and preventing. For the purposes of this disclosure, the terms include, but are not limited to, diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment may also include prolonging survival as compared to expected survival if not receiving treatment.

In certain embodiments the invention described herein provides for a rapid, relatively non-invasive, sensitive and specific method for detecting and/or diagnosing cancer, such as breast cancer, in a subject. The method in certain embodiments includes the isolation of a sample from a subject and analyzing the sample, according to methods described herein, to determine if the subject has cancer, e.g. breast cancer. Other embodiments described herein provide for methods of treating cancer by targeting expression and or activity of markers expressed in cancer cells. Additional embodiments include screening for compounds with anti-cancer activity by analyzing the effect of test compounds on cancerous cells, including the effect on the expression of and/or activity of markers disclosed herein.

### Methods of Diagnosing Cancer

Breast cancer may be detected in any type of sample, including, but not limited to, serum, blood, tissue and the like. The sample may be any type of sample as it is described herein obtained from any subject.

In some embodiments, the cancer may be selected from ductal carcinoma in situ (DCIS), invasive ductal carcinoma (IDC), medullary carcinoma, invasive lobular carcinoma (ILC), tubular carcinoma, mucinous carcinoma, inflammatory breast cancer (IBC), lobular carcinoma in situ (LCIS), male breast cancer, Paget's disease of the nipple, phyllodes tumors of the breast, recurrent and metastatic breast cancer, or a combination thereof.

In some embodiments the method of diagnosing breast cancer comprises obtaining a sample from a subject and analyzing the sample for expression level in the sample of one or more genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC72794 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU. Increased expression levels compared to a normal non-cancerous sample may indicate the subject has cancer. Expression levels of any of the above genes equal to or greater to that found in sample known to be positive for cancer, e.g. breast cancer, may also indicate that the subject has cancer.

In some embodiments, a method of diagnosing breast cancer may comprise detecting a level of the cancer associated protein in a subject. In some embodiments, a method of screening for cancer may comprise detecting a level of the cancer associated protein in a sample obtained from a subject. In some embodiments, the cancer associated protein is encoded by a nucleotide sequence selected from SEQ ID NOS: 1-70, a fraction thereof or a complementary sequence thereof.

In some embodiments, detecting the presence of a cancer associated sequence selected from SEQ ID NOS: 1-70 comprises contacting the sample obtained from a subject with an antibody or other type of capture reagent that specifically binds to the cancer associated sequence's protein and detecting the presence or absence of the binding to the cancer associated sequence's protein in the sample. Examples of assays that can be used, to detect binding to a protein encoded by a cancer associated sequence as described infra include, but are not limited to, an ELISA, a radioimmunoassay (RIA), flow cytometry and the like.

In some embodiments, a method of diagnosing a subject with breast cancer comprises detecting the presence of, and/or expression level of a cancer associated sequence selected from SEQ ID NOS: 1-70, wherein the presence of the cancer associated sequence and/or the expression level, indicates that the subject has breast cancer. Expression level of a cancer associated sequence may be analyzed by isolating a nucleic acid, such as mRNA from a sample obtained from a subject. In some embodiments, the method comprises detecting the presence or absence of a cancer associated sequence selected from SEQ ID NOS: 1-70, wherein the absence of the cancer associated sequence indicates that absence of breast cancer.

In some embodiments, the method of diagnosing breast cancer may comprise assaying gene expression of a subject in need thereof. In some embodiments, detecting a level of a cancer associated sequence may comprise isolating mRNA or protein from a sample obtained from a subject and analyzing the sample using techniques such as, but not limited to, PCR, mass spectroscopy, microarray or other detection techniques described herein or any technique known in the art.

In some embodiments, the present disclosure provides a method of diagnosing breast cancer, cancer, or a neoplastic condition in a subject, the method comprising obtaining a cancer associated sequence gene expression result of a cancer associated sequence selected from SEQ ID NOS: 1-70 from a sample derived from a subject; and diagnosing breast cancer or a neoplastic condition in the subject based on the cancer associated sequence gene expression result, wherein the subject is diagnosed as having breast cancer or a neoplastic condition if the cancer associated sequence is overexpressed or expressed at level found in a positive control such as sample known to be cancerous e.g. to be positively diagnosed as having breast cancer. A positive diagnosis can also be made by comparing the expression level of a cancer associated sequence with a normal sample obtained from a control subject who does not have cancer. Expression levels in the test sample that are greater than those found in the normal sample may indicate the test subject has cancer.

In some embodiments, the present disclosure provides methods of detecting cancer in a test sample, comprising: (i) detecting a level of activity of at least one polypeptide that is a gene product; and (ii) comparing the level of activity of the polypeptide in the test sample with a level of activity of polypeptide in a normal sample (obtained from a subject that does not have cancer), wherein an altered level of activity of the polypeptide in the test sample relative to the level of polypeptide activity in the normal sample is indicative of the presence of cancer in the test sample, wherein said gene product is a product of a gene selected from: C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941(XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, NMU or a combination thereof.

In some embodiments, the subject is diagnosed as not having breast cancer, cancer, or a neoplastic condition if the cancer associated sequence is not overexpressed or is expressed at a level below that which is found in a positive control (e.g. a sample known to be positive for cancer).

In some embodiments of the invention, the cancer that is diagnosed based upon a cancer associated sequence gene expression result or the absence or presence of a cancer associated sequence or protein, as described infra, is a cancer selected from the group consisting of ductal carcinoma in situ (DCIS), invasive ductal carcinoma (IDC), medullary carcinoma, invasive lobular carcinoma (ILC), tubular carcinoma, mucinous carcinoma, inflammatory breast cancer (IBC), lobular carcinoma in situ (LCIS), male breast cancer, Paget's disease of the nipple, phyllodes tumors of the breast, recurrent and metastatic breast cancer, or any combination thereof.

In some embodiments, the present invention provides methods of detecting or diagnosing cancer, such as breast cancer, comprising detecting the expression of a nucleic acid sequence selected from SEQ ID NO: 1-66, wherein a sample is contacted with a biochip comprising a sequence selected from SEQ ID NOS: 1-70, homologs thereof, combinations thereof, or a fragment thereof. The nucleic acid may be mRNA.

In some embodiments, the invention provides a method for detecting a cancer associated sequence with the expression of a polypeptide in a test sample, comprising detecting a level of expression of at least one polypeptide such as, without limitation, a cancer associated protein, or a fragment thereof. In some embodiments, the method comprises comparing the level of expression of the polypeptide in the test sample with a level of expression of polypeptide in a normal sample, wherein an altered level of expression of the polypeptide in the test sample relative to the level of polypeptide expression in the normal sample is indicative of the presence of cancer in the test sample. In some embodiments, the polypeptide expression is compared to a cancer sample, wherein the level of expression is at least the same as the cancer is indicative of the presence of cancer in the test sample. In some embodiments, the sample is a cell sample.

In some embodiments, the invention provides a method for detecting cancer by detecting the presence of an antibody in a test sample. The sample may be, for example, serum. In some embodiments, the antibody recognizes a polypeptide or an epitope thereof disclosed herein as a cancer associated sequence. In some embodiments, the antibody recognizes a polypeptide or epitope thereof encoded by a nucleic acid sequence disclosed herein. In some embodiments, the method comprises detecting a level of an antibody against an antigenic polypeptide such as, without limitation, a cancer associated protein, or an antigenic fragment thereof. In some embodiments, the method comprises comparing the level of the antibody in the test sample with a level of the antibody in the control sample, wherein an altered level of antibody in said test sample relative to the level of antibody in the control sample is indicative of the presence of cancer in the test sample. In some embodiments, the control sample is a sample derived from a normal cell or non-cancerous sample. In some embodiments, the control is derived from a cancer sample, and, therefore, in some embodiments, the method comprises comparing the levels of binding and/or the amount of antibody in the sample. Thus where the control is a negative control, a sample having a greater amount of antibody compared to the negative control may indicate the subject has cancer. Where the control is a positive control, a test sample with an amount of antibody present in the test sample equal to or greater than that found in the positive control may indicate the subject has cancer.

Also provided herein is a method for diagnosing or determining the propensity to cancers, for example, without limitation, ductal carcinoma in situ (DCIS), invasive ductal carcinoma (IDC), medullary carcinoma, invasive lobular carcinoma (ILC), tubular carcinoma, mucinous carcinoma, inflammatory breast cancer (IBC), lobular carcinoma in situ (LCIS), male breast cancer, Paget's disease of the nipple, phyllodes tumors of the breast, recurrent and metastatic breast cancer, or a combination thereof. The method of determining the propensity to develop cancer, such as breast cancer, may comprise measuring the level of expression of a cancer associated marker disclosed herein. Elevated levels of expression of the cancer associated sequences disclosed herein may indicate a propensity to develop cancer. Elevated levels may be determined by comparing the expression level of one or more cancer associated sequences disclosed herein in a test sample obtained from a subject with a sample known to be negative for cancer and/or a sample known to be positive for cancer.

In some embodiments, a method for diagnosing cancer or a neoplastic condition comprises a) determining the expression of one or more genes comprising a nucleic acid sequence selected from the group consisting of the human genomic and mRNA sequences described in Table 1, in a first sample type (e.g. tissue) of a first individual; and b) comparing said expression of said gene(s) from a second normal sample type from said first individual or a second unaffected individual; wherein a difference in said expression indicates that the first individual has cancer. In some embodiments, the expression is increased as compared to the normal sample. In some embodiments, the expression is decreased as compared to the normal sample.

In some embodiments, the invention also provides a method for detecting presence or absence of cancer cells in a subject. In some embodiments, the method comprises contacting one or more cells from the subject with an antibody as described herein. In some embodiments, the method comprises detecting a complex of a cancer associated protein and the antibody, wherein detection of the complex indicates with the presence of cancer cells in the subject.

In some embodiments, the present disclosure provides methods of diagnosing cancer or a neoplastic condition in a subject, the method comprising: a) determining the expression of one or more genes or gene products or homologs thereof; and b) comparing said expression of the one or more nucleic acid sequences from a second normal sample from said first subject or a second unaffected subject, wherein a difference in said expression indicates that the first subject has cancer, wherein the gene or the gene product is referred to as a gene selected from: Homo sapiens chromosome 1 open reading frame 64 (C1orf64), Homo sapiens hypothetical protein LOC338579, transcript variant 2 (LOC338579), Homo sapiens similar to protein expressed in prostate, ovary, testis, and placenta 14 isoform POTE-14A (LOC648879), Homo sapiens histone cluster 1, H4h (HIST1H4H), Homo sapiens achaete-scute complex homolog 1 (ASCL1), Homo sapiens collagen, type X, alpha 1 (COL10A1), Homo sapiens matrix metallopeptidase 11 (stromelysin 3) (MMP11), Homo sapiens Down syndrome critical region gene 6 (DSCR6), Homo sapiens cytochrome P450, family 4, subfamily Z, polypeptide 1 (CYP4Z1), Homo sapiens histone cluster 2, H4b (HIST2H4B), BX116033 NCI_CGAP_Lu24 Homo sapiens cDNA clone IMAGp998A155622 (BX116033), Homo sapiens chromosome 6 open reading frame 126 (C6orf126), Homo sapiens C-type lectin domain family 3, member A (CLEC3A), Homo sapiens histone cluster 2, H4a (HST2H4A), Homo sapiens serine hydrolase-like 2 (SERHL2), Homo sapiens hypothetical protein LOC401236 (FLJ23152), Homo sapiens ATP-binding cassette, sub-family C(CFTR/MRP), member 11 (ABCC11), transcript variant 3, Homo sapiens ankyrin repeat domain 30A (ANKRD30A), Homo sapiens cyclin N-terminal domain containing 2 (CNTD2), Homo sapiens collagen, type XI, alpha 1 (COL11A1), transcript variant A, Homo sapiens dehydrogenase/reductase (SDR family) member 2 (DHRS2), transcript variant 1, Homo sapiens histone cluster 1, H3f (HIST1H3F), Homo sapiens histone cluster 1, H3h (HIST1H3H), Homo sapiens histone cluster 2, H2ab (HIST2H2AB), Homo sapiens potassium channel, subfamily K, member 15 (KCNK15), Homo sapiens AARD protein (LOC441376), Homo sapiens similar to glycine-N-acyltransferase-like 1 (LOC643637), Homo sapiens hCG25653 (LOC646360), Homo sapiens protein tyrosine phosphatase, receptor type, T (PTPRT), transcript variant 2, Homo sapiens RUN domain containing 3A (RUNDC3A), Homo sapiens secretoglobin, family 2A, member 2 (SCGB2A2), Homo sapiens SLIT and NTRK-like family, member 6 (SLITRK6), Homo sapiens synaptophysin (SYP), Homo sapiens ubiquitin-conjugating enzyme E2C (UBE2C), transcript variant 3, Homo sapiens zinc finger protein 552 (ZNF552), Homo sapiens similar to calpain 8, transcript variant 4 (LOC388743), NMU (NM_{—}006681.1)(Homo sapiens neuromedin mRNA) or a combination thereof.

### Cancer Associated Sequences

In some embodiments, the present disclosure provides for nucleic acid and protein sequences that are associated with cancer, herein termed “cancer associated” or “CA” sequences. The cancer associated sequences may be for example, mRNA and/or protein that have been isolated. The cancer associated sequences may be a fragment of any of the cancer associated sequences described infra. The cancer associated sequences may be modified chemically relative to that found in a biological sample.

In some embodiments, the present disclosure provides nucleic acid and protein sequences that are associated with breast cancers or carcinomas such as, without limitation, ductal carcinoma in situ (DCIS), invasive ductal carcinoma (IDC), medullary carcinoma, invasive lobular carcinoma (ILC), tubular carcinoma, mucinous carcinoma, inflammatory breast cancer (IBC), lobular carcinoma in situ (LCIS), male breast cancer, Paget's disease of the nipple, phyllodes tumors of the breast, recurrent and metastatic breast cancer, or any combination thereof. In some embodiments, the present disclosure provides nucleic acid and protein sequences that are associated with cancers or carcinomas such as, without limitation, small cell lung carcinoma, metastatic cervix adenocarcinoma, urinary bladder carcinoma, metastatic prostate adenocarcinoma, uterus endometrial stromal sarcoma, stomach tumor adenocarcinoma, metastatic tonsil carcinoma, large intestine rectum tumor adenocarcinoma, metastatic stomach tumor, metastatic kidney tumor from transitional cell carcinoma, metastatic endometrial stromal sarcoma, pleura tumor malignant sarcoma, rectum adenocarcinoma, cartilage chondrosarcoma, pancreas neuroendocrine carcinoma, lung squamous carcinoma, kidney carcinoma, liver cholangiocarcinoma, bone osteosarcoma metastatic, gastroesophageal junction adenocarcinoma metastatic, thyroid gland carcinoma metastatic, ovary tumor, prostate adenocarcinoma, rectum metastatic tumor or a combination thereof. In some embodiments, the term “cancer associated sequences” may indicate that the nucleotide or protein sequences are differentially expressed, activated, inactivated or altered in cancers as compared to normal tissue. Cancer associated sequences may include those that are up-regulated (i.e. expressed at a higher level), as well as those that are down-regulated (i.e. expressed at a lower level), in cancers. Cancer associated sequences can also include sequences that have been altered (i.e., translocations, truncated sequences or sequences with substitutions, deletions or insertions, including, but not limited to, point mutations) and show either the same expression profile or an altered profile. In some embodiments, the cancer associated sequences may be from humans; however, as will be appreciated by those in the art, cancer associated sequences from other organisms may be useful in animal models of disease and drug evaluation; thus, other cancer associated sequences may be useful such as, without limitation, sequences from vertebrates, including mammals such as rodents (rats, mice, hamsters, guinea pigs, etc.), primates, and farm animals (including sheep, goats, pigs, cows, horses, etc.). Cancer associated sequences from other organisms may be obtained using the techniques outlined herein.

Cancer associated sequences of embodiments herein are disclosed, for example, in Table 1. These sequences were extracted from fold-change and filter analysis KC110729.5. Expression of these cancer associated sequences in normal and breast tumor tissues is disclosed in Table 2. Once expression was determined, the gene sequence results were further filtered by considering fold-change in cancer cell lines vs. normal tissue; general specificity; secreted or not, level of expression in cancer cell lines; and signal to noise ratio.

Cancer associated sequences may include polypeptides and/or polynucleotides. Accordingly, cancer associated sequences can include amino acid sequences and or nucleic acid sequences. Cancer associated sequences may include the sequences listed in Table 1. Cancer associated sequences may include SEQ ID NOS: 1-70. Cancer associated sequences may include sequences encoding one or more of C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1. In some embodiments, the cancer associated sequences may be DNA sequences encoding the above mRNA or the cancer associated protein or cancer associated polypeptide expressed by the above mRNA or homologs thereof. In some embodiments, the cancer associated sequence may be a mutant nucleic acid of the above disclosed sequences. In some embodiments, the homolog may have at least about 60%, at least about 65%, at least about 70%, at least about 75%, at least about 80%, at least about 85%, at least about 90%, at least about 95%, at least about 97%, at least about 98%, at least about 99%, at least about 99.5% identity with the disclosed polypeptide sequence.

In some embodiments, cancer associated sequences may include both nucleic acid and amino acid sequences. In some embodiments, the cancer associated sequences may include sequences having at least about 60% homology with the disclosed sequences. In some embodiments, the cancer associated sequences may have at least about 65%, about 70%, about 75%, about 80%, about 85%, about 90%, about 95%, about 97%, about 99%, about 99.8% homology with the disclosed sequences. In some embodiments, the cancer associated sequences may be “mutant nucleic acids”. As used herein, “mutant nucleic acids” refers to deletion mutants, insertions, point mutations, substitutions, translocations.

A nucleic acid of the present disclosure may include phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Brig et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more earbocyclic sugars are also included within one definition of nucleic acids (see Jenkins et al., Chem. Soc. Rev. (1995) pp 169-176). Several nucleic acid analogs are described in Rawls, C & E News Jun., 2, 1997 page 35. These modifications of the ribose-phosphate backbone may be done for a variety of reasons, for example to increase the stability and half-life of such molecules in physiological environments for use in anti-sense applications or as probes on a biochip.

As will be appreciated by those skilled in the art, such nucleic acid analogs may be used in some embodiments of the present disclosure. In addition, mixtures of naturally occurring nucleic acids and analogs can be made; alternatively, mixtures of different nucleic acid analogs, and mixtures of naturally occurring nucleic acids and analogs may be made.

In some embodiments, the nucleic acids may be single stranded or double stranded or may contain portions of both double stranded or single stranded sequence. As will be appreciated by those skilled in the art, the depiction of a single strand also defines the sequence of the other strand; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, for example, the subject units of a peptide nucleic acid, each containing a base, are referred to herein as a nucleoside.

In some embodiments, the cancer associated sequences may be recombinant nucleic acids. By the term “recombinant nucleic acid” herein refers to nucleic acid molecules, originally formed in vitro, in general, by the manipulation of nucleic acid by polymerases and endonucleases, in a form not normally found in nature. Thus a recombinant nucleic acid may also be an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it can replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein, a “polynucleotide” or “nucleic acid” is a polymeric form of nucleotides of any length, either ribonucleotides or deoxyribonucleotides. This term includes double- and single-stranded DNA and RNA. It also includes known types of modifications, for example, labels which are known in the art, methylation, “caps”, substitution of one or more of the naturally occurring nucleotides with an analog, internucleotide modifications-such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

Some embodiments also provide for polypeptides and antigens (e.g., cancer-associated polypeptides) associated with a variety of cancers as targets for diagnostic and/or therapeutic antibodies. These antigens may also be useful for drug discovery (e.g., small molecules) and for further characterization of cellular regulation, growth, and differentiation.

In some embodiments, the invention provides an isolated nucleic acid comprises at least 10, 12, 15, 20 or 30 contiguous nucleotides of a sequence selected from the group consisting of the cancer associated polynucleotide sequences disclosed in Table 1 and/or SEQ ID NOS: 1-70.

In some embodiments, the polynucleotide, or its complement or a fragment thereof, further comprises a detectable substance or label, is attached to a solid support, is prepared at least in part by chemical synthesis, is an antisense fragment, is single stranded, is double stranded or comprises a microarray.

In some embodiments, the invention provides an isolated polypeptide, encoded within an open reading frame of a cancer associated sequence selected from the polynucleotide sequences of SEQ ID NOS: 1-70 and/or shown in Table 1, or its complement. In some embodiments, the invention provides an isolated polypeptide, wherein said polypeptide comprises the amino acid sequence encoded by a polynucleotide selected from the group consisting of SEQ ID NOS: 1-70. In some embodiments, the invention provides an isolated polypeptide, wherein said polypeptide comprises the amino acid sequence encoded by a cancer associated polypeptide.

In some embodiments, the invention further provides an isolated polypeptide, comprising the amino acid sequence of an epitope of the amino acid sequence of a cancer associated polypeptide, wherein the polypeptide or fragment thereof may be attached to a solid support. In some embodiments the invention provides an isolated antibody (monoclonal or polyclonal) or antigen binding fragment thereof, that binds to such a polypeptide. The isolated antibody or antigen binding fragment thereof may be attached to a solid support, or further comprises a detectable label.

### Detection Methods for Analyzing Samples

The detection of the expression level of the one or more markers disclosed infra may be by any means known in the art. For example where the marker is a protein associated with breast cancer an ELISA may be used to detect the expression level of the marker. Other suitable assays for detecting the presence of a protein marker include a radioimmunoassay, a western blot, and an immunoprecipitation assay, such as a bead based assay, e.g. a magnetic bead based assay. In some embodiments the marker may be isolated from the sample before detection, but in other embodiments it is not isolated from the sample. In some embodiments the protein marker may be expressed in a cellular context (i.e. on the surface of the cell or within the cell). In these instances immunocytochemistry may be used to detect the marker. Alternatively, the flow cytometry can used to detect the marker. Where the marker is contained within the cell, the cells may be treated with a detergent to make the marker accessible to a detection reagent. Suitable detection reagents would include any molecule that specifically binds the marker, such as an antibody that specifically binds to an epitope on the marker.

Suitable agents for detecting a protein marker as disclosed infra include any specific binding partner of the breast cancer marker. For example the specific binding partner may be a protein that binds the breast cancer marker, such as an antibody. Other suitable specific binding partners may include a receptor that binds the breast cancer marker or an enzyme that specifically binds the breast cancer marker.

The cancer can also be diagnosed to a specific tissue type as well by visualizing the labeled molecule. The molecule can be visualized or detected using any method, such as but not limited to, MRI, CAT scan, PET scan, and the like. In some embodiments, an antibody can bind to the protein and then be detected. In some embodiments, the level of antibody binding can be quantified to determine whether the protein is overexpressed. Differential expression can also be determined by known methods. Accordingly, embodiments hereof provide a method for imaging structures in tissues and cells of a subject having cancer, is suspected of having cancer, or is undergoing a diagnostic procedure to determine if the person has cancer. If the imaging demonstrates that the cancer associated protein is overexpressed or differentially expressed then the patient is diagnosed as having cancer or suspected of having cancer. Other tests can also be done, such as but not limited to, a biopsy to confirm, or otherwise aid, the diagnosis.

The label molecules can also be labeled by, but not limited to, any radioisotopes that can be imaged with a PET or SPECT camera. For example, radiopharmaceuticals of various embodiments may be radiolabeled with radioisotopes such as, but not limited to, ⁷⁶Br, ¹²³I, ¹²⁵I, ¹³¹I, ^{99m}Tc, ¹¹C, ¹⁸F, or other gamma- or positron-emitting radionuclides. In other embodiments, the label molecules may be radiolabeled with a combination of radioisotopes.

In some embodiments the marker associated with breast cancer may be a nucleic acid, e.g. an mRNA molecule. The nucleic acid may be isolated from the sample. Detection of the nucleic acid may be by any means known in the art. For example the nucleic acid molecule may be detected by Southern blot or northern blot mass spectroscopy, microarray and the like. The nucleic acid may be detected using PCR, for example where the nucleic acid is an RNA molecule, such as an mRNA molecule, rtPCR may be used. The PCR may be quantitative PCR (e.g. qPCT) or real time PCR. The nucleic acid may be detected by in situ hybridization where the sample includes breast cancer cells.

The assays described above may include the use of a probe to detect the nucleic acid marker. Probes are described infra. Briefly, the probe may be a nucleic acid molecule ranging from 5-40, 10-35, 15-30 nucleotides long. The probe may be about 5, about 10, about 20, about 25, about 30, about 35 nucleotides long. The probe may include a portion of a gene encoding the breast cancer marker, or a complement of a gene encoding a breast cancer marker.

The gene expression levels may be represented as relative expression normalized to the ADPRT (Accession number NM_{—}001618.2,), GAPD (Accession number NM_{—}002046.2,), or other housekeeping genes known in the art. In the case of microarrayed probes of mRNA expression, the gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

### Identification and Uses of Cancer Associated Sequences

Microarray analysis of gene expression may be used to identify sequences associated with breast cancer. These identified sequences may then be used in a number of different ways, including diagnosis, prognosis, screening for modulators (including both agonists and antagonists), antibody generation (for immunotherapy and imaging), etc. However, as will be appreciated by those skilled in the art, sequences that are identified in one type of cancer may have a strong likelihood of being involved in other types of cancers as well. Thus, while the sequences outlined herein are initially identified as correlated with breast cancers, they may also be found in other types of cancers as well.

As will be appreciated by those skilled in the art, cancer associated sequences of embodiments herein may be useful to detect nucleic acids expression levels in a subject. They may be used in therapeutic applications as well. Further, the cancer associated sequences of embodiments herein may be used in screening applications; for example, generation of biochips comprising nucleic acid probes to the cancer associated sequences.

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic imitations are classified as either protooncogenes or tumor suppressor genes, depending on whether their mutant phenotypes are dominant or recessive, respectively.

Some embodiments of the invention are directed to cancer associated sequences (“target markers”). Some embodiments are directed to methods of identifying novel target markers useful in the diagnosis and treatment of cancer wherein expression levels of mRNAs, miRNAs, proteins, or protein post translational modifications including but not limited to phosphorylation and sumoylation are compared between five categories of cell types: (1) immortal pluripotent stem cells (such as embryonic stem (“ES”) cells, induced pluripotent stem (“iPS”) cells, and germ-line cells such as embryonal carcinoma (“EC”) cells) or gonadal tissues; (2) ES, iPS, or EC-derived clonal embryonic progenitor (“EP”) cell lines, (3) nucleated blood cells including but not limited to CD34+ cells and CD133+ cells; (4) normal mortal somatic adult-derived tissues and cultured cells including: skin fibroblasts, vascular endothelial cells, normal non-lymphoid and non-cancerous tissues, and the like, and (5) malignant cancer cells including cultured cancer cell lines or human tumor tissue. mRNAs, miRNAs, or proteins that are generally expressed (or not expressed) in categories 1, 3, and 5, or categories 1 and 5 but not expressed (or expressed) in categories 2 and 4 are candidate targets for cancer diagnosis and therapy. Some embodiments herein are directed to human applications, non-human veterinary applications, or a combination thereof.

In some embodiments, a method of identifying a target marker comprises the steps of: 1) obtaining a molecular profile of the mRNAs, miRNAs, proteins, or protein modifications of immortal pluripotent stem cells (such as embryonic stem (“ES”) cells, induced pluripotent stem (“iPS”) cells, and germ-line cells such as embryonal carcinoma (“EC”) cells); 2) ES, iPS, or EC-derived clonal embryonic progenitor (“EP”) cell lines malignant cancer cells including cultured cancer cell lines or human tumor tissues, and comparing those molecules to those present in mortal somatic cell types such as cultured clonal human embryonic progenitors, cultured somatic cells from fetal or adult sources, or normal tissue counterparts to malignant cancer cells. Target markers that are shared between pluripotent stem cells such as hES cells and malignant cancer cells, but are not present in a majority of somatic cell types may be candidate diagnostic markers and therapeutic targets.

### Methods of Analyzing Expression Data

It will be appreciated that there are various methods of obtaining expression data and uses of the expression data. For example, the expression data that can be used to detect or diagnose a subject with cancer can be obtained experimentally. In some embodiments, obtaining the expression data comprises obtaining the sample and processing the sample to experimentally determine the expression data. The expression data can comprise expression data for one or more of the cancer associated sequences described herein. The expression data can be experimentally determined by, for example, using a microarray or quantitative amplification method such as, but not limited to, those described herein. In some embodiments, obtaining expression data associated with a sample comprises receiving the expression data from a third party that has processed the sample to experimentally determine the expression data.

Detecting a level of expression or similar steps that are described herein may be done experimentally or provided by a third-party as is described herein. Therefore, for example, “detecting a level of expression” may refer to experimentally measuring the data and/or having the data provided by another party who has processed a sample to determine and detect a level of expression data. In some embodiments, the expression data may be detected experimentally and provided by a third party.

The comparison of gene expression on an mRNA level using Iliumina gene expression microarrays hybridized to RNA probe sequences (shown in Table 1) prepared from the diverse categories of cell types: 1) human embryonic stem (“ES”) cells, or gonadal tissues 2) ES, iPS, or EC-derived clonal embryonic progenitor (“EP”) cell lines, 3) nucleated blood cells including but not limited to CD34+ cells and CD 133+ cells; 4) Normal mortal somatic adult-derived tissues and cultured cells including: skin fibroblasts, vascular endothelial cells, normal non-lymphoid and non-cancerous tissues, and the like, and 5) malignant cancer cells including cultured cancer cell lines or human tumor tissue and filters was performed to detect genes that are generally expressed (or not expressed) in categories 1, 3, and 5, or categories 1 and 5 but not expressed (or expressed) in categories 2 and 4. Therapies in these cancers based on this observation would be based on reducing the expression of the above referenced transcripts up-regulated in cancer, or otherwise reducing the expression of the gene products.

Gene Expression Assays: Measurement of the gene expression levels may be performed by any known methods in the art, including but not limited to quantitative PCR, or microarray gene expression analysis, bead array gene expression analysis and Northern analysis. The gene expression levels may be represented as relative expression normalized to the ADPRT (Accession number NM_{—}001618.2; SEQ ID NO: 37), GAPD (Accession number NM_{—}002046.2; SEQ ID NO: 38), or other housekeeping genes known in the art. In the case of microarrayed probes of mRNA expression, the gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

Samples obtained from subjects may be analyzed by any method known in the art to determine if the subject has cancer. For example mRNA can be analyzed to determine the expression level of one or more cancer associated sequences described infra. RNA extraction: Cells of the present disclosure may be incubated with 0.05% trypsin and 0.5 mM EDTA, followed by collecting in DMEM (Gibco, Gaithersburg, Md.) with 0.5% BSA. Total RNA may be purified from cells using the RNeasy Mini kit (Qiagen, Hilden, Germany).

Micro RNAs and small RNAs may effect gene expression. Thus, cancer may be associated with the aberrant expression of micro RNAs (miRNA) or small RNAs. Total RNA or samples enriched for small RNA species may be isolated from cell cultures that undergo serum starvation prior to harvesting RNA to approximate cellular growth arrest observed in many mature tissues. Cellular growth arrest may be performed by changing to medium containing 0.5% serum for 5 days, with one medium change 2-3 days after the first addition of low serum medium. RNA may be harvested according to the vendor's instructions for Qiagen RNAeasy kits to isolate total RNA or Ambion mirVana kits to isolate RNA enriched for small RNA species. The RNA concentrations may be determined by spectrophotometry and RNA quality may be determined by denaturing agarose gel electrophoresis to visualize 28S and 18S RNA. Samples with clearly visible 28S and 18S bands without signs of degradation and at a ratio of approximately 2:1, 28S:18S may be used for subsequent miRNA analysis.

The miRNAs may be quantitated using a Human Panel TaqMan MicroRNA Assay from Applied Biosystems, Inc. This is a two-step assay that uses stein-loop primers for reverse transcription (RT) followed by real-time TaqMan®. A total of 330 miRNA assays may be performed to quantitate the levels of miRNA in the H9 human embryonic stem cell line, a differentiated fibroblast cell line, and nine cell lines differentiated from human embryonic stem cells. The assay includes two steps, reverse transcription (RT) and quantitative PCR. Real-time PCR may be performed on an Applied Biosystems 7500 Real-Time PCR System. The copy number per cell may be estimated based on the standard curve of synthetic mir-16 miRNA and assuming a total RNA mass of approximately 15 pg/cell.

The reverse transcription reaction may be performed using 1x cDNA archiving buffer, 3.35 units MMLV reverse transcriptase, 5 mM each dNTP, 1.3 units AB RNase inhibitor, 2.5 nM 330-plex reverse primer (RP), 3 ng of cellular RNA in a final volume of 5 μl. The reverse transcription reaction may be performed on a BioRad or MJ thermocycler with a cycling profile of 20° C. for 30 sec; 42° C. for 30 sec; 50° C. for 1 sec, for 60 cycles followed by one cycle of 85° C. for 5 min.

Real-time PCR. Two microlitres of 1:400 diluted Pre-PCR product may be used for a 20 ul reaction. All reactions may be duplicated. Because the method is very robust, duplicate samples may be sufficient and accurate enough to obtain values for miRNA expression levels. TaqMan universal PCR master mix of ABI may be used according to manufacturer's suggestion. Briefly, 1x TaqMan Universal Master Mix (ABI), 1 uM Forward Primer, 1 uM Universal Reverse Primer and 0.2 uM TaqMan Probe may be used for each real-time PCR. The conditions used may be as follows: 95° C. for 10 min, followed by 40 cycles at 95° C. for 15 s, and 60° C. for 1 min. All the reactions may be run on ABI Prism 7000 Sequence Detection System.

Microarray hybridization and data processing: cDNA samples and cellular total RNA (5 μg in each of eight individual tubes) may be subjected to the One-Cycle Target Labeling procedure for biotin labeling by in vitro transcription (IVT) (Affymetrix, Santa Clara, Calif.) or using the Illumina Total Prep RNA Labelling kit. For analysis on Affymetix gene chips, the cRNA may be subsequently fragmented and hybridized to the Human Genome U133 Plus 2.0 Array (Affymetrix) according to the manufacturer's instructions. The microarray image data may be processed with the GeneChip Scanner 3000 (Affymetrix) to generate CEL data. The CEL data may be then subjected to analysis with dChip software, which has the advantage of normalizing and processing multiple datasets simultaneously. Data obtained from the eight nonamplified controls from cells, from the eight independently amplified samples from the diluted cellular RNA, and from the amplified cDNA samples from 20 single cells may be normalized separately within the respective groups, according to the program's default setting. The model based expression indices (MBEI) may be calculated using the PM/MM difference mode with log-2 transformation of signal intensity and truncation of low values to zero. The absolute calls (Present, Marginal and Absent) may be calculated by the Affymetrix Microarray Software 5.0 (MAS 5.0) algorithm using the dChip default setting. The expression levels of only the Present probes may be considered for all quantitative analyses described below. The GEO accession number for the microarray data is GSE4309. For analysis on Illumina Human HT-12 v4 Expression Bead Chips, labeled eRNA may be hybridized according to the manufacturer's instructions.

Calculation of coverage and accuracy: A true positive is defined as probes called Present in at least six of the eight nonamplified controls, and the true expression levels are defined as the log-averaged expression levels of the Present probes. The definition of coverage is (the number of truly positive probes detected in amplified samples)/(the number of truly positive probes). The definition of accuracy is (the number of truly positive probes detected in amplified samples)/(the number of probes detected in amplified samples). The expression levels of the amplified and nonamplified samples may be divided by the class interval of 20.5 (20, 205, 21, 21.5 . . . ), where accuracy and coverage are calculated. These expression level bins may be also used to analyze the frequency distribution of the detected probes.

Analysis of gene expression profiles of cells: The unsupervised clustering and class neighbor analyses of the microarray data from cells may be performed using GenePattern software, which performs the signal-to-noise ratio analysis/T-test in conjunction with the permutation test to preclude the contribution of any sample variability, including those from methodology and/or biopsy, at high confidence. The analyses may be conducted on the 14,128 probes for which at least 6 out of 20 single cells provided Present calls and at least 1 out of 20 samples provided expression levels >20 copies per cell. The expression levels calculated for probes with Absent/Marginal calls may be truncated to zero. To calculate relative gene expression levels, the Ct values obtained with Q-PCR analyses may be corrected using the efficiencies of the individual primer pairs quantified either with whole human genome (BD Biosciences) or plasmids that contain gene fragments. The relative expression levels may be further transformed into copy numbers with a calibration line calculated using the spike RNAs included in the reaction mixture (log₁₀[expression level]=1.05×log₁₀[copy number]+4.65). The Chi-square test for independence may be performed to evaluate the association of gene expressions with Gata4, which represents the difference between cluster 1 and cluster 2 determined by the unsupervised clustering and which is restricted to PE at later stages. The expression levels of individual genes measured with Q-PCR may be classified into three categories: high (>100 copies per cell), middle (10-100 copies per cell), and low (<10 copies per cell). The Chi-square and P-values for independence from Gata4 expression may be calculated based on this classification. Chi squared is defined as follows: Ψ2=ΣΣ (n fij−fi fj)²/n fi fj, where i and j represent expression level categories (high, middle or low) of the reference (Gata4) and the target gene, respectively; fi, fj, and fij represent the observed frequency of categories i, j and ij, respectively; and n represents the sample number (n=24). The degrees of freedom may be defined as (r−1)×(c−1), where r and c represent available numbers of expression level categories of Gata4 and of the target gene, respectively.

### Cancer Therapeutics and Methods of Treating Cancer

In some embodiments the invention provides a method for inhibiting growth of cancer cells in a subject. In some embodiments, the method comprises administering to the subject an effective amount of a pharmaceutical composition as described herein. In some embodiments the invention provides a method for delivering a therapeutic agent to cancer cells in a subject, the method comprising: administering to the subject an effective amount of a pharmaceutical composition according to the invention.

In some embodiments the invention provides a method of treating cancer, such as breast cancer. In some embodiments, the cancer may be selected from ductal carcinoma in situ (DCIS), invasive ductal carcinoma (IDC), medullary carcinoma, invasive lobular carcinoma (ILC), tubular carcinoma, mucinous carcinoma, inflammatory breast cancer (IBC), lobular carcinoma in site (LCIS), male breast cancer, Paget's disease of the nipple, phyllodes tumors of the breast, recurrent and metastatic breast cancer, or a combination thereof.

In some embodiments, breast cancers expressing one of the cancer associated sequences may be treated by antagonizing the cancer associated sequence's activity. In some embodiments, a method of treating breast cancer may comprise administering a therapeutic such as, without limitation, antibodies that antagonize the ligand binding to the cancer associated sequence, small molecules that inhibit the cancer associated sequence's expression or activity, siRNAs directed towards the cancer associated sequence, or the like. In some embodiments, technologies such as ELISA, as well as other detection techniques described herein, may be used to screen for breast cancer.

In some embodiments, the present disclosure provides methods of treating cancer in a subject, the method comprising administering to a subject having cancer an agent that inhibits activity of a cancer associated sequence selected from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1 or a combination thereof. In some embodiments, the agent comprises an antibody that specifically binds to a cancer associated sequence selected from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1 or a combination thereof.

In some embodiments, a method of treating cancer may comprise administering an antibody against the protein to a subject in need thereof. In some embodiments, the antibody may be a monoclonal antibody or a polyclonal antibody. In some embodiments, the antibody may be a humanized or a recombinant antibody. Antibodies can be made that specifically bind to this region using known methods and any method is suitable. In some embodiments, the antibody specifically binds to C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK 15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a fragment thereof. For example the antibody may bind to a specific epitope found on any of the proteins encoded for by the cancer associated sequences described herein. In some embodiments, the epitope comprises about 1-10, 1-20, 1-30, 3-10, or 3-15 residues of the cancer associated sequence. In some embodiments, the epitope is not linear.

In some embodiments, the antibody binds to the regions described herein or a peptide with at least 90, 95, or 99% homology or identity to the region. In some embodiments, the fragment of the regions described herein is 5-10 residues in length. In some embodiments, the fragment of the regions (e.g. epitope) described herein are 3-5 residues in length. The fragments are described based upon the length provided. In some embodiments, the epitope is about 3, 4, 5, 6, 7, 8, 9, 10, 11, 12, 13, 14, 15, or 20 residues in length.

In some embodiments, the sequence to which the antibody binds may include both nucleic acid and amino acid sequences. In some embodiments, the sequence to which the antibody binds may include sequences having at least about 60% homology with the disclosed sequences. In some embodiments, the sequence to which the antibody binds may have at least about 65%, about 70%, about 75%, about 80%, about 85%, about 90%, about 95%, about 97%, about 99%, about 99.8% homology with the disclosed sequences. In some embodiments, the sequences may be referred to as “mutant nucleic acids” or “mutant peptide sequences.”

In some embodiments, the method further comprises treating the subject diagnosed with breast cancer with an antibody that binds to a cancer associated sequence selected from SEQ ID NOS: 1-70 and inhibits the growth or progression of the breast cancer.

Information relating to expression of the receptor can also be useful in determining therapies aimed at up or down-regulating the cancer associated sequence's signaling using agonists or antagonists.

In some embodiments, a method of treating cancer (e.g. breast or other types of cancer) comprises detecting the presence of a cancer associated sequence's receptor and administering a cancer treatment. The cancer treatment may be any cancer treatment e.g., one that is specific to the inhibiting the action of a cancer associated sequence. For example, various cancers are tested to determine if a specific molecule is present before giving a cancer treatment. In some embodiments, therefore, a sample would be obtained from the patient and tested for the presence of a cancer associated sequence or the overexpression of a cancer associated sequence as described herein. In some embodiments, if a cancer associated sequence is found to be overexpressed a breast cancer treatment or therapeutic is administered to the subject. The breast cancer treatment may be a conventional non-specific treatment, such as chemotherapy, or the treatment may comprise of a specific treatment that only targets the activity of the cancer associated sequence or the receptor to which the cancer associated sequence binds. These treatments can be, for example, an antibody that specifically binds to the cancer associated sequence and inhibits its activity.

In some embodiments, a method of treating cancer may comprise administering an agent that interferes with the synthesis, secretion, receptor binding or receptor signaling of cancer associated proteins or its receptors.

In some embodiments, the cancer cell may be targeted specifically with a therapeutic based upon the differentially expressed gene or gene product. For example, in some embodiments, the differentially expressed gene product may be an enzyme, which can convert an anti-cancer prodrug into its active form. Therefore, in normal cells, where the differentially expressed gene product is not expressed or expressed at significantly lower levels, the prodrug may be either not activated or activated in a lesser amount, and may be, therefore less toxic to normal cells. Therefore, the cancer prodrug may, in some embodiments, be given in a higher dosage so that the cancer cells can metabolize the prodrug, which will, for example, kill the cancer cell, and the normal cells will not metabolize the prodrug or not as well, and, therefore, be less toxic to the patient. An example of this is where tumor cells overexpress a metalloprotease, which is described in Atkinson et al., British Journal of Pharmacology (2008) 153, 1344-1352, which is hereby incorporated by reference in its entirety and for the method of specifically targeting cancer cells. Using proteases to target cancer cells is also described in Carl et al., PNAS, Vol. 77, No. 4, pp. 2224-2228, April 1980, which is hereby incorporated by reference in its entirety and for the method of specifically targeting cancer cells. For example, doxorubicin or other type of chemotherapeutic can be linked to a peptide sequence that is specifically cleaved or recognized by the differentially expressed gene product. The doxorubicin or other type of chemotherapeutic is then cleaved from the peptide sequence and is activated such that it can kill or inhibit the growth of the cancer cell whereas in the normal cell the chemotherapeutic is never internalized into the cell or is not metabolized as efficiently, and is, therefore, less toxic.

In some embodiments, a method of treating breast cancer may comprise gene knockdown of one or more cancer associated sequences described herein. Gene knockdown refers to techniques by which the expression of one or more of an organism's genes is reduced, either through genetic modification (a change in the DNA of one of the organism's chromosomes such as, without limitation, chromosomes encoding cancer associated sequences) or by treatment with a reagent such as a short DNA or RNA oligonucleotide with a sequence complementary to either an mRNA transcript or a gene. In some embodiments, the oligonucleotide used may be selected from RNase-H competent antisense, such as, without limitation, ssDNA oligonucleotides, ssRNA oligonucleotides, phosphorothioate oligonucleotides, or chimeric oligonucleotides; RNase-independent antisense, such as morpholino oligonucleotides, 2′-O-methyl phosphorothioate oligonucleotides, locked nucleic acid oligonucleotides, or peptide nucleic acid oligonucleotides; RNAi oligonucleotides, such as, without limitation, siRNA duplex oligonucleotides, or shRNA oligonucleotides; or any combination thereof. In some embodiments, a plasmid may be introduced into a cell, wherein the plasmid expresses either an antisense RNA transcript or an shRNA transcript. The oligo introduced or transcript expressed may interact with the target niRNA (ex. SEQ ID NOS: 1-70) by complementary base pairing (a sense-antisense interaction).

The specific mechanism of silencing may vary with the oligo chemistry. In some embodiments, the binding of a oligonucleotide described herein to the active gene or its transcripts may cause decreased expression through blocking of transcription, degradation of the mRNA transcript (e.g. by small interfering RNA (siRNA) or RNase-11 dependent antisense) or blocking either mRNA translation, pre-mRNA splicing sites or nuclease cleavage sites used for maturation of other functional RNAs such as miRNA (e.g. by Morpholino oligonucleotides or other RNase-H independent antisense). For example, RNase-H competent antisense oligonucleotides (and antisense RNA transcripts) may form duplexes with RNA that are recognized by the enzyme RNase-H, which cleaves the RNA strand. As another example, RNase-independent oligonucleotides may bind to the mRNA and block the translation process. In some embodiments, the oligonucleotides may bind in the 5′-UTR and halt the initiation complex as it travels from the 5′-cap to the start codon, preventing ribosome assembly. A single strand of RNAi oligonucleotides may be loaded into the RISC complex, which catalytically cleaves complementary sequences and inhibits translation of some mRNAs bearing partially-complementary sequences. The oligonucleotides may be introduced into a cell by any technique including, without limitation, electroporation, microinjection, salt-shock methods such as, for example, CaCl2 shock; transfection of anionic oligo by cationic lipids such as, for example, Lipofectamine; transfection of uncharged oligonucleotides by endosomal release agents such as, for example, Endo-Porter; or any combination thereof. In some embodiments, the oligonucleotides may be delivered from the blood to the cytosol using techniques selected from nanoparticle complexes, virally-mediated transfection, oligonucleotides linked to octaguanidinium dendrimers (Morpholino oligonucleotides), or any combination thereof.

In some embodiments, a method of treating breast cancer may comprise treating cells to knockdown or inhibit expression of a gene encoding the mRNA disclosed in SEQ ID NOS: 1-70. The method may comprise culturing hES cell-derived clonal embryonic progenitor cell lines CM02 and EN13 (see U.S. Patent Publication 2008/0070303, entitled “Methods to accelerate the isolation of novel cell strains from pluripotent stem cells and cells obtained thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each of which is incorporated by reference herein in its entirety) with a retrovirus expressing silencing RNA directed to a cancer-associated sequence. In some embodiments, the method may further comprise confirming down-regulation by qPCR. In some embodiments, the method further comprises cryopreserving the cells. In some embodiments, the method further comprises reprogramming the cells. In some embodiments, the method comprises cryopreserving or reprogramming the cells within two days by the exogenous administration of OCT4, MYC, KLF4, and SOX2 (see Takahashi and Yamanaka 2006 Aug. 25; 126(4):663-76; U.S. patent application Ser. No. 12/086,479, published as US2009/0068742 and entitled “Nuclear Reprogramming Factor”, each of which is incorporated herein by reference) and by the method described in PCT/US06/30632, published as WO/2007/019398 and entitled “Improved Methods of Reprogramming Animal Somatic Cells”, incorporated by reference herein in its entirety. In some embodiments, the method may comprise culturing mammalian differentiated cells under conditions that promote the propagation of ES cells. In some embodiments, any convenient ES cell propagation condition may be used, e.g., on feeders or in feeder free media capable of propagating ES cells. In some embodiments, the method comprises identifying cells from ES colonies in the culture. Cells from the identified ES colony may then be evaluated for ES markers, e.g., Oct4, TRA 1-60, TRA 1-81, SSEA4, etc., and those having ES cell phenotype may be expanded. Control lines that have not been preconditioned by the knockdown may be reprogrammed in parallel to demonstrate the effectiveness of the preconditioning. In some embodiments, a method for treating cancer comprises administering to a subject in need thereof a therapeutic agent modulating the activity of a cancer associated protein, wherein the cancer associated protein is encoded by a nucleic acid comprising a nucleic acid sequence selected from the group consisting of the human nucleic acid sequences in Table 1 and further wherein the therapeutic agent binds to the cancer associated protein.

In some embodiments, a method of treating cancer comprises administering an antibody (e.g. monoclonal antibody, human antibody, humanized antibody, recombinant antibody, chimeric antibody, and the like) that specifically binds to a cancer associated protein that is expressed on a cell surface. In some embodiments, the antibody binds to an extracellular domain of the cancer associated protein. In some embodiments, the antibody binds to a cancer associated protein differentially expressed on a cancer cell surface relative to a normal cell surface, or, in some embodiments, to at least one human cancer cell line. In some embodiments, the antibody is linked to a therapeutic agent.

### Screening for Anti-Cancer Agents

In some embodiments, a method of identifying an anti-cancer agent is provided, wherein the method comprises contacting a candidate agent to a sample; and determining the cancer associated sequence's activity in the sample. In some embodiments, the candidate agent is identified as an anti-cancer agent if the cancer associated sequence's activity is reduced in the sample after the contacting. In some embodiments, the candidate agent is a candidate antibody. In some embodiments, the method comprises contacting a candidate antibody that binds to the cancer associated sequence with a sample, and assaying for the cancer associated sequence's activity, wherein the candidate antibody is identified as an anti-cancer agent if the cancer associated sequence activity is reduced in the sample after the contacting. A cancer associated sequence's activity can be any activity of the cancer associated sequence.

In some embodiments, the present disclosure provides methods of identifying an anti-cancer (e.g. breast cancer) agent, the method comprising contacting a candidate agent to a cell sample; and determining activity of a cancer associated sequence selected from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1 or a combination thereof in the cell sample, wherein the candidate agent is identified as an anti-cancer agent if the cancer associated sequence's activity is reduced in the cell sample after the contacting.

In some embodiments, the present disclosure provides methods of identifying an anti-cancer agent, the method comprising contacting a candidate antibody that binds to a cancer associated sequence selected from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL 10A1 or a combination thereof with a cell sample, and assaying for the cancer associated sequence's activity, wherein the candidate antibody is identified as an anti-cancer agent if the cancer associated sequence's activity is reduced in the cell sample after the contacting.

In some embodiments, a method of screening drug candidates includes comparing the level of expression of the cancer-associated sequence in the absence of the drug candidate to the level of expression in the presence of the drug candidate. Expression level may be determined, for example, by measuring the mRNA levels of one or more cancer associated sequences disclosed infra. Alternatively expression levels may be determined by measuring the expression level of one or more proteins encoded for by the cancer sequences disclosed infra.

Some embodiments are directed to a method of screening for a therapeutic agent capable of binding to a cancer-associated sequence (nucleic acid or protein), the method comprising combining the cancer-associated sequence and a candidate therapeutic agent, and determining the binding of the candidate agent to the cancer-associated sequence.

Further provided herein is a method for screening for a therapeutic agent capable of modulating the activity of a cancer-associated sequence. In some embodiments, the method comprises combining the cancer-associated sequence and a candidate therapeutic agent, and determining the effect of the candidate agent on the bioactivity of the cancer-associated sequence. An agent that modulates the bioactivity of a cancer associated sequence may be used as a therapeutic agent capable of modulating the activity of a cancer-associated sequence.

A method of screening for anticancer activity, the method comprising: (a) contacting a cell that expresses a cancer associated gene which transcribes a cancer associated sequence selected from SEQ ID NOS: 1-70, homologs thereof, combinations thereof, or fragments thereof with an anticancer drug candidate; (b) detecting an effect of the anticancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has anticancer activity.

In some embodiments, a method of evaluating the effect of a candidate cancer drug may comprise administering the drug to a patient and removing a cell sample from the patient. The expression profile of the cell is then determined. In some embodiments, the method may further comprise comparing the expression profile of the patient to an expression profile of a healthy individual. In some embodiments, the expression profile comprises measuring the expression of one or more or any combination thereof of the sequences disclosed herein. In some embodiments, where the expression profile of one or more or any combination thereof of the sequences disclosed herein is modified (increased or decreased) the candidate cancer drug is said to be effective.

The pattern of gene expression in a particular living cell may be characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-326 (1991); Weinberg, Science, 254: 1138-1146 (1991)). Accordingly, some embodiments herein provide for polynucleotide and polypeptide sequences involved in cancer and, in particular, in oncogenesis.

In some embodiments, the methods comprise targeting a marker that is expressed at abnormal levels in breast cancer tissue in comparison to normal somatic tissue. In some embodiments, the marker may include SEQ ID NOS: 1-70 or any combination thereof.

In some embodiments, the invention provides a method of screening for anticancer activity comprising: (a) providing a cell that expresses a cancer associated gene that encodes a nucleic acid sequence selected from the group consisting of the cancer associated sequences shown in Table 1 (SEQ ID NOS: 1-70), or fragment thereof, (b) contacting the cell, which can be derived from a cancer cell with an anticancer drug candidate; (c) monitoring an effect of the anticancer drug candidate on an expression of the cancer associated sequence in the cell sample, and optionally (d) comparing the level of expression in the absence of said drug candidate to the level of expression in the presence of the drug candidate. The drug candidate may be an inhibitor of transcription, a G-protein coupled receptor antagonist, a growth factor antagonist, a serine-threonine kinase antagonist, a tyrosine kinase antagonist. In some embodiments, where the candidate modulates the expression of the cancer associated sequence the candidate is said to have anticancer activity. In some embodiments, the anticancer activity is determined by measuring cell growth. In some embodiments, the candidate inhibits or retards cell growth and is said to have anticancer activity. In some embodiments, the candidate causes the cell to die, and thus, the candidate is said to have anticancer activity.

In some embodiments, the present invention provides a method of screening for activity against breast cancer. In some embodiments, the method comprises contacting a cell that overexpresses a cancer associated gene which is complementary to a cancer associated sequence selected from SEQ ID NOS: 1-70, homologs thereof, combinations thereof, or fragments thereof with a breast cancer drug candidate. In some embodiments, the method comprises detecting an effect of the breast cancer drug candidate on an expression of the cancer associated polynucleotide in the cell or an effect on the cell's growth or viability. In some embodiments, the method comprises comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against a breast cancer cell that overexpresses a cancer associated gene, wherein said gene comprises a sequence that is a sequence selected from SEQ ID NOS: 1-70, or complementary thereto, homologs thereof, combinations thereof, or fragments thereof. In some embodiments, the drug candidate is selected from a transcription inhibitor, a G-protein coupled receptor antagonist, a growth factor antagonist, a serine-threonine kinase antagonist, or a tyrosine kinase antagonist.

In some embodiments, the invention provides a method for screening for a therapeutic agent capable of modulating the activity of a cancer associated sequence, wherein said sequence can be encoded by a nucleic acid comprising a nucleic acid sequence selected from the group consisting of the polynucleotide sequences SEQ ID NOS: 1-70 and/or shown in Table 1, said method comprising: a) combining said cancer associated sequence and a candidate therapeutic agent; and b) determining the effect of the candidate agent on the bioactivity of said cancer associated sequence. In some embodiments, the therapeutic agent: affects the expression of the cancer associated sequence; affects the activity of the cancer associated sequence. In some embodiments, the cancer associated sequence is a cancer associated protein. In some embodiments, the cancer associated sequence is a cancer associated nucleic acid molecule.

[symbol]

### Immune Response Against Cancer

The cancer associated sequences disclosed infra may be used as antigens to stimulate an immune response in a subject.

In some embodiments, antigen presenting cells (APCs) may be used to activate T lymphocytes in vivo or ex vivo, to elicit an immune response against cells expressing a cancer associated sequence. APCs are highly specialized cells and may include, without limitation, macrophages, monocytes, and dendritic cells (DCs). APCs may process antigens and display their peptide fragments on the cell surface together with molecules required for lymphocyte activation. In some embodiments, the APCs may be dendritic cells. DCs may be classified into subgroups, including, e.g., follicular dendritic cells, Langerhans dendritic cells, and epidermal dendritic cells.

Some embodiments are directed to the use of cancer associated polypeptides and polynucleotides encoding a cancer associated sequence, a fragment thereof; or a mutant thereof; and antigen presenting cells (such as, without limitation, dendritic cells), to elicit an immune response against cells expressing a cancer-associated polypeptide sequence, such as, without limitation, cancer cells, in a subject. In some embodiments, the method of eliciting an immune response against cells expressing a cancer associated sequence comprises (1) isolating a hematopoietic stem cell, (2) genetically modifying the cell to express a cancer associated sequence, (3) differentiating the cell into DCs; and (4) administering the DCs to the subject (e.g., human patient). In some embodiments, the method of eliciting an immune response includes (1) isolating DCs (or isolation and differentiation of DC precursor cells), (2) pulsing the cells with a cancer associated sequence, and; (3) administering the DCs to the subject. These approaches are discussed in greater detail, infra. In some embodiments, the pulsed or expressing DCs may be used to activate T lymphocytes ex vivo. These general techniques and variations thereof may be within the skill of those in the art (see, e.g., WO97/29182; WO 97/04802; WO 97/22349; WO 96/23060; WO 98/01538; Hsu et al., 1996, Nature Med. 2:52-58), and that still other variations may be discovered in the future. In some embodiments, the cancer associated sequence is contacted with a subject to stimulate an immune response. In some embodiments, the immune response is a therapeutic immune response. In some embodiments, the immune response is a prophylactic immune response. For example, the cancer associated sequence can be contacted with a subject under conditions effective to stimulate an immune response. The cancer associated sequence can be administered as, for example, a DNA molecule (e.g. DNA vaccine), RNA molecule, or polypeptide, or any combination thereof. Administering a sequence to stimulate an immune response was known, but the identity of which sequences to use was not known prior to the present disclosure. Any sequence or combination of sequences disclosed herein or a homolog thereof can be administered to a subject to stimulate an immune response.

In some embodiments, dendritic cell precursor cells are isolated for transduction with a cancer associated sequence, and induced to differentiate into dendritic cells. The genetically modified DCs express the cancer associated sequence, and may display peptide fragments on the cell surface.

In some embodiments, the cancer associated sequence expressed comprises a sequence of a naturally occurring protein. In some embodiments, the cancer associate sequence does not comprise a naturally occurring sequence. As already noted, fragments of naturally occurring proteins may be used; in addition, the expressed polypeptide may comprise mutations such as deletions, insertions, or amino acid substitutions when compared to a naturally occurring polypeptide, so long as at least one peptide epitope can be processed by the DC and presented on a MHC class I or II surface molecule. In some embodiments, it may be desirable to use sequences other than “wild type,” in order to, for example, increase antigenicity of the peptide or to increase peptide expression levels. In some embodiments, the introduced cancer associated sequences may encode variants such as polymorphic variants (e.g., a variant expressed by a particular human patient) or variants characteristic of a particular cancer (e.g., a cancer in a particular subject).

In some embodiments, a cancer associated expression sequence may be introduced (transduced) into DCs or stem cells in any of a variety of standard methods, including transfection, recombinant vaccinia viruses, adeno-associated viruses (AAVs), retroviruses, etc.

In some embodiments, the transformed DCs of the invention may be introduced into the subject (e.g., without limitation, a human patient) where the DCs may induce an immune response. Typically, the immune response includes a cytotoxic T-lymphocyte (CTL) response against target cells bearing antigenic peptides (e.g., in a MHC class I/peptide complex). These target cells are typically cancer cells.

In some embodiments, when the DCs are to be administered to a subject, they may preferably isolated from, or derived from precursor cells from, that subject (i.e., the DCs may administered to an autologous subject). However, the cells may be infused into HLA-matched allogeneic or HLA-mismatched allogeneic subject. In the latter case, immunosuppressive drugs may be administered to the subject.

In some embodiments, the cells may be administered in any suitable manner. In some embodiments, the cell may be administered with a pharmaceutically acceptable carrier (e.g., saline). In some embodiments, the cells may be administered through intravenous, intra-articular, intramuscular, intradermal, intraperitoneal, or subcutaneous routes. Administration (i.e., immunization) may be repeated at time intervals. Infusions of DC may be combined with administration of cytokines that act to maintain DC number and activity (e.g., GM-CSF, IL-12).

In some embodiments, the dose administered to a subject may be a dose sufficient to induce an immune response as detected by assays which measure T cell proliferation, T lymphocyte cytotoxicity, and/or effect a beneficial therapeutic response in the patient over time, e.g., to inhibit growth of cancer cells or result in reduction in the number of cancer cells or the size of a tumor.

In some embodiments, DCs are obtained (either from a patient or by in vitro differentiation of precursor cells) and pulsed with antigenic peptides having a cancer associated sequence. The pulsing results in the presentation of peptides onto the surface MHC molecules of the cells. The peptide/MHC complexes displayed on the cell surface may be capable of inducing a MHC-restricted cytotoxic T-lymphocyte response against target cells expressing cancer associated polypeptides (e.g., without limitations, cancer cells).

In some embodiments, cancer associated sequences used for pulsing may have at least about 6 or 8 amino acids and fewer than about 30 amino acids or fewer than about 50 amino acid residues in length. In some embodiments, an immunogenic peptide sequence may have from about 8 to about 12 amino acids. In some embodiments, a mixture of human protein fragments may be used; alternatively a particular peptide of defined sequence may be used. The peptide antigens may be produced by de novo peptide synthesis, enzymatic digestion of purified or recombinant human peptides, by purification of the peptide sequence from a natural source (e.g., a subject or tumor cells from a subject), or expression of a recombinant polynucleotide encoding a human peptide fragment.

In some embodiments, the amount of peptide used for pulsing DC may depend on the nature, size and purity of the peptide or polypeptide. In some embodiments, an amount of from about 0.05 ug/ml to about 1 mg/ml, from about 0.05 ug/ml to about 500 ug/ml, from about 0.05 ug/ml to about 250 ug/ml, from about 0.5 ug/ml to about 1 mg/ml, from about 0.5 ug/ml to about 500 ug/ml, from about 0.5 ug/ml to about 250 ug/ml, or from about 1 ug/ml to about 100 ug/ml of peptide may be used. After adding the peptide antigen(s) to the cultured DC, the cells may then be allowed sufficient time to take up and process the antigen and express antigen peptides on the cell surface in association with either class I or class II MHC. In some embodiments, the time to take up and process the antigen may be about 18 to about 30 hours, about 20 to about 30 hours, or about 24 hours.

Numerous examples of systems and methods for predicting peptide binding motifs for different MHC Class I and II molecules have been described. Such prediction could be used for predicting peptide motifs that will bind to the desired MHC Class I or H molecules. Examples of such methods, systems, and databases that those of ordinary skill in the art might consult for such purpose include: Peptide Binding Motifs for MHC Class I and II Molecules; William E. Biddison, Roland Martin, Current Protocols in Immunology, Unit II (DOI: 10.1002/0471142735.ima01is36; Online Posting Date: May, 2001).

Biddison provides an overview of the use of peptide-binding motifs to predict interaction with a specific MHC class I or II allele, and gives examples for the use of MHC binding motifs to predict T-cell recognition.

Table 3 provides an exemplary result for a HLA peptide motif search at the NIH Center for Information Technology website, BioInformatics and Molecular Analysis Section (http://www-bimas.cit.nih.gov/cgi-bin/molbio/ken_parker_comboform). Full length HIST1H4H peptide sequence (SEQ ID NO: 39) was used as the search query.

TABLE 3
EXEMPLARY RESULT FOR HLA PEPTIDE MOTIF SEARCH

| User Parameter and Scoring | | | |
|---|---|---|---|
| Information | | | |
| Method selected to mimit the number of | | | Explicit number |
| results | | | |
| Number of results requested | | | 20 |
| HLA molecule type selected | | | A_0201 |
| Length selected for subsequences to be | | | 9 |
| scored | | | |
| Echoing mode selected for input sequence | | | Y |
| Echoing format | | | Numbered lines |
| Length of user's input peptide sequence | | | 369 |
| Number of subsequence scores calculated | | | 361 |
| Number of top-scoring subsequences | | | 20 |
| reported back in scoring output table | | | |
| | | | Score (estimate of |
| | Scoring | | half time of |
| | Results | Subsequence | disassociation of a |
| | Start | residue | molecule containing |
| Rank | Position | listing | this subsequence |
| 1 | 310 | SLLKFLAKV | 2249.173 |
| | | (SEQ ID NO: 71) | |
| 2 | 183 | MLLVFGIDV | 1662.432 |
| | | (SEQ ID NO: 72) | |
| 3 | 137 | KVTDLVQFL | 339.313 |
| | | (SEQ ID NO: 73) | |
| 4 | 254 | GLYDGMMEHL | 315.870 |
| | | (SEQ ID NO: 74) | |
| 5 | 228 | ILILSIIFI | 224.357 |
| | | (SEQ ID NO: 141) | |
| 6 | 296 | FLWGPRAHA | 189.678 |
| | | (SEQ ID NO: 75) | |
| 7 | 245 | VIWEALNMM | 90.891 |
| | | (SEQ ID NO: 76) | |
| 8 | 308 | KMSILKFLA | 72.836 |
| | | (SEQ ID NO: 77) | |
| 9 | 166 | KNYEDHFPL | 37.140 |
| | | (SEQ ID NO: 78) | |
| 10 | 201 | FVLVTSLGL | 31.814 |
| | | (SEQ ID NO: 79) | |
| 11 | 174 | ILFSEASEC | 31.249 |
| | | (SEQ ID NO: 80) | |
| 12 | 213 | GMLSDVQSM | 30.534 |
| | | (SEQ ID NO: 81) | |
| 13 | 226 | ILILILSII | 16.725 |
| | | (SEQ ID NO: 82) | |
| 14 | 225 | GILILILSI | 12.208 |
| | | (SEQ ID NO: 83) | |
| 15 | 251 | NMMGLYDGM | 9.758 |
| | | (SEQ ID NO: 84) | |
| 16 | 88 | QIACSSPSV | 9.563 |
| | | (SEQ ID NO: 85) | |
| 17 | 66 | LIPSTPEEV | 7.966 |
| | | (SEQ ID NO: 86) | |
| 18 | 220 | SMPKTGILI | 7.535 |
| | | (SEQ ID NO: 142) | |
| 19 | 233 | IIFIEGYCT | 6.445 |
| | | (SEQ ID NO: 87) | |
| 20 | 247 | WEALNMGL | 4.395 |
| | | (SEQ ID NO: 88) | |

One skilled in the art of peptide-based vaccination may determine which peptides would work best in individuals based on their HLA alleles (e.g., due to “MHC restriction”). Different HLA alleles will bind particular peptide motifs (usually 2 or 3 highly conserved positions out of 8-10) with different energies which can be predicted theoretically or measured as dissociation rates. Thus, a skilled artisan may be able to tailor the peptides to a subject's HLA profile.

In some embodiments, the present disclosure provides methods of eliciting an immune response against cells expressing a cancer associated sequence comprising contacting a subject with a cancer associated sequence under conditions effective to elicit an immune response in the subject, wherein said cancer associated sequence comprises a sequence or fragment thereof a gene selected from: C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2 orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a combination thereof.

In some embodiments, implementation of an immunotherapy strategy for treating, reducing the symptoms of, or preventing cancer or neoplasms, (e.g., a vaccine) may be achieved using many different techniques available to the skilled artisan.

Immunotherapy or the use of antibodies for therapeutic purposes has been used in recent years to treat cancer. Passive immunotherapy involves the use of monoclonal antibodies in cancer treatments. See, for example, Cancer: Principles and Practice of Oncology, 6th Edition (2001) Chapt, 20 pp. 495-508. Inherent therapeutic biological activity of these antibodies include direct inhibition of tumor cell growth or survival, and the ability to recruit the natural cell killing activity of the body's immune system. These agents may be administered alone or in conjunction with radiation or chemotherapeutic agents. Alternatively, antibodies may be used to make antibody conjugates where the antibody is linked to a toxic agent and directs that agent to the tumor by specifically binding to the tumor.

### Treating Cancer by Targeting DSCR6

Some embodiments herein are directed to a method of treating cancer in a subject, the method comprising administering to a subject in need thereof a therapeutic agent modulating the activity of a cancer associated protein, wherein the cancer associated protein is encoded by a nucleic acid comprising a nucleic acid sequence selected from DSCR6 (SEQ ID NO: 2), homologs thereof, combinations thereof, or a fragment thereof. In some embodiments, the therapeutic agent binds to the cancer associated protein. In some embodiments, the therapeutic agent is an antibody. In some embodiments, wherein the antibody may be a monoclonal antibody or a polyclonal antibody. In some embodiments, the antibody is a humanized or human antibody. In some embodiments, a method of treating cancer may comprise gene knockdown of DSCR6 (SEQ ID NO: 2). In some embodiments, a method of treating cancer may comprise treating cells to knockdown or inhibit expression of a gene encoding the mRNA disclosed in SEQ ID NO: 2. In some embodiments, the cancer is selected from small cell lung carcinoma, metastatic cervix adenocarcinoma, urinary bladder carcinoma, metastatic prostate adenocarcinoma, uterus endometrial stromal sarcoma, stomach tumor adenocarcinoma, metastatic tonsil carcinoma, large intestine rectum tumor adenocarcinoma, metastatic stomach tumor, metastatic kidney tumor from transitional cell carcinoma, metastatic endometrial stromal sarcoma, pleura tumor malignant sarcoma, rectum adenocarcinoma, cartilage chondrosarcoma, pancreas neuroendocrine carcinoma, lung squamous carcinoma, kidney carcinoma, liver cholangiocarcinoma, bone osteosarcoma metastatic, gastroesophageal junction adenocarcinoma metastatic, thyroid gland carcinoma metastatic, ovary tumor, prostate adenocarcinoma, rectum metastatic tumor or a combination thereof.

In some embodiments, the cancers treated by modulating the activity or expression of DSCR6 or the gene product thereof is a cancer classified by site or by histological type. Cancers classified by site include, but are not limited to, cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias).

Other type of cancers, classified by histological type, that may be associated with DSCR6 or include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangloendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fimgoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleuikemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia. Other types of cancers are also described herein and encompassed by the embodiments of the present invention. DSCR6 expression can be used to diagnose or treat cancer generally or for specific cancers as described herein including, but not limited to, the cancers described in the Example section.

In some embodiments, a method of diagnosing a subject with cancer comprises obtaining a sample and detecting the presence of a cancer associated sequence selected from SEQ ID NO: 2 wherein the presence of the cancer associated sequence indicates the subject has cancer. In some embodiments, detecting the presence of a cancer associated sequence selected from SEQ ID NO: 2 comprises contacting the sample with an antibody or other type of capture reagent that specifically binds to the cancer associated sequence's protein and detecting the presence or absence of the binding to the cancer associated sequence's protein in the sample. In some embodiments, the cancer is breast cancer. In some embodiments, the cancer is selected from small cell lung carcinoma, metastatic cervix adenocarcinoma, urinary bladder carcinoma, metastatic prostate adenocarcinoma, uterus endometrial stromal sarcoma, stomach tumor adenocarcinoma, metastatic tonsil carcinoma, large intestine rectum tumor adenocarcinoma, metastatic stomach tumor, metastatic kidney tumor from transitional cell carcinoma, metastatic endometrial stromal sarcoma, pleura tumor malignant sarcoma, rectum adenocarcinoma, cartilage chondrosarcoma, pancreas neuroendocrine carcinoma, lung squamous carcinoma, kidney carcinoma, liver cholangiocarcinoma, bone osteosarcoma metastatic, gastroesophageal junction adenocarcinoma metastatic, thyroid gland carcinoma metastatic, ovary tumor, prostate adenocarcinoma, rectum metastatic tumor or a combination thereof.

The detection of DSCR6 can, in some embodiments, be used to detect or diagnose cancers that are classified by histological type. In some embodiments, the cancers are Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma sv/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleuikemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia. Other types of cancers are also described herein and encompassed by the embodiments of the present invention.

### Expressing Cancer Associated Sequences in Cells

The cancer associated sequences disclosed herein may be used in research to develop cancer therapeutics or to study cellular mechanisms involved in carcinogenesis. Expression of the cancer associated sequences, either at the RNA level or the protein level may be achieved by transfecting the sequences into a target cell. Alternatively, proteins encoded for by the cancer associated sequences may be directly transported into cells as described below.

Electroporation may be used to introduce the cancer associated nucleic acids described herein into mammalian cells (Neumann, E, et al. (1982) EMBO J. 1, 841-845), plant and bacterial cells, and may also be used to introduce proteins (Marrero, M. B. et al. (1995) J. Biol. Chem, 270, 15734-15738; Nolkrantz, K. et al, (2002) Anal. Chem. 74, 4300-4305; Rui, M. Et al. (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery may be dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines may remain refractory even to electroporation unless partially activated.

Microinjection may be used to introduce femtoliter volumes of DNA directly into the nucleus of a cell (Capecehi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res, 55, 3490-3494; Theiss, C, and Meller, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes firsthand. Microinjection has the advantage of introducing macromolecules directly into the cell, thereby bypassing exposure to potentially undesirable cellular compartments such as low-pH endosomes.

Several proteins and small peptides have the ability to transduce or travel through biological membranes independent of classical receptor-mediated or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. In some embodiments, protein transduction domains (PTDs) from these proteins may be fused to other macromolecules, peptides or proteins such as, without limitation, a cancer associated polypeptide to successfully transport the polypeptide into a cell (Schwarze, S. R. et al. (2000) Trends Cell Biol. 10, 290-295). Exemplary advantages of using fusions of these transduction domains is that protein entry is rapid, concentration-dependent and appears to work with difficult cell types (Fenton, M. et al. (1998) J. Immunol. Methods 212, 41-48).

In some embodiments, liposomes may be used as vehicles to deliver oligonucleotides, DNA (gene) constructs and small drug molecules into cells (Zabner, J. et al. (1995) J. Biol. Chem. 270, 18997-19007; Feigner, P. L. et al. (1987) Proc. Natl. Acad. Set. USA 84, 7413-7417). Certain lipids, when placed in an aqueous solution and sonicated, form closed vesicles consisting of a circularized lipid bilayer surrounding an aqueous compartment. The vesicles or liposomes of embodiments herein may be formed in a solution containing the molecule to be delivered. In addition to encapsulating DNA in an aqueous solution, cationic liposomes may spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Feigner, J. H. et al. (1994) J. Biol. Chem. 269, 2550-2561). The cationic Liposome strategy has also been applied successfully to protein delivery (Zelphati, O. et al. (2001) J. Biol. Chem. 276, 35103-35110). Because proteins are more heterogeneous than DNA, the physical characteristics of the protein, such as its charge and hydrophobicity, may influence the extent of its interaction with the cationic lipids.

### Capture Reagents and Antibodies

In some embodiments the invention provides for capture reagents such as antibodies. The capture reagents may be used in diagnostic applications, therapeutic applications, research applications or drug screening applications and the like.

In some embodiments, the capture reagent has a KD equal or less than 10⁻⁹ M, 10⁻¹⁰ M, or 10⁻¹¹ M for its binding partner (e.g. antigen). In some embodiments, the capture reagent has a Ka greater than or equal to 10⁹ M⁻¹ for its binding partner. Capture reagent can also refer to, for example, antibodies. Intact antibodies, also known as immunoglobulins, are typically tetrameric glycosylated proteins composed of two light (L) chains of approximately 25 kDa each, and two heavy (H) chains of approximately 50 kDa each. Two types of light chain, termed lambda and kappa, exist in antibodies. Depending on the amino acid sequence of the constant domain of heavy chains, immunoglobulins are assigned to five major classes: A, D, E, G, and M, and several of these may be further divided into subclasses (isotypes), e.g., IgG 1, IgG2, IgG3, IgG4, IgA1, and IgA2. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated CH1. The VH and VL domains consist of four regions of relatively conserved sequences named framework regions (FR1, FR2, FR3, and FR4), which form a scaffold for three regions of hypervariable sequences (complementarity determining regions, CDRs). The CDRs contain most of the residues responsible for specific interactions of the antibody or antigen binding protein with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody or antigen binding protein-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, Eds. Harlow et al., 1988. One of skill in the art will recognize that each subunit structure, e.g., a CH, VH, CL, VL, CDR, and/or FR structure, comprises active fragments. For example, active fragments may consist of the portion of the VH, VL, or CDR subunit that binds the antigen, i.e., the antigen-binding fragment, or the portion of the CH subunit that binds to and/or activates an Fc receptor and/or complement.

Non-limiting examples of binding fragments encompassed within the term “antigen-specific antibody” used herein include: (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH I domains; (iv) an Fv fragment consisting of the VL and VH domains of a single arm of an antibody, (v) a dAb fragment, which consists of a VH domain; and (vi) an isolated CDR. Furthermore, although the two domains of the Fv fragment, VL and VH, are coded for by separate genes, they may be recombinantly joined by a synthetic linker, creating a single protein chain in which the VI, and VH domains pair to form monovalent molecules (known as single chain Fv (scFv)). The most commonly used linker is a 15-residue (Gly₄Ser)₃ peptide, but other linkers are also known in the art. Single chain antibodies are also intended to be encompassed within the terms “antibody or antigen binding protein,” or “antigen-binding fragment” of an antibody. The antibody can also be a polyclonal antibody, monoclonal antibody, chimeric antibody, antigen-binding fragment, Fc fragment, single chain antibodies, or any derivatives thereof.

Antibodies can be obtained using conventional techniques known to those skilled in the art, and the fragments are screened for utility in the same manner as intact antibodies. Antibody diversity is created by multiple germline genes encoding variable domains and a variety of somatic events. The somatic events include recombination of variable gene segments with diversity (D) and joining (J) gene segments to make a complete VH domain, and the recombination of variable and joining gene segments to make a complete VL domain. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells undergo somatic mutation. Based on the estimated number of germline gene segments, the random recombination of these segments, and random VH-VL pairing, up to 1.6×10⁷ different antibodies may be produced (Fundamental Immunology, 3rd ed. (1993), ed. Paul, Raven Press, New York, N.Y.). When other processes that contribute to antibody diversity (such as somatic mutation) are taken into account, it is thought that upwards of 1×10¹° different antibodies may be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

Antibody or antigen binding protein molecules capable of specifically interacting with the antigens, epitopes, or other molecules described herein may be produced by methods well known to those skilled in the art. For example, monoclonal antibodies can be produced by generation of hybridomas in accordance with known methods. Hybridomas formed in this manner can then be screened using standard methods, such as enzyme-linked immunosorbent assay (ELISA) and Biacore analysis, to identify one or more hybridomas that produce an antibody that specifically interacts with a molecule or compound of interest. As an alternative to preparing monoclonal antibody-secreting hybridomas, a monoclonal antibody to a polypeptide of the present disclosure may be identified and isolated by screening a recombinant combinatorial immunoglobulin library (e.g., an antibody phage display library) with a polypeptide of the present disclosure to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody or antigen binding protein display libraries can be found in the literature.

Examples of chimeric antibodies include, but are not limited to, humanized antibodies. The antibodies described herein can also be human antibodies. In some embodiments, the capture reagent comprises a detection reagent. The detection reagent can be any reagent that can be used to detect the presence of the capture reagent binding to its specific binding partner. The capture reagent can comprise a detection reagent directly or the capture reagent can comprise a particle that comprises the detection reagent. In some embodiments, the capture reagent and/or particle comprises a color, colloidal gold, radioactive tag, fluorescent tag, or a chemiluminescent substrate. The particle can be, for example, a viral particle, a latex particle, a lipid particle, or a fluorescent particle.

The capture reagents (e.g. antibody) of the present disclosure can also include an anti-antibody, i.e. an antibody that recognizes another antibody but is not specific to an antigen, such as, but not limited to, anti-IgG, anti-IgM, or ant-IgE antibody. This non-specific antibody can be used as a positive control to detect whether the antigen specific antibody is present in a sample.

### Administration of Therapeutics and Pharmaceutical Compositions

Modes of administration for a therapeutic (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the therapeutic of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present disclosure can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcelhilose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions of the present disclosure can also be formulated in rectal compositions such as suppositories or retention enemas, e.g., containing conventional suppository bases such as cocoa butter or other glycerides.

In addition to the formulations described previously, the therapeutic of the present disclosure can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present disclosure, for example, can be applied to a plaster, or can be applied by transdermal, therapeutic systems that are consequently supplied to the organism.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearylfumarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Kits

Also provided by the subject invention are kits and systems for practicing the subject methods, as described above, such components configured to diagnose cancer in a subject, treat cancer in a subject, or perform basic research experiments on cancer cells (e.g., either derived directly from a subject, grown in vitro or ex vivo, or from an animal model of cancer. The various components of the kits may be present in separate containers or certain compatible components may be pre-combined into a single container, as desired.

The subject systems and kits may also include one or more other reagents for performing any of the subject methods. The reagents may include one or more matrices, solvents, sample preparation reagents, buffers, desalting reagents, enzymatic reagents, denaturing reagents, probes, polynucleotides, vectors (e.g., plasmid or viral vectors), etc., where calibration standards such as positive and negative controls may be provided as well. As such, the kits may include one or more containers such as vials or bottles, with each container containing a separate component for carrying out a sample processing or preparing step and/or for carrying out one or more steps for producing a normalized sample according to the present disclosure.

In some embodiments, the invention provides a kit for diagnosing the presence of cancer in a test sample, said kit comprising at least one polynucleotide that selectively hybridizes to a cancer associated polynucleotide sequence shown in Table 1, or its complement. In another embodiment the invention provides an electronic library comprising a cancer associated polynucleotide, a cancer associated polypeptide, or fragment thereof, shown in Table 1. The kit may include an antibody that specifically binds to one or more proteins encoded by the cancer associated sequences disclosed infra.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Some embodiments are directed to a biochip comprising a nucleic acid segment which encodes a cancer associated protein. In some embodiments, a biochip comprises a nucleic acid molecule which encodes at least a portion of a cancer associated protein. In some embodiments, the cancer associated protein is encoded by a sequence selected from SEQ ID NOS: 1-70, homologs thereof, combinations thereof, or a fragment thereof. In some embodiments, the nucleic acid molecule specifically hybridizes with a nucleic acid sequence selected from SEQ ID NOS: 1-70. In some embodiments, the biochip comprises a first and second nucleic molecule wherein the first nucleic acid molecule specifically hybridizes with a first sequence selected from SEQ ID NOS: 1-70 and the second nucleic acid molecule specifically hybridizes with a second sequence selected from SEQ ID NOS: 1-70, wherein the first and second sequences are not the same sequence.

In addition to the subject database, programming and instructions, the kits may also include one or more control samples and reagents, e.g., two or more control samples for use in testing the kit.

### Additional Embodiments of the Invention

Embodiments of the disclosure are directed to methods of diagnosis, prognosis and treatment of cancer, including but not limited to breast cancer. The methods may be used for diagnosing and/or treating, for example, ductal carcinoma in situ (DCIS), invasive ductal carcinoma (IDC), medullary carcinoma, invasive lobular carcinoma (ILC), tubular carcinoma, mucinous carcinoma, inflammatory breast cancer (ILC), lobular carcinoma in situ (LCIS), male breast cancer, Paget's disease of the nipple, phyllodes tumors of the breast, recurrent and metastatic breast cancer, or a combination thereof.

In some embodiments, the methods comprise targeting a marker that is expressed at abnormal levels in breast tumor tissue in comparison to normal somatic tissue. In some embodiments, the marker may comprise a sequence selected from SEQ ID NOS: 1-70, complement thereof, or a combination thereof. In some embodiments, the methods for the treatment of cancer and related pharmaceutical preparations and kits are provided. Some embodiments are directed to methods of treating breast cancer comprising administering a composition including a therapeutic that affects the expression, abundance or activity of a target marker. In some embodiments, the target marker may include SEQ ID NOS: 1-70 or any combination thereof.

Some embodiments are directed to methods of detecting breast cancer comprising detecting a level of a target marker associated with the breast cancer. In some embodiments, the target marker may include SEQ ID NOS: 1-70, a complement thereof or any combination thereof.

Some embodiments herein provide antigens (i.e. cancer-associated polypeptides) associated with breast cancer as targets for diagnostic and/or therapeutic antibodies. In some embodiments, these antigens may be useful for drug discovery (e.g., small molecules) and for further characterization of cellular regulation, growth, and differentiation.

Some embodiments describe a method of diagnosing breast cancer in a subject, the method comprising: (a) obtaining a sample from a subject; (b) determining the expression of one or more genes or gene products or homologs thereof in the sample; and (c) comparing the expression of the one or more nucleic acid sequences from a second normal sample from the first subject or a second subject who does not have cancer, wherein a difference in the expression indicates that the first subject has breast cancer, wherein the gene or the gene product is referred to as a gene selected from: C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1 or a combination thereof.

Some embodiments describe a method of eliciting an immune response against cells expressing a cancer associated sequence comprising contacting a subject with a cancer associated sequence under conditions effective to elicit an immune response in the subject, wherein the cancer associated sequence comprises a sequence or fragment thereof a gene selected from: C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a combination thereof.

Some embodiments describe a method of detecting breast cancer in a test sample, comprising: (i) obtaining a sample from a subject; (ii) detecting a level of activity of at least one polypeptide that is a gene product in the sample; and (iii) comparing the level of activity of the polypeptide in the test sample with a level of activity of polypeptide in a normal sample (e.g. a sample obtained from a subject that does not have cancer), wherein an altered level of activity of the polypeptide in the test sample relative to the level of polypeptide activity in the normal sample is indicative of the presence of cancer in the test sample, wherein the gene product is a product of a gene selected from: C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a combination thereof.

Some embodiments herein are directed to a method of treating cancer in a subject, the method comprising administering to a subject in need thereof a therapeutic agent modulating the activity of a cancer associated protein, wherein the cancer associated protein is encoded by a nucleic acid comprising a nucleic acid sequence selected from DSCR6 (SEQ ID NO: 2), homologs thereof, combinations thereof, or a fragment thereof. In some embodiments, the therapeutic agent binds to the cancer associated protein. In some embodiments, the therapeutic agent is an antibody. In some embodiments the antibody may be a monoclonal antibody or a polyclonal antibody. In some embodiments, the antibody is a humanized or human antibody. In some embodiments, a method of treating cancer may comprise gene knockdown of DSCR6 (SEQ ID NO: 2). In some embodiments, a method of treating cancer may comprise treating cells to knockdown or inhibit expression of a gene encoding the mRNA disclosed in SEQ ID NO: 2. In some embodiments, the cancer is selected from small cell lung carcinoma, metastatic cervix adenocarcinoma, urinary bladder carcinoma, metastatic prostate adenocarcinoma, uterus endometrial stromal sarcoma, stomach tumor adenocarcinoma, metastatic tonsil carcinoma, large intestine rectum tumor adenocarcinoma, metastatic stomach tumor, metastatic kidney tumor from transitional cell carcinoma, metastatic endometrial stromal sarcoma, pleura tumor malignant sarcoma, rectum adenocarcinoma, cartilage chondrosarcoma, pancreas neuroendocrine carcinoma, lung squamous carcinoma, kidney carcinoma, liver cholangiocarcinoma, bone osteosarcoma metastatic, gastroesophageal junction adenocarcinoma metastatic, thyroid gland carcinoma metastatic, ovary tumor, prostate adenocarcinoma, rectum metastatic tumor or a combination thereof.

In some embodiments, a method of diagnosing a subject with cancer comprises obtaining a sample and detecting the presence of a cancer associated sequence selected from SEQ ID NO: 2 wherein the presence of the cancer associated sequence indicates the subject has breast cancer. In some embodiments, detecting the presence of a cancer associated sequence selected from SEQ ID NO: 2 comprises contacting the sample with an antibody or other type of capture reagent that specifically binds to the cancer associated sequence's protein and detecting the presence or absence of the binding to the cancer associated sequence's protein in the sample. In some embodiments, the cancer is selected from small cell lung carcinoma, metastatic cervix adenocarcinoma, urinary bladder carcinoma, metastatic prostate adenocarcinoma, uterus endometrial stromal sarcoma, stomach tumor adenocarcinoma, metastatic tonsil carcinoma, large intestine rectum tumor adenocarcinoma, metastatic stomach tumor, metastatic kidney tumor from transitional cell carcinoma, metastatic endometrial stromal sarcoma, pleura tumor malignant sarcoma, rectum adenocarcinoma, cartilage chondrosarcoma, pancreas neuroendocrine carcinoma, lung squamous carcinoma, kidney carcinoma, liver cholangiocarcinoma, bone osteosarcoma metastatic, gastroesophageal junction adenocarcinoma metastatic, thyroid gland carcinoma metastatic, ovary tumor, prostate adenocarcinoma, rectum metastatic tumor or a combination thereof.

In some embodiments, the present invention provides methods of treating cancer in a subject, the method comprising administering to a subject in need thereof a therapeutic agent that modulates the activity of DSCR6 or homologs thereof, wherein the therapeutic agent treats the cancer in the subject.

In some embodiments, the present invention provides methods of diagnosing cancer in a subject, the method comprising determining the expression of DSCR6 (SEQ ID NO: 2) from a sample; and diagnosing cancer in the subject based on expression DSCR6, wherein the subject is diagnosed as having cancer if DSCR6 is overexpressed.

In some embodiments, the present invention provides methods of detecting cancer in a test sample, the method comprising: (i) detecting a level of an antibody, wherein the antibody binds to an antigenic polypeptide encoded by a nucleic acid sequence comprising SEQ ID NO: 2, homologs thereof, combinations thereof, or a fragment thereof; and (ii) comparing the level of the antibody in the test sample with a level of the antibody in a control sample, wherein an altered level of antibody in the test sample relative to the level of antibody in the control sample is indicative of the presence of cancer in the test sample.

In some embodiments, the present invention provides methods of detecting cancer in a test sample, comprising: (i) detecting a level of activity of at least one polypeptide that is encoded by a nucleic acid comprising a nucleic acid sequence of SEQ ID NO: 2, homologs thereof, combinations thereof, or a fragment thereof; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample.

In some embodiments, the present invention provides methods of detecting cancer in a test sample, the method comprising: (i) detecting a level of expression of at least one polypeptide that is encoded by a nucleic acid comprising a nucleic acid sequence of SEQ ID NOs: 2, homologs thereof, combinations thereof, or a fragment thereof; and (ii) comparing the level of expression of the polypeptide in the test sample with a level of expression of polypeptide in a normal sample, wherein an altered level of expression of the polypeptide in the test sample relative to the level of polypeptide expression in the normal sample is indicative of the presence of cancer in the test sample.

In some embodiments, the present invention provides methods of detecting cancer in a test sample, the method comprising: (i) detecting a level of expression of a nucleic acid sequence comprising SEQ ID NOs: 2, homologs thereof, mutant nucleic acids thereof, combinations thereof, or a fragment thereof; and (ii) comparing the level of expression of the nucleic acid sequence in the test sample with a level of expression of nucleic acid sequence in a normal sample, wherein an altered level of expression of the nucleic acid sequence in the test sample relative to the level of nucleic acid sequence expression in the normal sample is indicative of the presence of cancer in the test sample.

In some embodiments, the present invention provides methods of screening for activity against cancer, the method comprising: (a) contacting a cell that expresses a cancer associated gene comprising a sequence of SEQ ID NO: 2, a complement thereof, homologs thereof, combinations thereof, or fragments thereof with a cancer drug candidate; (b) detecting an effect of the cancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has activity against cancer.

In some embodiments, the present invention provides methods of screening for activity against cancer, the method comprising: (a) contacting a cell that overexpresses a cancer associated gene comprising a sequence of SEQ ID NO: 2, a complement thereof, homologs thereof, combinations thereof, or fragments thereof with a cancer drug candidate; (b) detecting an effect of the cancer drug candidate on an expression of the cancer associated polynucleotide in the cell or an effect on cell growth or viability; and (c) comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against cancer cell that overexpresses a cancer associated gene comprising the sequence of SEQ ID NO: 2, a complement thereof, homologs thereof, combinations thereof, or fragments thereof.

In some embodiments, the present invention provides methods of diagnosing cancer in a subject, the method comprising: a) determining the expression of one or more nucleic acid sequences, wherein the one or more nucleic acid sequences comprises a sequence of SEQ ID NO: 2, homologs thereof, combinations thereof, or fragments thereof in a first sample of a first subject; and b) comparing the expression of the one or more nucleic acid sequences from a second normal sample from the first subject or a second unaffected subject, wherein a difference in the expression of SEQ ID NO: 2 indicates that the first subject has cancer.

In some embodiments, the present invention provides methods of diagnosing cancer in a subject, the method comprising: a) determining the expression of one or more genes or gene products or homologs thereof in a subject; and b) comparing the expression of the one or more genes or gene products or homologs thereof in the subject to the expression of one or more genes or gene products or homologs thereof from a normal sample from the subject or a normal sample from an unaffected subject, wherein a difference in the expression indicates that the subject has breast cancer, wherein the one or more genes or gene products comprises DSCR6.

In some embodiments, the present invention provides methods of detecting cancer in a test sample, comprising: (i) detecting a level of activity of at least one polypeptide; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein the polypeptide is a gene product of DSCR6.

In some embodiments, the present invention provides methods of diagnosing cancer in a subject, the method comprising: obtaining one or more gene expression results for one or more sequences, wherein the one or more sequences comprises SEQ ID NO: 2, from a sample derived from a subject; and diagnosing cancer in the subject based on the one or more gene expression results, wherein the subject is diagnosed as having cancer if one or more genes is overexpressed.

Other embodiments provide a method of detecting breast cancer in a subject comprising a) obtaining a sample from a subject b) contacting the sample obtained from the subject with one or more agents that detect expression of the markers encoded for by genes MMP11, Col10A, C10rf64, Col11A, POTEG, and FSIP1 or a complement thereof; c) contacting a non-cancerous cell, with the one or more agents from b); and d) comparing the expression level of the markers encoded for by genes MMP11, Col10A, C10rf64, Col11A, POTEG, and FSIP1 or a complement thereof in the sample obtained from the subject with the expression level of one the markers encoded for by genes MMP11, Col10A, C10rf64, Col11A, POTEG, and FSIP1 in the non-cancerous cell, wherein higher expression of at least one of the markers encoded for by genes MMP11, Col10A, C10rf64, Col11A, POTEG, and FSIP1 in the sample compared to the non-cancerous cell, indicates the subject has breast cancer.

Yet other embodiments provide a method of detecting breast cancer in a subject comprising a) obtaining a sample from a subject b) contacting the sample obtained from the subject with one or more agents that detect expression of the markers encoded for by genes FSIP1, Col10A, MMP11, NMU, and C1orf64, or a complement thereof; c) contacting a non-cancerous cell, with the one or more agents from b); and d) comparing the expression level of the markers encoded for by genes FSIP1, Col10A, MMP11, NMU, and C1orf64 or a complement thereof in the sample obtained from the subject with the expression level of one the markers encoded for by genes FSIP1, Col10A, MMP11, NMU, and C1orf64 in the non-cancerous cell, wherein higher expression of at least one of the markers encoded for by genes FSIP1, Col10A, MMP11, NMU, and C1orf64 in the sample compared to the non-cancerous cell, indicates the subject has breast cancer.

Embodiments illustrating the method and materials used may be further understood by reference to the following non-limiting examples.

### Example 1

### C1orf64

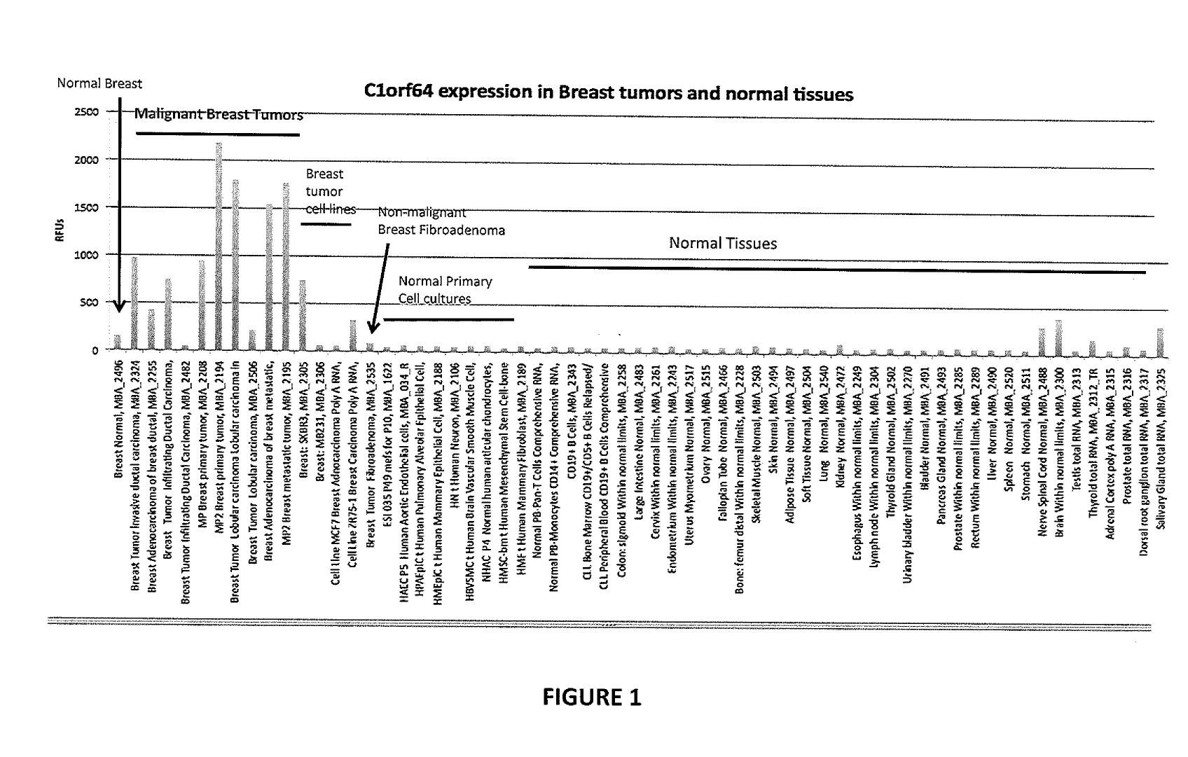

C1orf64 (Accession number NM_{—}178840.2; SEQ ID NO: 1) is an uncharacterized open reading frame encoding a putative uncharacterized protein of 169 amino acids. We disclose here that C1orf64 is a novel marker for breast tumors, including but not limited to breast infiltrating ductal carcinomas, breast lobular carcinomas, and metastatic breast tumors. As shown in FIG. 1, C1orf64 expression is assayed by Illumina microarray, a probe specific for C1orf64 (probe sequence GATCCGCTAAGGGGCATCTGAAACATCCGTCGAG TGGCAGAGGCAGGATA (SEQ ID NO; 89); Illumina probe ID ILMN_{—}2066088) detects-strong gene expression (>500 RFUs) in breast infiltrating ductal carcinomas, breast lobular carcinoma and metastatic breast tumors, while expression in normal breast tissue and a non-malignant breast adenocarcinoma is low (151 and 84 RFUs respectively). Expression of C1orf64 in a wide variety of normal tissues including colon, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, liver, spleen, stomach, spinal cord, brain, testis, thyroid, adrenal cortex, dorsal root ganglion, salivary gland and diverse nucleated blood cells is generally low (in most normal tissues <100 RFUs, in all normal tissues <400 RFUs). As shown in FIG. 1, the expression of C1orf64 is also low in a large variety of normal primary human cell cultures including but not limited to mammary epithelial cells, neurons, articular chondrocytes, mammary fibroblasts and mesenchymal stem cells. The specificity of elevated C1orf64 expression in malignant tumors of the breast shown herein demonstrates that C1orf64 is a useful marker for the diagnosis of breast cancer and a target for therapeutic intervention in breast cancer treatment.

### Example 2

### LOC648879

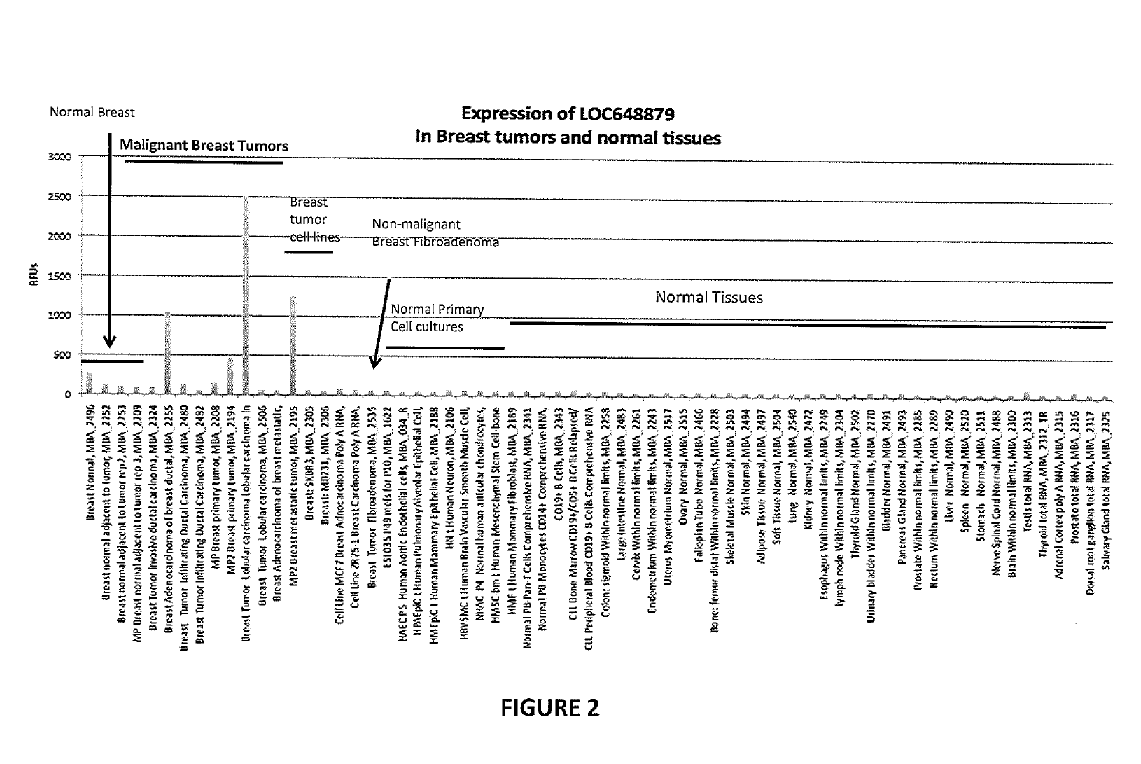

LOC648879 (Accession number XM_{—}937958.1; SEQ ID NO: 4) is an uncharacterized open reading frame. We disclose here that LOC648879 is a novel marker for breast tumors, including but not limited to breast infiltrating ductal carcinomas, breast lobular carcinomas, and metastatic breast tumors. As shown in FIG. 2, LOC648879 expression is assayed by Illumina microarray, a probe specific for LOC648879 (probe sequence GCGCCATCTTGCCCTGTAGATCATTTTGGGGACACCTCCAGTATTTCATG (SEQ ID NO; 90); Illumina probe ID ILMN_{—}1739233) detects strong gene expression (>1000 RFUs) in diverse malignant breast tumors including but not limited to breast infiltrating ductal carcinoma, breast lobular carcinoma and metastatic breast tumors, while expression in normal breast tissue and a non-malignant breast adenocarcinoma is low (average of 151 and 71 RFUs respectively). Expression of LOC648879 in a wide variety of normal tissues including colon, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, liver, spleen, stomach, spinal cord, brain, thyroid, adrenal cortex, dorsal root ganglion, salivary gland and diverse nucleated blood cells is low (<100 RFUs), with normal testis showing slightly more expression at 112 RFUs. As shown in FIG. 2, the expression of LOC648879 is also low (<100 RFUs) in a large variety of normal primary human cell cultures including but not limited to mammary epithelial cells, neurons, articular chondrocytes, mammary fibroblasts and mesenchymal stem cells. The specificity of elevated LOC648879 expression in malignant tumors of the breast shown herein demonstrates that LOC648879 is a marker for the diagnosis of breast cancer and a target for therapeutic intervention in breast cancer treatment.

### Example 3

### HIST1H4H

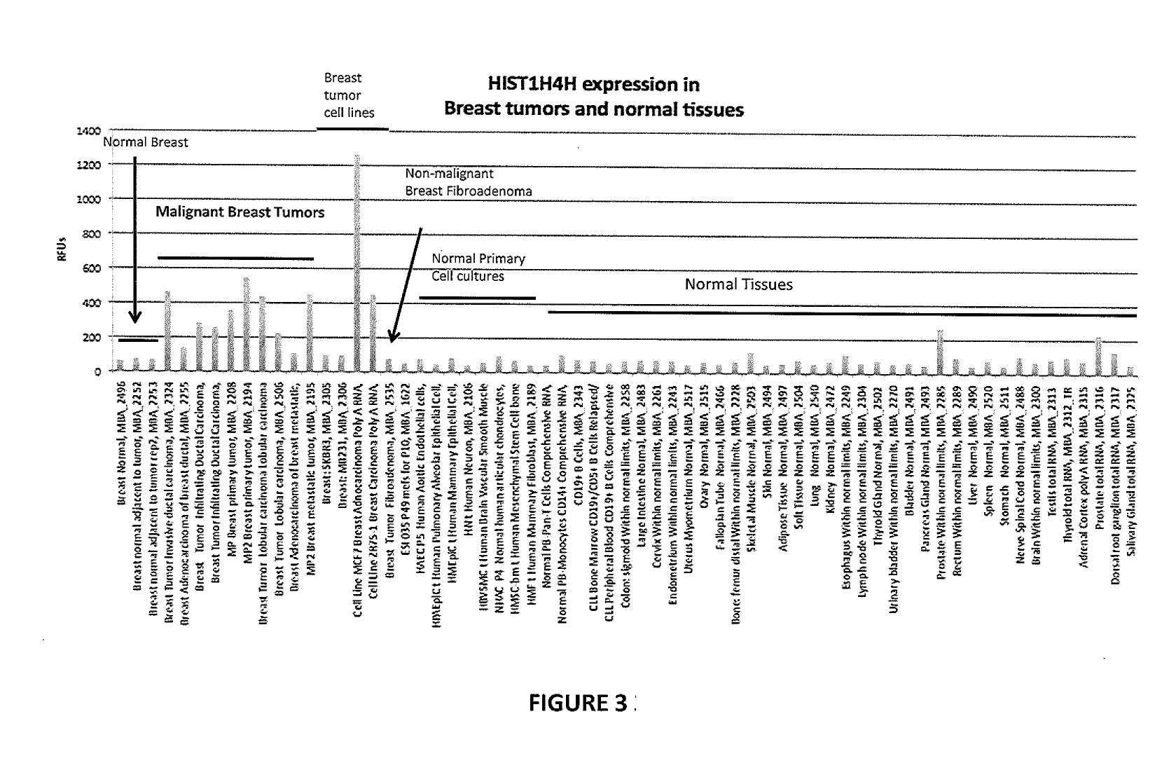

HIST1H4H (Accession number NM_{—}003543.3; SEQ ID NO: 5) encodes a member of the histone H4 family. Histone proteins are responsible for chromatin structure in eukaryotes. Unexpectedly, we show here that HIST1H4H has low levels of expression in most normal human tissues and normal primary human cell cultures while it is surprisingly specifically elevated in malignant breast tumors and breast tumor cell lines. As shown in FIG. 3, expression is assayed by Illumina microarray, a probe specific for HIST1H4H (probe sequence CGCACTCTTTACGGCTTCGGTGGCTAAGGCTCCTGCTTGCTGCACTC TTA(SEQ ID NO; 91); Illumina probe ID ILMN_{—}1751120) detects strong gene expression (>400 RFUs) in diverse malignant breast tumors including but not limited to breast infiltrating, ductal carcinoma, breast lobular carcinoma and metastatic breast tumors, while expression in normal breast tissue and a non-malignant breast adenocarcinoma is low (<75 and 78 RFUs respectively). Expression of HIST1H4H in a wide variety of normal tissues including colon, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, liver, spleen, stomach, spinal cord, brain, thyroid, testis, adrenal cortex, dorsal root ganglion, salivary gland and diverse nucleated blood cells is low (<100 RFUs), with normal skeletal muscle and prostate showing slightly more expression at 125 and 264 RFUs respectively. As shown in FIG. 3, the expression of HIST1H4H is also low (<100 RFUs) in a large variety of normal primary human cell cultures including but not limited to mammary epithelial cells, neurons, articular chondrocytes, mammary fibroblasts and mesenchymal stem cells. The specificity of elevated HIST1H4H expression in malignant tumors of the breast shown herein demonstrates that HIST1H4H is a marker for the diagnosis of breast cancer and a target for therapeutic intervention in breast cancer treatment.

### Example 4

### HIST2H4B

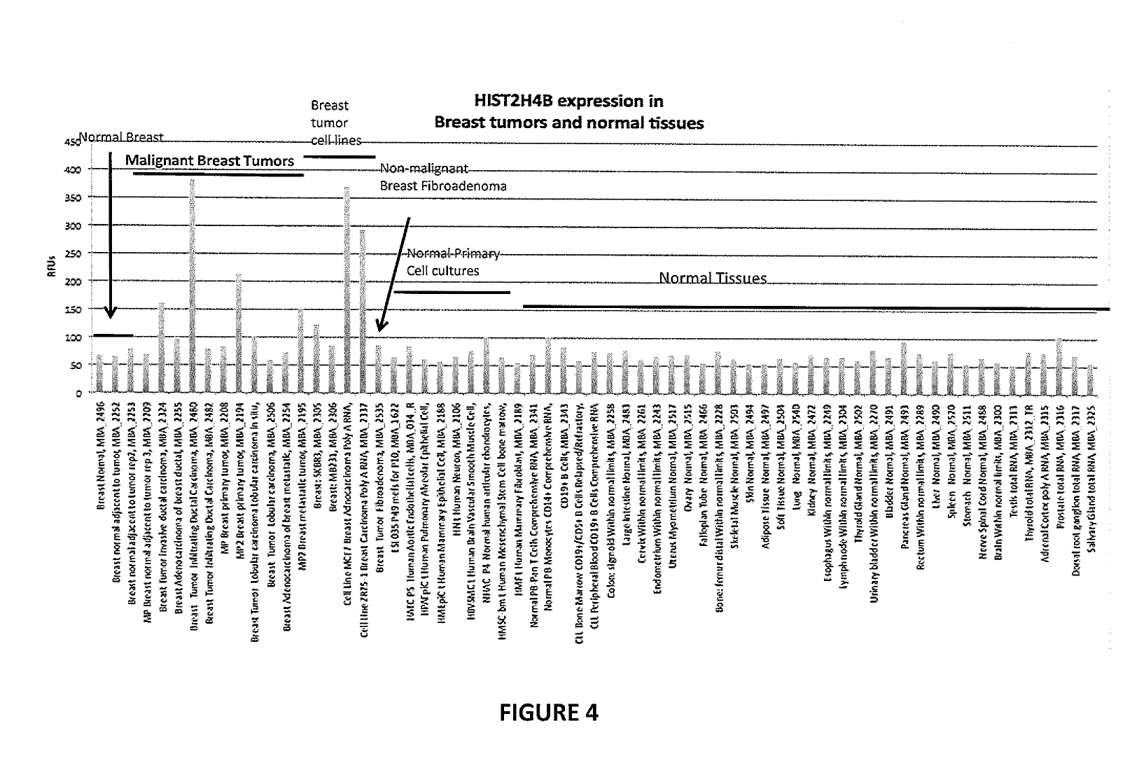

HIST2H4B (Accession number NM_{—}001034077.4; SEQ ID NO: 10) encodes a member of the histone H4 family. Histone proteins are responsible for chromatin structure in eukaryotes. Unexpectedly, we show here that HIST2H4B has low levels of expression in most normal human tissues and normal primary human cell cultures while it is surprisingly specifically elevated in malignant breast tumors and breast tumor cell lines. As shown in FIG. 4, expression is assayed by Illumina microarray, a probe specific for HIST2H4B (probe sequence GTGTTCCTGGAGAATGTGATTCGGGACGCAGTCACCTACACCGAGCACGC (SEQ ID NO; 92); Illumina probe ID ILMN_{—}3238233) detects strong gene expression (>100 RFUs) in diverse malignant breast tumors including but not limited to breast infiltrating ductal carcinoma, and metastatic breast tumors, while expression in normal breast tissue and a non-malignant breast adenocarcinoma is low (<79 and 86 RFUs respectively). Expression of HIST2′-14B in a wide variety of normal tissues including colon, cervix, endometrium, uterus myometrium, ovary, fallopian tube, bone, skeletal muscle, skin, adipose tissue, soft tissue, lung, kidney, esophagus, skeletal muscle, lymph node, thyroid, urinary bladder, pancreas, prostate, rectum, liver, spleen, stomach, spinal cord, brain, thyroid, testis, adrenal cortex, dorsal root ganglion, salivary gland and diverse nucleated blood cells is low (<100 RFUs), with normal prostate showing slightly more expression at 104 RFUs. As shown in FIG. 4, the expression of HIST2H4B is also low (<100 RFUs) in a large variety of normal primary human cell cultures including but not limited to mammary epithelial cells, neurons, articular chondrocytes, mammary fibroblasts and mesenchymal stem cells. The specificity of elevated HIST2H4B expression in malignant tumors of the breast shown herein demonstrates that HIST2H4B is a marker for the diagnosis of breast cancer and a target for therapeutic intervention in breast cancer treatment.

### Example 5

### BX116033

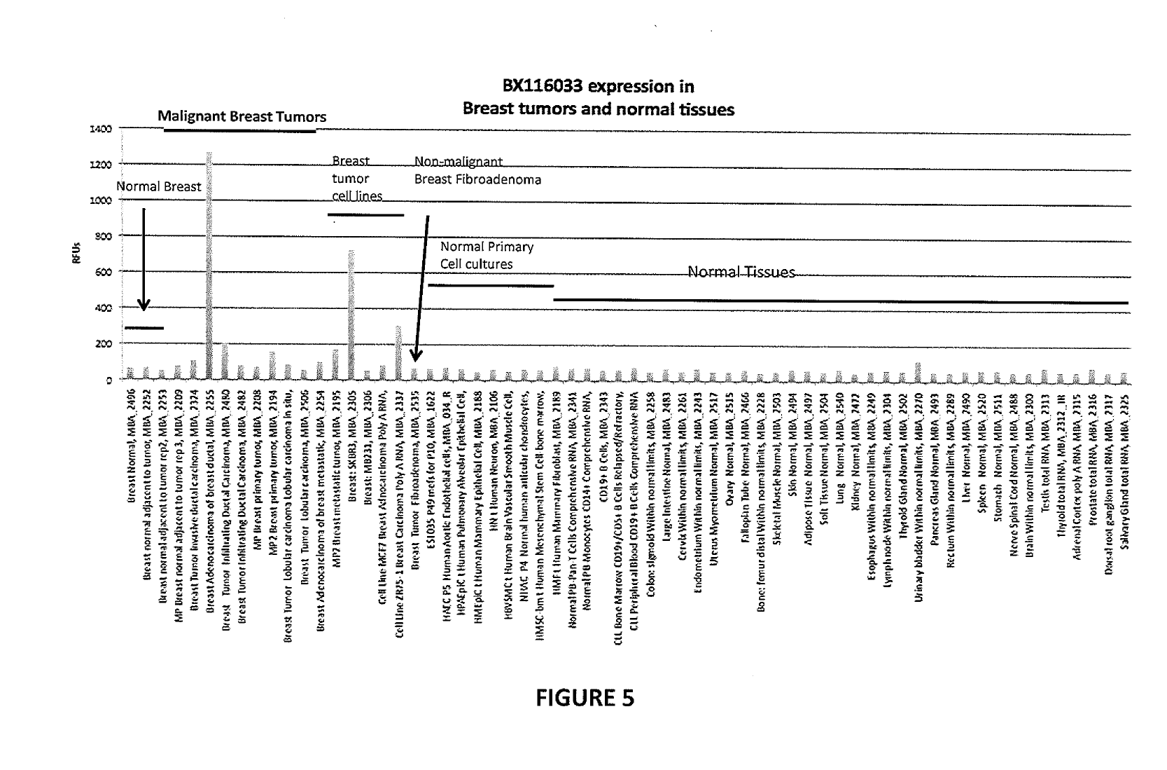

BX116033 (Accession number BX116033; SEQ ID NO: 11) encodes an uncharacterized transcript. We show here that BX116033 has low levels of expression in most normal human tissues and normal primary human cell cultures while it is surprisingly specifically elevated in malignant breast tumors and breast tumor cell lines. As shown in FIG. 5, expression is assayed by Illumina microarray, a probe specific for BX116033 (probe sequence TGCCGTATTCTTGGTGTCTGGAGCAGTGCCTGACCTGTGGCGGGTGC TTA; (SEQ ID NO; 93) Illumina probe ID ILMN_{—}1863962) detects strong gene expression (>100 RFUs) in diverse malignant breast tumors including but not limited to breast infiltrating ductal carcinoma, and metastatic breast tumors, while expression in normal breast tissue and a non-malignant breast adenocarcinoma is low (<70 RFUs). Expression of BX116033 in a wide variety of normal tissues including colon, cervix, endometrium, uterus myometrium, ovary, fallopian tube, bone, skeletal muscle, skin, adipose tissue, soft tissue, lung, kidney, esophagus, skeletal muscle, lymph node, thyroid, prostate, pancreas, prostate, rectum, liver, spleen, stomach, spinal cord, brain, thyroid, testis, adrenal cortex, dorsal root ganglion, salivary gland and diverse nucleated blood cells is low (<80 RFUs), with urinary bladder showing slightly more expression at 118 RFUs. As shown in FIG. 5, the expression of BX116033 is also low (<80 RFUs) in a large variety of normal primary human cell cultures including but not limited to mammary epithelial cells, neurons, articular chondrocytes, mammary fibroblasts and mesenchymal stem cells. The specificity of elevated BX116033 expression in malignant tumors of the breast shown herein demonstrates that BX116033 is a marker for the diagnosis of breast cancer and a target for therapeutic intervention in breast cancer treatment.

### Example 6

### DSCR6

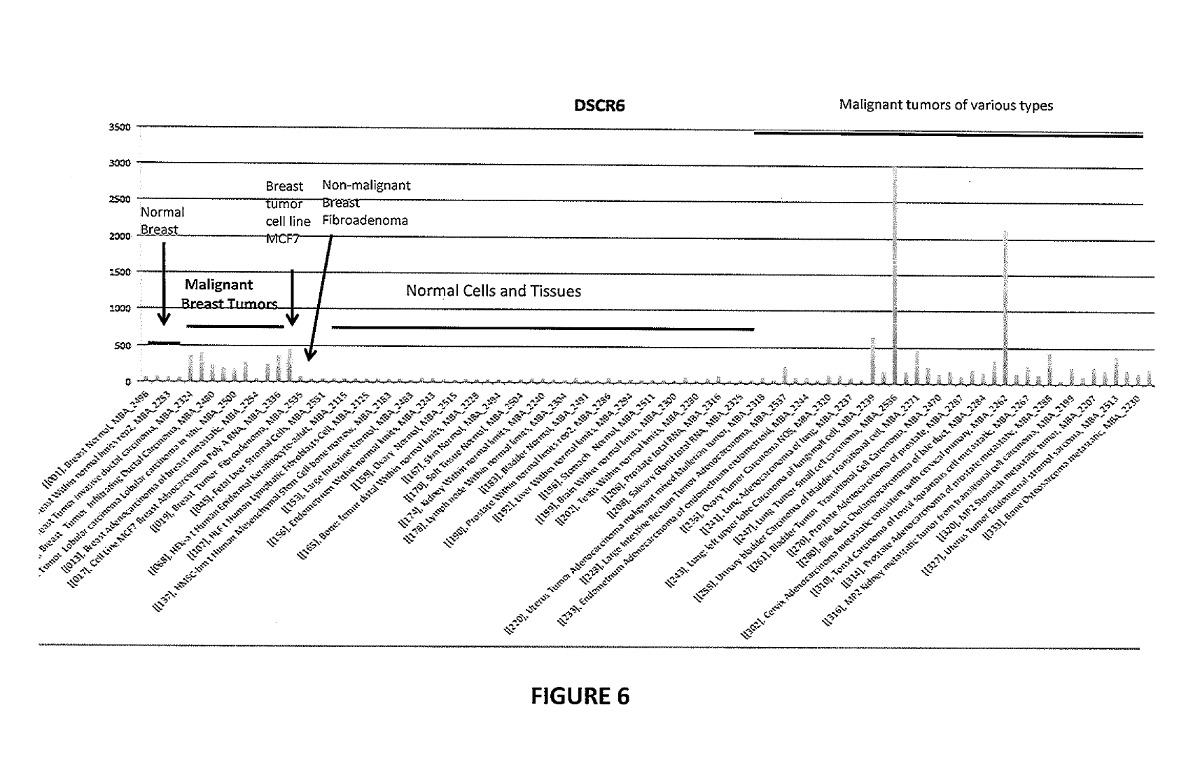

DSCR6, Down Syndrome Critical Region Gene 6 (Accession number NM_{—}018962.1; SEQ ID NO: 2) encodes a protein of unknown function that is expressed only in limited tissues at low levels (Shibuya K, et al., PMID 10814524). We disclose here that DSCR6 is a novel marker for breast tumors and malignant tumors of diverse tissues of origin, including but not limited to breast infiltrating ductal carcinomas, breast lobular carcinomas, and metastatic breast tumors. As shown in FIG. 6, DSCR6 expression is assayed by Illumina microarray, a probe specific for DSCR6 (probe sequence TAGGGAGTAGAACCGTCTCTCTTCTTAGTTGG TGACTGTTTGGGGCCTGG(SEQ ID NO; 94); Illumina probe ID ILMN_{—}1709257) detects strong gene expression (>185 RFUs) in breast infiltrating ductal carcinomas and breast lobular carcinomas, while expression in normal breast tissue and a non-malignant breast adenocarcinoma is low (<80 RFUs). Expression of DSCR6 in a wide variety of normal tissues including colon, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, liver, spleen, stomach, spinal cord, brain, testis, thyroid, adrenal cortex, dorsal root ganglion, salivary gland and diverse nucleated blood cells is generally low (in normal tissues <100 RFUs). The specificity of elevated DSCR6 expression in malignant tumors of the breast shown herein demonstrates that DSCR6 is a useful marker for the diagnosis of breast cancer and a target for therapeutic intervention in breast cancer treatment.

DSCR6 expression is also elevated in malignant tumors of diverse origin including but not limited to small cell lung carcinoma, metastatic cervix adenocarcinoma, urinary bladder carcinoma, metastatic prostate adenocarcinoma, uterus endometrial stromal sarcoma, stomach tumor adenocarcinoma, metastatic tonsil carcinoma, large intestine rectum tumor adenocarcinoma, metastatic stomach tumor, metastatic kidney tumor from transitional cell carcinoma, metastatic endometrial stromal sarcoma, pleura tumor malignant sarcoma, rectum adenocarcinoma, cartilage chondrosarcoma, pancreas neuroendocrine carcinoma, lung squamous carcinoma, kidney carcinoma, liver cholangiocarcinoma, bone osteosarcoma metastatic, gastroesophageal junction adenocarcinoma metastatic, thyroid gland carcinoma metastatic, ovary tumor, prostate adenocarcinoma, and rectum metastatic tumor (>100 RFUs). The elevated expression of DSCR6 in diverse malignant tumors with only limited low expression in normal tissues indicates that DSCR6 is a useful marker for the diagnosis of malignant tumors and is a target for therapeutic intervention in the treatment of malignant tumors.

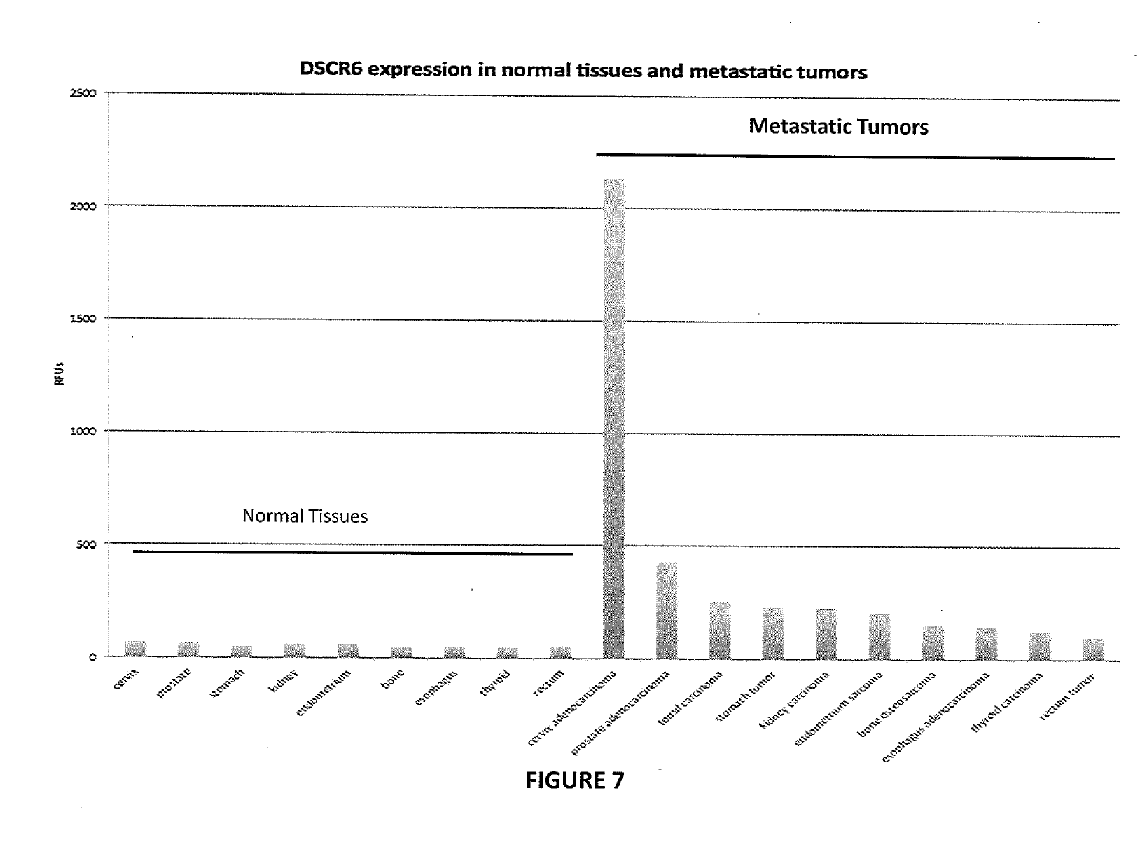

As shown in FIG. 7, DSCR6 expression is elevated in metastatic tumors of diverse tissues of origin including but not limited to cervix, prostate, tonsil, stomach, kidney, endometrium, bone, gastroesophageal junction, thyroid, and rectum (all >100 RFUs, FIG. 7), while the expression levels of DSCR6 in normal cervix, prostate, tonsil, stomach, kidney, endometrium, bone, esophagus, thyroid, and rectum are low (<100 RFUs, FIG. 7). The elevated expression of DSCR6 in diverse metastatic tumors indicates that DSCR6 is a useful marker for the diagnosis of metastatic tumors in general and a target for therapeutic intervention for metastatic disease.

### Example

### 7POTEC

POTEC (Accession number NM_{—}001137671.1) encodes POTE ankyrin domain family member C. It is disclosed here that POTEC is a novel marker for breast tumors. As shown in FIG. 1, POTEC expression was assayed by Illumina microarray, a probe specific for POTEC (probe sequence CTGCCTGGTGGGGTAAGGTCCCCAGAAAGGATCTCATCGTCAT GCTCAGG (SEQ ID NO: 95); Illumina probe ID ILMN_{—}1753868) detected strong gene expression (>140 RFUs) in breast tumor lobular carcinoma, breast infiltrating ductal carcinoma and metastatic breast tumor. In contrast, expression of POTEC in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, liver, spleen, stomach, spinal cord, brain, thyroid, and salivary gland was generally low (<140 RFUs), with the exception of testis (218 RFUs). The specificity of elevated POTEC expression in malignant tumors of breast origin shown herein demonstrates that POTEC is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target POTEC can be identified using the methods described herein and therapeutics that target POTEC include, but are not limited to, antibodies that modulate the activity of POTEC. The manufacture and use of antibodies are described herein.

### Example 8

### FSIP1

FSIP1 (Accession number NM 152597.4) encodes Homo sapiens fibrous sheath interacting protein 1. Surprisingly, it is disclosed here that FSIP1 is a novel marker for breast tumors. As shown in FIG. 2, FSIP1 expression was assayed by Iluminamicroarray, a probe specific for FSIP1 (probe sequence GGTGGTCACTGGGAATTTTTGCTGTGGCCCTGCT TTTCCTTCTTCCCACT (SEQ ID NO: 96); Illumina probe ID ILMN_{—}1716925) detected strong gene expression (>125 RFUs) in breast tumor lobular carcinoma, breast infiltrating ductal carcinoma and metastatic breast tumor. In contrast, expression of FSIP1 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, thyroid, and salivary gland was generally low (<125 RFUs), with the exception of liver (191 RFUs). The specificity of elevated FSIP1 expression in malignant tumors of breast origin shown herein demonstrates that FSIP1 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target FSIP1 can be identified using the methods described herein and therapeutics that target FSIP1 include, but are not limited to, antibodies that modulate the activity of FSIP1. The manufacture and use of antibodies are described herein.

### Example 9

### GFRA1

GFRA1 (Accession number NM_{—}005264.3) encodes Homo sapiens GDNF family receptor alpha 1. Surprisingly, it is disclosed here that GFRA1 is a novel marker for breast tumors. As shown in FIG. 3, GFRA1 expression was assayed by Illumina microarray, a probe specific for GFRA1 (probe sequence TCCCTGAACGACACTCTCCTAATCCTAAGCCTTACCTGAGTGAGAAGCCC (SEQ ID NO: 97); Illumina probe ID ILMN_{—}2334359) detected strong gene expression (>215 RFUs) in breast tumor lobular carcinoma, breast infiltrating ductal carcinoma and metastatic breast tumor. In contrast, expression of GFRA1 in a wide variety of normal tissues including normal breast, colon, rectum, cervix, endometrium, uterus myometrium, ovary, fallopian tube, bone, skeletal muscle, skin, adipose tissue, soil tissue, lung, kidney, esophagus, lymph node, thyroid, urinary bladder, pancreas, prostate, rectum, spleen, stomach, spinal cord, brain, testis, liver, thyroid, and salivary gland was generally low (<215 RFUs). The specificity of elevated GFRA1 expression in malignant tumors of breast origin shown herein demonstrates that GFRA1 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target GFRA1 can be identified using the methods described herein and therapeutics that target GFRA1 include, but are not limited to, antibodies that modulate the activity of GFRA1. The manufacture and use of antibodies are described herein.

### Example 10

### LOC647333 (POTEF, POTEE and POTEK)

The POTE gene family encodes a number of homologous proteins with ankyrin repeats. Surprisingly, it is disclosed here that POTEF, POTEE and POTEK (Accession numbers NM_{—}001099771.2, NM_{—}001083538.1 and NR_{—}033885.1) are novel markers for breast tumors. As shown in FIG. 4, POTEF, POTEE AND POTEK expression was assayed by Illumina microarray, a probe specific for the conserved region (LOC647333; XM_{—}936396.1) between three POTE family members: POTEF, POTEE and POTEK (probe sequence ATGGTGGTTGAGGTTGATTCCATGCCGGCTGCCTCTTCTGTGAAGAAGCC (SEQ ID NO: 98); Illumina probe ID ILMN_{—}1814643) detected strong gene expression (>200 RFUs) in breast tumor lobular carcinoma, breast infiltrating ductal carcinoma and metastatic breast tumor. In contrast, expression of POTEF, POTEE and POTEK in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<200 RFUs). The specificity of elevated POTEF, POTEE and POTEK expression in malignant tumors of breast origin shown herein demonstrates that POTEF, POTEE and POTEK is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target POTEF, POTEE AND POTEK can be identified using the methods described herein and therapeutics that target POTEF, POTEE AND POTEK include, but are not limited to, antibodies that modulate the activity of POTEF, POTEE AND POTEK. The manufacture and use of antibodies are described herein.

### Example 11

### C2orf27A

C2ORF27A (Accession number NM_{—}013310.3) encodes Homo sapiens chromosome 2 open reading frame 27A. Surprisingly, it is disclosed here that C2ORF27A is a novel marker for breast tumors. As shown in FIG. 5, C2ORF27A expression was assayed by Illumina microarray, a probe specific for C2ORF27A (probe sequence CCAACATGCTCTAATGCTTCAGATTCAAGTGCTTTTTCCACTGTTT CCCC (SEQ ID NO: 99); Illumina probe ID ILMN_{—}1684726) detected strong gene expression (>300 RFUs) in breast tumor lobular carcinoma, breast infiltrating ductal carcinoma and metastatic breast tumor. In contrast, expression of C2ORF27A in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<70 RFUs). The specificity of elevated C2ORF27A expression in malignant tumors of breast origin shown herein demonstrates that C2ORF27A is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target C2ORF27A can be identified using the methods described herein and therapeutics that target C2ORF27A include, but are not limited to, antibodies that modulate the activity of C2ORF27A. The manufacture and use of antibodies are described herein.

### Example 12

### LOC727941

LOC727941 (Accession number XR_{—}037440.1) encodes an uncharacterized protein. Surprisingly, it is disclosed here that LOC727941 is a novel marker for breast tumors. As shown in FIG. 6, LOC727941 expression was assayed by IIlumina microarray, a probe specific for LOC727941 (probe sequence GGGGTTTCACCTCAAACATCATAAAGGTGCTTCCTGCAGTAGGCGTTGGC (SEQ ID NO 100); Illumina probe ID ILMN_{—}3283936) detected strong gene expression (>130 RFUs) in breast tumor lobular carcinoma and breast infiltrating ductal carcinoma. In contrast, expression of LOC727941 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<80 RFUs). The specificity of elevated LOC727941 expression in malignant tumors of breast origin shown herein demonstrates that LOC727941 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target LOC727941 can be identified using the methods described herein and therapeutics that target LOC727941 include, but are not limited to, antibodies that modulate the activity of LOC727941. The manufacture and use of antibodies are described herein.

### Example 13

### NBPF22P

NBPF22P (Accession number NR_{—}003719.1) encodes Homo sapiens neuroblastoma breakpoint family, member 22 (pseudogene). Surprisingly, it is disclosed here that NBPF22P is a novel marker for breast tumors. As shown in FIG. 7, NBPF22P expression was assayed by Illumina microarray, a probe specific for NBPF22P (probe sequence GCAGGCAGAGAAGGCCCAGTGTGTCCATCCCCAATGCGGTGATACTAGGA (SEQ ID NO: 101); Illumina probe ID ILMN_{—}241634) detected strong gene expression (>100 RFUs) in breast tumor lobular carcinoma, breast infiltrating ductal carcinoma and metastatic breast tumor. In contrast, expression of NBPF22P in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, liver, thyroid, and salivary gland was generally low (<80 RFUs), with the exception of testis (189 RFUs). The specificity of elevated NBPF22P expression in malignant tumors of breast origin shown herein demonstrates that NBPF22P is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target NBPF22P can be identified using the methods described herein and therapeutics that target NBPF22P include, but are not limited to, antibodies that modulate the activity of NBPF22P. The manufacture and use of antibodies are described herein.

### Example 14

### POTEG

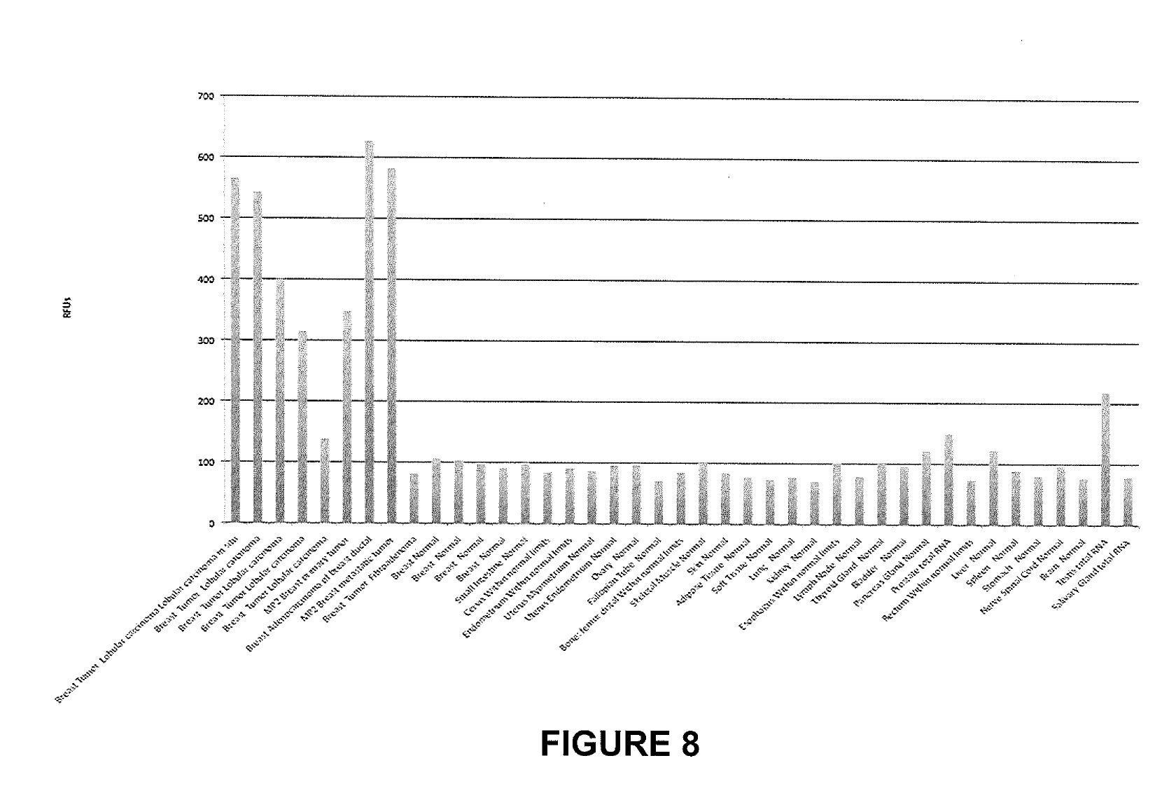

POTEG (Accession number NM_{—}1001005356.2) encodes POTE ankyrin domain family member G. Surprisingly, it is disclosed here that POTEG is a novel marker for breast tumors. As shown in FIG. 8, POTEG expression was assayed by Illumina microarray, a probe specific for POTEG (probe sequence AGAGGACAGCTCTGACAAAGGCCGTACAATGCCGGGAAGATG AATGTGCG (SEQ ID NO: 102); Illumina probe ID ILMN_{—}3242191) detected strong gene expression (>200 RFUs) in breast tumor lobular carcinoma, breast infiltrating ductal carcinoma and metastatic breast tumor. In contrast, expression of POTEG in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<120 RFUs), with the exception of prostate (228 RFUs). The specificity of elevated POTEG expression in malignant tumors of breast origin shown herein demonstrates that POTEG is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target POTEG can be identified using the methods described herein and therapeutics that target POTEG include, but are not limited to, antibodies that modulate the activity of POTEG. The manufacture and use of antibodies are described herein.

### Example 15

### RET

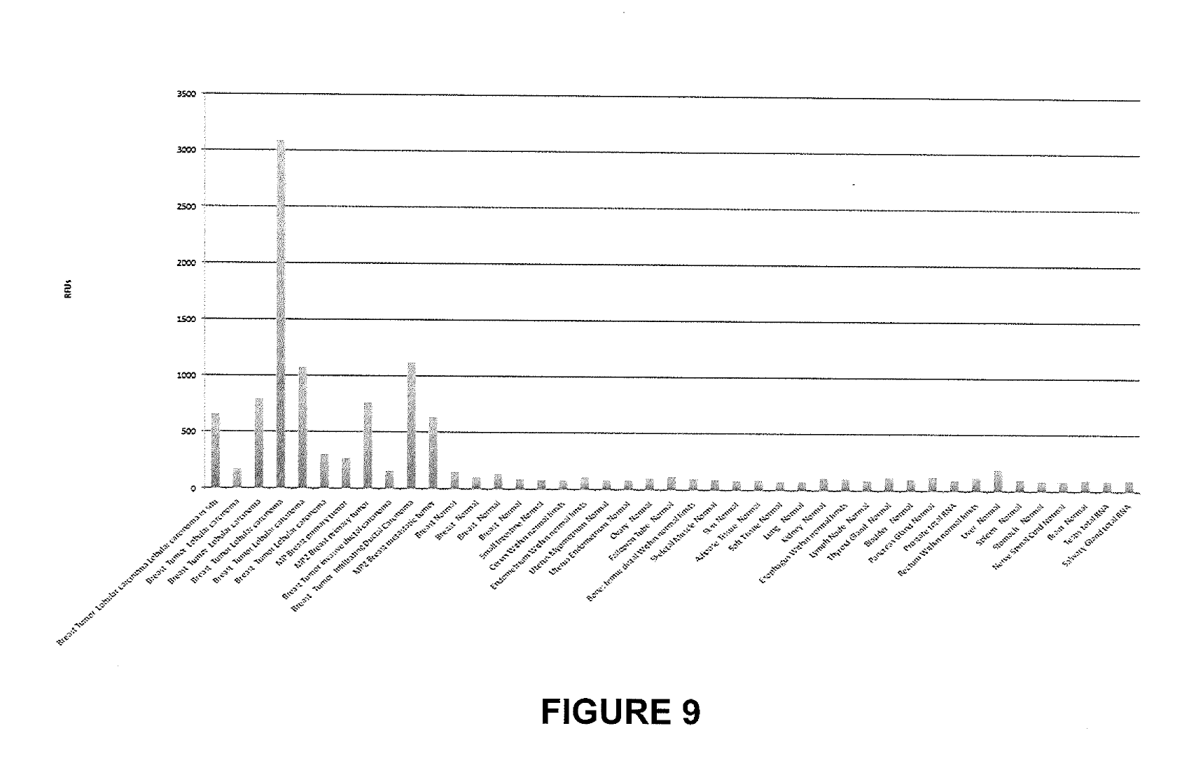

RET (Accession number NM_{—}020630.4) encodes Homo sapiens ret proto-oncogene. Surprisingly, it is disclosed here that RET is a novel marker for breast tumors. As shown in FIG. 9, RET expression was assayed by Illumina microarray, a probe specific for RET (probe sequence GGGAGGACGCACCCCCACTGCTGTTTTCACATCCYTTCCCTTACCCACC T (SEQ ID NO: 103); Illumina probe ID ILMN_{—}1655610) detected strong gene expression (>105 RFUs) in breast tumor lobular carcinoma and breast infiltrating ductal carcinoma. In contrast, expression of RET in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<105 RFUs). The specificity of elevated RET expression in malignant tumors of breast origin shown herein demonstrates that RET is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target RET can be identified using the methods described herein and therapeutics that target RET include, but are not limited to, antibodies that modulate the activity of RET. The manufacture and use of antibodies are described herein.

### Example 16

### TMEM145

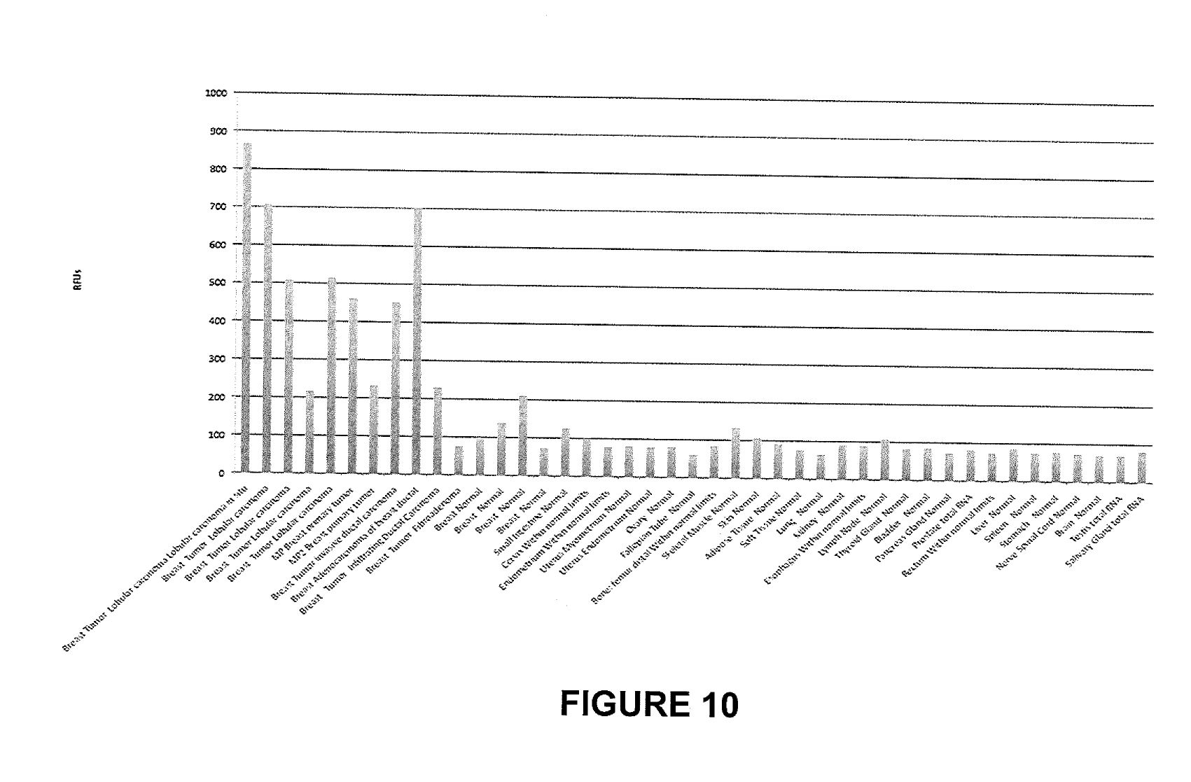

TMEM145 (Accession number NM 173633.2) encodes Homo sapiens transmembrane protein 145. Surprisingly, it is disclosed here that TMEM145 is a novel marker for breast tumors. As shown in FIG. 10, TMEM145 expression was assayed by Illumina microarray, a probe specific for TMEM145 (probe sequence CGGGGGCCTTCCCTCGGGTCCCTGGCAGAAAGACATTTTACCCCTTCTTG (SEQ ID NO: 104); Illumina probe ID ILMN_{—}1789112) detected strong gene expression (>170 RFUs) in breast tumor lobular carcinoma and breast infiltrating ductal carcinoma. In contrast, expression of TMEM145 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, testis, liver, thyroid, and salivary gland was generally low (<170 RFUs) with the exception of brain and spinal cord (1051 and 245 RFUs respectively. The specificity of elevated TMEM145 expression in malignant tumors of breast origin shown herein demonstrates that TMEM145 is a marker for the diagnosis of breast cancer (e.g. including but not limited to, breast tumor lobular carcinoma, breast infiltrating ductal carcinoma and metastatic breast tumors), and is a target far therapeutic intervention in breast cancer.

Therapeutics that target TMEM145 can be identified using the methods described herein and therapeutics that target TMEM145 include, but are not limited to, antibodies that modulate the activity of TMEM145. The manufacture and use of antibodies are described herein.

### Example 17

### LOC727941

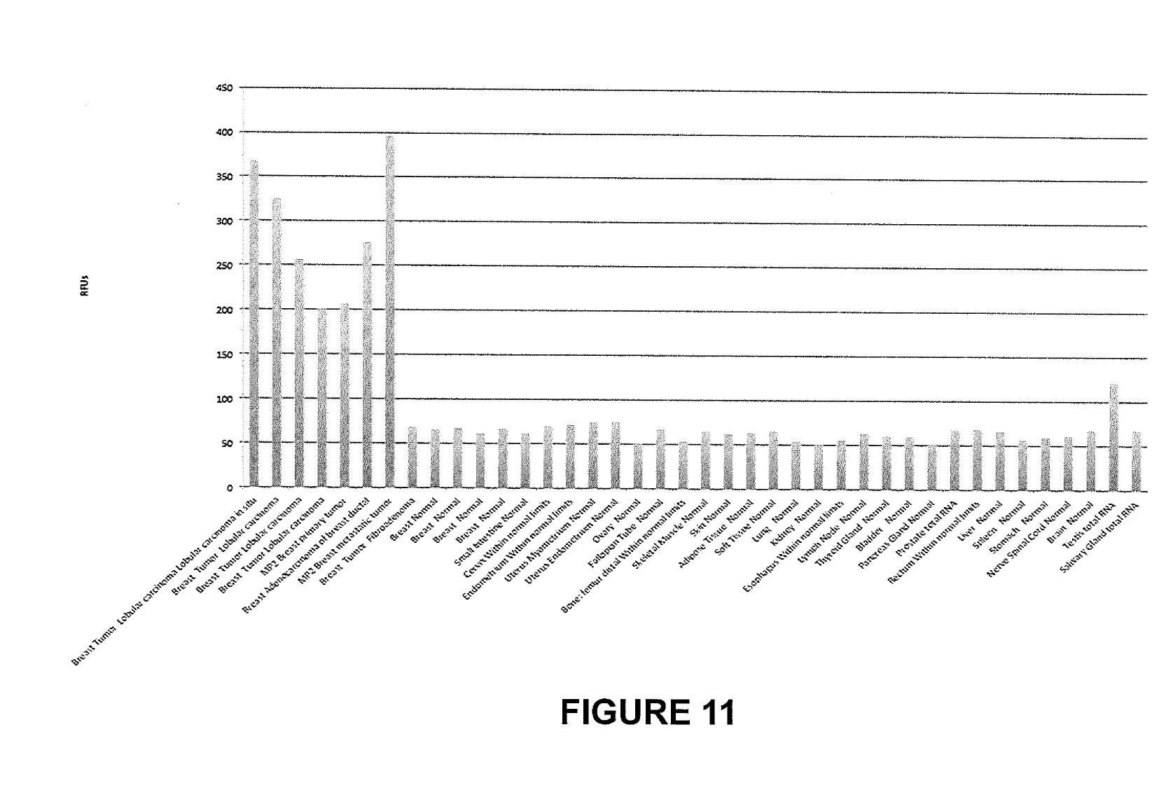

LOC727941 (Accession number XR_{—}037165.1) encodes an uncharacterized transcript similar to mitochondrial Ca2+-dependent solute carrier. Surprisingly, it is disclosed here that LOC727941 is a novel marker for breast tumors. As shown in FIG. 11, LOC727941 expression was assayed by Illumina microarray, a probe specific for LOC727941 (probe sequence GTGAACCTTATTAGAACTCGCATGCAGGCTTCAGCCCCAGTGGAAAAAGG (SEQ ID NO: 105); Illumina probe ID ILMN_{—}3201563) detected strong gene expression (>150 RFUs) in breast tumor lobular carcinoma and breast infiltrating ductal carcinoma. In contrast, expression of LOC727941 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<100 RFUs). The specificity of elevated LOC727941 expression in malignant tumors of breast origin shown herein demonstrates that LOC727941 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target LOC727941 can be identified using the methods described herein and therapeutics that target LOC727941 include, but are not limited to, antibodies that modulate the activity of LOC727941. The manufacture and use of antibodies are described herein.

### Example 18

### NAT1

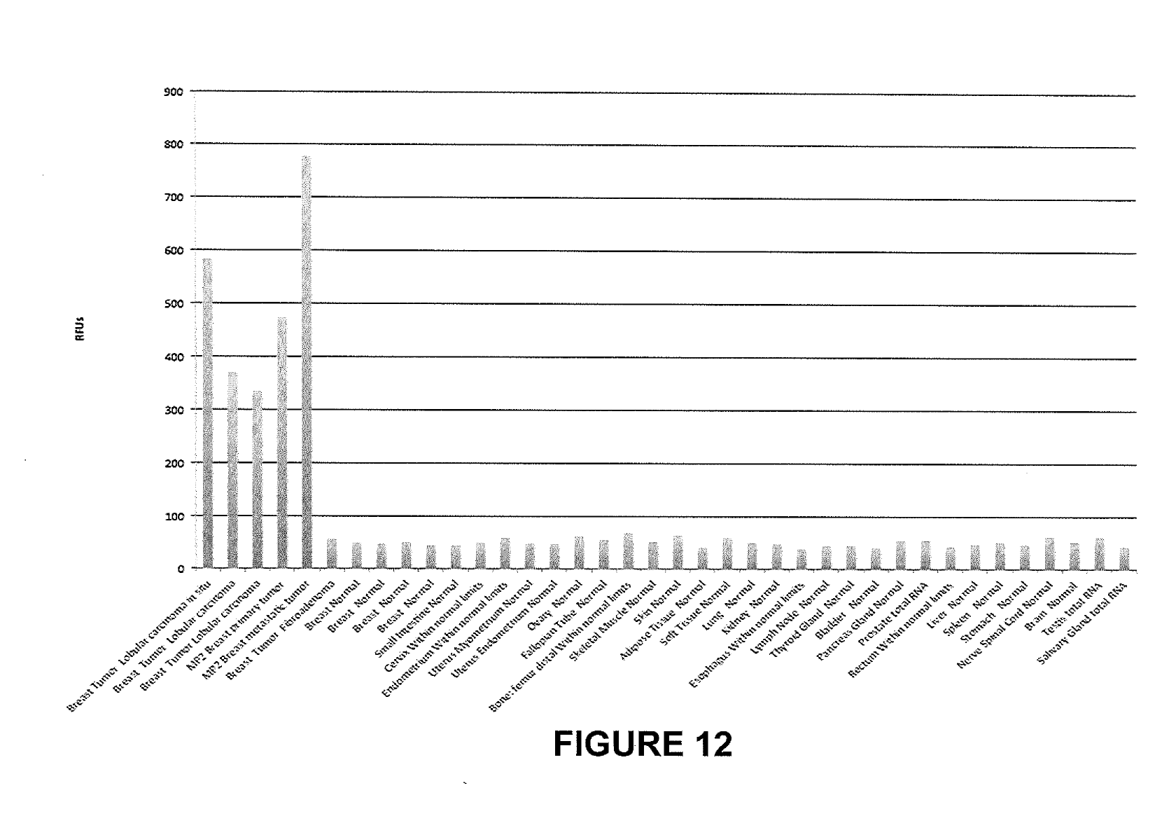

NAT1 (Accession number NM_{—}000662.4) encodes Homo sapiens N-acetyltransferase 1 (arylamine N-acetyltransferase). Surprisingly, it is disclosed here that NAT1 is a novel marker for breast tumors. As shown in FIG. 12, NAT1 expression was assayed by Illumina microarray, a probe specific for NAT1 (probe sequence GCCGGCTGAAATAACCTGAATTCAAGCCAGGAAGAAGCAGCAATCTGTCT (SEQ ID NO: 106); Illumina probe ID ILMN_{—}1743055) detected strong gene expression (>70 RFUs) in breast tumor lobular carcinoma and breast infiltrating ductal carcinoma. In contrast, expression of NAT1 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<70 RFUs). The specificity of elevated NAT1 expression in malignant tumors of breast origin shown herein demonstrates that NAT1 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target NAT1 can be identified using the methods described herein and therapeutics that target NAT1 include, but are not limited to, antibodies that modulate the activity of NAT1. The manufacture and use of antibodies are described herein.

### Example 19

### NXPH1

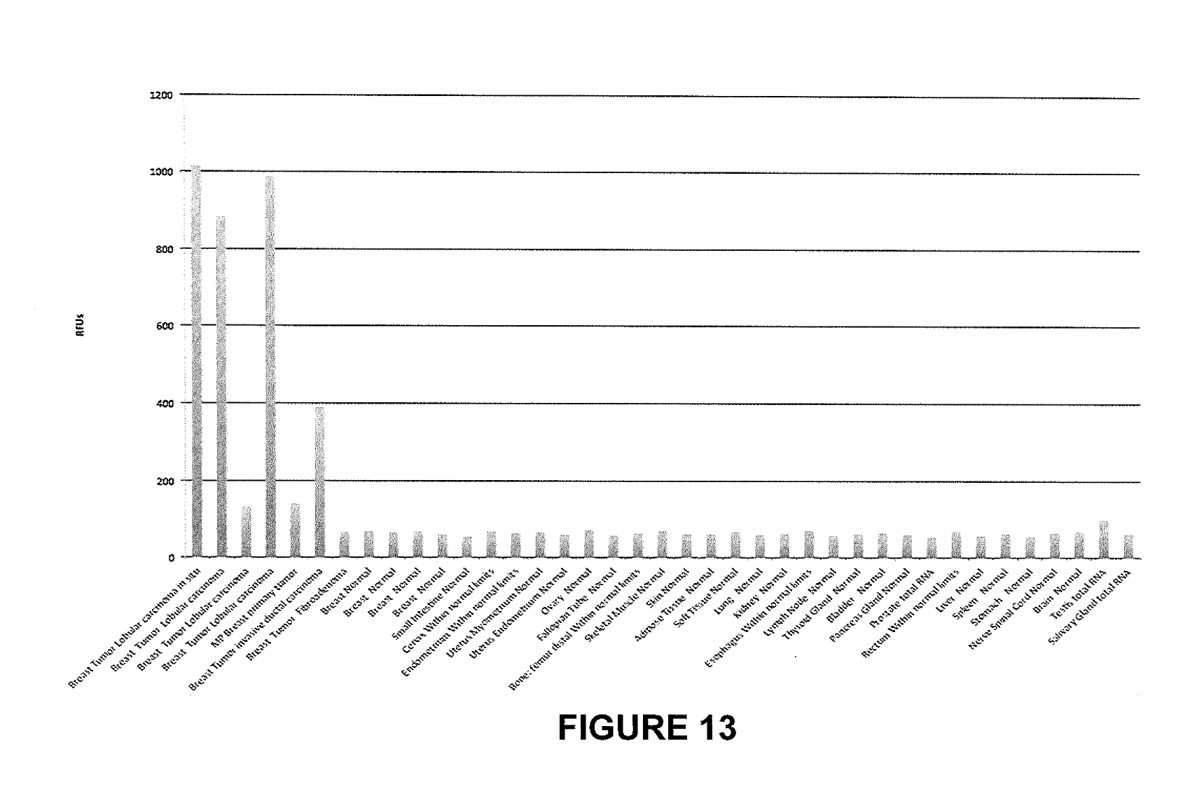

NXPH1 (Accession number NM_{—}152745.2) encodes Homo sapiens neurexophilin 1. Surprisingly, it is disclosed here that NXPH1 is a novel marker for breast tumors. As shown in FIG. 13, NXPH1 expression was assayed by Illumina microarray, a probe specific for NXPH1 (probe sequence CAAAGTGGTCCAAGATGGCTCTTTTTTCTTTGAAAGGGGCCTGTTCTCAG (SEQ ID NO: 107); Illumina probe ID ILMN_{—}1764271) detected strong gene expression (>299 RFUs) in breast tumor lobular carcinoma and breast infiltrating ductal carcinoma. In contrast, expression of NXPH1 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<299 RFUs). The specificity of elevated NXPH1 expression in malignant tumors of breast origin shown herein demonstrates that NXPH1 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target NXPH1 can be identified using the methods described herein and therapeutics that target NXPH1 include, but are not limited to, antibodies that modulate the activity of NXPH1. The manufacture and use of antibodies are described herein.

### Example 20

### SERHL2

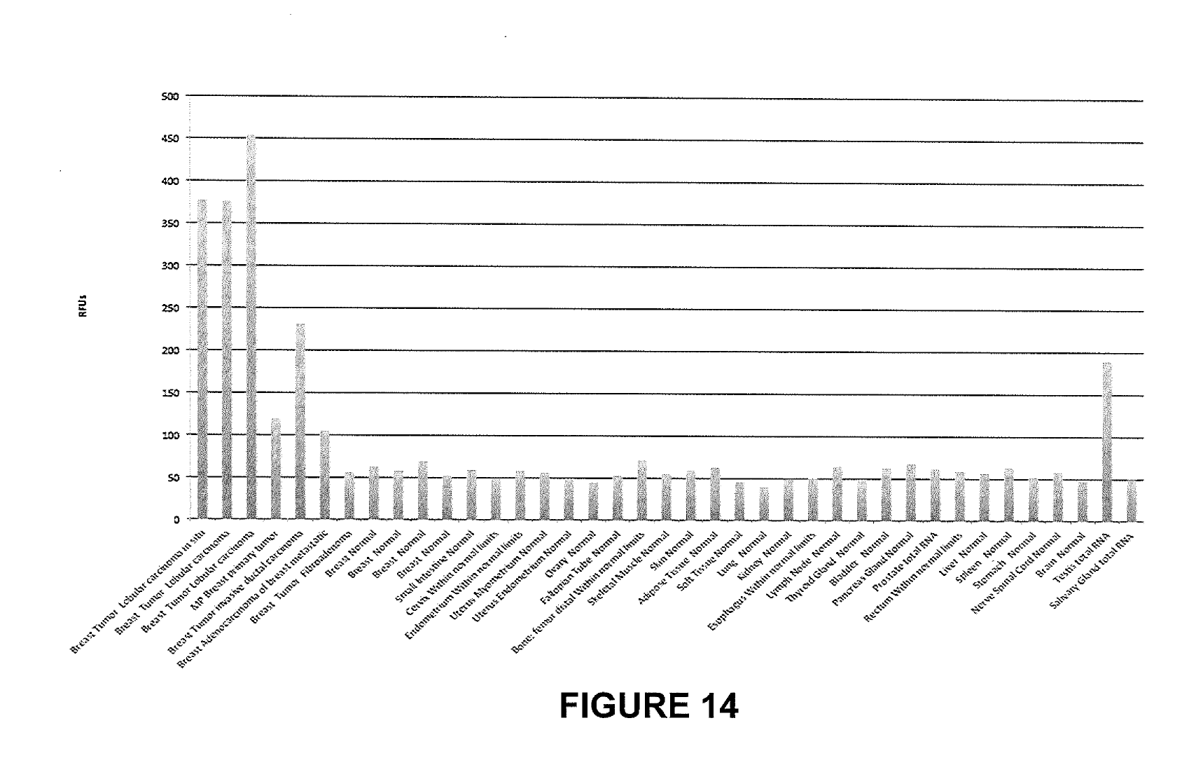

SERHL2 (Accession number NM_{—}014509.3) encodes Homo sapiens serine hydrolase-like 2. Surprisingly, it is disclosed here that SERHL2 is a novel marker for breast tumors. As shown in FIG. 14, SERHL2 expression was assayed by Illumina microarray, a probe specific for SERHL2 (probe sequence CATGATAGACACGATGAAATCCACCCTCAAAGAG CAGTTCCAGTTTGTGG (SEQ ID NO: 108); Illumina probe ID ILMN_{—}2231299) detected strong gene expression (>145 RFUs) in breast tumor lobular carcinoma, breast infiltrating ductal carcinoma and metastatic breast tumor. In contrast, expression of SERHL2 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<145 RFUs). The specificity of elevated SERHL2 expression in malignant tumors of breast origin shown herein demonstrates that SERHL2 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target SERHL2 can be identified using the methods described herein and therapeutics that target SERHL2 include, but are not limited to, antibodies that modulate the activity of SERHL2. The manufacture and use of antibodies are described herein.

### Example 21

### SYCP2

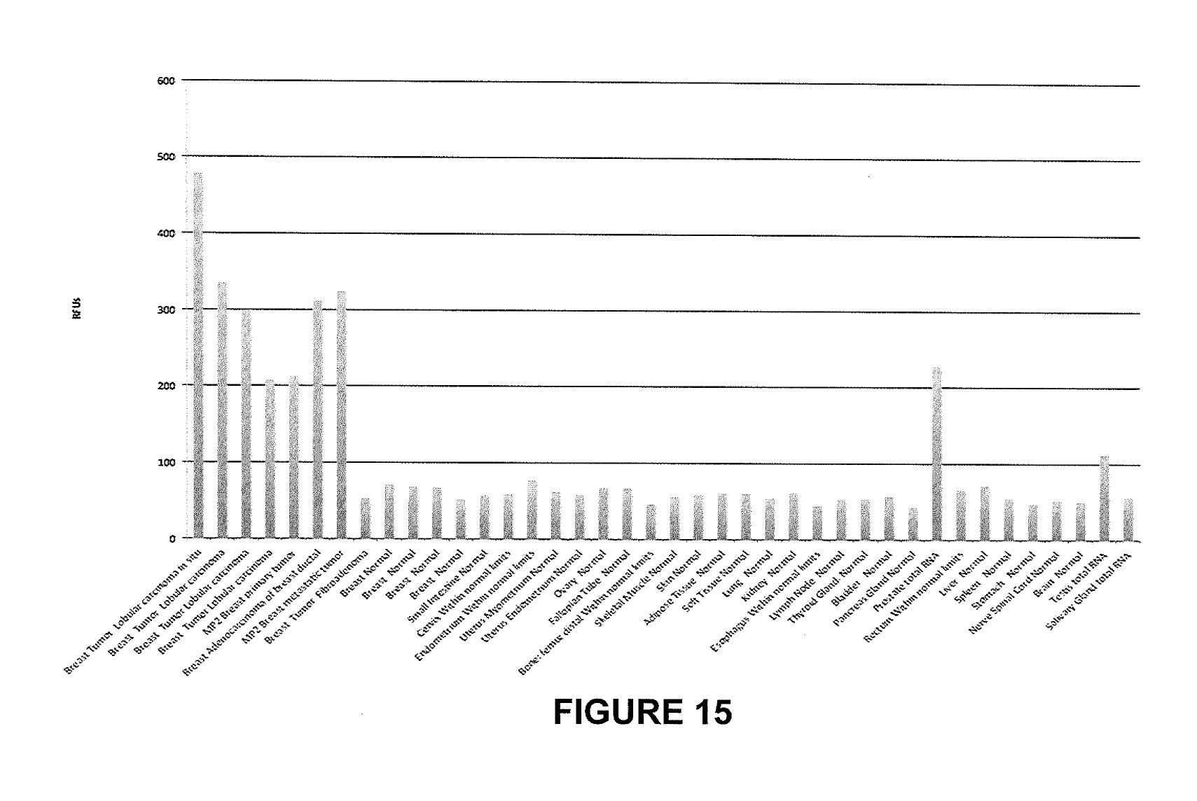

SYCP2 (Accession number NM_{—}014258.2) encodes Homo sapiens synaptonemal complex protein 2. Surprisingly, it is disclosed here that SYCP2 is a novel marker for breast tumors. As shown in FIG. 15, SYCP2 expression was assayed by Illumina microarray, a probe specific for SYCP2 (probe sequence GGATGAGAGGGAACCACTATAACATGAGTCCAAGCCCAGAAGACTTCTGT (SEQ ID NO: 109); Illumina probe ID ILMN_{—}2095704) detected strong gene expression (>154 RFUs) in breast tumor lobular carcinoma, breast infiltrating ductal carcinoma and metastatic breast tumor. In contrast, expression of SYCP2 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<154 RFUs). The specificity of elevated SYCP2 expression in malignant tumors of breast origin shown herein demonstrates that SYCP2 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target SYCP2 can be identified using the methods described herein and therapeutics that target SYCP2 include, but are not limited to, antibodies that modulate the activity of SYCP2. The manufacture and use of antibodies are described herein.

### Example 22

### DS9687

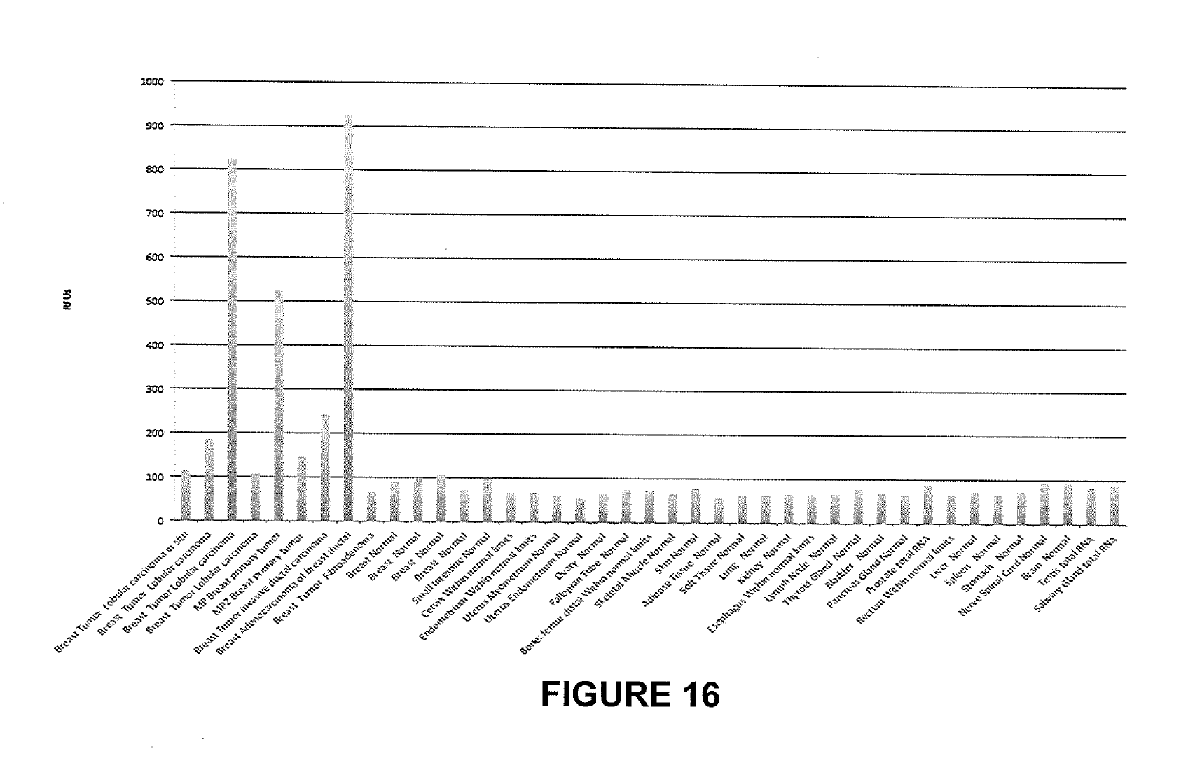

Accession number DS9687 encodes D59687 Clontech human fetal brain polyA+ mRNA (#6535) Homo sapiens cDNA clone GEN-056E10 5, mRNA sequence. Surprisingly, it is disclosed here that DS9687 is a novel marker for breast tumors. As shown in FIG. 16, DS9687 expression was assayed by Illumina microarray, a probe specific for DS9687 (probe sequence CCTCCACCCTACAAGGTGTGTGCTTGTAACTCAAATTTCCATTTGAGTAA (SEQ ID NO: 109); Illumina probe ID ILMN_{—}1840294) detected strong gene expression (>100 RFUs) in breast tumor lobular carcinoma, breast primary tumor (infiltrating ductal carcinoma) and metastatic breast tumor. In contrast, expression of DS9687 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<100 RFUs). The specificity of elevated DS9687 expression in malignant tumors of breast origin shown herein demonstrates that DS9687 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target DS9687 can be identified using the methods described herein and therapeutics that target DS9687 include, but are not limited to, antibodies that modulate the activity of DS9687. The manufacture and use of antibodies are described herein.

### Example 23

### CYP4Z1

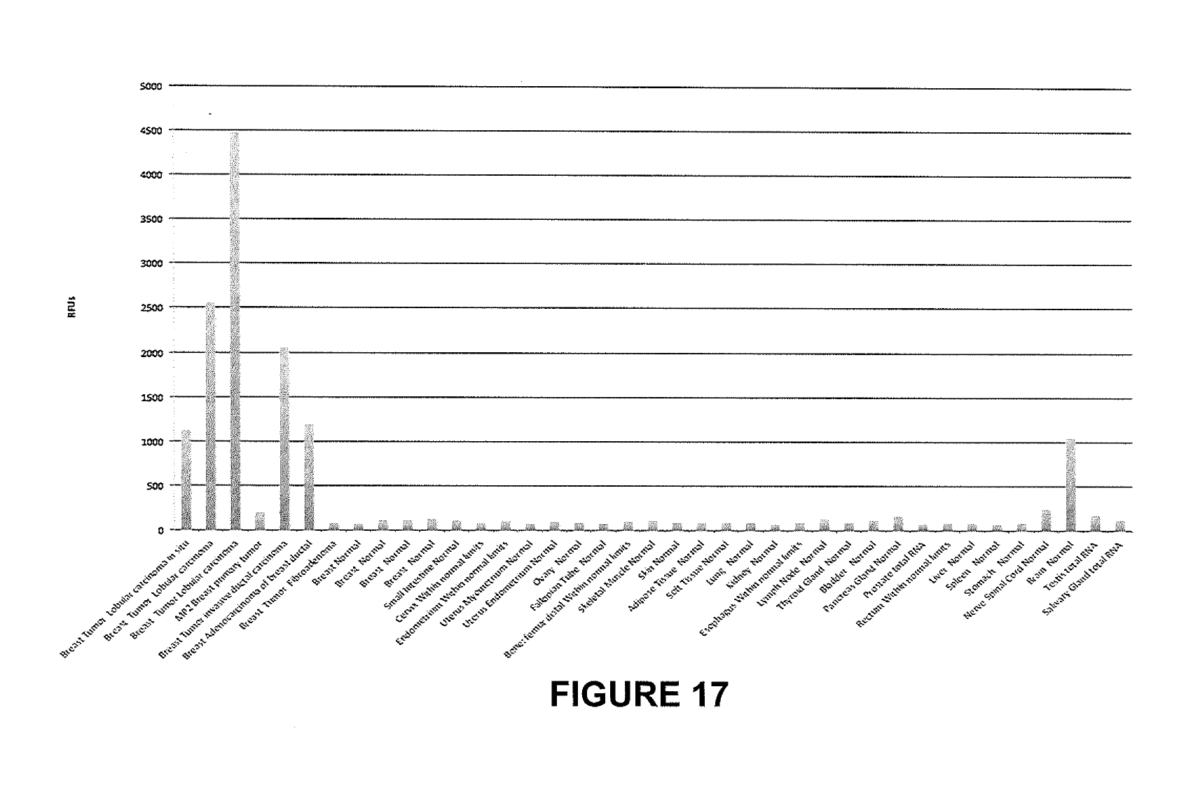

CYP4Z1 (Accession number NM_{—}178134.2) encodes Homo sapiens cytochrome P450, family 4, subfamily Z, polypeptide 1. Surprisingly, it is disclosed here that CYP4Z1 is a novel marker for breast tumors. As shown in FIG. 17, CYP4Z1 expression was assayed by Illumina microarray, a probe specific for CYP4Z1 (probe sequence CACCACGATGT GCATCAAGGAATGCCTCCGCCTCTACGCACCGGTAGTAA (SEQ ID NO: 110); Illumina probe ID ILMN_{—}2359698) detected strong gene expression (>100 RFUs) in breast tumor lobular carcinoma, breast infiltrating ductal carcinoma and metastatic breast tumor. In contrast, expression of CYP4Z1 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<100 RFUs). The specificity of elevated CYP4Z1 expression in malignant tumors of breast origin shown herein demonstrates that CYP4Z1 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target CYP4Z1 can be identified using the methods described herein and therapeutics that target CYP4Z1 include, but are not limited to, antibodies that modulate the activity of CYP4Z1. The manufacture and use of antibodies are described herein.

### Example 24

### LOC730024

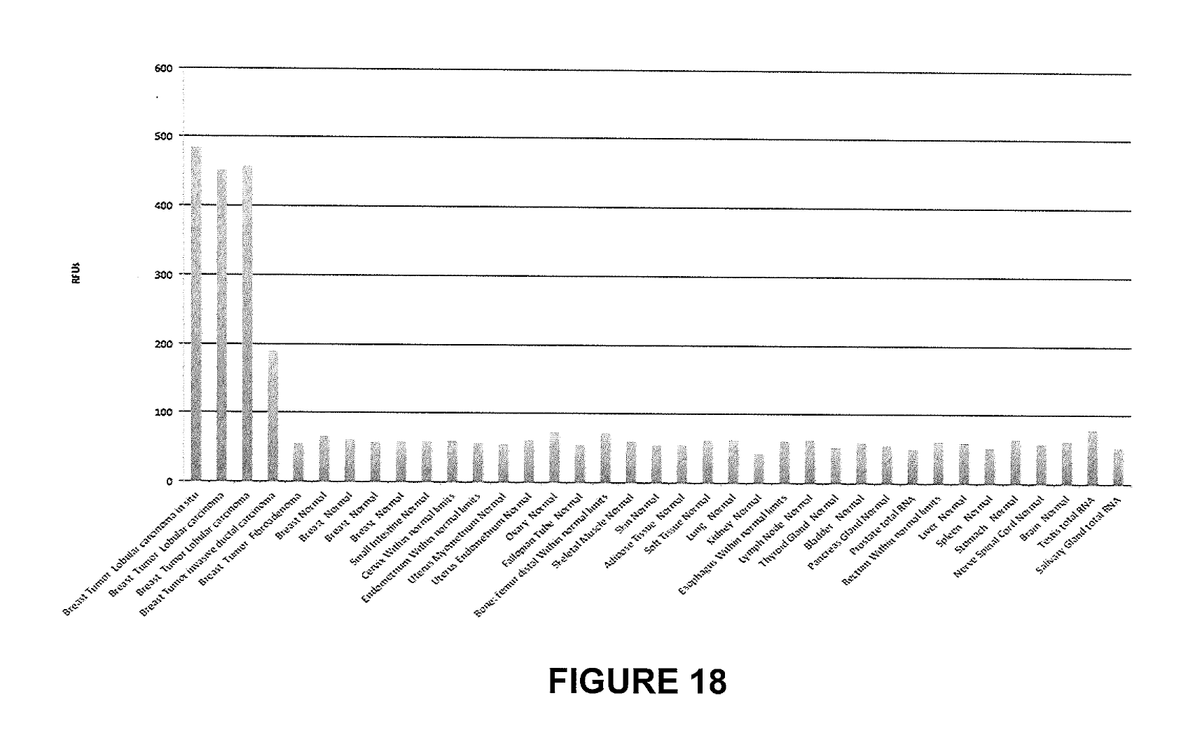

LOC730024 (Accession number XR_{—}015755.1) encodes Homo sapiens similar to male sterility domain containing I. Surprisingly, it is disclosed here that LOC730024 is a novel marker for breast tumors. As shown in FIG. 18, LOC730024 expression was assayed by Illumina microarray, a probe specific for LOC730024 (probe sequence GCTGAGT CAATGGCTTTGAGAATGTCACTGCATATGGGAGATTGAGGCCC (SEQ ID NO: 111); IIlumina probe ID ILMN_{—}1674747) detected strong gene expression (>105 RFUs) in breast tumor lobular carcinoma. In contrast, expression of LOC730024 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<105 RFUs). The specificity of elevated LOC730024 expression in malignant tumors of breast origin shown herein demonstrates that LOC730024 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target LOC730024 can be identified using the methods described herein and therapeutics that target LOC730024 include, but are not limited to, antibodies that modulate the activity of LOC730024. The manufacture and use of antibodies are described herein.

### Example 25

### NOS1AP

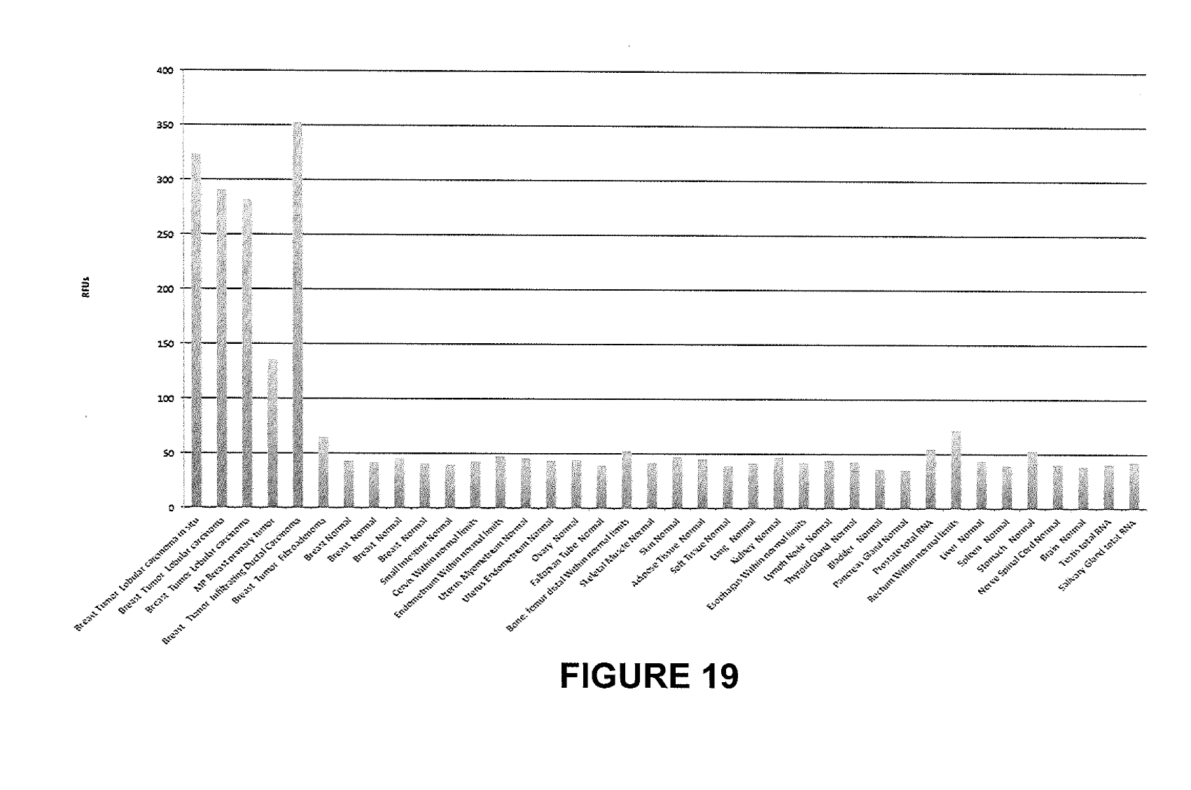

NOS1AP (Accession number NM_{—}014697.1) encodes Homo sapiens nitric oxide synthase 1 (neuronal) adapter protein. Surprisingly, it is disclosed here that NOS1AP is a novel marker for breast tumors. As shown in FIG. 19, NOS1AP expression was assayed by Illumina micromay, a probe specific for NOS1AP (probe sequence CTTTTGCAGCACTTTACCTCTCTGAAAGCCCCAGAGGACCAGAGCCCCC (SEQ ID NO: 112); Illumina probe ID ILMN_{—}1710315) detected strong gene expression (>100 RFUs) in breast tumor lobular carcinoma, breast infiltrating ductal carcinoma and metastatic breast tumor. In contrast, expression of NOS1AP in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, testis, liver, thyroid, and salivary gland was generally low (<100 RFUs) with the exception of brain (165 RFUs). The specificity of elevated NOS1AP expression in malignant tumors of breast origin shown herein demonstrates that NOS1AP is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target NOS1AP can be identified using the methods described herein and therapeutics that target NOS1AP include, but are not limited to, antibodies that modulate the activity of NOS1AP. The manufacture and use of antibodies are described herein.

### Example 26

### UGT2B28

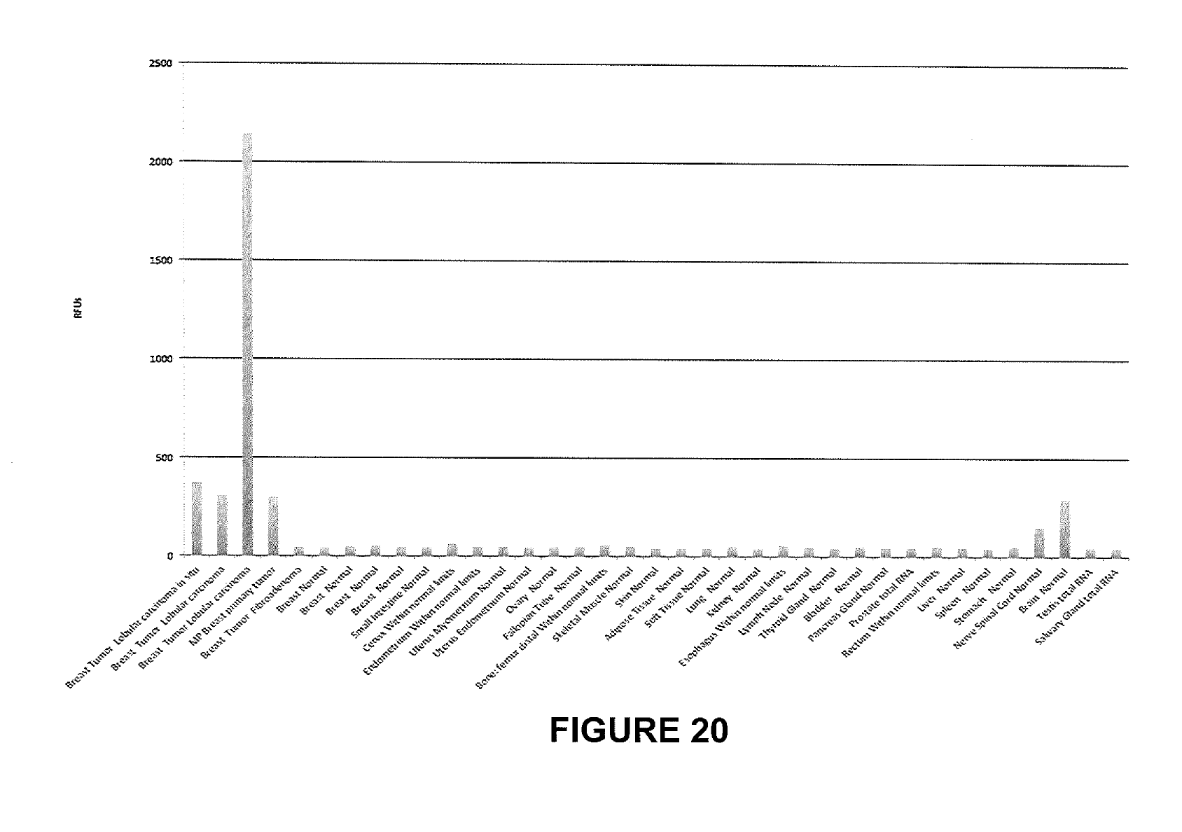

UGT2B28 (Accession number NM_{—}053039.1) encodes Homo sapiens UDP glucuronosyltransferase 2 family, polypeptide B28. Surprisingly, it is disclosed here that UGT2B28 is a novel marker for breast tumors. As shown in FIG. 20, UGT2B28 expression was assayed by Illumina microarray, a probe specific for UGT2B28 (probe sequence GTGATGTGCCACAAAGGAGCCAAACACCTTCGAGTTGC AGCCCGTGACCT (SEQ ID NO: 113); Illumina probe ID ILMN_{—}1781859) detected strong gene expression (>220 RFUs) in breast tumor lobular carcinoma, breast infiltrating ductal carcinoma and metastatic breast tumor. In contrast, expression of UGT2B28 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<220 RFUs). The specificity of elevated UGT2B28 expression in malignant tumors of breast origin shown herein demonstrates that UGT2B28 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target UGT2B28 can be identified using the methods described herein and therapeutics that target UGT2B28 include, but are not limited to, antibodies that modulate the activity of UGT2B28. The manufacture and use of antibodies are described herein.

### Example 27

### GRM4

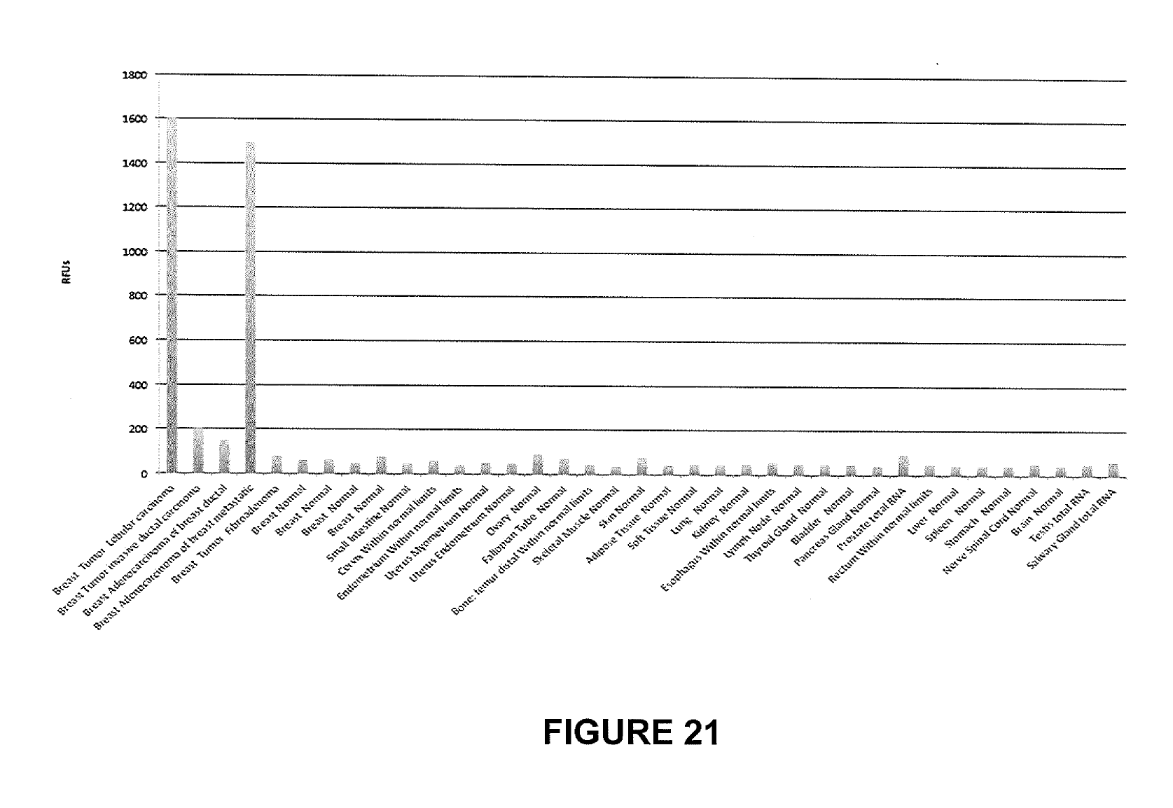

GRM4 (Accession number NM_{—}000841.1) encodes Homo sapiens glutamate receptor, metabotropic 4. Surprisingly, it is disclosed here that GRM4 is a novel marker for breast tumors. As shown in FIG. 21, GRM4 expression was assayed by Illumina microarray, a probe specific for GRM4 (probe sequence TCGAGTGTGTTGCCAAGTGCTGCGTCCTCCTG GTGGCCTCTGTGTGTGTC (SEQ ID NO: 114); Illumina probe ID ILMN_{—}1752843) detected strong gene expression (>100 RFUs) in breast tumor lobular carcinoma, breast infiltrating ductal carcinoma and metastatic breast tumor. In contrast, expression of GRM4 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<100 RFUs). The specificity of elevated GRM4 expression in malignant tumors of breast origin shown herein demonstrates that GRM4 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target GRM4 can be identified using the methods described herein and therapeutics that target GRM4 include, but are not limited to, antibodies that modulate the activity of GRM4. The manufacture and use of antibodies are described herein.

### Example 28

### FLJ30428

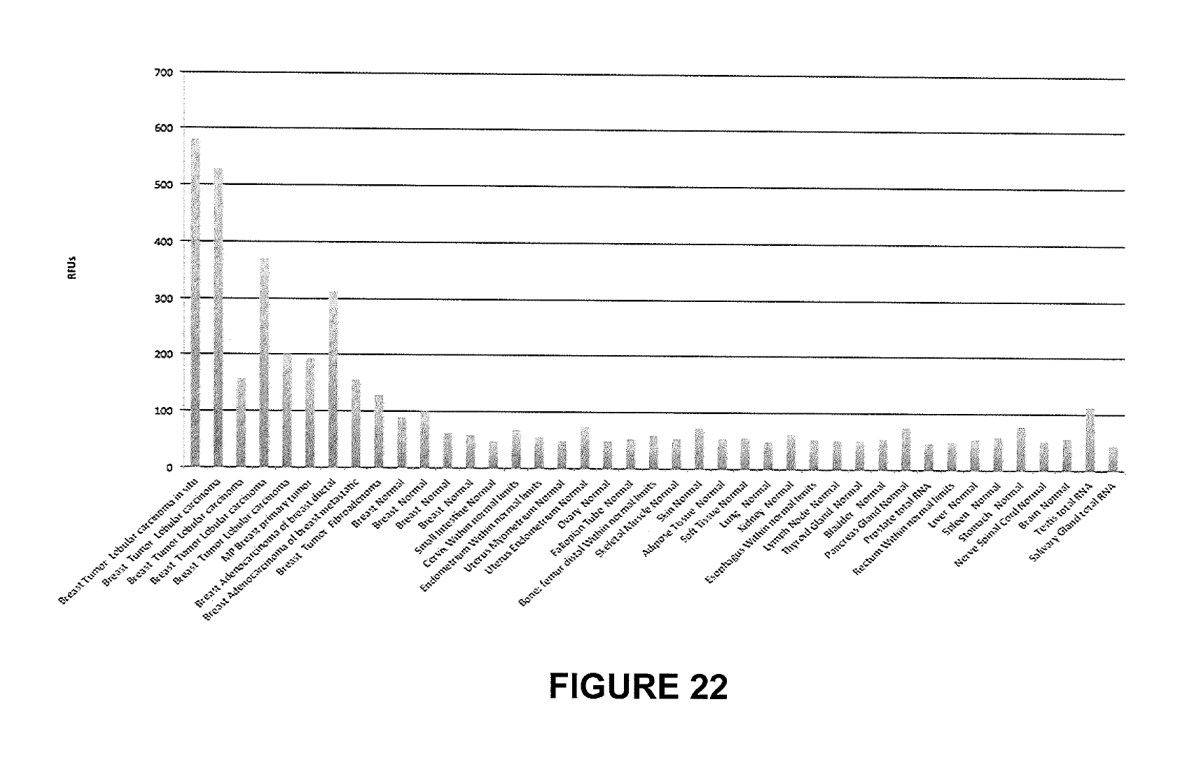

FLJ30428 (Accession number XM_{—}496597.4) encodes Homo sapiens similar to hypothetical protein A230046P18; cDNA sequence BC055759. Surprisingly, it is disclosed here that FLJ30428 is a novel marker for breast tumors. As shown in FIG. 22, FLJ30428 expression was assayed by Illumina microarray, a probe specific for FLJ30428 (probe sequence TTGGGACACTAACCATCCAGGTCAAGAAAAGTCACATGCCATAGCCATGG (SEQ ID NO: 115); Illumina probe ID ILMN_{—}3184067) detected strong gene expression (>305 RFUs) in breast tumor lobular carcinoma and breast infiltrating ductal carcinoma. In contrast, expression of FLJ30428 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<305 RFUs). The specificity of elevated FLJ30428 expression in malignant tumors of breast origin shown herein demonstrates that FLJ30428 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target FLJ30428 can be identified using the methods described herein and therapeutics that target FLJ30428 include, but are not limited to, antibodies that modulate the activity of FLJ30428. The manufacture and use of antibodies are described herein.

### Example 29

### LOC440905

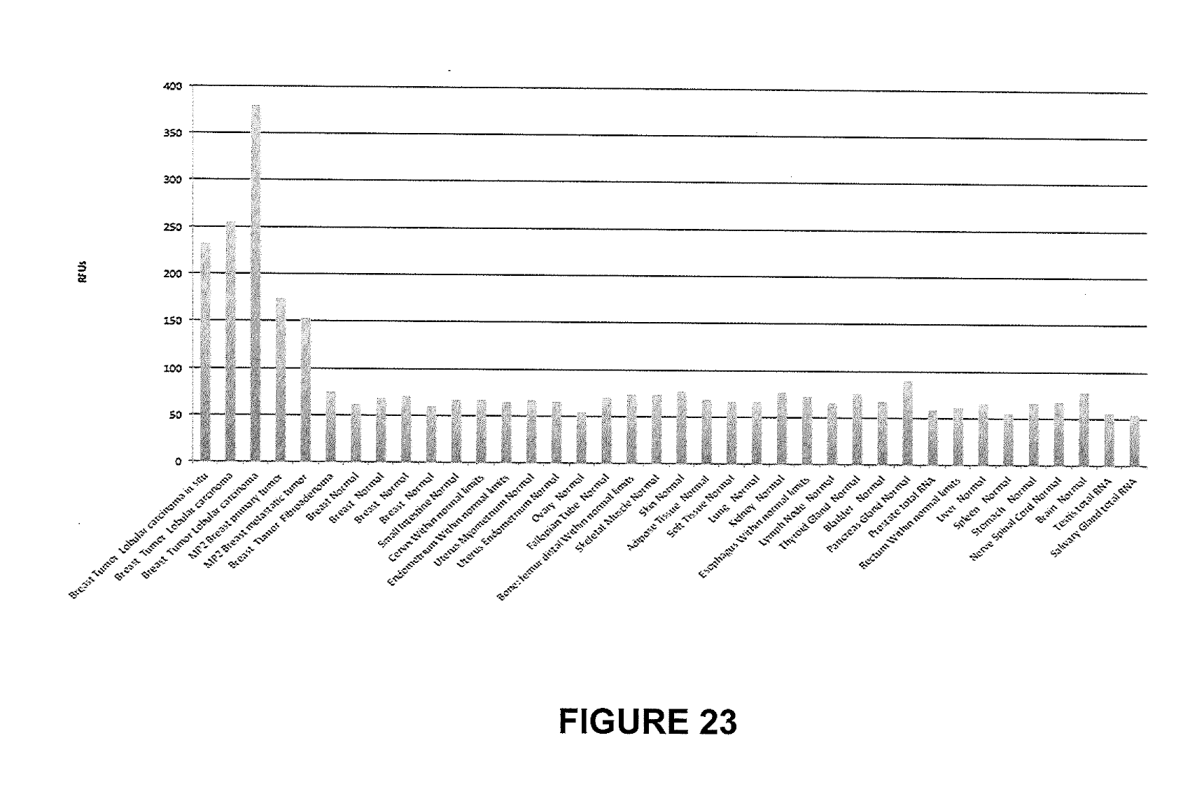

LOC440905 (Accession number NM_{—}001013711.1) encodes Homo sapiens hypothetical protein LOC440905. Surprisingly, it is disclosed here that LOC440905 is a novel marker for breast tumors. As shown in FIG. 23, LOC440905 expression was assayed by Illumina microarray, a probe specific for LOC440905 (probe sequence TGTGATC AAGACTCAGAAGCACGAACAGTATTGCCCTCTGTGTTAGCCCC (SEQ ID NO: 116); Illumina probe ID ILMN_{—}1677764) detected strong gene expression (>200 RFUs) in breast tumor lobular carcinoma. In contrast, expression of LOC440905 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, liver, thyroid, and salivary gland was generally low (<200 RFUs) with the exception of testis (238 RFUs). The specificity of elevated LOC440905 expression in malignant tumors of breast origin shown herein demonstrates that LOC440905 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target LOC440905 can be identified using the methods described herein and therapeutics that target LOC440905 include, but are not limited to, antibodies that modulate the activity of LOC440905. The manufacture and use of antibodies are described herein.

### Example 30

### LOC642460

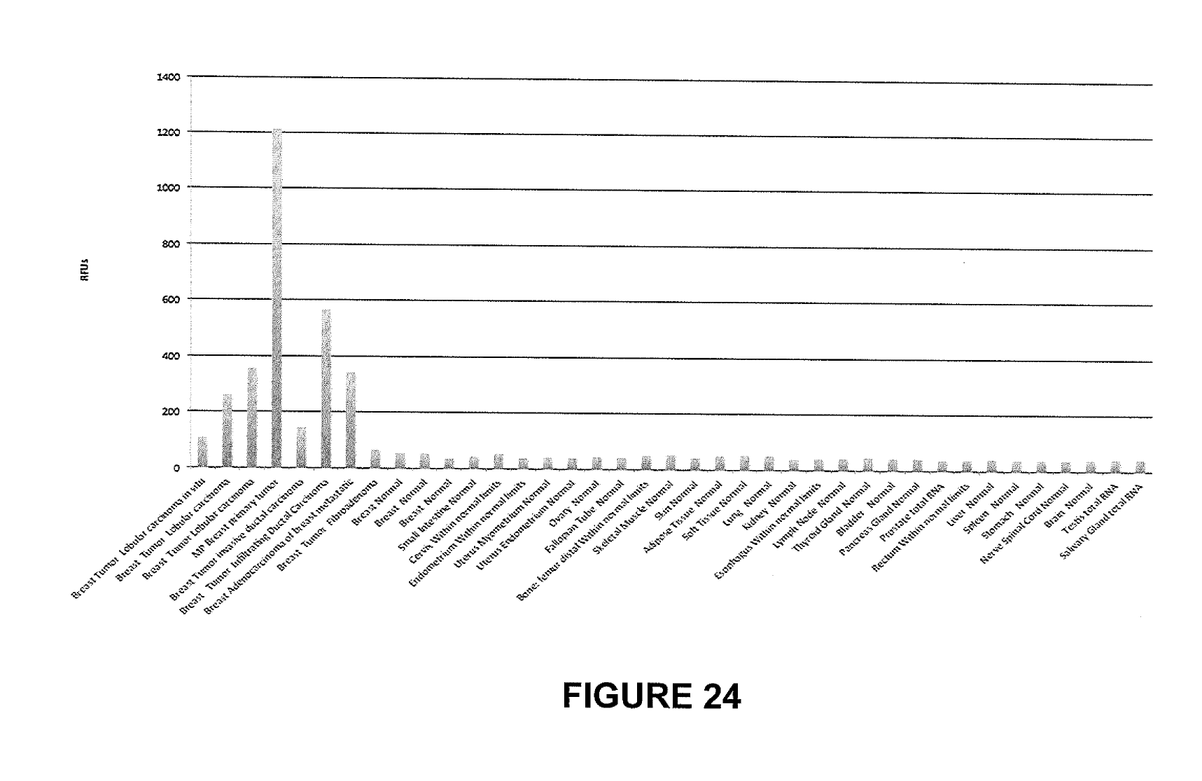

LOC642460 (Accession number XR_{—}016169.1) encodes Homo sapiens similar to ankyrin repeat domain 30A. Surprisingly, it is disclosed here that LOC642460 is a novel marker for breast tumors. As shown in FIG. 24, LOC642460 expression was assayed by Illumina microarray, a probe specific for LOC642460 (probe sequence AAGCCTACCTGTGGAAGGAAAGTTTCTCTTCCAAATAAAGCCTTA GAATT (SEQ ID NO: 117); Illumina probe ID ILMN_{—}1672000) detected strong gene expression (>120 RFUs) in breast tumor lobular carcinoma, breast infiltrating ductal carcinoma and metastatic breast tumor. In contrast, expression of LOC642460 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<120 RFUs). The specificity of elevated LOC642460 expression in malignant tumors of breast origin shown herein demonstrates that LOC642460 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target LOC642460 can be identified using the methods described herein and therapeutics that target LOC642460 include, but are not limited to, antibodies that modulate the activity of LOC642460. The manufacture and use of antibodies are described herein.

### Example 31

### MTL5

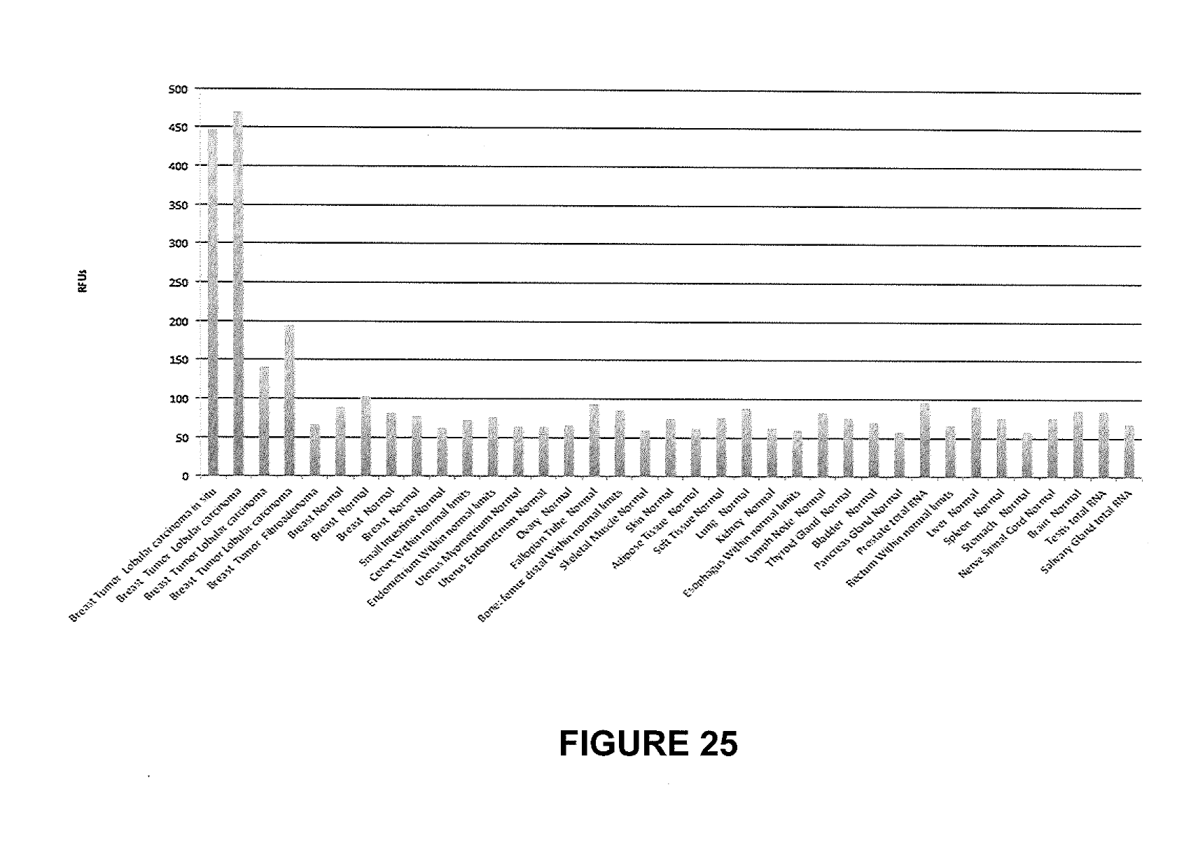

MTL5 (Accession number NM_{—}004923.3) encodes Homo sapiens metallothionein-like 5, testis-specific (tesmin). Surprisingly, it is disclosed here that MTL5 is a novel marker for breast tumors. As shown in FIG. 25, MTL5 expression was assayed by Illumina microarray, a probe specific for MTL5 (probe sequence AGATATTTCCCCAGAGGCACGCGAACTGTCAGTCTTTCCTAAGGCCCCCG (SEQ ID NO: 118); Illumina probe ID ILMN_{—}1661778) detected strong gene expression (>100 RFUs) in breast tumor lobular carcinoma, breast infiltrating ductal carcinoma and metastatic breast tumor. In contrast, expression of MTL5 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, liver, thyroid, and salivary gland was generally low (<100 RFUs), with the exception of testis (569 RFUs). The specificity of elevated MTL5 expression in malignant tumors of breast origin shown herein demonstrates that MTL5 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target MTL5 can be identified using the methods described herein and therapeutics that target MTL5 include, but are not limited to, antibodies that modulate the activity of MTL5. The manufacture and use of antibodies are described herein.

### Example 32

### GRPR

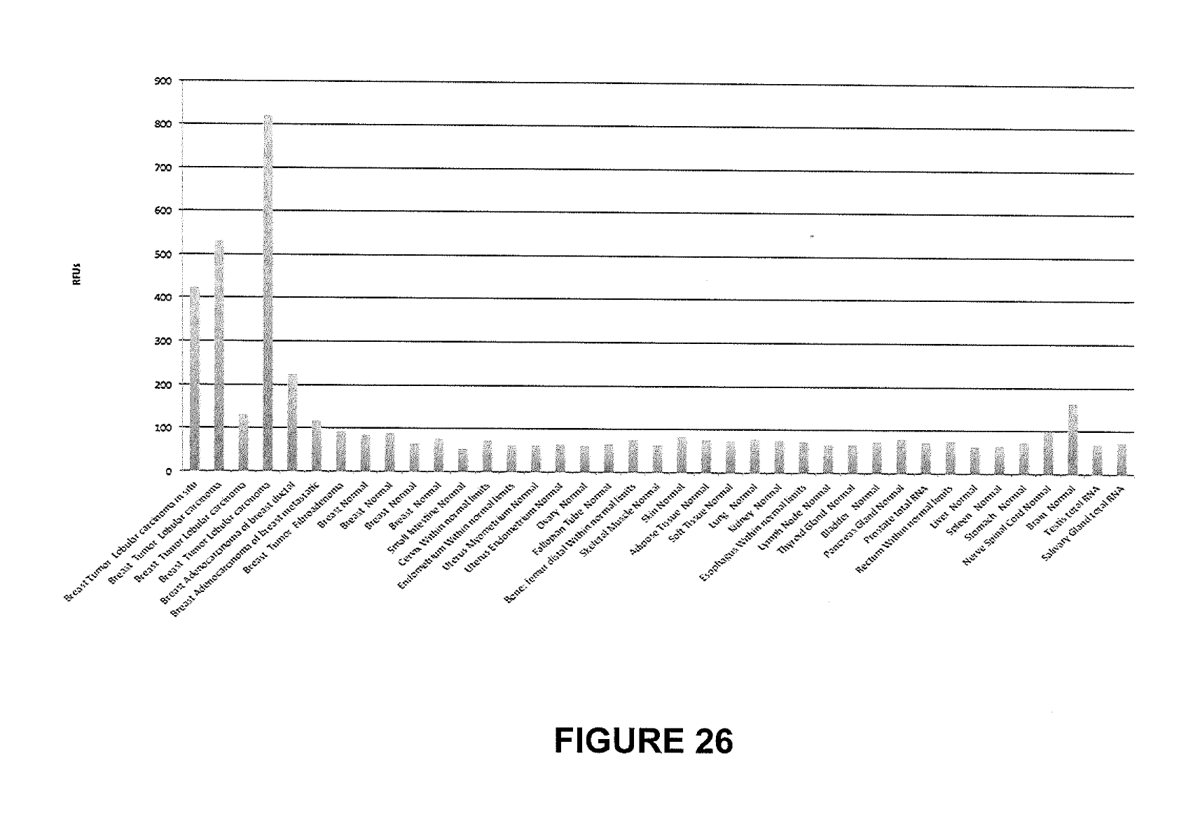

GRPR (Accession number NM_{—}005314.2) encodes Homo sapiens gastrin-releasing peptide receptor. Surprisingly, it is disclosed here that GRPR is a novel marker for breast tumors. As shown in FIG. 26, GRPR expression was assayed by Illumina microarray, a probe specific for GRPR (probe sequence GGAGGTTTTGTTTGCTGCTACGGTTTTAATCAT CCAGGGTGCCATTCCAC (SEQ ID NO: 119); Illumina probe ID ILMN_{—}2119123) detected strong gene expression (>140 RFUs) in breast tumor lobular carcinoma, breast primary tumor (infiltrating ductal carcinoma) and metastatic breast tumor. In contrast, expression of GRPR in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, prostate, rectum, spleen, stomach, spinal cord, brain, testis, liver, thyroid, and salivary gland was generally low (<140 RFUs), with the exception of pancreas (396 RFUs). The specificity of elevated GRPR expression in malignant tumors of breast origin shown herein demonstrates that GRPR is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target GRPR can be identified using the methods described herein and therapeutics that target GRPR include, but are not limited to, antibodies that modulate the activity of GRPR. The manufacture and use of antibodies are described herein.

### Example 33

### COL10A1

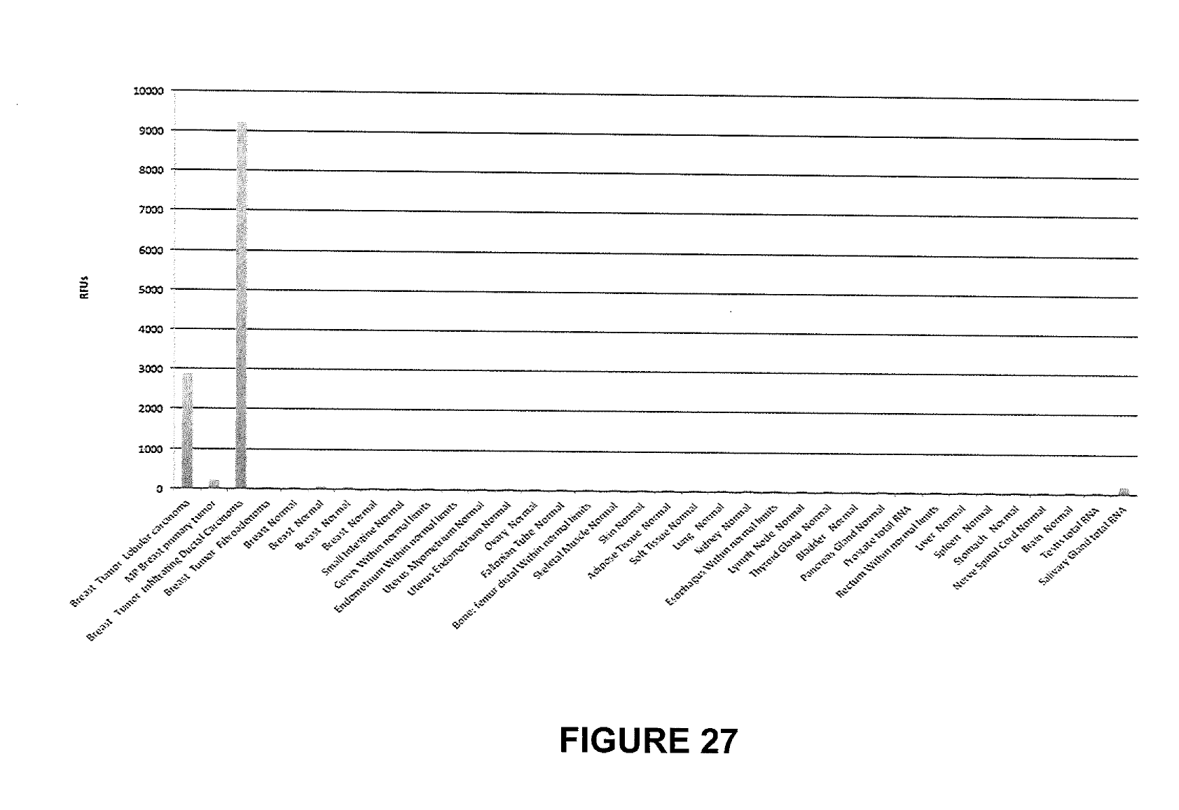

COL10A1 (Accession number NM_{—}000493.3) encodes Homo sapiens collagen, type X, alpha 1. Surprisingly, it is disclosed here that COL10A1 is a novel marker for breast tumors. As shown in FIG. 27, COL10A1 expression was assayed by Illumina microarray, a probe specific for COL10A1 (probe sequence CCCCTAAAATATTTCTGATGGTGCACTACTCTGAGGCCTGTATGGCCCCT (SEQ ID NO: 120); Illumina probe IDILMN_{—}1672776) detected strong gene expression (>100 RFUs) in breast tumor lobular carcinoma, breast infiltrating ductal carcinoma and metastatic breast tumor. In contrast, expression of COL10A1 in a wide variety of normal tissues including normal breast, colon, rectum, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, spleen, stomach, spinal cord, brain, testis, liver, thyroid, and salivary gland was generally low (<100 RFUs), with the exception of bone (487 RFUs). The specificity of elevated COL10A1 expression in malignant tumors of breast origin shown herein demonstrates that COL 10A 1 is a marker for the diagnosis of breast cancer (e.g. including but not limited to, breast tumor lobular carcinoma, breast infiltrating ductal carcinoma and metastatic breast tumors), and is a target for therapeutic intervention in breast cancer.

Therapeutics that target COL10A1 can be identified using the methods described herein and therapeutics that target COL10A1 include, but are not limited to, antibodies that modulate the activity of COL10A 1. The manufacture and use of antibodies are described herein.

### Example 34

### VCR Analysis of Tissue Samples for Cancer Markers

qPCR was performed on breast tumors of different stages. Normal breast tissue served as a negative control. Positive controls were specific known tumors previously assayed by microarray. Additionally, tissue adjacent to the tumor site iii patients was also analyzed. Expression of the following genes was investigated: ASCL1, BX116033, C1orf64; COL10A1; DSCR6; FLJ23152; GRM4; TMEM145_{—}1101; POTEG; AND FSIP.

Total RNA was extracted with the RNeasy Mini Kit (Qiagen) and cDNA generated using the SuperScript III reverse transcriptase in combination with random hexamer primers alone or in combination with oligo-dT primers (all reverse transcription components from Invitrogen/Life Technologies). PCRs were carried out on a 7900HT Sequence Detection System or a 7500 Real Time PCR System (Applied Biosystems/Life Technologies) utilizing SYBR Green or TaqMan chemistries. The primers used for the PCR reactions are listed in Tables 7 and 8. PCR parameters were: activation at 50° C. for 2 minutes; denature at 95° C. for 10 minutes; followed by 40-42 cycles of 95° C. for 15 seconds and 60° C. for 1 minute (72° C. for amplicons >than 120 bp) followed by dissociation at 95° C. for 15 seconds; 60° C. for 15 seconds, and 95° C. for 15 seconds.

Primers are provided in the Table below:

| Gene Marker | Forward Primer | Forward Primer Sequence |
|---|---|---|
| ASCL1 | JK1095-ASCL1-F | AATGGACTTTGGAAGCAGGGTGATC |
| | | (SEQ ID NO: 121) |
| BX116033 | JK1107-BTX116-F | GAAATAATTTTTCCCTCTCAAGCTCTAAGTC |
| | | (SEQ ID NO: 122) |
| C1ORF64 | JK1089-C1orf64-F | AGACCAGCTCCGGTGGGAAGC |
| | | (SEQ ID NO: 123) |
| COL10A1 | ES577-COL10A1-F | GGGCCTCAATGGACCCACCG |
| | | (SEQ ID NO: 124) |
| DSCR6 | JK1066-DSCR6-F | ATCCAGACACCTGGAGATGCTG |
| | | (SEQ ID NO: 125) |
| FLJ23152 | JK1087-FLJ23152F | TCACGTTGACTACGACTGAGAAGCC |
| | | (SEQ ID NO: 126) |
| POTEG | JK1081-POTEG-F | TGCTCTACATCTGGCCTCTGCC |
| | | (SEQ ID NO: 127) |
| TMEM145 | JK1101TMEM145F | TCTTTGCTGCCTATACCCTCTGGTTC |
| | | (SEQ ID NO: 128) |
| GRM4 | JK1099-GRM4-F | AAGAGGATGTCCGTACGAGGCTTC |
| | | (SEQ ID NO: 129) |
| FSIP1 | JK1357-FSIP1-F | CAGATGAGCAGGAGACTAAAGATGC |
| | | (SEQ ID NO: 130) |
| Gene Marker | Reverse Primer | Reverse Primer Sequence |
| ASCL1 | JK1096-ASCL1-R | TAGTTGGCGATGGGGTTGGTTGAC |
| | | (SEQ ID NO: 131) |
| BX116033 | JK1108-BTX116-R | TGCCGTATTCTTGGTGTCTGGAGC |
| | | (SEQ ID NO: 132) |
| C1ORF64 | JK1090-C1orf64-R | CCCACAGAACACGATGTAAGTCTTC |
| | | (SEQ ID NO: 133) |
| COL10A1 | ES578-COL10A1-R | CTGGGCCTTTGGCCTGCCTT |
| | | (SEQ ID NO: 134) |
| DSCR6 | JK1067-DSCR6-R | ACTCCGCAGGTATICTTGACGC |
| | | (SEQ ID NO: 135) |
| FLJ23152 | JK1088-FLJ23152-R | GTCTTCCTGCGCTCCTCGGC |
| | | (SEQ ID NO: 136) |
| POTEG | JK1082-POTEG-R | AGCAACATTAACGCACATTCATCTTCCC |
| | | (SEQ ID NO: 137) |
| TMEM145 | JK1102-TMEM145- | GTGCTGCGGGAAGGCCTTGTC |
| | R | (SEQ ID NO: 138) |
| GRM4 | JK1100-GRM4-R | CCAATTCGCTCACGGTTGGTGC |
| | | (SEQ ID NO: 139) |
| FSIP1 | JK1358-FSIP1-R | CACTGAACAATTACACCCCAAGTCTC |
| | | (SEQ ID NO: 140) |

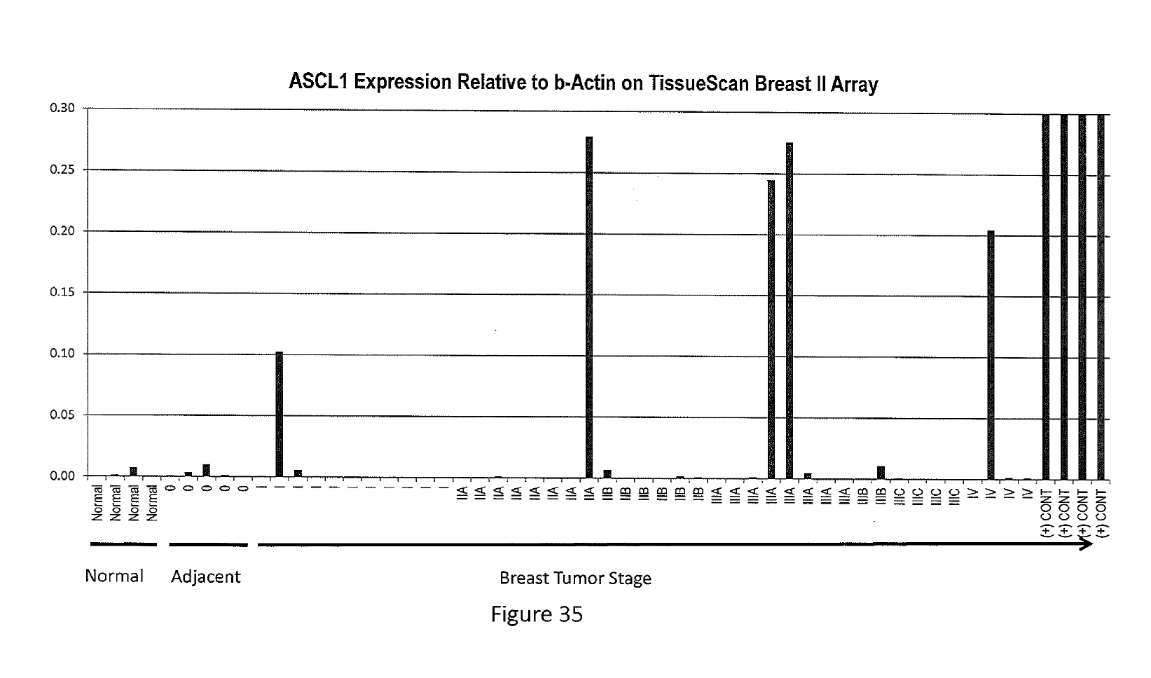

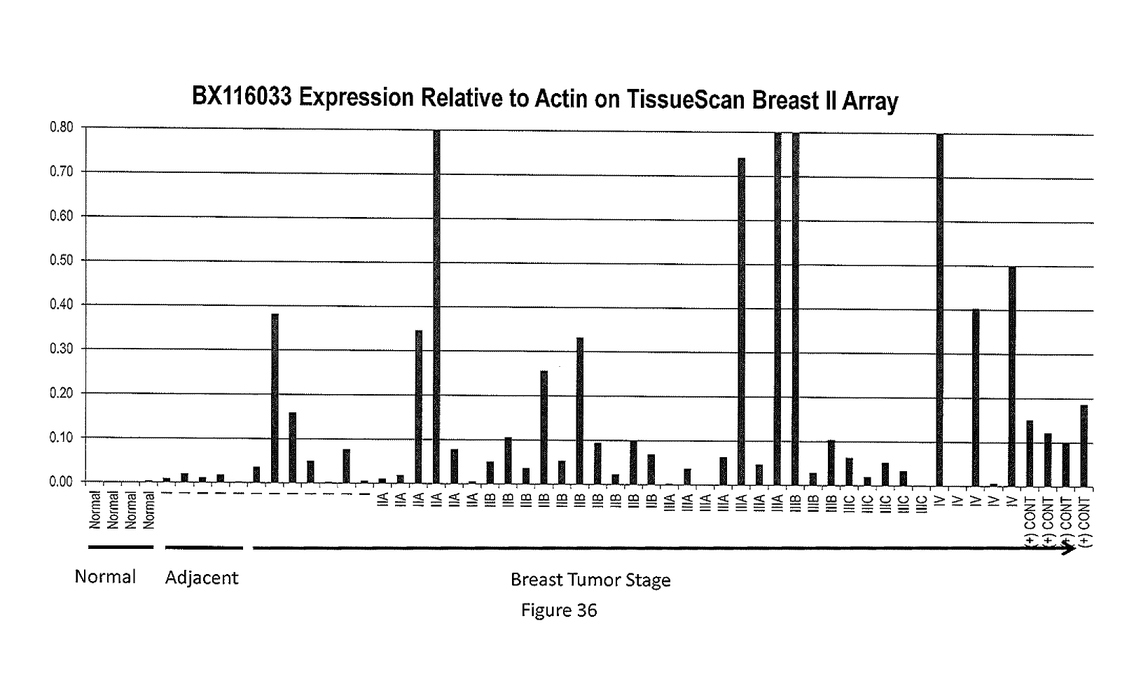

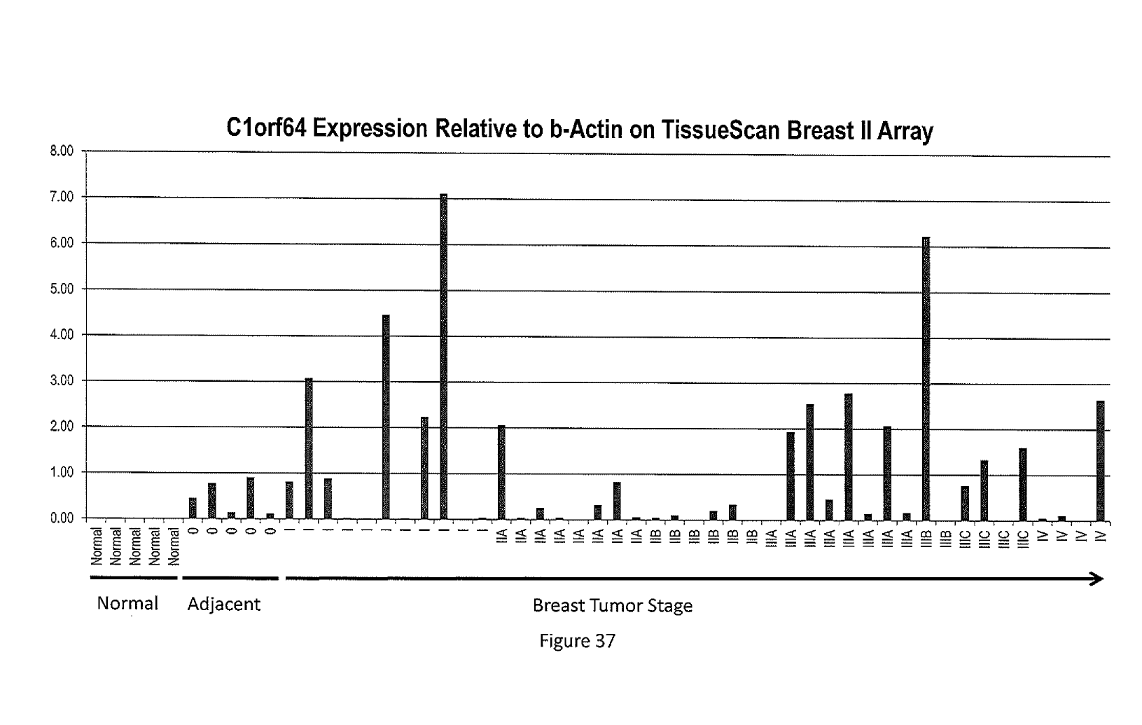

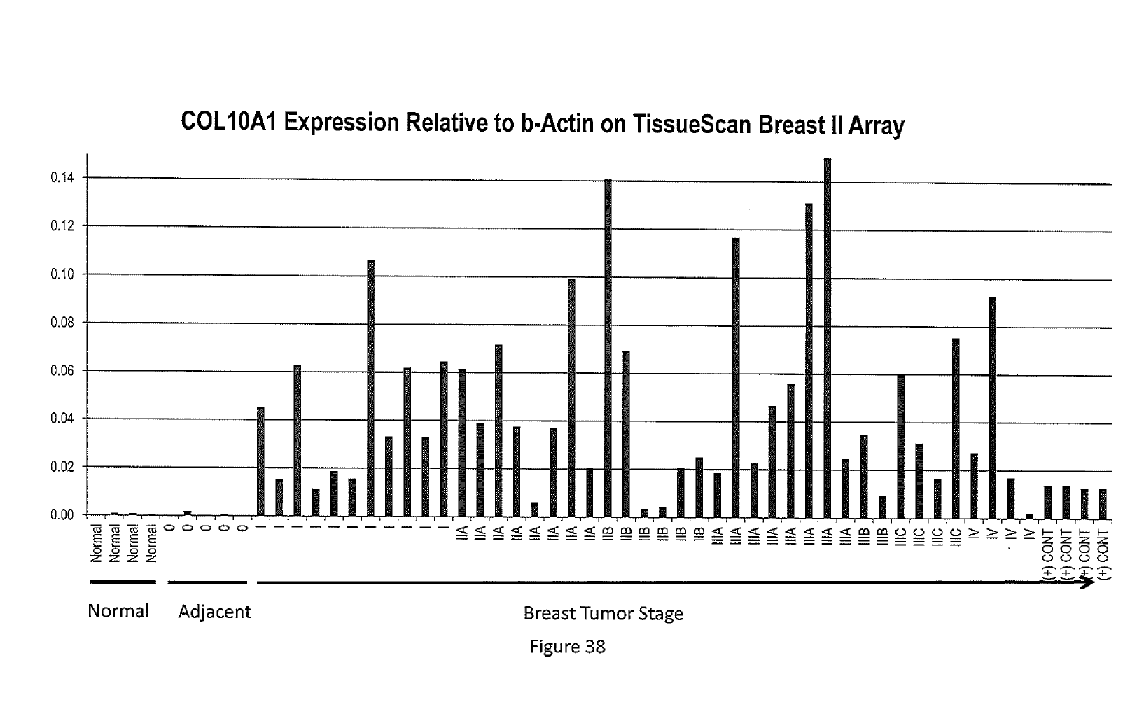

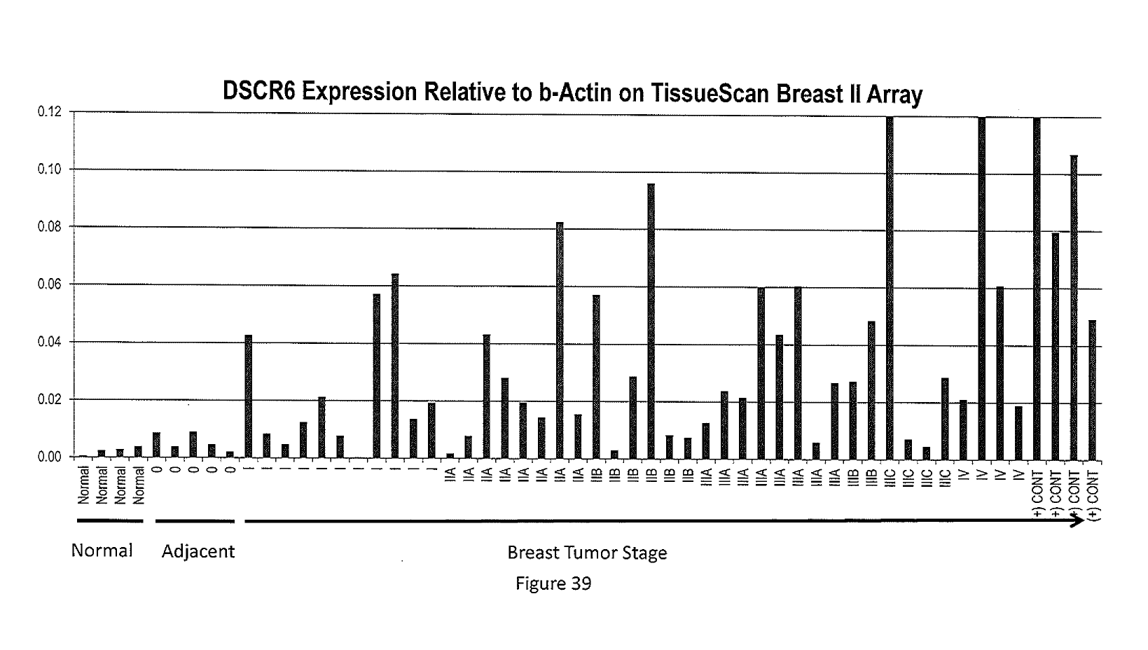

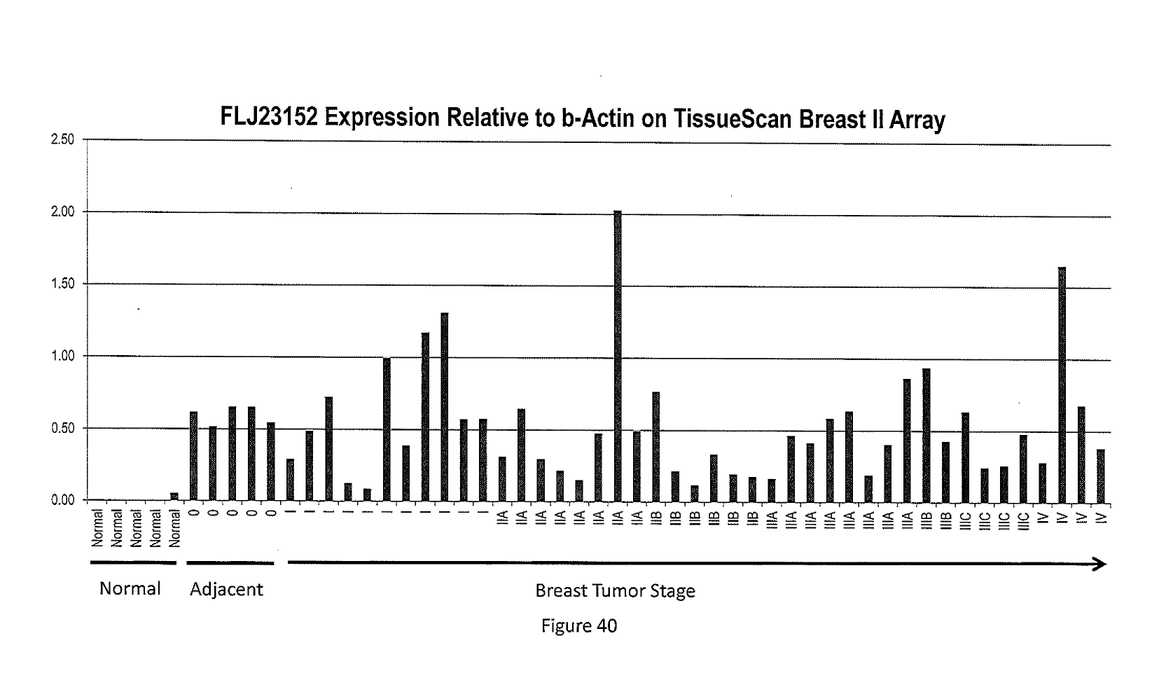

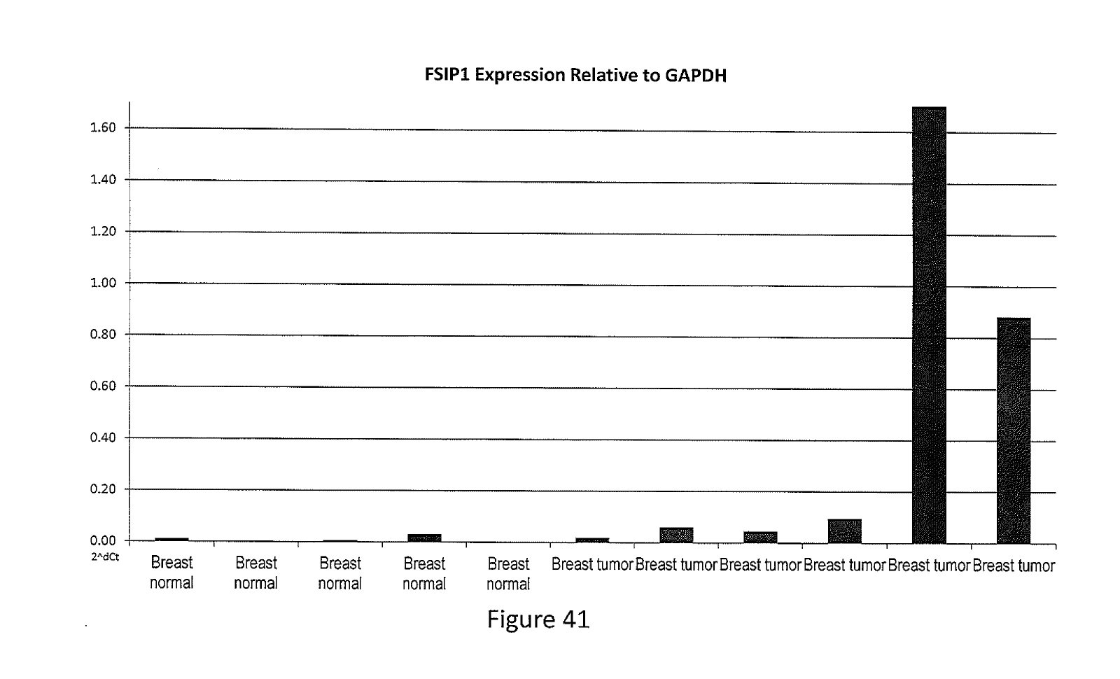

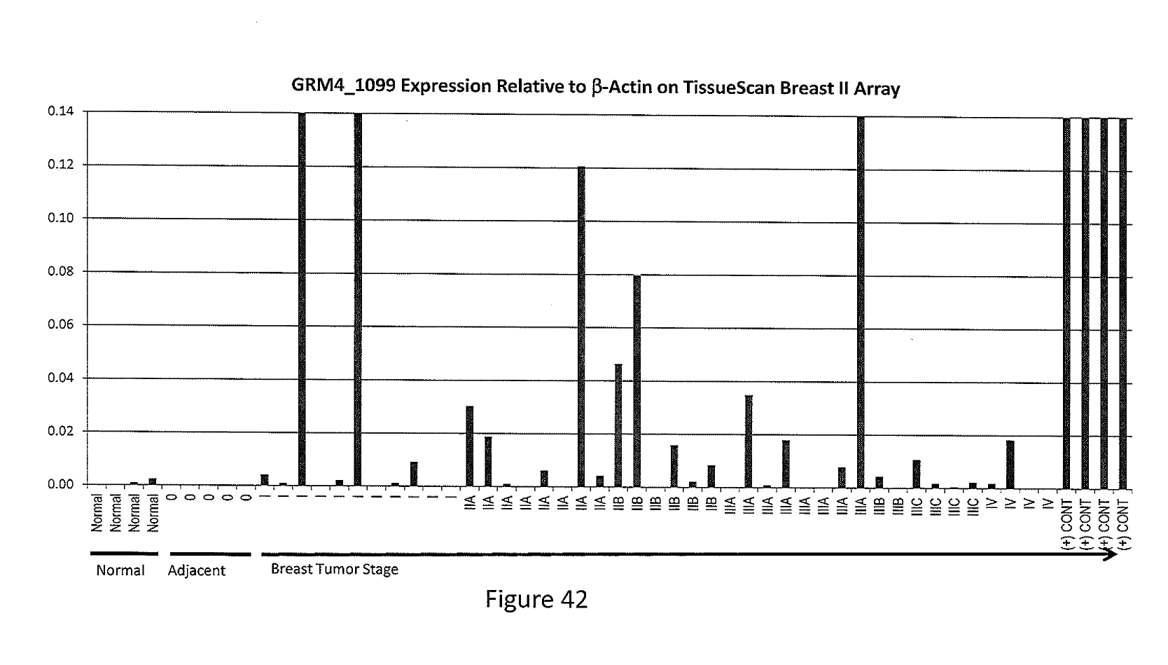

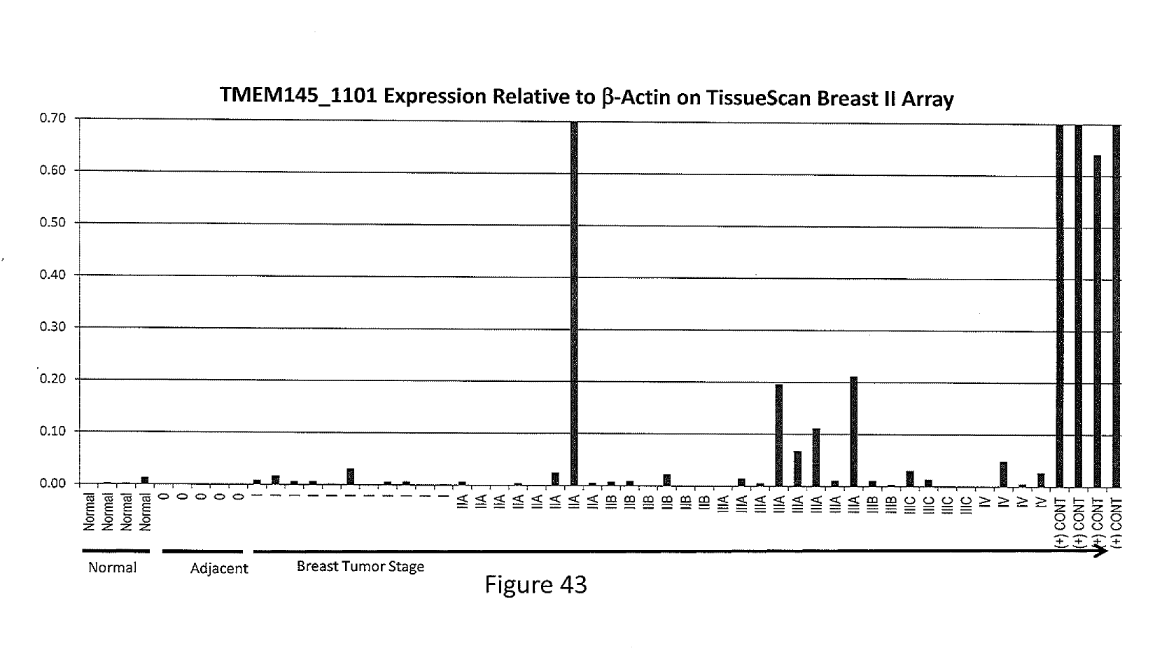

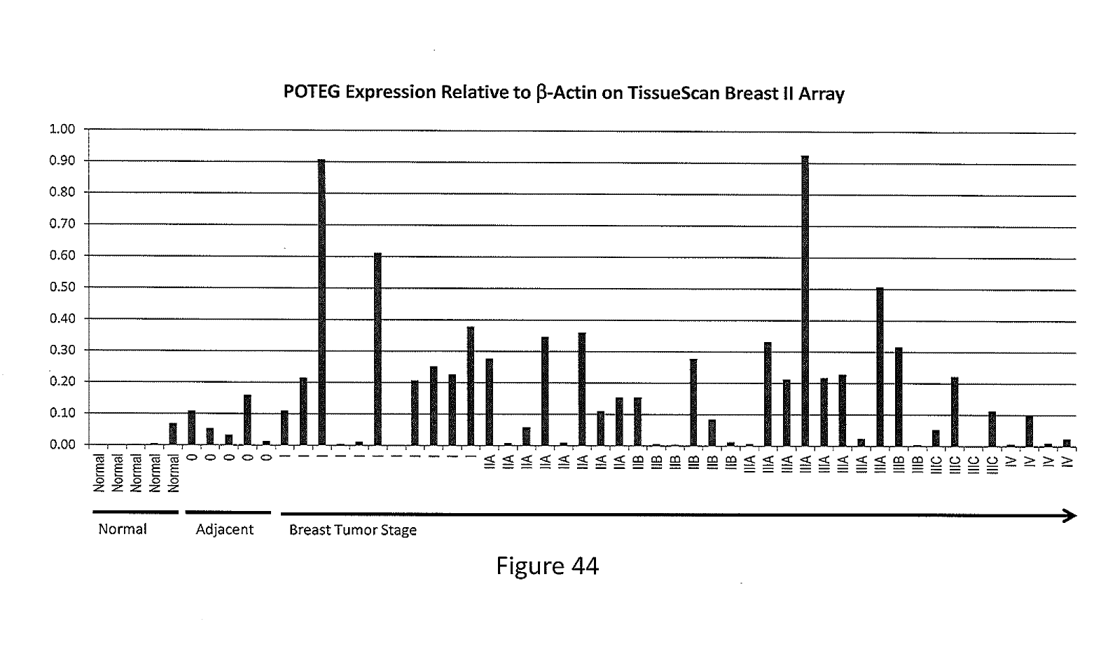

The results are presented in FIGS. 35-44 and showed that expression of ASCL1, BX116033, C1orf64; COL10A1; DSCR6; FLJ23152; GRM4; TMEM145_{—}1101; POTEG; AND FSIP were all elevated in tissue samples obtained from breast cancer patients compared to normal breast tissue.

### Example 36

### Detection of Collagen X in Breast Tumors by Immunoflourescent Cytochemistry

Paraffin embedded tissue sections of normal breast tissue (donors with no history of cancer) were obtained from Asterand (Detroit, Mich.). Paraffin-embedded tissue sections of breast cancers were obtained from OriGene (Rockville, Md.). The sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95° C. 40 minutes using an IHC-Steamer Set (IHC World #IW-1102). Immunostaining was performed using a monoclonal mouse anti-human collagen X antibody (Sigma Aldrich #C7974) at a 1:50 dilution in combination with a rabbit anti-human CD31 polyclonal antibody (Abeam #32457) at a 1:50 dilution. Primary antibodies were detected using an Alexa Fluor 488 donkey anti-rabbit IgG (Life Sciences #21206) and an Alexa Fluor 594 goat anti-mouse IgM (Life Sciences #21044) at a 1:200 dilution.

Vectashield mounting medium with DAPI was used to preserve the stained samples (Vector Laboratories #14-1200). Images were taken with an exposure time of 200 milliseconds using a Nikon Eclipse TE2000-U at a magnification of 10,000 and an X-Cite 120 fluorescence illumination system (Lumen Dynamics).

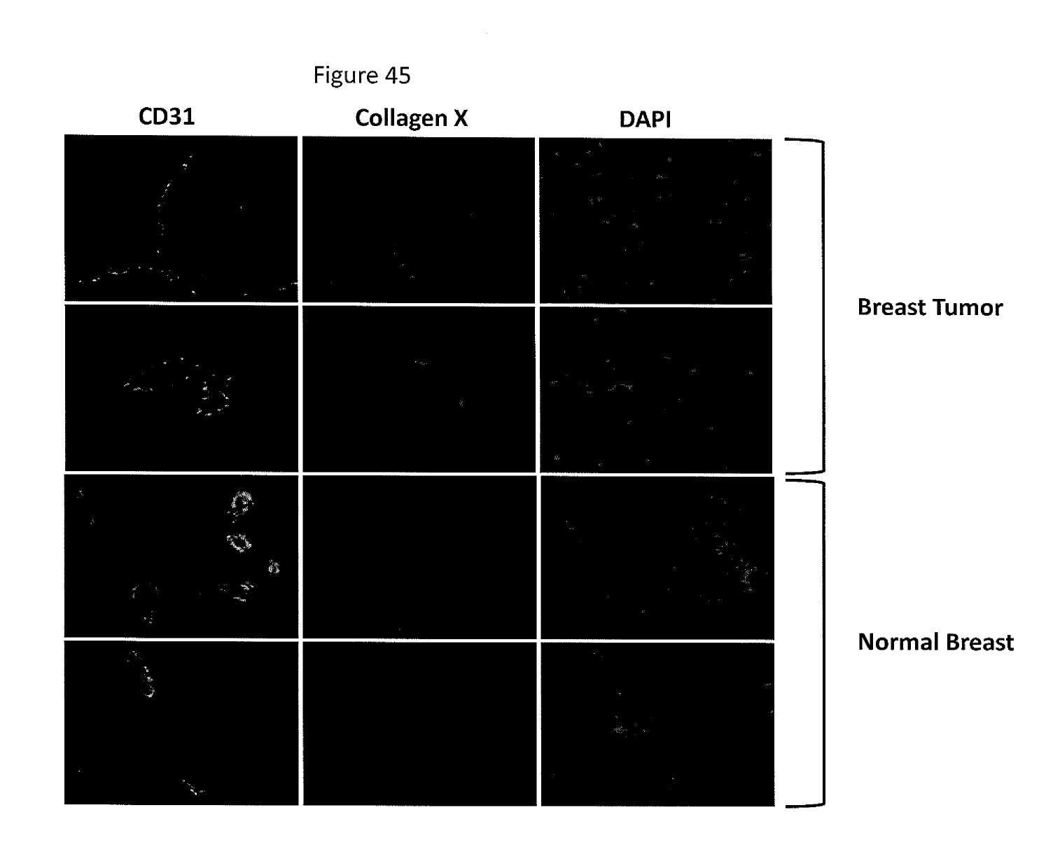

The results are shown in FIG. 45 and demonstrate that CollagenX protein is detected in breast tumor samples by ICC, but not normal breast tissue.

### Example 37

### Detection of MMP11 in Breast Tumors by Immunoflourescent Cytochemistry

Paraffin embedded tissue sections were obtained from Asterand (Detroit, Mich.). These specimens included: Normal breast tissue (donors with no history of cancer), fibroadenoma of the breast, and breast ductal cell carcinoma. Prior to the staining with antibodies, the sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95° C. 40 minutes using an IHC-Steamer Set (IHC World #IW-1102). Immunostaining was performed using a polyclonal rabbit anti-human MMP11 antibody (Abeam #ab52904) at a 1:100 dilution. The primary antibody was detected using an Alexa Fluor 594 Donkey anti-rabbit IgG (Life Sciences #A21207) at a 1:200 dilution.

Vectashield mounting medium with DAPI was used to preserve the stained samples (Vector Laboratories #H-1200). Images were taken with an exposure time of 400 milliseconds using a Nikon Eclipse TE2000-U at a magnification of 10,000 and an X-Cite 120 fluorescence illumination system (Lumen Dynamics).

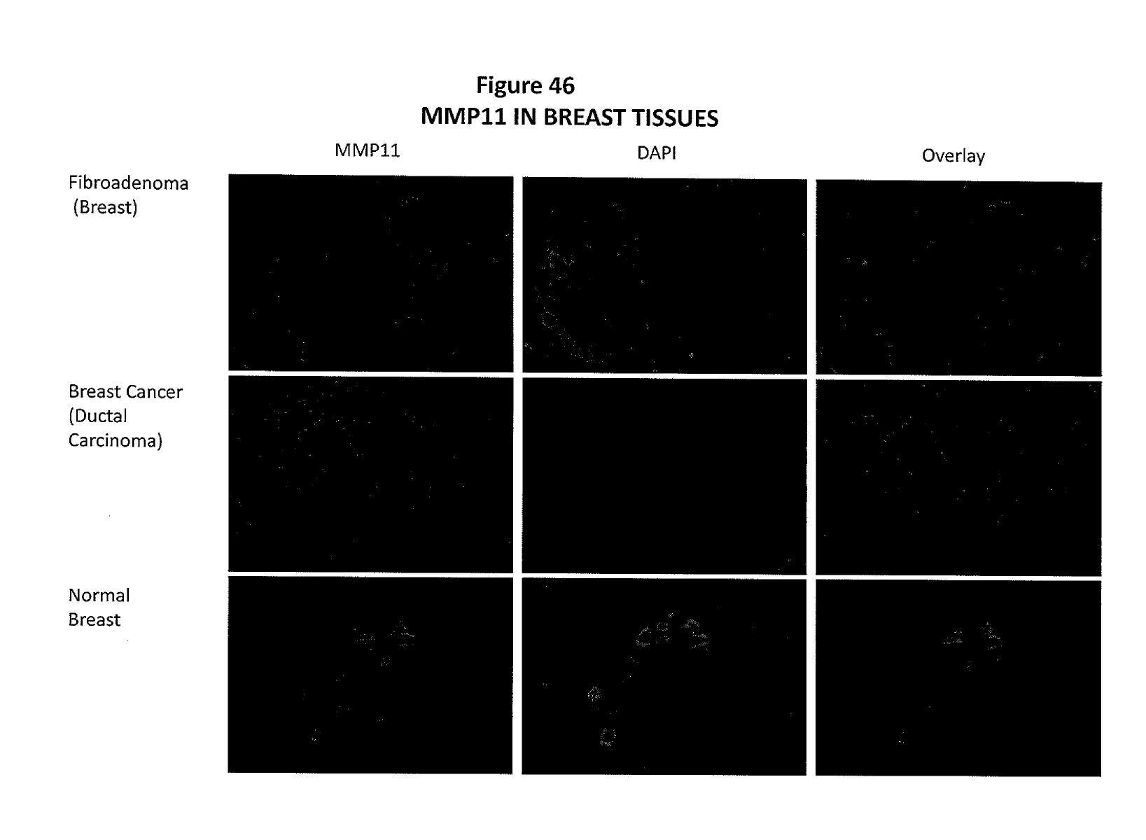

The results are shown in FIG. 46 and demonstrate that MMP11 protein is detected in breast tumor samples by ICC, but not normal breast tissue.

### Example 38

### Serum Detection Level of ANKRD30A, C1ORF64, COL10A1, MMP11, COL11A1 and POTEG in Breast Cancer Patients

Levels of the proteins ANKRD30A, C1ORF64, COL10A1, MMP11, COL11A1 and POTEG were assayed in serum obtained from breast cancer patients using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

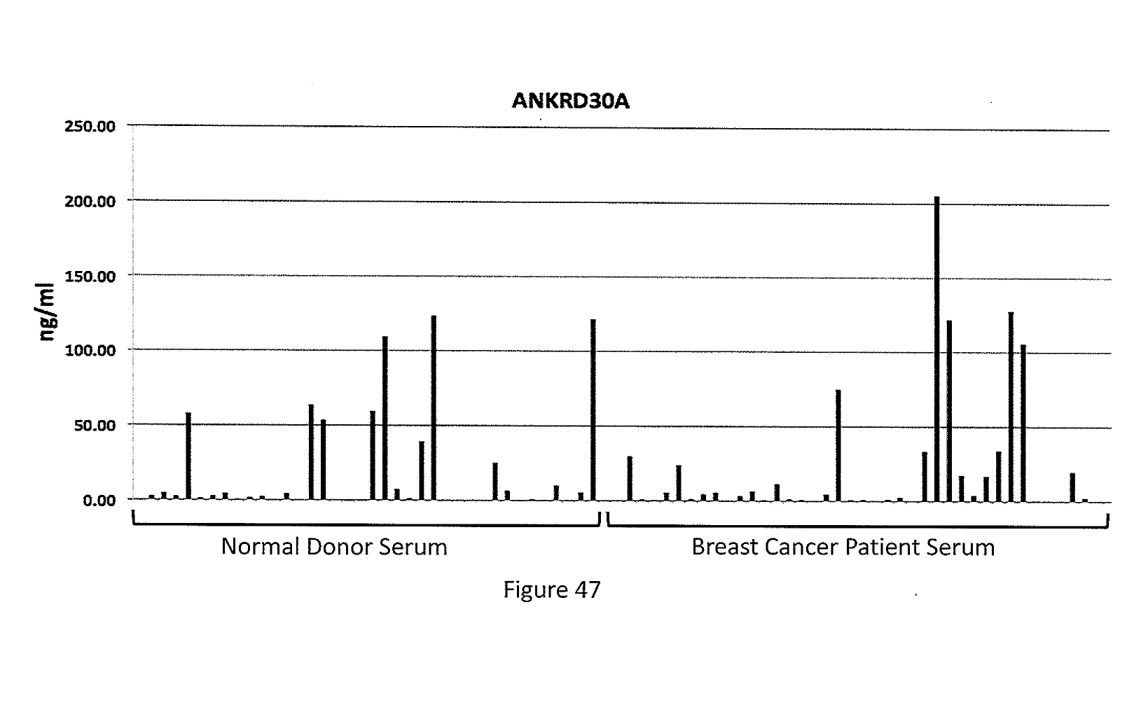

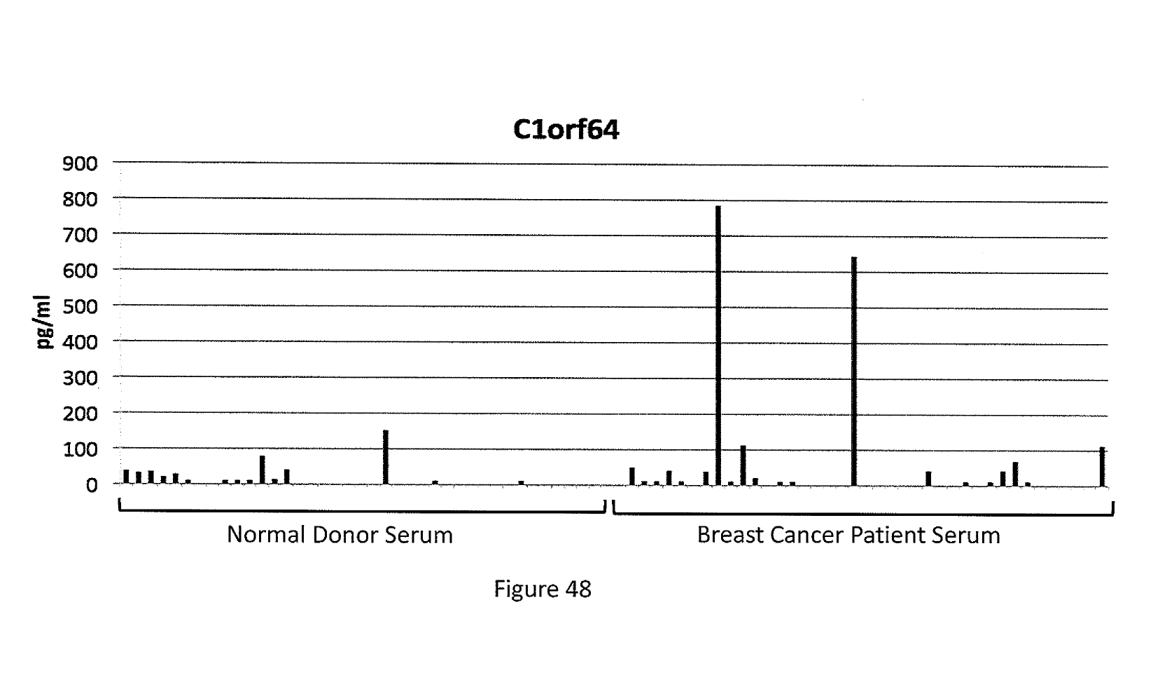

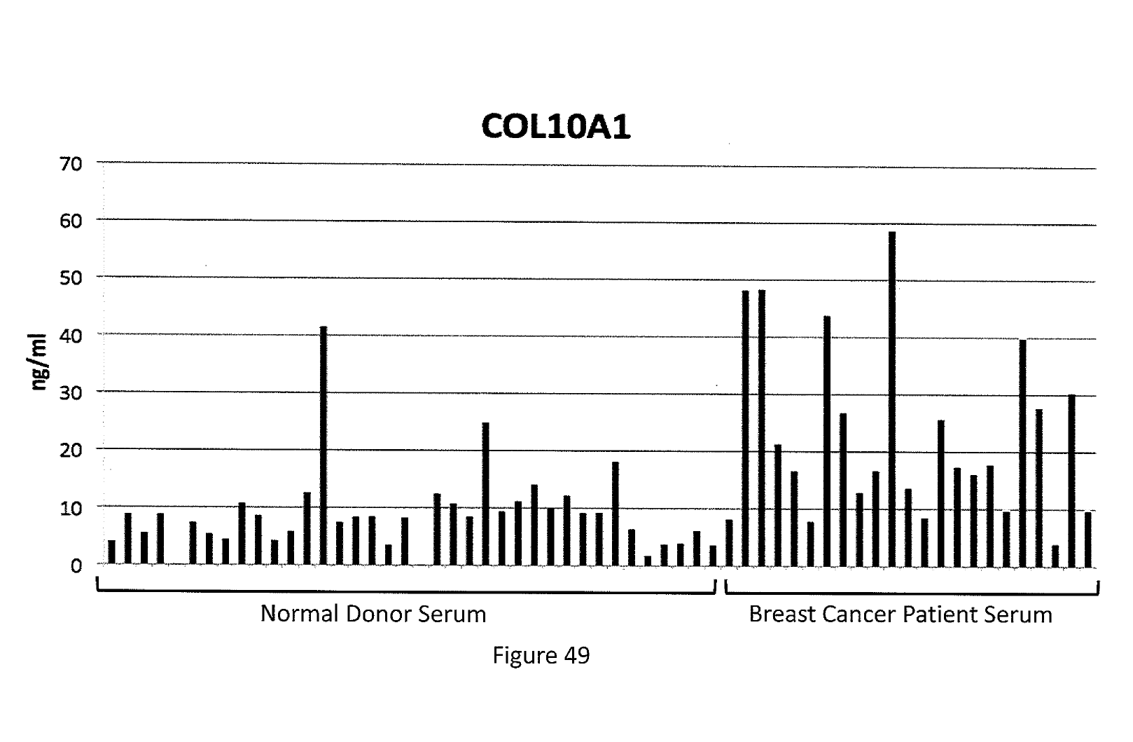

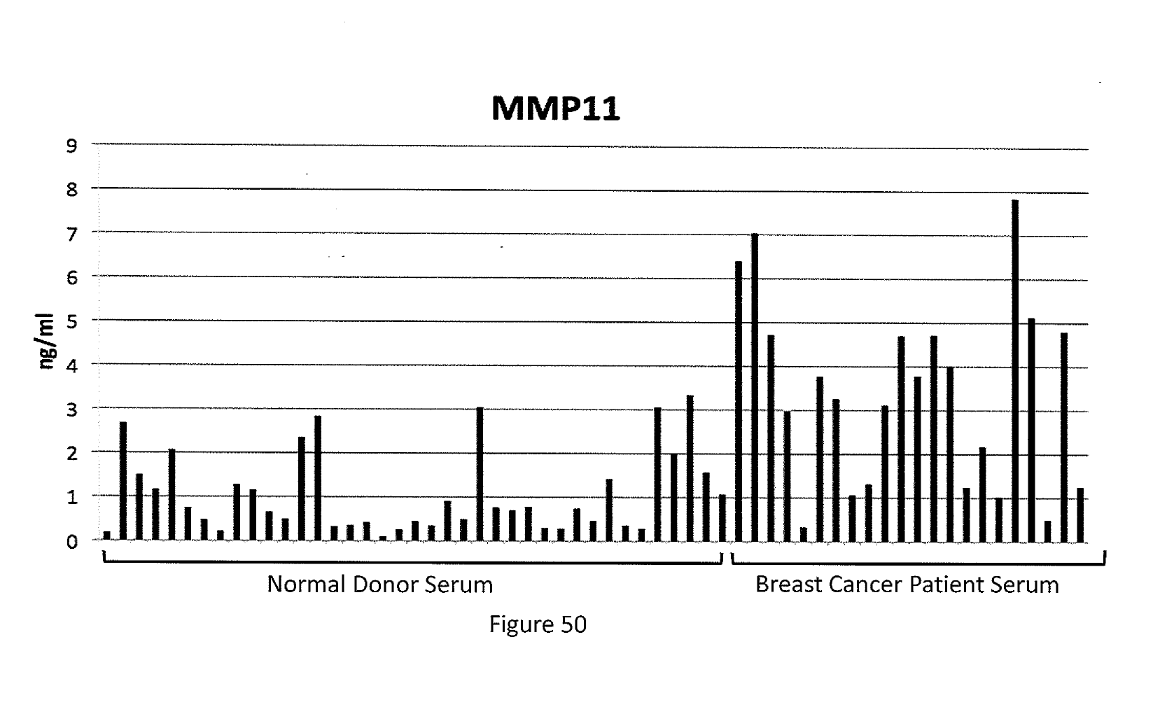

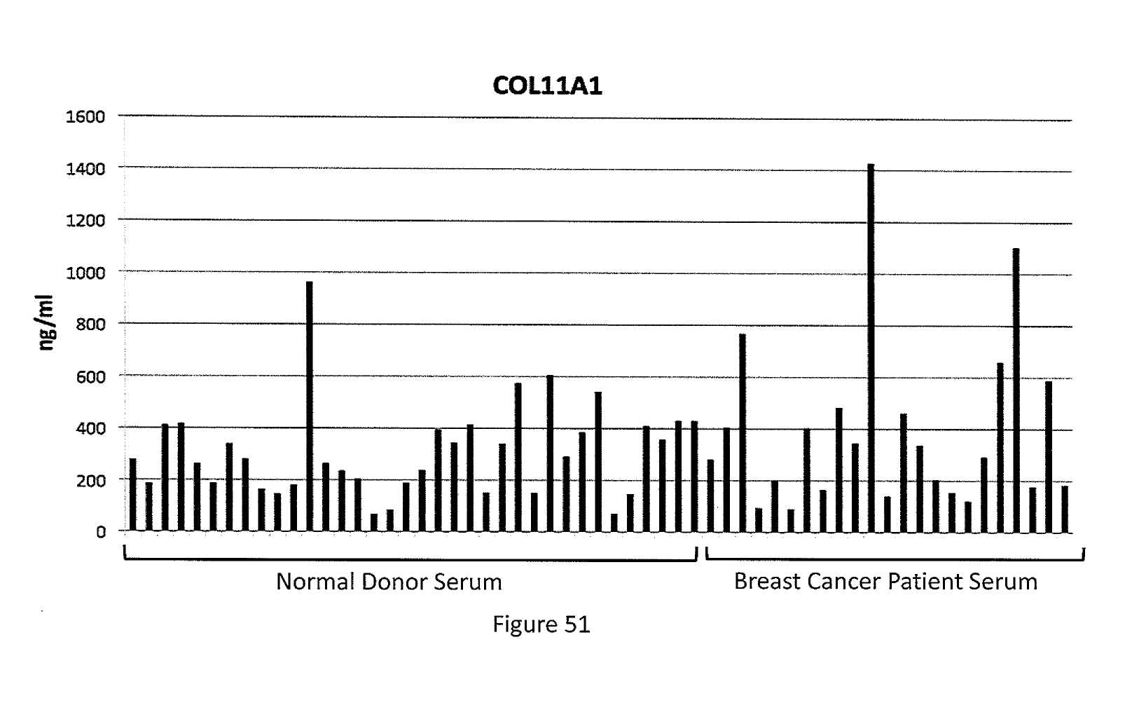

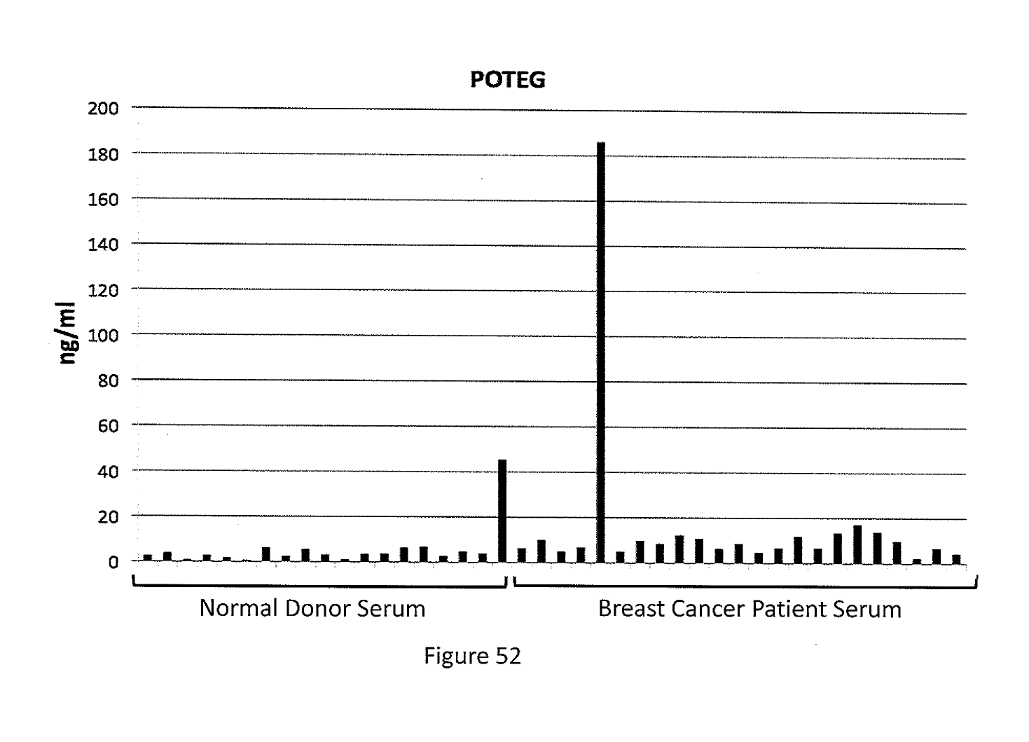

The results shown in FIGS. 47-52 indicated that elevated levels of ANKRD30A, C1ORF64, COL10A1, MMP11, COL11A1 and POTEG were detected in the serum of breast cancer patients relative to normal donor serum.

### Example 39

### Detection of FSIP1 in Breast Cancer Tissue

Paraffin embedded tissue sections of true normal breast (not adjacent normal to a tumor), fibroadenoma of the breast, and breast tumors (ductal carcinoma) were obtained from Asterand. The sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95° C. 40 minutes using an IHC-Steamer Set (IHC World #IW-1102). Immunostaining was performed incubating over night at 4 C with a polyclonal rabbit anti-human FSIP1 antibody (Novus Biologicals #NBP1-56460) at a 1:100 dilution in IHC-Tek antibody dilution buffer (IHC World #IW-1001). The antibody was washed out by incubating the slides 30 minutes in IHC-Tek washing buffer (IHC World #IW-1201), with a change of buffer every 10 minutes. Subsequently the slides were incubated one hour with Alexa Fluor 594 goat anti-rabbit IgG (Life sciences #21207) at a 1:200 dilution in antibody dilution buffer. After this incubation time, the slides were washed as described above, and Vectashield mounting medium with DAPI was used to preserve the stained samples (Vector Laboratories #H-1200). Images were taken with an exposure time of 200 milliseconds using a Nikon Eclipse TE2000-U at a magnification of 10,000 and an X-Cite 120 fluorescence illumination system (Lumen Dynamics).

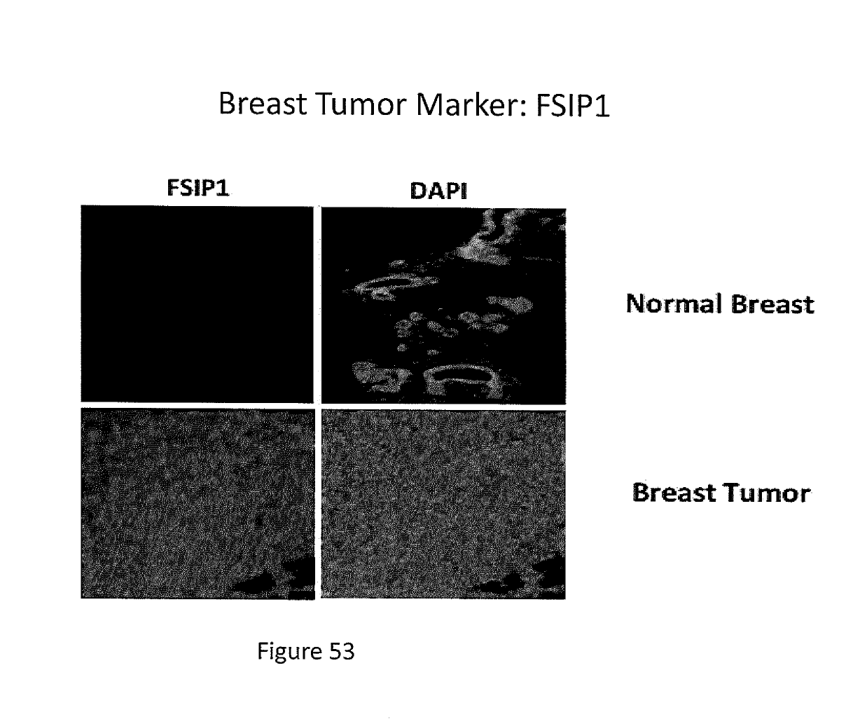

The results are shown in FIG. 53 and demonstrate that FSIP1 is expressed in breast tumor tissue, but is not expressed in normal breast tissue.

### Example 12

### Serum Detection Level of NMU in Cancer

Levels of the protein NMU were assayed in serum using a USCN ELISA kit (USCN) according to the manufacturer's instructions. In brief; 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 tunes with 350 μL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

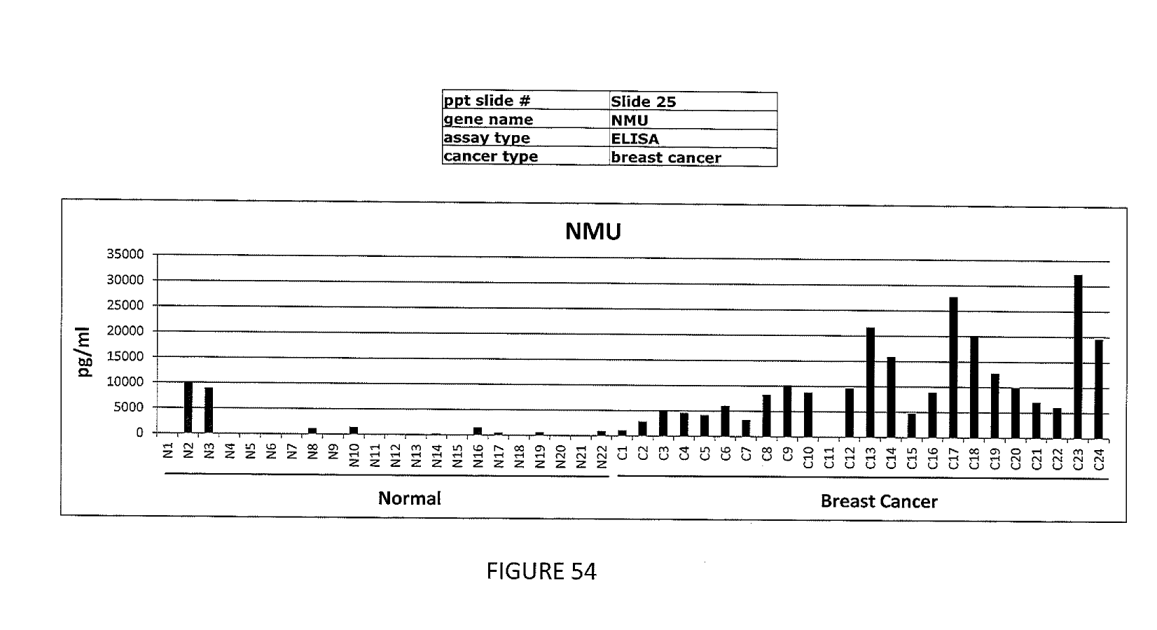

The results shown in FIG. 54 indicated that NMU was elevated in serum obtained from subjects breast cancer compared to normal subjects.

1. A method of detecting breast cancer in a subject comprising a) obtaining a sample from a subject b) contacting the sample obtained from the subject with one or more agents that detect expression of the markers encoded for by genes FSIP1, Col10A, MMP11, NMU, and C1orf64, or a complement thereof; c) contacting a non-cancerous cell, with the one or more agents from b); and d) comparing the expression level of the markers encoded for by genes FSIP1, Col10A, MMP11, NMU, and C1orf64 or a complement thereof in the sample obtained from the subject with the expression level of one the markers encoded for by genes FSIP1, Col10A, MMP11, NMU, and C1orf64 in the non-cancerous cell, wherein higher expression of at least one of the markers encoded for by genes FSIP1, Col10A, MMP11, NMU, and C1orf64 in the sample compared to the non-cancerous cell, indicates the subject has breast cancer.

2. A method of detecting breast cancer in a subject comprising a) obtaining a sample from a subject b) contacting the sample obtained from the subject with one or more agents that detect expression of one or more of the markers encoded for by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a complement thereof; c) contacting a non-cancerous cell, e.g. a non-cancerous cell from breast tissue, with the one or more agents from b); and d) comparing the expression level of one or more of the markers encoded for by genes chosen from C1orf64, LOC338579, LOC648879, HIST11-14H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a complement thereof in the sample obtained from the subject with the expression level of one or more of the markers encoded for by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a complement thereof in the non-cancerous cell, wherein a higher level of expression of one or more of the markers encoded for by genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL10A1, or a complement thereof in the sample compared to the non-cancerous cell indicates that the subject has breast cancer.

3. The method of claim 2, wherein the subject is a human.

4. The method of claim 2, wherein the sample is a bodily fluid.

5. The method of claim 4, wherein the bodily fluid is serum.

6. The method of claim 2, wherein the agent binds to one of the markers.

7. The method of claim 2, wherein agent is a nucleic acid.

8. The method of claim 7, wherein the nucleic acid is chosen from DNA and RNA.

9. The method of claim 2, wherein the agent is a protein.

10. The method of claim 2, wherein the protein is an antibody.

11. The method of claim 2, wherein the sample is a tissue sample.

12. The method of claim 2 further comprising isolating at least on molecule from the sample.

13. The method of claim 12, wherein the molecule is a nucleic acid encoding one of the markers.

14. The method of claim 11, wherein the molecule is a protein encoded for by one or more of the genes chosen from C1orf64, LOC338579, LOC648879, HIST1H4H, ASCL1, COL10A1, MMP11, DSCR6, CYP4Z1, HIST2H4B, BX116033, C6orf126, CLEC3A, HIST2H4A, SERHL2, FLJ23152, ABCC11, ANKRD30A, CNTD2, COL11A1, DHRS2, HIST1H3F, HIST1H3H, HIST2H2AB, KCNK15, LOC441376, LOC643637, LOC646360, PTPRT, RUNDC3A, SCGB2A2, SLITRK6, SYP, UBE2C, ZNF552, LOC388743, POTEC, FSIP1, GFRA1, LOC647333, POTEF, POTEE, POTEK, C2orf27A, LOC727941 (XR_{—}037440.1), NBPF22P, POTEG, RET, TMEM145, LOC727941 (XR_{—}037165.1), NAT1, NXPH1, SERHL2, SYCP2, D59687, CYP4Z1, LOC730024, NOS1AP, UGT2B28, GRM4, FLJ30428, LOC440905, LOC642460, MTL5, GRPR, COL 10A1.

15. A method of detecting breast cancer in a subject comprising a) obtaining a sample from a subject b) contacting the sample obtained from the subject with one or more agents that detect expression of the markers encoded for by genes MMP11, Col10A, C10rf64, Col11A, POTEG, and FSIP1 or a complement thereof; e) contacting a non-cancerous cell, with the one or more agents from b); and d) comparing the expression level of the markers encoded for by genes MMP11, Col10A, C10rf64, Col11A, POTEG, and FSIP1 or a complement thereof in the sample obtained from the subject with the expression level of one the markers encoded for by genes MMP11, Col10A, C10rf64, Col11A, POTEG, and FSIP1 in the non-cancerous cell, wherein higher expression of at least one of the markers encoded for by genes MMP11, Col10A, C10rf64, Col11A, POTEG, and FSIP1 in the sample compared to the non-cancerous cell, indicates the subject has breast cancer.

16. The method of claim 15, wherein the subject is human.

17. The method of claim 15, wherein the sample is a bodily fluid.

18. The method of claim 17, wherein the bodily fluid is serum.


SEQUENCE LISTING (142 sequences)

SEQ ID NO: 1 - DNA, 948 bp, Homo sapiens
acatcctggaagagtggcctaggacagctcctctcctgccagagctaggcaggcgccgaagtagccgcatggccccgtcagaagatcccagggactggagagccaacctcaaaggcaccatccgtgagacaggcctggagaccagctccggtgggaagctggctggccatcagaagaccgtccccacggctcacctgacttttgttattgactgcacccacgggaagcagctctccctggcagcaaccgcatcaccaccccaagcccccagtcccaatcgagggcttgtcaccccaccaatgaagacttacatcgtgttctgtggggaaaactggccccatctgactcgggtgacccccatgggtgggggatgccttgcccaggccagggccaccctgccgctctgcagagggtctgtggcctcagcttccttcccagtcagcccgctctgcccccaggaggttcccgaggctaaggggaaacccgtgaaggctgcgcctgtgaggtcttcaacttggggaacagtcaaggactcactgaaagccctctcctcttgtgtctgtgggcaggccgattagctggaagggccgggctctgatgcccagaggctgcaattcccagggcctggccctgcttccccagctaagcaggagtcttttgtgcttgagccaaggaaacatcattagatccgctaaggggcatctgaaacatccgtcgagtggcagaggcaggataagtcacctgcacatgaagagactcattcattcatacagcaaatattactggtacatcttccacatgccaggccctgcaaagtgctggggagataccatggttttcctggagctggtatttttggggtggagggaacccaccctgaataaataaagtaacccaataaataaagaagatgatttcgaaaaaaaaaaaaaaaaaaaaaaaaaaaaa

SEQ ID NO: 2 - DNA, 2262 bp, Homo sapiens
ctccgacccgggcttttccttcgtaccctgcggccccctccgcacccctcacggagctcctcgggtcctgccccctcccagcgcttgcccgcgccgccccgcccccgtttttaaacctggcgcgcgggtaggtgagcgcgttagcccgagtggatctaggcgcgctcgtaggccggcgccgcagcaaggggcgcgggctccgccggcaccatggagcccgaagcggcggccggagcccggaaggcgcgggggcgcggctgtcactgccccggggacgctccctggaggcctccgccaccgcgcgggccggagagccccgcgccgtggcgaccttggatccagacacctggagatgctgagctgaccagaactggaaggccgcttgaacctagggctgaccaacacacttttggatcaaagggagcctttgggtttcagcatcctgtaagagtctatttacccatgtcaaagcgtcaagaatacctgcggagttccggggagcaagtactggccagtttcccagtgcaagccacgattgacttctacgacgatgagtctactgagtctgcttccgaagctgaagagccagaggaaggacccccacccctccatcttctgccccaggaggtgggaggtcggcaggaaaatggcccagggggaaagggcagagaccagggcatcaaccaagggcagcgatcctcaggagggggtgaccactggggggagggtccgctccctcaaggtgtctcctcaaggggtggcaagtgctcctcatccaaatgaatcagtctctgtctcctgggccctgctctgggacctgcccctcacgttctcttggggacacccgagccaggacactacgcatccctgctgagtgtgcagaggctagaggcttctcgggcagcccctggcctgcacactactcatgacagaaagtcagcttttacttttctttcccctggcaattcgtttttggtgcactcatgcatgcctttgagaaaggattctaggagaaagagaaggtctatgtcaacagagttgttatctcatagagccagttttcaaagctccttctgcattgtcactcactgatcaggtgatgaattcttcctagatagtcgcccactccacctcctacttaacctgagactcattatttagctatttctgcttttgtaaaaataattcagatattaaactccaattttaatctatcatccaagggtagatgtagttgcttagtagcattttggaaaaaaaagaaaaaaaggtttggtttggttttgtgacggagttttgctcttgtctcccaggctggagtgcagtgacacaatctcggctcactgcaacctccacttcttgggttcaagtgattctcctgtctcagcctcccaagtggctgggattgcaggcatgagctaccacgcctggctaattttttgtatttttagtagagactcggtttcaccatgttggtcaggctggctttgaactcctgacctcaggtgatccacccgccttggcctcccaaagtgctgggattacaggcgtgagccactgagcccggccaaaaaaaaatgtatttattaaaaaaaaatttttttaacccgccgaataattcccataagagtaacaaaaaggaccagcctgaccagcgtggagaaactctgtctctactaaaaatacaaaattagccaggcgtgatggctcatgcctgtgatcccagctactcgggaggctgaggcaggagaatggctggaacccgggaggcggaggttgccgtgagtcgagatcgcaccattgcattccagcctgggcaacaagagcgaaactccgtctcaaaaaaaaaaaaaaaaaaaaaaagaaagaaaagagtaacagaaagatagggatttttaggagacaattagataaaaatgtatgtgatgactgtataaggaggcctgtgtgtatgaatttatagggagtagaaccgtctctcttcttagttggtgactgtttggggcctggtattttaatagatgaacactacttttttaattttttatttttttaaccctacccttaacccatgaacactatttttaattaaagtatctgatgtaaaattattttgagtttttaatattttgataaacgtgtattcctgaaactttttgacttacttatcttacatgtggtgtcttcctgtatatagtacattatatcaatttctacttgaaataaatattttgaaaaaaaaaaaaaaaaaaaaaaaaaaaa

SEQ ID NO: 3 - DNA, 607 bp, Homo sapiens
gaggcacatgccttgggagctaagtggcagctatacttcaccactatggcagtaaagatgactataaagactggcgggggatggatcctttcaaatgtacctgagctgggatgcccagcttgtggaacgcacagcaggaggtgagcagtggccaaaggaacatctgaaggaacacctgatgaggctgcacccttggcggaaagaacacctgacatggctgaaagcttggtggaaaaaccacctgatgaggctgcacccttggtggagggaacagctgacaaaattcaatgtttggggaaagcaacatctggaaagtttgaacagtcagcagaagaaacacctaagaaaattatgaggactgcaaaagaaacatctgagaaatttgcatggccagcaaaagaaagacctaggaagatcacatgggaggaaaaagaaacatctgtaaagactgaatgcgtggcaggagtaatacctaataaaactgaagttttggaaaaaggaacatctgagatgctcacgtgtcctacaaaagaaacatctacaaaagcaagtacaaatgaaggaagcaacaaagacagcaactgaacaacaagaaaatgatattgga

SEQ ID NO: 4 - DNA, 201 bp, Homo sapiens
atgacatctgatggcgccatcttgccctgtagatcattttggggacacctccagtatttcatgaaaattaattttttttctagtgatgaacaaaatgatactcagaagcaactttctgaagaacagaacactggaatattacaagatgagattctgattcatgaagaaaagcagatagaagtggctgaaaatgaattctga

SEQ ID NO: 5 - DNA, 374 bp, Homo sapiens
atgtctggccgtggtaaaggtggaaaaggtttgggtaagggaggagctaagcgtcatcgcaaggttttgcgcgataacatccagggcatcactaagccagctatccggcgccttgctcgtcgcggcggtgtcaagcgaatttctggccttatctatgaggagactcgtggtgttctgaaggtgttcctggagaacgtgattcgtgacgctgtcacttacacagagcacgccaaacgcaagaccgtgacagcaatggatgtggtctacgcgctgaagcgacagggacgcactctttacggcttcggtggctaaggctcctgcttgctgcactcttattttcattttcaaccaaaggcccttttcagggccgccca

SEQ ID NO: 6 - DNA, 2482 bp, Homo sapiens
cttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctccccgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgccttttttttcttactgttttattacaaacttacaaaaatatgtataaccctgttttatacaaactagtttcgtaataaaactttttcctttttttaaaatgaaaaaaaaaaaaaaaaaa

SEQ ID NO: 7 - DNA, 3307 bp, Homo sapiens
caccttctgcactgctcatctgggcagaggaagcttcagaaagctgccaaggcaccatctccaggaactcccagcacgcagaatccatctgagaatatgctgccacaaataccctttttgctgctagtatccttgaacttggttcatggagtgttttacgctgaacgataccaaatgcccacaggcataaaaggcccactacccaacaccaagacacagttcttcattccctacaccataaagagtaaaggtatagcagtaagaggagagcaaggtactcctggtccaccaggccctgctggacctcgagggcacccaggtccttctggaccaccaggaaaaccaggctacggaagtcctggactccaaggagagccagggttgccaggaccaccgggaccatcagctgtagggaaaccaggtgtgccaggactcccaggaaaaccaggagagagaggaccatatggaccaaaaggagatgttggaccagctggcctaccaggaccccggggcccaccaggaccacctggaatccctggaccggctggaatttctgtgccaggaaaacctggacaacagggacccacaggagccccaggacccaggggctttcctggagaaaagggtgcaccaggagtccctggtatgaatggacagaaaggggaaatgggatatggtgctcctggtcgtccaggtgagaggggtcttccaggccctcagggtcccacaggaccatctggccctcctggagtgggaaaaagaggtgaaaatggggttccaggacagccaggcatcaaaggtgatagaggttttccgggagaaatgggaccaattggcccaccaggtccccaaggccctcctggggaacgagggccagaaggcattggaaagccaggagctgctggagccccaggccagccagggattccaggaacaaaaggtctccctggggctccaggaatagctgggcccccagggcctcctggctttgggaaaccaggcttgccaggcctgaagggagaaagaggacctgctggccttcctgggggtccaggtgccaaaggggaacaagggccagcaggtcttcctgggaagccaggtctgactggaccccctgggaatatgggaccccaaggaccaaaaggcatcccgggtagccatggtctcccaggccctaaaggtgagacagggccagctgggcctgcaggataccctggggctaagggtgaaaggggttcccctgggtcagatggaaaaccagggtacccaggaaaaccaggtctcgatggtcctaagggtaacccagggttaccaggtccaaaaggtgatcctggagttggaggacctcctggtctcccaggccctgtgggcccagcaggagcaaagggaatgcccggacacaatggagaggctggcccaagaggtgcccctggaataccaggtactagaggccctattgggccaccaggcattccaggattccctgggtctaaaggggatccaggaagtcccggtcctcctggcccagctggcatagcaactaagggcctcaatggacccaccgggccaccagggcctccaggtccaagaggccactctggagagcctggtcttccagggccccctgggcctccaggcccaccaggtcaagcagtcatgcctgagggttttataaaggcaggccaaaggcccagtctttctgggacccctcttgttagtgccaaccagggggtaacaggaatgcctgtgtctgcttttactgttattctctccaaagcttacccagcaataggaactcccataccatttgataaaattttgtataacaggcaacagcattatgacccaaggactggaatctttacttgtcagataccaggaatatactatttttcataccacgtgcatgtgaaagggactcatgtttgggtaggcctgtataagaatggcacccctgtaatgtacacctatgatgaatacaccaaaggctacctggatcaggcttcagggagtgccatcatcgatctcacagaaaatgaccaggtgtggctccagcttcccaatgccgagtcaaatggcctatactcctctgagtatgtccactcctctttctcaggattcctagtggctccaatgtgagtacacacagagctaatctaaatcttgtgctagaaaaagcattctctaactctaccccaccctacaaaatgcatatggaggtaggctgaaaagaatgtaatttttattttctgaaatacagatttgagctatcagaccaacaaaccttccccctgaaaagtgagcagcaacgtaaaaacgtatgtgaagcctctcttgaatttctagttagcaatcttaaggctctttaaggttttctccaatattaaaaaatatcaccaaagaagtcctgctatgttaaaaacaaacaacaaaaaacaaacaacaaaaaaaaaattaaaaaaaaaaacagaaatagagctctaagttatgtgaaatttgatttgagaaactcggcatttcctttttaaaaaagcctgtttctaactatgaatatgagaacttctaggaaacatccaggaggtatcatataactttgtagaacttaaatacttgaatattcaaatttaaaagacactgtatcccctaaaatatttctgatggtgcactactctgaggcctgtatggcccctttcatcaatatctattcaaatatacaggtgcatatatacttgttaaagctcttatataaaaaagccccaaaatattgaagttcatctgaaatgcaaggtgctttcatcaatgaaccttttcaaacttttctatgattgcagagaagctttttatatacccagcataacttggaaacaggtatctgacctattcttatttagttaacacaagtgtgattaatttgatttctttaattccttattgaatcttatgtgatatgattttctggatttacagaacattagcacatgtaccttgtgcctcccattcaagtgaagttataatttacactgagggtttcaaaattcgactagaagtggagatatattatttatttatgcactgtactgtatttttatattgctgtttaaaacttttaagctgtgcctcacttattaaagcacaaaatgttttacctactccttatttacgacgcaataaaataacatcaatagatttttaggctgaattaatttgaaagcagcaatttgctgttctcaaccattctttcaaggcttttcattgttcaaagttaataaaaaagtaggacaataaagtgaaaaaaaaaaaaaaaaaa

SEQ ID NO: 8 - DNA, 2276 bp, Homo sapiens
aagcccagcagccccggggcggatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtcattaaacacagttgttttctaaaaaaaaaaaaaaaa

SEQ ID NO: 9 - DNA, 1907 bp, Homo sapiens
agaatggagccctcctggcttcaggaactcatggctcaccccttcttgctgctgatcctcctctgcatgtctctgctgctgtttcaggtaatcaggttgtaccagaggaggagatggatgatcagagccctgcacctgtttcctgcaccccctgcccactggttctatggccacaaggagttttacccagtaaaggagtttgaggtgtatcataagctgatggaaaaatacccatgtgctgttcccttgtgggttggaccctttacgatgttcttcagtgtccatgacccagactatgccaagattctcctgaaaagacaagatcccaaaagtgctgttagccacaaaatccttgaatcctgggttggtcgaggacttgtgaccctggatggttctaaatggaaaaagcaccgccagattgtgaaacctggcttcaacatcagcattctgaaaatattcatcaccatgatgtctgagagtgttcggatgatgctgaacaaatgggaggaacacattgcccaaaactcacgtctggagctctttcaacatgtctccctgatgaccctggacagcatcatgaagtgtgccttcagccaccagggcagcatccagttggacagtaccctggactcatacctgaaagcagtgttcaaccttagcaaaatctccaaccagcgcatgaacaattttctacatcacaacgacctggttttcaaattcagctctcaaggccaaatcttttctaaatttaaccaagaacttcatcagttcacagagaaagtaatccaggaccggaaggagtctcttaaggataagctaaaacaagatactactcagaaaaggcgctgggattttctggacatacttttgagtgccaaaagcgaaaacaccaaagatttctctgaagcagatctccaggctgaagtgaaaacgttcatgtttgcaggacatgacaccacatccagtgctatctcctggatcctttactgcttggcaaagtaccctgagcatcagcagagatgccgagatgaaatcagggaactcctaggggatgggtcttctattacctgggaacacctgagccagatgccttacaccacgatgtgcatcaaggaatgcctccgcctctacgcaccggtagtaaacatatcccggttactcgacaaacccatcacctttccagatggacgctccttacctgcaggaataactgtgtttatcaatatttgggctcttcaccacaacccctatttctgggaagaccctcaggtctttaaccccttgagattctccagggaaaattctgaaaaaatacatccctatgccttcataccattctcagctggattaaggaactgcattgggcagcattttgccataattgagtgtaaagtggcagtggcattaactctgctccgcttcaagctggctccagaccactcaaggcctccccagcctgttcgtcaagttgtcctcaagtccaagaatggaatccatgtgtttgcaaaaaaagtttgctaattttaagtcctttcgtataagaattaatgagacaattttcctaccaaaggaagaacaaaaggataaatataatacaaaatatatgtatatggttgtttgacaaattatataacttaggatacttctgactggttttgacatccattaacagtaattttaatttctttgctgtatctggtgaaacccacaaaaacacctgaaaaaactcaagctgacttccactgcgaagggaaattattggtttgtgtaactagtggtagagtggctttcaagcatagtttgatcaaaactccactcagtatctgcattacttttatctctgcaaatatctgcatgatagctttattctcagttatctttccccataataaaaaatatctgccacc

SEQ ID NO: 10 - DNA, 396 bp, Homo sapiens
agaagctgtctatcgggctccagcggtcatgtccggcagaggaaagggcggaaaaggcttaggcaaagggggcgctaagcgccaccgcaaggtcttgagagacaacattcagggcatcaccaagcctgccattcggcgtctagctcggcgtggcggcgttaagcggatctctggcctcatttacgaggagacccgcggtgtgctgaaggtgttcctggagaatgtgattcgggacgcagtcacctacaccgagcacgccaagcgcaagaccgtcacagccatggatgtggtgtacgcgctcaagcgccaggggcgcaccctgtacggcttcggaggctaggccgccgctccagctttgcacgtttcgatcccaaaggccctttttagggccgacca

SEQ ID NO: 11 - DNA, 488 bp, Homo sapiens
ttttttttttttttaaacaaacttcagcttcttagacgggctttcagggctacagaaatcgctcacgttttacacccgcttatgtaagtgtgtgtgcgctggagcgggtgtacacaggcatgttcactgcacctgtatacaggtgcaggcgtttcaggggggcattagctacacagcatttctcaaccttttggaagctggggtaatttggctctgaaacctgttccctgggtcgtgccctcttgcggatccaggagagcagggacatttgcctgtgcgttcactgccgtattcttggtgtctggagcagtgcctgacctgtggcgggtgcttagtgagttatccgtgcaatgaatgaatgaatcaatgaatgaatgaatgaatgaatgaacgaaccagccggaaccttgctggccgttgactgaaaatgtctggattcaattgactgaatcaaacagacttagagcttgagagggaaaaattatttc

SEQ ID NO: 12 - DNA, 488 bp, Homo sapiens
gcccatggccgcagccctggcgctcgtggcgggggtcctgtcgggggcggtgctgcccctctggagcgcgcttccgcaatataaaaagaaaatcacagacaggtgcttccaccactctgagtgctacagtggctgctgcctcatggacttggactccggtggagccttctgtgcccccagggccagaataaccatgatctgcttgccccagtggttggaactcttcaagggcagggattgcatcatattcatctatgaagcacctacccccagcttagtatctgcacataaccaagggagctaccaacatcatctgcccttgccggatgggcttgacgtgcatatccaaggacttgatgtgttcccgccggtgccatatgatttagaggaagatgcaggctggtcactgctcccttggggccataggccctggttgccaccaacttgctccaaatccagctcctgagacattaaagtcacttcctgtc

SEQ ID NO: 13 - DNA, 1915 bp, Homo sapiens
ggcaacatggctcagcaggcttgccccagagccatggcaaagaatggacttgtaatttgcatcctggtgatcaccttactcctggaccagaccaccagccacacatccagattaaaagccaggaagcacagcaaacgtcgagtgagagacaaggatggagatctgaagactcaaattgaaaagctctggacagaagtcaatgccttgaaggaaattcaagccctgcagacagtctgtctccgaggcactaaagttcacaagaaatgctaccttgcttcagaaggtttgaagcatttccatgaggccaatgaagactgcatttccaaaggaggaatcctggttatccccaggaactccgacgaaatcaacgccctccaagactatggtaaaaggagcctgccaggtgtcaatgacttttggctgggcatcaatgacatggtcacggaaggcaagtttgttgacgtcaacggaatcgctatctccttcctcaactgggaccgtgcacagcctaacggtggcaagcgagaaaactgtgtcctgttctcccaatcagctcagggcaagtggagtgatgaggcctgtcgcagcagcaagagatacatatgcgagttcaccatccctaaataggtctttctccaatgtgtcctccaagcaagattcatcataacttataggttcatgatctctaagatcaagtaaaaatcataatttttacttattaaaaaattgcaacacaagatcaatgtccatagcaatatgatagcatcagccaattttgctaacacatttctttgggattttgcccttcctggggtataggggatcagaaatattgatccatgtgcacgcagataaaatggcttctgctaaacagactaaaatctttctctctagtctttctcacttgtacaaacccagtttgttttcaaaaaatcacagtagcaatgcaactcatcactctagaaaagcaagcttaggctacctgaaagattttcccttggaagtttagcgtatgtttgactaacaaaaattccctacatcagagactctaggtgctatataatccaaaaacttttcagcctgttgctcattctgtcccatgctggcaataataccttgtcagcccattacccttattttgaattgctccatctcctggtgggacttgtatcttgtctgccatatcagaacacaaacccctgaagaggttctgatttgattttttttttttcttcatgcctaccctttttttggaagtttccagccgcaatttgaaatgaaatgacaaggtgtatatttgatcaattttcattcccaccattgcattacaacctctaacttaaatgggtaaccctaaggcatatcaaagaagcagattgcatgataaacggaaatagaaaaaaagaacctacatttattttgctttagcatccttactctcaccttttatgagattgagagtggacttacatttccttttttacattttcgtatatttattttttttagccatcattatatgtttaagtctattatgggcaaccaatctttggaagctgaaaactgaatttaaagaatgctatcttggaaaattgcatacgtctgtgcaattttttattctgcctagtgctattctgcttgtttaactagattgtacaaaataacttcattgcttaatatcaaattacaaagtttagacttggagggaaatgggctttttagaagcaaacaattttaaatatattttgttcttcaaataaatagtgtttaaacattgaatgtgttttgtgaacaatatcccactttgcaaactttaactacacatgcttggaattaagttttagctgttttcattgctcaataataaagcctgaattctgatcaataaaaaaaaaaaaaaaaaaaaaaaaaaaaa

SEQ ID NO: 14 - DNA, 396 bp, Homo sapiens; identical to SEQ ID NO: 10

SEQ ID NO: 15 - DNA, 1374 bp, Homo sapiens
ggacagcttggagatagggcccggaattgcgggcgtcactctgctcctgcgacctagccaggcgtgagggagtgacagcagcgcattcgcgggacgagagcgatgagtgagaacgccgcaccaggtctgatctcagagctgaagctggctgtgccctggggccacatcgcagccaaagcctggggctccctgcagggccctccagttctctgcctgcacggctggctggacaatgccagctccttcgacagactcatccctcttctcccgcaagacttttattacgttgccatggatttcggaggtcatgggctctcgtcccattacagcccaggtgtcccatattacctccagacttttgtgagtgagatccgaagagttgtggcagccttgaaatggaatcgattctccattctgggccacagcttcggtggcgtcgtgggcggaatgtttttctgtaccttccccgagatggtggataaacttatcttgctggacacgccgctctttctcctggaatcagatgaaatggagaacttgctgacctacaagcggagagccatagagcacgtgctgcaggtagaggcctcccaggagccctcgcacgtgttcagcctgaagcagctgctgcagaggttactgaagagcaatagccacttgagtgaggagtgcggggagcttctcctgcaaagaggaaccacgaaggtggccacaggtctggttctgaacagagaccagaggctcgcctgggcagagaacagcattgacttcatcagcagggagctgtgtgcgcattccatcaggaagctgcaggcccatgtcctgttgatcaaagcagtccacggatattttgattcaagacagaattactctgagaaggagtccctgtcgttcatgatagacacgatgaaatccaccctcaaagagcagttccagtttgtggaagtcccaggcaatcactgtgtccacatgagcgaaccccagcacgtggccagtatcatcagctccttcttacagtgcacacacatgctcccagcccagctgtagctctgggcctggaactatgaagacctagtgctcccagactcaacactgggactctgagttcctgagccccacaacaaggccagggatggtggggacaggcctcactagtcttgaggcccagcctaggatggtagtcaggggaaggagcgagattccaacttcaacatctgtgacctcaagggggagacagagtctgggttccagggctgctttctcctggctaataataaatatccagccagctggaggaaggaagggcaggctgggcccacctagcctttccctgctgcccaactggatggaaaataaaaggttcttgtattctca

SEQ ID NO: 16 - DNA, 2085 bp, Homo sapiens
gtttaacattacagatatccttaacatatatatgtgcagagtagatatctgggaagacacacactgaactctttacaaaggttcatgctaaggactgggattgggggccatgtggttaaatagaggataaggagatgatttatgtatcagttatattttttataatcaatctatattatcttaaaactaatttttaaaacaaaacaccttaaaactttcttcataaatactttatgacaattttacttattttggaactagaaattataattagaaagtcatcattactgctttgaactgttgaaagggtagttgatcatatgaagtgggatttttcccccgctgtcatgaaaatgtttgtcaagttcctccagcaaagcccagaacagagttgttgttcagtaatcgttaattatctctttctaccacaaaaccagaagcaatgagaactaatctctatcccagatgaaagtaacttgtagcttgtggaatattataaaatcatcaggttgaactcttcaccttaaactacaaattcacccccattctccactgcttttcatcttatattcattctcaagattttttccttctaagggttcatcttctatgcagtctgagaacacctgcattcatagggcctttttgtaatagcctggtgcttttaaggcatctaaaagatcttatcgttataattctgttgttatcaactttcacgttggtttttttaatctgcgcctggaaagttagatttttagaatttactttattcatttatttatttatttatttagagacagggtctcgctctgttgccaaggctggagtgcagtggaacgatcttggctcactgcaacctccacctcctgggctcaagcgatcctccaccctcagcctcccaagtagctgggactacgggcacacaccaccatgcctggctaattttttgtatttttgtagagacaaggtttcactatgttgcccaggctggcctcaaactccggagctcaagcagtcttcccaccttggcctcccaaagtgccgggattacaggcatgagccactgcacccagactagaatttacattttaaactgtttattctaactcttttgcccttctcttctgccagtgaatatgattgtttcttcatgtaaaaagatgttttattccagttgactacgactgagaagccattgagaaagccaccttccagactgaaaaaactcaagatcaaaaagcaagtgaaggatttcacaatgaaggacatcgaggagaagatggaggctgccgaggagcgcaggaagactaaagaagaagaaataagaaaaaggctacggagtgaccgacttttgccttcagccaatcactcagattcagctgaattagatggggccgaggttgcatttgccaaaggacttcaaagggtgaggtctgctggatttgaaccatctgacctgcagggaggaaaaccattgaagaggaagaagagtaaatgtgatgcaaccttgattgatagaaacgaaagtgatgaaagttttggggtcgtggagtcagacatgtcctacaaccaagcagatgacatagtctactaagccattttttgtgaatttcataagaaagcattcattctccccatttgtgacatttgtagtatgtctcatattctttgactgactgacctcattccactgggatttctgccttgggcttaaggatgattgtgtgggctgcacaggctgaaggttagttgagtaaattaagtagctatgctagcttttaaaaaaagagcgagggggagacttgaccagcatagatatttggcactctcctttgtgtggcttcaaacattatggagatgtctttaattcatttataagtgccttcagtttacattaataagttcgtgccaaatgaaatccttcctgtttactcctgccttgtggcggggtccatcctctgctgctcctttaaaacaaattgcatggatttgtattaagtattttaatggcttatatgttttttatgctgtatgtctatggttttataatttttttctctgtgttgtgaaggaataaagcaatgt

SEQ ID NO: 17 - DNA, 4476 bp, Homo sapiens
aggagagcctcccggtgtatttgaataaaccaggttggcaaatcatactatagctgaaagaattggcaggaactgaaaatgactaggaagaggacatactgggtgcccaactcttctggtggcctcgtgaatcgtggcatcgacataggcgatgacatggtttcaggacttatttataaaacctatactctccaagatggcccctggagtcagcaagagagaaatcctgaggctccagggagggcagctgtcccaccgtgggggaagtatgatgctgccttgagaaccatgattcccttccgtcccaagccgaggtttcctgccccccagcccctggacaatgctggcctgttctcctacctcaccgtgtcatggctcaccccgctcatgatccaaagcttacggagtcgcttagatgagaacaccatccctccactgtcagtccatgatgcctcagacaaaaatgtccaaaggcttcaccgcctttgggaagaagaagtctcaaggcgagggattgaaaaagcttcagtgcttctggtgatgctgaggttccagagaacaaggttgattttcgatgcacttctgggcatctgcttctgcattgccagtgtactcgggccaatattgattataccaaagatcctggaatattcagaagagcagttggggaatgttgtccatggagtgggactctgctttgccctttttctctccgaatgtgtgaagtctctgagtttctcctccagttggatcatcaaccaacgcacagccatcaggttccgagcagctgtttcctcctttgcctttgagaagctcatccaatttaagtctgtaatacacatcacctcaggagaggccatcagcttcttcaccggtgatgtaaactacctgtttgaaggggtgtgctatggacccctagtactgatcacctgcgcatcgctggtcatctgcagcatttcttcctacttcattattggatacactgcatttattgccatcttatgctatctcctggttttcccactggcggtattcatgacaagaatggctgtgaaggctcagcatcacacatctgaggtcagcgaccagcgcatccgtgtgaccagtgaagttctcacttgcattaagctgattaaaatgtacacatgggagaaaccatttgcaaaaatcattgaagacctaagaaggaaggaaaggaaactattggagaagtgcgggcttgtccagagcctgacaagtataaccttgttcatcatccccacagtggccacagcggtctgggttctcatccacacatccttaaagctgaaactcacagcgtcaatggccttcagcatgctggcctccttgaatctccttcggctgtcagtgttctttgtgcctattgcagtcaaaggtctcacgaattccaagtctgcagtgatgaggttcaagaagtttttcctccaggagagccctgttttctatgtccagacattacaagaccccagcaaagctctggtctttgaggaggccaccttgtcatggcaacagacctgtcccgggatcgtcaatggggcactggagctggagaggaacgggcatgcttctgaggggatgaccaggcctagagatgccctcgggccagaggaagaagggaacagcctgggcccagagttgcacaagatcaacctggtggtgtccaaggggatgatgttaggggtctgcggcaacacggggagtggtaagagcagcctgttgtcagccatcctggaggagatgcacttgctcgagggctcggtgggggtgcagggaagcctggcctatgtcccccagcaggcctggatcgtcagcgggaacatcagggagaacatcctcatgggaggcgcatatgacaaggcccgatacctccaggtgctccactgctgctccctgaatcgggacctggaacttctgccctttggagacatgacagagattggagagcggggcctcaacctctctggggggcagaaacagaggatcagcctggcccgcgccgtctattccgaccgtcagatctacctgctggacgaccccctgtctgctgtggacgcccacgtggggaagcacatttttgaggagtgcattaagaagacactcagggggaagacggtcgtcctggtgacccaccagctgcagtacttagaattttgtggccagatcattttgttggaaaatgggaaaatctgtgaaaatggaactcacagtgagttaatgcagaaaaaggggaaatatgcccaacttatccagaagatgcacaaggaagccacttcggacatgttgcaggacacagcaaagatagcagagaagccaaaggtagaaagtcaggctctggccacctccctggaagagtctctcaacggaaatgctgtgccggagcatcagctcacacaggaggaggagatggaagaaggctccttgagttggagggtctaccaccactacatccaggcagctggaggttacatggtctcttgcataattttcttcttcgtggtgctgatcgtcttcttaacgatcttcagcttctggtggctgagctactggttggagcagggctcggggaccaatagcagccgagagagcaatggaaccatggcagacctgggcaacattgcagacaatcctcaactgtccttctaccagctggtgtacgggctcaacgccctgctcctcatctgtgtgggggtctgctcctcagggattttcaccaaagtcacgaggaaggcatccacggccctgcacaacaagctcttcaacaaggttttccgctgccccatgagtttctttgacaccatcccaataggccggcttttgaactgcttcgcaggggacttggaacagctggaccagctcttgcccatcttttcagagcagttcctggtcctgtccttaatggtgatcgccgtcctgttgattgtcagtgtgctgtctccatatatcctgttaatgggagccataatcatggttatttgcttcatttattatatgatgttcaagaaggccatcggtgtgttcaagagactggagaactatagccggtctcctttattctcccacatcctcaattctctgcaaggcctgagctccatccatgtctatggaaaaactgaagacttcatcagccagtttaagaggctgactgatgcgcagaataactacctgctgttgtttctatcttccacacgatggatggcattgaggctggagatcatgaccaaccttgtgaccttggctgttgccctgttcgtggcttttggcatttcctccaccccctactcctttaaagtcatggctgtcaacatcgtgctgcagctggcgtccagcttccaggccactgcccggattggcttggagacagaggcacagttcacggctgtagagaggatactgcagtacatgaagatgtgtgtctcggaagctcctttacacatggaaggcacaagttgtccccaggggtggccacagcatggggaaatcatatttcaggattatcacatgaaatacagagacaacacacccaccgtgcttcacggcatcaacctgaccatccgcggccacgaagtggtgggcatcgtgggaaggacgggctctgggaagtcctccttgggcatggctctcttccgcctggtggagcccatggcaggccggattctcattgacggcgtggacatttgcagcatcggcctggaggacttgcggtccaagctctcagtgatccctcaagatccagtgctgctctcaggaaccatcagattcaacctagatccctttgaccgtcacactgaccagcagatctgggatgccttggagaggacattcctgaccaaggccatcatccttatcgatgaagccacagcctccattgacatggagacagacaccctgatccagcgcacaatccgtgaagccttccagggctgcaccgtgctcgtcattgcccaccgtgtcaccactgtgctgaactgtgaccacatcctggttatgggcaatgggaaggtggtagaatttgatcggccggaggtactgcggaagaagcctgggtcattgttcgcagccctcatggccacagccacttcttcactgagataaggagatgtggagacttcatggaggctggcagctgagctcagaggttcacacaggtgcagcttcgaggcccacagtctgcgaccttcttgtttggagatgagaacttctcctggaagcaggggtaaatgtagggggggtggggattgctggatggaaaccctggaataggctacttgatggctctcaagaccttagaaccccagaaccatctaagacatgggattcagtgatcatgtggttctccttttaacttacatgctgaataattttataataaggtaaaagcttatagttttctgatctgtgttagaagtgttgcaaatgctgtactgactttgtaaaatataaaactaaggaaaactc

SEQ ID NO: 18 - DNA, 4338 bp, Homo sapiens
atgttttgggacagtggactaaactttgccaaaaagtcctctcactctcgtaggactgctctacactgggcctgtgtcaatggccatgaggaagtagtaacatttctggtagacagaaagtgccagcttgacgtccttgatggcgaacacaggacacctctgatgaaggctctacaatgccatcaggaggcttgtgcaaatattctgatagattctggtgccgatataaatctcgtagatgtgtatggcaacacggctctccattatgctgtttatagtgagattttgtcagtggtggcaaaactgctgtcccatggtgcagtcatcgaagtgcacaacaaggctagcctcacaccacttttactatccataacgaaaagaagtgagcaaattgtggaatttttgctgataaaaaatgcaaatgcgaatgcagttaataagtataaatgcacagccctcatgcttgctgtatgtcatggatcatcagagatagttggcatgcttcttcagcaaaatgttgacgtctttgctgcagatatatgtggagtaactgcagaacattatgctgttacttgtggatttcatcacattcatgaacaaattatggaatatatacgaaaattatctaaaaatcatcaaaataccaatccagaaggaacatctgcaggaacacctgatgaggctgcacccttggcggaaagaacacctgacacagctgaaagcttggtggaaaaaacacctgatgaggctgcacccttggtggaaagaacacctgacacggctgaaagcttggtggaaaaaacacctgatgaggctgcatccttggtggagggaacatctgacaaaattcaatgtttggagaaagcgacatctggaaagttcgaacagtcagcagaagaaacacctagggaaattacgagtcctgcaaaagaaacatctgagaaatttacgtggccagcaaaaggaagacctaggaagatcgcatgggagaaaaaagaagacacacctagggaaattatgagtcccgcaaaagaaacatctgagaaatttacgtgggcagcaaaaggaagacctaggaagatcgcatgggagaaaaaagaaacacctgtaaagactggatgcgtggcaagagtaacatctaataaaactaaagttttggaaaaaggaagatctaagatgattgcatgtcctacaaaagaatcatctacaaaagcaagtgccaatgatcagaggttcccatcagaatccaaacaagaggaagatgaagaatattcttgtgattctcggagtctctttgagagttctgcaaagattcaagtgtgtatacctgagtctatatatcaaaaagtaatggagataaatagagaagtagaagagcctcctaagaagccatctgccttcaagcctgccattgaaatgcaaaactctgttccaaataaagcctttgaattgaagaatgaacaaacattgagagcagatccgatgttcccaccagaatccaaacaaaaggactatgaagaaaattcttgggattctgagagtctctgtgagactgtttcacagaaggatgtgtgtttacccaaggctacacatcaaaaagaaatagataaaataaatggaaaattagaagagtctcctaataaagatggtcttctgaaggctacctgcggaatgaaagtttctattccaactaaagccttagaattgaaggacatgcaaactttcaaagcagagcctccggggaagccatctgccttcgagcctgccactgaaatgcaaaagtctgtcccaaataaagccttggaattgaaaaatgaacaaacattgagagcagatgagatactcccatcagaatccaaacaaaaggactatgaagaaaattcttgggatactgaggtactgtgttctttattttctcacctctgcatgtgtcaccctcaaattatttttgatgtttttcagtatatgcttaatggagaatctataaagaaaagcatgaggaataggaaaccttgtgggagtataataacaagagtattggttgagtagtcttttgaaaaatataaattatattttcacaaatagaactccttgagtccccttatggcagtcaagctacagcagtcacaacggcggaaagacatagaaagtatctgacctcttagaaacaagcagctgctgcctgatgagagttactttttgaaatatgcacgagtgaatttttttgtgaagagtctctgtgagactgtttcacagaaggatgtgtgtttacccaaggctacacatcaaaaagaaatagataaaataaaggaaaattagcaagagtctcctgataatgatggttttctgaaggctccctgcagaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaacgttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgagattgactttaaaccaagaagaagagaagagaagaaatgccgatatattaaatgaaaaaattagggaagaattaggaagaatcgaagagcagcataggaaagagttagaagtgaaacaacaacttgaacaggctctcagaatacaagatatagaattgaagagtgtagaaagtaatttgaatcaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgccggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcatcaccttatgttgaaaatcttaccaatagtctgtgtcaacagaatacttattttagaagaaaaattcatgatttcttcctgaagcctacagacataaaataacagtgtgaagaattacttgttcacgaattgcataaagctgcacaggattcccatctaccctgatgatgcagcagacatcattcaatccaaccagatcgtgccactgtactccagcctgggcgacagagtgagactccatctcaaaaa

SEQ ID NO: 19 - DNA, 1784 bp, Homo sapiens
aacaccctcctggaggatgctggtgagaggcagggaccaggggtccggctcccggctcgggcctatcgttaggcgctgggcccccaggccctctcctttgcagagtctcgctgcctccctcgacgcagagccttcaagcgccgcagtccccgacggcttccccgcgggccccactgtctccccaagacgcctggcgaggccgccggggctggaggaggcgctgagcgcgctggggctgcagggagaacgcgagtacgccggggacatcttcgccgaagtcatggagtacctgggtctggctggtgacacactttatctggcggttcacctgcttgattcctacctgagcgctggccgcgtgcgtctacatcgcctgcagctgctgggcgtggcttgcctgtttgtggcgtgcaaaatggaagagtgcgtgcttcccgaggaaactgaggtccggaacttggggcctttccagggcagggagtaaagagcccggatccaagactccttcacctccccccgcatcccccatctgcagcccgccttcctctgcctcctgagcgcggactccttctcacgggcggagctgctgcgcgccgagcgtcgcatcctgagccgcctggatttccggctgcaccaccccggcccgctgctgtgcctcgggctgctggccgcgctggcagggagcagcccccaggtgatgttacttgccacctacttcctggagctgtctttgctggaggccgaggcggcgggatgggagccgggtcgtcgtgcggctgcggctctgagcctggcgcaccgcttgctcgacggggcgggctccaggctccagccagaactttacaggtgtagtcttggcggaggaagtgtatggggtcaccgcagcttcagggacttaccttcctggtcatttttacggtctcggagaatgagagacaattattgaggaggaggtggcacctagacttgatttttctgggtgtgggagagaatggggtgggaggacccccattagtccagatctggggtctcttaaccttgccccagagtggaagatagtctcccaggcccagaagatgctcctattgccttccagggctgagaacgaaggatcacccaggcctcgagtcctccatctctgtatctggtggtggatggggtgttcctttagctgctagggccgctaaccatgctaccaggtggcagggcgaaccatggtttccctcagcctgtgcacccagcatagagaggatggctgcccatcctcagctcccctccttgcttcctcgagtgttctgactccgcactagccgcgccctgtaggaagaatagggtgtccacctctccccggtgctcgcctagtcactccagttgaagacgggacgcgtgcccgatctcaagagagcccccgacccgtccgtggggaaccacatcgacgcttcttctcagcctccagtctccagttccaaggatgggtcatctccaaccacttgccctgcctcagtttctccatctccctgctgcagccccgaggaactgggcaccctcgagccgtgcatggcccgcgctgcgctccgaggtcccgcgccgggtcgcgccgcagtcttcctcaagtatgcgcggccccagcgccagggggccagccttgccgccgcctgcctgctccgccgcctccagtctgagcctccctgagtactgggactcagtcacaaaaaaatcaacaacaaaaaacaaaaccctcccagtgtgtgtccgtctctcatctcaataaaagaatttatttt

SEQ ID NO: 20 - DNA, 7290 bp, Homo sapiens
acacagtactctcagcttgttggtggaagcccctcatctgccttcattctgaaggcagggcccggcagaggaaggatcagagggtcgcggccggagggtcccggccggtggggccaactcagagggagaggaaagggctagagacacgaagaacgcaaaccatcaaatttagaagaaaaagccctttgactttttccccctctccctccccaatggctgtgtagcaaacatccctggcgataccttggaaaggacgaagttggtctgcagtcgcaatttcgtgggttgagttcacagttgtgagtgcggggctcggagatggagccgtggtcctctaggtggaaaacgaaacggtggctctgggatttcaccgtaacaaccctcgcattgaccttcctcttccaagctagagaggtcagaggagctgctccagttgatgtactaaaagcactagattttcacaattctccagagggaatatcaaaaacaacgggattttgcacaaacagaaagaattctaaaggctcagatactgcttacagagtttcaaagcaagcacaactcagtgccccaacaaaacagttatttccaggtggaactttcccagaagacttttcaatactatttacagtaaaaccaaaaaaaggaattcagtctttccttttatctatatataatgagcatggtattcagcaaattggtgttgaggttgggagatcacctgtttttctgtttgaagaccacactggaaaacctgccccagaagactatcccctcttcagaactgttaacatcgctgacgggaagtggcatcgggtagcaatcagcgtggagaagaaaactgtgacaatgattgttgattgtaagaagaaaaccacgaaaccacttgatagaagtgagagagcaattgttgataccaatggaatcacggtttttggaacaaggattttggatgaagaagtttttgagggggacattcagcagtttttgatcacaggtgatcccaaggcagcatatgactactgtgagcattatagtccagactgtgactcttcagcacccaaggctgctcaagctcaggaacctcagatagatgagtatgcaccagaggatataatcgaatatgactatgagtatggggaagcagagtataaagaggctgaaagtgtaacagagggacccactgtaactgaggagacaatagcacagacggaggcaaacatcgttgatgattttcaagaatacaactatggaacaatggaaagttaccagacagaagctcctaggcatgtttctgggacaaatgagccaaatccagttgaagaaatatttactgaagaatatctaacgggagaggattatgattcccagaggaaaaattctgaggatacactatatgaaaacaaagaaatagacggcagggattctgatcttctggtagatggagatttaggcgaatatgatttttatgaatataaagaatatgaagataaaccaacaagcccccctaatgaagaatttggtccaggtgtaccagcagaaactgatattacagaaacaagcataaatggccatggtgcatatggagagaaaggacagaaaggagaaccagcagtggttgagcctggtatgcttgtcgaaggaccaccaggaccagcaggacctgcaggtattatgggtcctccaggtctacaaggccccactggaccccctggtgaccctggcgataggggccccccaggacgtcctggcttaccaggggctgatggtctacctggtcctcctggtactatgttgatgttaccgttccgttatggtggtgatggttccaaaggaccaaccatctctgctcaggaagctcaggctcaagctattcttcagcaggctcggattgctctgagaggcccacctggcccaatgggtctaactggaagaccaggtcctgtgggggggcctggttcatctggggccaaaggtgagagtggtgatccaggtcctcagggccctcgaggcgtccagggtccccctggtccaacgggaaaacctggaaaaaggggtcgtccaggtgcagatggaggaagaggaatgccaggagaacctggggcaaagggagatcgagggtttgatggacttccgggtctgccaggtgacaaaggtcacaggggtgaacgaggtcctcaaggtcctccaggtcctcctggtgatgatggaatgaggggagaagatggagaaattggaccaagaggtcttccaggtgaagctggcccacgaggtttgctgggtccaaggggaactccaggagctccagggcagcctggtatggcaggtgtagatggccccccaggaccaaaagggaacatgggtccccaaggggagcctgggcctccaggtcaacaagggaatccaggacctcagggtcttcctggtccacaaggtccaattggtcctcctggtgaaaaaggaccacaaggaaaaccaggacttgctggacttcctggtgctgatgggcctcctggtcatcctgggaaagaaggccagtctggagaaaagggggctctgggtccccctggtccacaaggtcctattggatacccgggcccccggggagtaaagggagcagatggtgtcagaggtctcaagggatctaaaggtgaaaagggtgaagatggttttccaggattcaaaggtgacatgggtctaaaaggtgacagaggagaagttggtcaaattggcccaagaggggaagatggccctgaaggacccaaaggtcgagcaggcccaactggagacccaggtccttcaggtcaagcaggagaaaagggaaaacttggagttccaggattaccaggatatccaggaagacaaggtccaaagggttccactggattccctgggtttccaggtgccaatggagagaaaggtgcacggggagtagctggcaaaccaggccctcggggtcagcgtggtccaacgggtcctcgaggttcaagaggtgcaagaggtcccactgggaaacctgggccaaagggcacttcaggtggcgatggccctcctggccctccaggtgaaagaggtcctcaaggacctcagggtccagttggattccctggaccaaaaggccctcctggaccacctgggaaggatgggctgccaggacaccctgggcaacgtggggagactggatttcaaggcaagaccggccctcctgggccagggggagtggttggaccacagggaccaaccggtgagactggtccaataggggaacgtgggcatcctggccctcctggccctcctggtgagcaaggtcttcctggtgctgcaggaaaagaaggtgcaaagggtgatccaggtcctcaaggtatctcagggaaagatggaccagcaggattacgtggtttcccaggggaaagaggtcttcctggagctcagggtgcacctggactgaaaggaggggaaggtccccagggcccaccaggtccagttggctcaccaggagaacgtgggtcagcaggtacagctggcccaattggtttaccagggcgcccgggacctcagggtcctcctggtccagctggagagaaaggtgctcctggagaaaaaggtccccaagggcctgcagggagagatggagttcaaggtcctgttggtctcccagggccagctggtcctgccggctcccctggggaagacggagacaagggtgaaattggtgagccgggacaaaaaggcagcaagggtgacaagggagaaaatggccctcccggtcccccaggtcttcaaggaccagttggtgcccctggaattgctggaggtgatggtgaaccaggtcctagaggacagcaggggatgtttgggcaaaaaggtgatgagggtgccagaggcttccctggacctcctggtccaataggtcttcagggtctgccaggcccacctggtgaaaaaggtgaaaatggggatgttggtcccatggggccacctggtcctccaggcccaagaggccctcaaggtcccaatggagctgatggaccacaaggacccccagggtctgttggttcagttggtggtgttggagaaaagggtgaacctggagaagcagggaacccagggcctcctggggaagcaggtgtaggcggtcccaaaggagaaagaggagagaaaggggaagctggtccacctggagctgctggacctccaggtgccaaggggccaccaggtgatgatggccctaagggtaacccgggtcctgttggttttcctggagatcctggtcctcctggggaacctggccctgcaggtcaagatggtgttggtggtgacaagggtgaagatggagatcctggtcaaccgggtcctcctggcccatctggtgaggctggcccaccaggtcctcctggaaaacgaggtcctcctggagctgcaggtgcagagggaagacaaggtgaaaaaggtgctaagggggaagcaggtgcagaaggtcctcctggaaaaaccggcccagtcggtcctcagggacctgcaggaaagcctggtccagaaggtcttcggggcatccctggtcctgtgggagaacaaggtctccctggagctgcaggccaagatggaccacctggtcctatgggacctcctggcttacctggtctcaaaggtgaccctggctccaagggtgaaaagggacatcctggtttaattggcctgattggtcctccaggagaacaaggggaaaaaggtgaccgagggctccctggaactcaaggatctccaggagcaaaaggggatgggggaattcctggtcctgctggtcccttaggtccacctggtcctccaggtttaccaggtcctcaaggcccaaagggtaacaaaggctctactggacccgctggccagaaaggtgacagtggtcttccagggcctcctgggtctccaggtccacctggtgaagtcattcagcctttaccaatcttgtcctccaaaaaaacgagaagacatactgaaggcatgcaagcagatgcagatgataatattcttgattactcggatggaatggaagaaatatttggttccctcaattccctgaaacaagacattgagcatatgaaatttccaatgggtactcagaccaatccagcccgaacttgtaaagacctgcaactcagccatcctgacttcccagatggtgaatattggattgatcctaaccaaggttgctcaggagattccttcaaagtttactgtaatttcacatctggtggtgagacttgcatttatccagacaaaaaatctgagggagtaagaatttcatcatggccaaaggagaaaccaggaagttggtttagtgaatttaagaggggaaaactgctttcatacttagatgttgaaggaaattccatcaatatggtgcaaatgacattcctgaaacttctgactgcctctgctcggcaaaatttcacctaccactgtcatcagtcagcagcctggtatgatgtgtcatcaggaagttatgacaaagcacttcgcttcctgggatcaaatgatgaggagatgtcctatgacaataatccttttatcaaaacactgtatgatggttgtgcgtccagaaaaggctatgaaaagactgtcattgaaatcaatacaccaaaaattgatcaagtacctattgttgatgtcatgatcaatgactttggtgatcagaatcagaagttcggatttgaagttggtcctgtttgttttcttggctaagattaagacaaagaacatatcaaatcaacagaaaatataccttggtgccaccaacccattttgtgccacatgcaagttttgaataaggatggtatagaaaacaacgctgcatatacaggtaccatttaggaaataccgatgcctttgtgggggcagaatcacatggcaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccatctgactctaattttatgagaattgcagaactctgatggcaataaatatatgtattatgaaaaaataaagttgtaatttctgatgactctaagtccctttctttggttaataataaaatgcctttgtatatattgatgttgaagagttcaattatttgatgtcgccaacaaaattctcagagggcaaaaatctggaagacttttggaagcacactctgatcaactcttctctgccgacagtcattttgctgaatttcagccaaaaatattatgcattttgatgctttattcaaggctatacctcaaactttttcttctcagaatccaggatttcacaggatacttgtatatatggaaaacaagcaagtttatatttttggacagggaaatgtgtgtaagaaagtatattaacaaatcaatgcctccgtcaagcaaacaatcatatgtatactttttttctacgttatctcatctccttgttttcagtgtgcttcaataatgcaggttaatattaaagatggaaattaagcaattatttatgaatttgtgcaatgttagattttcttatcaatcaagttcttgaatttgattctaagttgcatattataacagtctcgaaaattattttacttgcccaacaaatattacttttttcctttcaagataattttataaatcatttgacctacctaattgctaaatgaataacatatggtggactgttattaagagtatttgttttaagtcattcaggaaaatctaaacttttttttccactaaggtatttactttaaggtagcttgaaatagcaatacaatttaaaaattaaaaactgaattttgtatctattttaagtaatatatgtaagacttgaaaataaatgttttatttcttatataaagtgttaaattaattgataccagatttcactggaacagtttcaactgataatttatgacaaaagaacatacctgtaatattgaaattaaaaagtgaaatttgtcataaagaatttcttttatttttgaaatcgagtttgtaaatgtccttttaagaagggagatatgaatccaataaataaactcaagtcttggctacctgg

SEQ ID NO: 21 - DNA, 1688 bp, Homo sapiens
ctctggttcccttccacgctgtggaagctttgttcttttggtcttcatgataaatcttgctgctgctcactcgttgggtccgtgccacctttaagagctgtaacactcaccgcgaaggtctgcaacttcactcctggggccagcaagaccacgaatgcaccgagaggaatgaacaactctggacacaccatctttaagaaccgtaatactcaccgcaagggtctgcaacttcattcttgaagtcagtgaggccaagaacccatcaattccgtacacattttggtgactttgaagagactgtcacctatcaccaagtggtgagactattgccaagcagtgagactattgccaagtggtgagaccatcaccaagcggtgagactatcacctatcgccaagtggcctgattcagcaggaagcatctcagacaccaaccactatgctgtcagcagttgcccggggctaccagggctggtttcatccctgtgctaggctttctgtgaggatgagcagcaccgggatagacaggaagggcgtcctggctaaccgggtagccgtggtcacggggtccaccagtgggatcggctttgccatcgcccgacgtctggcccgggacggggcccacgtggtcatcagcagccggaagcagcagaacgtggaccgggccatggccaagctgcagggggaggggctgagtgtggcgggcattgtgtgccacgtggggaaggctgaggaccgggagcagctggtggccaaggccctggagcactgtgggggcgtcgacttcctggtgtgcagcgcaggggtcaaccctctggtagggagcactctggggaccagtgagcagatctgggacaagatcctaagtgtgaacgtgaagtccccagccctgctgctgagccagttgctgccctacatggagaacaggaggggtgctgtcatcctggtctcttccattgcagcttataatccagtagtggcgctgggtgtctacaatgtcagcaagacagcgctgctgggtctcactagaacactggcattggagctggcccccaaggacatccgggtaaactgcgtggttccaggaattatcaaaactgacttcagcaaagtggtgaggattggtttcatgggaatgagtctctctggaagaacttcaaggaacatcatcagctgcagaggattggggagtcagaggactgtgcaggaatcgtgtccttcctgtgctctccagatgccagctacgtcaacggggagaacattgcggtggcaggctactccactcggctctgagaggagtgggggcggctgcgtagctgtggtcccaggcccaggagcctgagggggtgtctaggtgatcatttggatctggaggcagagtctgccattctgccagactagcaatttgggggcttactcatgctaggcttgaggaagaagaaaaacgcttcggcattctccttaggacttatctgcttgtagatttggctgatccaattaacatgtggggttcttggtgtgggtctggggagctgaaggattttatggagctggtgctttggaggaatcttaagggaaaggagtagaagctcaggcctttgaaggatttcagctcctcctctctgtaatttgtgctttaagcattttttttcctaaaataaactcaaatttatcctcaaaaaaaaaaaa

SEQ ID NO: 22 - DNA, 466 bp, Homo sapiens
ctatggcacgcacgaagcaaacagctcgtaagtccactggcggcaaagccccgcgcaagcagctggccactaaggcggctcgcaaaagcgcgccagccaccggtggcgtgaagaagccccaccgctacaggcctggtactgtcgccctccgtgaaatccgccgctatcagaaatcgactgagctactgattcgcaagctaccattccagcgtctggtacgtgagatcgcgcaggacttcaagaccgacctgcgcttccagagctcggctgtgatggcgctgcaggaggcctgcgaggcttacctggtggggctctttgaggacaccaacctgtgtgctatccacgccaagcgagtgactatcatgcccaaggacatccagctcgctcgccgcattcgcggagagagggcataagttgtactgagggtgtgcgccaacttaaaccaaaggctcttttcagagccacc

SEQ ID NO: 23 - DNA, 473 bp, Homo sapiens
ctctgaaggcatggcgcgtacgaagcagactgctcgcaagtccaccggcggcaaggctccgcgcaagcagctggccaccaaggcggctcggaagagcgctccggccaccggcggtgtcaagaagccccatcgctatcggcctggtacagtggctctccgcgagattcgccgctaccagaagtccaccgagctgctgatcagaaagctgccttttcagcgtctggtgcgtgagatcgcgcaggacttcaagaccgacttgcgcttccagagctccgcggtgatggcgctgcaagaggcatgcgaggcctacctggtggggctctttgaggacaccaacctgtgcgccatccacgccaagcgggtgactatcatgcccaaggacatccagctcgcacgtcgtatccgcggcgagagggcttgagtctcaaggactcactgattacatacccaaaggctcttttcagagccaccca

SEQ ID NO: 24 - DNA, 448 bp, Homo sapiens
atgtcaggacgcggaaagcagggaggcaaggcccgcgctaaggccaagtcgcgctcgtcccgcgctggtctccagttcccggtggggcgagtgcaccgcttgctgcgcaaaggcaactacgcggagcgggtcggggcaggcgccccggtgtacctggcggcggtcctcgagtacctgaccgcggaaattctggagctggcgggcaacgcggctcgggacaacaagaagacgcgcatcatccctcgccatctgcaactagccgtgaggaatgacgaagagctcaacaagttactcgggggtgtcaccattgcccagggcggcgtcttgcccaatatccaggctgtcctgttgcccaagaaaacggagagtcacaagcctggcaagaacaagtaattaagaggcttgacaccatactcattcaccccaaaggctcttttaagagccacca

SEQ ID NO: 25 - DNA, 1286 bp, Homo sapiens
ggagcgcgcggtccgggcacacggagcaggttgggaccgcggcgggtaccggggccggggcgccatgcggaggccgagcgtgcgcgcggccgggctggtcctgtgcaccctgtgttacctgctggtgggcgctgctgtcttcgacgcgctcgagtccgaggcggaaagcggccgccagcgactgctggtccagaagcggggcgctctccggaggaagttcggcttctcggccgaggactaccgcgagctggagcgcctggcgctccaggctgagccccaccgcgccggccgccagtggaagttccccggctccttctacttcgccatcaccgtcatcactaccatcgggtacggccacgccgcgccgggtacggactccggcaaggtcttctgcatgttctacgcgctcctgggcatcccgctgacgctggtcactttccagagcctgggcgaacggctgaacgcggtggtgcggcgcctcctgttggcggccaagtgctgcctgggcctgcggtggacgtgcgtgtccacggagaacctggtggtggccgggctgctggcgtgtgccgccaccctggccctcggggccgtcgccttctcgcacttcgagggctggaccttcttccacgcctactactactgcttcatcaccctcaccaccatcggcttcggcgacttcgtggcactgcagagcggcgaggcgctgcagaggaagctcccctacgtggccttcagcttcctctacatcctcctggggctcacggtcattggcgccttcctcaacctggtggtcctgcgcttcctcgttgccagcgccgactggcccgagcgcgctgcccgcccccccagcccgcgccccccgggggcgcccgagagccgtggcctctggctgccccgccgcccggcccgctccgtgggctccgcctctgtcttctgccacgtgcacaagctggagaggtgcgcccgcgacaacctgggcttttcgcccccctcgagcccgggggtcgtgcgtggcgggcaggctcccaggcctggggcccggtggaagtccatctgacaaccccacccaggccagggtcgaatctggaatgggagggtctggcttcagctatcagggcaccctccccagggattggaaacggatgacgggcctctaggcggtcttctgccacgagcagtttctcattactgtctgtggctaagtcccctccctcctttccaaaaatatattacagtcacaccataaaaaaaaaaaaaaaaaaaaaaaaaaaaaaaaaaaaaaaaaa

SEQ ID NO: 26 - DNA, 751 bp, Homo sapiens
atgggccccggggacttccgccgctgcagagagagaatttcccaggggctccagggactcccaggtagagcggagctttggttcccacctcgtcccgcgtgcgacttcttcggggacggcaggagcacggacatccaggaggaggccctcgccgccagcccgctgctggaggacctcagacgacggctgacgcgcgccttccagtgggcggtgcagcgcgcgatctcgaggcgcgtgcaggaggcggcggcggcggcggcggcgcgggaggagcagagctggacgggcgttgaggccaccctggccaggctgcgggcggagctggtggaaatgcatttccaaaaccaccagctggctagaactttactggacctaaacatgaaagtgcagcaattgaaaaaggagtatgaactggaaattacatcagactcccaaagcccaaaagatgatgctgcgaatccggaataaagaaatgcacacgcaagggctgggcgcggtggctcacgcctgtaatcccagcactttgggaggccgaggcgggcggatcaagacgtcaggagattgagaccatcctggctaacactgtgaaaccctgcctctactaaaaatacaaaaaattagccagacgtggtggcaggcacctgtagtccctgctactcaggagtctgaggcaggagaatggcgtgaacccaggagacagagcttgcagtgagccgaaatcctgccactgcactccagccctgggtgacag

SEQ ID NO: 27 - DNA, 1568 bp, Homo sapiens
cttaatattcacttttaattcattccaccagagaggaaatgcatgggttttggataggactctgtcatttatccctgtaagaacttgagaaatttatttagtctctctaagctttggcgtattcaaaatgaagacgatactaacagtacctacatcatgagtgttacttgaatggagatgtgtaaagtgcattttaatttcatctggtcctttaacattttcccttcacttctcttcagagttttttcttgaagggctcgaggactgaagcatcccacaaaatgattctattgaataattctgagcagctgctggccctattcaaatctttagcaaggagcattcctgagtccctgaaggtgtatggctctctgtttcacatcaatcacgggaaccccttcaacatggaggtgttggtggactcctggcccgggtatcagatggttattatccgacctcaaaaacaggagatgactgatgacatggattcatacactaatgtatatcgtatattctccaaagaccctcaaaaatcacaagaagttttgaaaaattctgagatcataaactggaaacagaaactccaaatccaaggttttcaagaaagtttaggtgaggggataaaagcagctgcattttcaaattcagtgaaggtagagcattcgagagcactcctctttgttacggaagatatcctgaagctctatgccaccaataaaagcaagcttggaagctgggctgagacaggccacccagatgacgaattggagagcgagactcccaattttaagtatgcccagctgaatgtgtcttattctgggctggtaaatgacaactggaagctagggatgaataagaggagcctgcattacatcaagcgctgcctaggagccctgccagcagtctgtatgctgggcccagagggggtcccggtctcatgggtaaccatggacccttcttgtgaaataggaatgggctacagtgtggaaaaataccgaaggagaggcaacgcgacacggatgatggtgcgatacatgaagtatctgtgtcagaagaatattccattttacggctctgtgctggaagaaaatcaaggcgccatcagaatgaataaggcactaggtttccttgaggcctcctgtcagtggcaccaatggacctgctacccacagaatcttgttccgttgtagacaatgaagctgcttagcaatcttgggcaagccatctcttaatattaaagcagacaccacagaatagctttcttcacttacaaatgttgattgggcatttatgatatggcaggaactccttctcacatggagacctgatgttaaaggacacagccatgctcttgaggagcttacaatccaagctggaggcaggggagggtatagtctttaaatatgcttaagtgttgtagggaaggacagagttaccaataaacatgtaactagaaagccaggctcagttcttacctctgggaatcagaactctttatgaaacttgattgatagaatctactatctggaagataattgaagaactttaataaaattgtcaatagaata

SEQ ID NO: 28 - DNA, 653 bp, Homo sapiens
gttgaagagatgagtgcggggctcatctatccctggaattgtctttcccacaatccctgacacagaatatgagccatacaggaattctgaagaaatgggtctcttgccacctcccagtaaaagattattttttaaaaaaaaaaggctctgctttgacctgaagtattttatctatcctcagtctcaggacactgttgatggaattaaggccaagcacatctgcaaaaaagacattgctggaggaggtgcaaagagctggaaaccaagtctccagtcctgggaaaagcagtggtatggaaaagcaatggaaagagcattttgaaaatgccattccactgttttctggcctttatgatttctgctgagaaatccactgttagtctgatggggtctccttcatagcaccaatgacctgaagagccttgttgaaggaagactccatctgatgactcagagcaagtattttttagtgtgttattgttattagcagaaagagggccataaaatacatggggcaagctgaatatatcttaggcaaaagaagaaaatattcaaattcttatgttattttatctaattattttatctctttttgtgtgtgacttataatgtgtgtattgtattaataaaagtatataaacatgtagtttaca

SEQ ID NO: 29 - DNA, 12644 bp, Homo sapiens
cctcccgcctcagttcgcgccgcgcctcggcttggaacgcaggagcgccggctccgggagcccgagcggagccagccgcgcgcacagccagcggccgcgccggcgatgcggggccaccccgcgcccgccccagtcccggccccggcccccgcgggaaggggctgagctgcccgccgccgcccggatggcgagcctcgccgcgctcgccctcagcctgctcctgaggctgcagctgccgccactgcccggcgcccgggctcagagcgccgcaggtggctgttcctttgatgagcactacagcaactgtggttatagtgtggctctagggaccaatgggttcacctgggagcagattaacacatgggagaaaccaatgctggaccaggcagtgcccacaggatctttcatgatggtgaacagctctgggagagcctctggccagaaggcccaccttctcctgccaaccctgaaggagaatgacacccactgcatcgacttccattactacttctccagccgtgacaggtccagcccaggggccttgaacgtctacgtgaaggtgaatggtggcccccaagggaaccctgtgtggaatgtgtccggggtcgtcactgagggctgggtgaaggcagagctcgccatcagcactttctggccacatttctatcaggtgatatttgaatccgtctcattgaagggtcatcctggctacatcgccgtggacgaggtccgggtccttgctcatccatgcagaaaagcacctcattttctgcgactccaaaacgtggaggtgaatgtggggcagaatgccacatttcagtgcattgctggtgggaagtggtctcagcatgacaagctttggctccagcaatggaatggcagggacacggccctgatggtcacccgtgtggtcaaccacaggcgcttctcagccacagtcagtgtggcagacactgcccagcggagcgtcagcaagtaccgctgtgtgatccgctctgatggtgggtctggtgtgtccaactacgcggagctgatcgtgaaagagcctcccacgcccattgctcccccagagctgctggctgtgggggccacatacctgtggatcaagccaaatgccaactccatcatcggggatggccccatcatcctgaaggaagtggaatatcgcaccaccacaggcacgtgggcagagacccacatagtcgactctcccaactataagctgtggcatctggaccccgatgttgagtatgagatccgagtgctcctcacacgaccaggtgaggggggtacgggaccgccagggcctcccctcaccaccaggaccaagtgtgcagatccggtacatggcccacagaacgtggaaatcgtagacatcagagcccggcagctgaccctgcagtgggagcccttcggctacgcggtgacccgctgccatagctacaacctcaccgtgcagtaccagtatgtgttcaaccagcagcagtacgaggccgaggaggtcatccagacctcctcccactacaccctgcgaggcctgcgccccttcatgaccatccggctgcgactcttgctgtctaaccccgagggccgaatggagagcgaggagctggtggtgcagactgaggaagacgttccaggagctgttcctctagaatccatccaaggggggccctttgaggagaagatctacatccagtggaaacctcccaatgagaccaatggggtcatcacgctctacgagatcaactacaaggctgtcggctcgctggacccaagtgctgacctctcgagccagagggggaaagtgttcaagctccggaatgaaacccaccacctctttgtgggtctgtacccagggaccacctattccttcaccatcaaggccagcacagcaaagggctttgggccccctgtcaccactcggattgccaccaaaatttcagctccatccatgcctgagtacgacacagacaccccattgaatgagacagacacgaccatcacagtgatgctgaaacccgctcagtcccggggagctcctgtcagtgtttatcagctggttgtcaaggaggagcgacttcagaagtcacggagggcagctgacattattgagtgcttttcggtgcccgtgagctatcggaatgcctccagcctcgattctctacactactttgctgctgagttgaagcctgccaacctgcctgtcacccagccatttacagtgggtgacaataagacatacaatggctactggaaccctcctctctctcccctgaaaagctacagcatctacttccaggcactcagcaaagccaatggagagaccaaaatcaactgtgttcgtctggctacaaaaggtgcctccacccagaattctaacactgtggagccagagaagcaggtggacaacaccgtgaagatggctggcgtgatcgctggcctcctcatgttcatcatcattctcctgggcgtgatgctcaccatcaaaaggagaagaaatgcttattcctactcctattacttgaagctggccaagaagcagaaggagacccagagtggagcccagagggagatggggcctgtggcctctgccgacaaacccaccaccaagctcagcgccagccgcaatgatgaaggcttctcttctagttctcaggacgtcaacggattcacagatggcagccgcggggagctttcccagcccaccctcacgatccagactcatccctaccgcacctgtgaccctgtggagatgagctacccccgggaccagttccaacccgccatccgggtggctgacttgctgcagcacatcacgcagatgaagagaggccagggctacgggttcaaggaggaatacgaggccttaccagaggggcagacagcttcgtgggacacagccaaggaggatgaaaaccgcaataagaatcgatatgggaacatcatatcctacgaccattcccgggtgaggctgctggtgctggatggagacccgcactctgactacatcaatgccaactacattgacggataccatcgacctcggcactacattgcgactcaaggtccgatgcaggagactgtaaaggacttttggagaatgatctggcaggagaactccgccagcatcgtcatggtcacaaacctggtggaagtgggcagggtgaaatgtgtgcgatactggccagatgacacggaggtctacggagacattaaagtcaccctgattgaaacagagcccctggcagaatacgtcatacgcaccttcacagtccagaagaaaggctaccatgagatccgggagctccgcctcttccacttcaccagctggcctgaccacggcgttccctgctatgccactggccttctgggcttcgtccgccaggtcaagttcctcaaccccccggaagctgggcccatagtggtccactgcagtgctggggctgggcggactggctgcttcattgccattgacaccatgcttgacatggccgagaatgaaggggtggtggacatcttcaactgcgtgcgtgagctccgggcccaaagggtcaacctggtacagacagaggagcaatatgtgtttgtgcacgatgccatcctggaagcgtgcctctgtggcaacactgccatccctgtgtgtgagttccgttctctctactacaatatcagcaggctggacccccagacaaactccagccaaatcaaagatgaatttcagaccctcaacattgtgacaccccgtgtgcggcccgaggactgcagcattgggctcctgccccggaaccatgataagaatcgaagtatggacgtgctgcctctggaccgctgcctgcccttccttatctcagtggacggagaatccagcaattacatcaacgcagcactgatggatagccacaagcagcctgccgccttcgtggtcacccagcaccctctacccaacaccgtggcagacttctggaggctggtgttcgattacaactgctcctctgtggtgatgctgaatgagatggacactgcccagttctgtatgcagtactggcctgagaagacctccgggtgctatgggcccatccaggtggagttcgtctccgcagacatcgacgaggacatcatccacagaatattccgcatctgtaacatggcccggccacaggatggttatcgtatagtccagcacctccagtacattggctggcctgcctaccgggacacgcccccctccaagcgctctctgctcaaagtggtccgacgactggagaagtggcaggagcagtatgacgggagggagggacgtactgtggtccactgcctaaatgggggaggccgtagtggaaccttctgtgccatctgcagtgtgtgtgagatgatccagcagcaaaacatcattgacgtgttccacatcgtgaaaacactgcgtaacaacaaatccaacatggtggagaccctggaacagtataaatttgtatacgaggtggcactggaatatttaagctccttttagctcaatgggatggggaacctgccggagtccagaggctgctgtgaccaagcccccttttgtgtgaatggcagtaactgggctcaggagctctgaggtggcaccctgcctgactccaaggagaagactggtggccctgtgttccacggggggctctgcaccttctgaggggtctcctgttgccgtgggagatgctgctccaaaaggcccaggcttccttttcaacctaaccagccacagccaagggcccaagcagaagtacacccacaagcaaggccttggatttctggctcccagaccacctgcttttgttctgagtttgtggatctcttggcaagccaactgtgcaggtgctggggagtgggaggctcccctgccctccttctccttaggagtggaggagatgtgtgttctgctcctctacgtcatggaaaagattgaggctcttgggggtcactgctctgctgccccctgcaacctccttcaggggcctctggcaccagacatttgcagtctggaccagtgtgaccttacgatgttccctaggccacaagagaggccccccatcctcacacctaacctgcatggggcttcgcccacaaccattctgtaccccttccccagcctgggccttgaccgtccagcattcactggccggccagctgtgtccacagcagtttttgataaaggtgttctttgcttttttgtgtggtcagtgggagggggtggaactgcagggaacttctctgctcctccttgtctttgtaaaaagggaccacctccctggggcagggcttgggctgacctgtaggatgtaacccctgtgtttctttggtggtagctttctttggaagagacaaacaagataagatttgattattttccaaagtgtatgtgaaaagaaactttcttttggagggtgtaaaatcttagtctcttatgtcaaaaagaagggggcgggggagtttgagtatgtacctctaagacaaatctctcgggccttttattttttcctggcaatgtccttaaaagctcccaccctgggacagcatgccactgagcaaggagagatgggtgagcctgaagatggtccctttggtttctggggcaaatagagcaccagctttgtgcataatttggatgtccaaatttgaactccttcctaaagaaacccagcagccaccttgaaaaaggccattgtggagcccattatactttgatttaaaataggccaagagaatcaggcctggagatctagggtcttgtccaaagtgtgagtgagtcaatgagagggaaccaacatttgctaagtctctactgtatgccagggatcatgcttggcactttccataggacatttcacacagtccttagaacccccaggagagagctactgacttgttatcatctccatttgatcatctcctccaatgaggaaacccacgcaccttccttagtaatgaaatcctgggttccaaaggggcaggtaatggcaatgagacttctccgtgctgttttcttcatcttctctaagccaagcaattattttatggagggaaaataaggccagaaacttctgagcagataactccacaaatggaaatttagtactttcttcctgatgccagttcttctgggaagcgcagaatttcagatatattttagtaacacattcccagctccccaggaaagccagtctcatctaatttcttagtcagtaaaaacaattccctgttccttcaggctatgaatggaccagccagggaaactctcgaccttgatctctagccagtgcttaggcccaatatctgacagcctcaggtgggctgggacctaggaagctccatcttgaaggctggtctagccccagacagggcatgaggggcagagaattcaagaaggtacagctttggccctcaagagcccactgtatgctggggaaatggaaccatggtgcagtagtgtggagtggatgagtgttccatgagcctaggagcaagaaagtctcttcggcctcgggcttcctggagaaggggacgtccattcctgctgggtcttaacaagcataaaaaggaaaaaaaggaaactcaggcaaagggatccatatgtgcaatggcaaagaaatgtgaaaaggcattgggagaagcagtctgggggaggccagcccagtgcgggcacagcacaacacggggagcagcaagagatgagccagggtccaggagacagatgcccatcgcgagtacagactttgtcctattggcaacaaggagtccatggagctttagagagatgcactcagcttcgtgttggccaagactccttctgggccaatggggctgcctcttttcctttcatcagacactgtgaaaacattcccttaagcgtgcactttttaatatcacatctatttgtctgtctgctcattgttttgttgctggaactaaatatgcaatggatcatgagactcagattctatgagaaacccagggtctctgctttaccacggagcagggtcaccaacccagatctccaggcccatgaggatggaacatgaaaggagccgacaaaagttgcttccattggcatgggctctggagctgtccagaagtccagggacaccagacttgatcaaggaagggctgtcactttagaggttcaaaaggaagtgcctcaaagcaaaggcaagcaaaggaaccccacgatgaacttgctcttttcctttgatgagcctctccccaggtgtatttcagcagaccccggggacccacccccactgggcctgctggcctccctcggctccagcccaatgccccagctggccttccccagcctgcaaggagcctgtagcatggcaaatctgcctgctgtatgctattttcttagatcttggtacatccagacaggatgagggtggagggagagctatttaacacaaatcctaagatttttttctgctcaggaaggggtgaaatagctggcagatacaaaagacagtggcttttatcattttaaatggtaggaatttaaggtgtgacttcagggagaaacaaacttgcaaaaaaaaaaaatctcaggccatgttggggtaacccagcaagggccagtgatgatttcccccagctcatccccttattttcccacaacccaaccattctctaaagcaggacagtgaataggtcttaggccagtgcacacaggaagaaattgaggcttatggatggggatgacttccctaagatcccatgggacaaggatgtggcaaggcttggatgagatggggcaccagtgcccaggaatttgaacattttcctttacccaggaaatctccggagccaacaccaccacccccagggggtctccccaccccaccccatttacagggtgagctcagcctgtcatgagcagaggaaaatattattaatgctctctgagtctttacaacaggagctcttacctcatagatgtgggctctgtttggggaagatgcaaggaagtaatgagaagcccaggaaatttctccacctgtgtttatggcctaaatagcttcaggatgtatcttagctgcactccaacattgcatcctttctggggtgaagaatctgggccaaccaggggtccttgggcctctagaaggccacagtaggcctctctttgtgggaatggaaggggacagtttgcttttagtgctggccctctctgtgggtgtggcctgcaaaggaaccaacagaccctatgctggggactctaacatgtgagctcattaaattcttccagcattctaaaggagggtttgtgattgtcaccatttactgatgaggaaactaaggctcctaggggagaaatcacttgcccacagttccacagctagtgagtgaatgaaccaggatttaaaccggttttttctcactacagagacaatatttttccaccattgtatctcacatttttcccaggaggttacccataacagaagagactagagtggaacagatacgtcagtggataaagctcaaagcaaacaacagtaagcttaaaattccttcatagtctcatgttttacgttcacaattcatgcaaaatttgcattccactttctgatttagccttgttggttttaatatgactctatgaatatttcaaaaaaaaatgtgctctgttcctcatgttgttctgttctgttcaccccgctatgacggaccctaggtcagctggtcttcagcttgaccctagaattgactctaggagcagtgaccctgctgcctcccagagccagttataggctcaagatcaagaccaactgaccttctcctaggcagctcctttggtgtgtgggtgctctgacctcactgttcatgaggggacctcaactaaggcatcttccagttgggtgctggaaggaacccattaactcacactagaatgatgaggatttgctcatctggcgtggagaaggatgagcccacaaaaccctaaagggaaaagagaagctggacacagctgtactcagcagattcctgaatgctaggctggaaagtggtgcctgttgtccaagtggagtcacatggttgctaatgtgggcaagtctgaggacacacttcatgagcagctggggtctggaaggctcctcactttaccctagccacacataattactgggtgcctacagcacctagcaccttggagggggcactattaggaaatcgagattactatggcacaattaattcctgggtaaggcatggggttgtggtggacagagctcagtctttagtttgaacgaaaacatacatacatgaaaaacatacatgaaaaaaggaccctcatcaacattagaaggggtagatttggagcactttaggcaggaaaacaggaacgcaaggccaggaaactggaacccagtgaatactcagaaccgaggatgcagatgacttatttagcaaaatggtcacttctgtgacatagctggagaaaggatgggtaacagcttgccagagccacttggaacaagggcaaatctcagtgtctggggcaaaagatgatgcatttccctctgacccatcatgtttattcatcctccactccccattgccacactagctcttgctgtaagtcctcaccaggatctacatttcctcgtcgctggtgggaaccccttagagtacatagaggtatcagtccagtaagactgctctacacaacagaagtgaggcccagggagtagcagccaggcccttatcctgttacctctgcaggagtgactgcccaacccagatccagagacattgaaggaaatgataattccttggtacctcactgccttgggacaaaatgaagaaagccacccttccttaggctgcagcttgccactcctgggctgggtaaacaggtcatcagcaccaagctcaaccaggagtaacactctggaagacatgggtgagcccaagaggaagcatgaacaggacgctgttcctaagtcatgtcaacaggttgtgctgggccaggatccccagggaaaaaaatggtcaacccaactggagggtaggttagaagaaaaaaaacataaacgtggatagtcatgtcatctcaaatccctgacttggcttccccattacttaacagtctgagctccttcttagcctgtgaccagcttcaaatcacagccaagtaaaacaaggaaataggaaaagtaaatccaactagaagagacaagctgagattcagatttgtttactcctcccatgcaaagtttccctgttggaggttttccatgtatacatgtctagaagtgatagaatgcaaggccttggctttgtcttgcagggatctgcctttgaggtcatagactgaacagcagggagagaggttagtggtggagtgtggggggagctgttctagctccagtttcttctgacacatttttcaggatcatggatctgatcctccgaagcacagcagagatatctaagccatatttgtgcacatgagcagactcttctagttttttagtaaccagggatgggcttttgcatggcactgactatagagatgtcttgtagagatcaagccagtcttttgcatcccacctgcccacctccagaagagatgggaaaaggtcatcaaagggcattcaccaactgaaatccactcatgaatgttaggtctctaaaaggaggcatcaacactcacaatggtagcctccaaacctagcatcccacctatctaagagctcaggggtggtccactggggcagatacaagggaagtgcaagggctcaggatgaaagaaaatctattgggaagagttttaggggcttgatcattatggggcttccttctatatctgagaactgctctgggtggtgagatgtggactctgatccttaattggaatgttcggagaatgagtgtctggtggccttgaagtgttggacagaaaagtatcagtataaaagcctggagctcagggtaattaatgtagttcatggttccttagtgagcaggactcttggatgtggaggagaaagggtcataggaagtaaaccaccaaaattacaaaattgagtctctgtacaattacttcagtgcctttgggcttatgaatacaaatcagtgggccttctctatgatggtccaacaaactctcagtgtccaccctgtccctgtatctcccatggaagatgaataatgtcaggtgttctttgggtcaaaggccccagggcagtctggaggcttagagggcagagtggtgtcattccatgtaaagttaggcttctgaggggtcaggcagaatatggtgtccatatcttccatagctctgcagattcttggatgaagtcaagcacagtttgctagacccaggtcactcctctgagtataactaggacccatgagtgaaacttaatagctgtaaggaagaacctgctgtctgccagagaggataagctgcccatctcagcagctgtctaaaagaaggcaggtgtctctttaaagggaagagaagcattggtgaaatggatttcaggtcacttccattccagatgggtgagatcttgtggagctgggatcatgtttgaactcattcatacctgtagagcacgaatccaagtagattgtgtttggtctgtacaggctgaagccccctgctctcccacccaagtgcccccactgagcaggccaacatgctgttgtggccacatatactgggctgatccaggctggttatcaccaaacagcaaaccatagggaacagctgctttgccatagacccaatacccatgtagatctctcatgagagcagccataactcagacccactgaccaacagggccatgagtgacagccagaaccagtgaaggtccaagtaggacacagagcagggcttttcttaccatacacattatctccagaggttatttctaccccactccctattcaaggcctgttggagcacactgcaaaagcaaaagcacagtaactcaatttacacatgattataatcatttccagtgcacacatttcatcaccaggtggatcctgagctagcccatgtaaatccgggttaacccatattggtaatcatactcaaaagcacttttcaccctacattctactagccaatcaaagacaaagagttgtggcctctaccattgccttggcttctggacaccctcacaagctatcccaaggttcccgctcaactccagggaggctgacatcttcacatccactgggcatataatattgcatgagaccaaagtctccacactctttgcagcctcctccatgaatcccaatggcctgcacttgtacagtttgggtgtttgatagataaagcacgtatgagaagagaaaacaaaataaatcaactttttaaaaaagccagcactgtgctgtcaatgttttttttttcttttcaattctagctcagaaaagcagaaggtaaataatgtcaggtcaatgaatatcagatatattttttgactgtacattacagtgaagtgtaatctttttacacctgcaagtccatcttatttattcttgtaaatgttccctgacaatgtttgtaatatggctgtgttaaaaaatctatacaataaagctgtgaccctgagattcatgttttcctaagataaaaaaaa

SEQ ID NO: 30 - DNA, 1842 bp, Homo sapiens
acggggccccggctcagcaccccggagagctccctccccggccttgttttgctctggcatcgccgccgggggagggagcgagggggccgggcaccgggcgcaaaggcagggggatggccatggaagggttgaggggccccgtcggacagagggcccagccgtgatccagcgacgggtttggggctccgggaggggtgggggggcagcgggcggcggcagcagtggccgcacatctggatggaagcgagctttgtccagaccaccatggctctggggctgtcctccaagaaagcgtcctctcgcaacgtggctgtggagcgtaagaacctgatcaccgtgtgcaggttctctgtgaaaacgctgctggagaagtacacagcggagcccatcgatgactcatcggaggagtttgtcaattttgcagccattttagagcagatcctcagccaccgcttcaaagcctgtgccccagcaggtccagtgagctggttcagctcagacgggcagcggggcttttgggactatatccggctggcctgcagcaaagtgcccaacaactgtgtgagcagcatcgagaacatggagaacatcagcacagcccgggccaagggccgggcatggatccgggtggcactgatggagaagcgcatgtcagaatacatcaccacggctctgcgtgacacccggaccaccagacggttctatgactctggagccatcatgctgcgggatgaagccaccatcctcaccggaatgctgatcggactgagcgccatcgacttcagcttctgtctaaagggggaagtcctggacgggaagacccccgtggtcatcgattacacgccctacctaaagttcacgcagagctacgactacctgacggacgaggaggagcggcacagcgccgagagcagcacgagcgaggacaactcgcccgagcacccgtacctcccgctcgtcaccgacgaggacagctggtacagcaagtggcacaagatggagcagaagttccgcatcgtctacgcgcagaagggctacctggaggagctggtgcgtctgcgcgagtcgcagctgaaggacctggaggcggagaaccggcggcttcagctgcagctggaggaggcggcggcgcagaaccagcgcgagaaacgggagctggaaggcgtgatcctggagctgcaggagcagctgacaggtctgatccccagtgaccacgcccctctggcccagggttccaaggagctcactacacccctggtcaatcaatggccctcactgggaacgcttaatggggccgagggcgccagcaactccaagctctaccggagacacagcttcatgagcacggagccgctgtcagctgaagccagtctgagctcggactcccagcgcctgggagagggcacgcgggacgaggagccctggggtcccatcggaagctcagagccaaattagtggctcccttcgagcgaatgcccaggacttcaacgcatgcactttgtgttgacctcatccctggcttcaccttggtttttcccatcctagttctccctatgcctgaatatcctgtcttttcttttttataagcaaccacactgtattggatgaccctagatcttctttgagacaaggcaggctgtggccatgtagccccatcacactgtgtttgtgattgtctgtgtgtctgtctcccccaccagactgtgagctccatgagggcagggaccgtgtcttgtccgttctctgtatccccagtgcttggaacagagcgagtgctcactgtgtatttaataaatggacaaagagagaggatgaccctcaaaaaaaaaaaaaaaaaaaaaa

SEQ ID NO: 31 - DNA, 502 bp, Homo sapiens
acagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 32 - DNA, 4199 bp, Homo sapiens
agaaaaggaggaaggagaacattgctgtagcttggatctacaacctaagaaagcaagagtgatcaatctcagctctgttaaacatcttgtttacttactgcattcagcagcttgcaaatggttaactatatgcaaaaaagtcagcatagctgtgaagtatgccgtgaattttaattgagggaaaaagggacaattgcttcaggatgctctagtatgcactctgcttgaaatattttcaatgaaatgctcagtattctatctttgaccagaggttttaactttatgaagctatgggacttgacaaaaagtgatatttgagaagaaagtacgcagtggttggtgttttcttttttttaataaaggaattgaattactttgaacacctcttccagctgtgcattacagataacgtcaggaagagtctctgctttacagaatcggatttcatcacatgacaacatgaagctgtggattcatctcttttattcatctctccttgcctgtatatctttacactcccaaactccagtgctctcatccagaggctcttgtgattctctttgcaattgtgaggaaaaagatggcacaatgctaataaattgtgaagcaaaaggtatcaagatggtatctgaaataagtgtgccaccatcacgacctttccaactaagcttattaaataacggcttgacgatgcttcacacaaatgacttttctgggcttaccaatgctatttcaatacaccttggatttaacaatattgcagatattgagataggtgcatttaatggccttggcctcctgaaacaacttcatatcaatcacaattctttagaaattcttaaagaggatactttccatggactggaaaacctggaattcctgcaagcagataacaattttatcacagtgattgaaccaagtgcctttagcaagctcaacagactcaaagtgttaattttaaatgacaatgctattgagagtcttcctccaaacatcttccgatttgttcctttaacccatctagatcttcgtggaaatcaattacaaacattgccttatgttggttttctcgaacacattggccgaatattggatcttcagttggaggacaacaaatgggcctgcaattgtgacttattgcagttaaaaacttggttggagaacatgcctccacagtctataattggtgatgttgtctgcaacagccctccattttttaaaggaagtatactcagtagactaaagaaggaatctatttgccctactccaccagtgtatgaagaacatgaggatccttcaggatcattacatctggcagcaacatcttcaataaatgatagtcgcatgtcaactaagaccacgtccattctaaaactacccaccaaagcaccaggtttgataccttatattacaaagccatccactcaacttccaggaccttactgccctattccttgtaactgcaaagtcctatccccatcaggacttctaatacattgtcaggagcgcaacattgaaagcttatcagatctgagacctcctccgcaaaatcctagaaagctcattctagcgggaaatattattcacagtttaatgaagtctgatctagtggaatatttcactttggaaatgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaagttttaccaccacatattttttcaggggttcctctaactaaggtaaatcttaaaacaaaccagtttacccatctacctgtaagtaatattttggatgatcttgatttgctaacccagattgaccttgaggataacccctgggactgctcctgtgacctggttggactgcagcaatggatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaaagaaacaagtagatgagcaaatgagagacaacagtcctgtgcatcttcagtacagcatgtatggccataaaaccactcatcacactactgaaagaccctctgcctcactctatgaacagcacatggtgagccccatggttcatgtctatagaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatagatggagagtttgagggctttcgcagaaatgctgtgattctgttttaagtccataccttgtaaataagtgccttacgtgagtgtgtcatcaatcagaacctaagcacagcagtaaactatggggaaaaaaaaagaagaagaaaaagaaactcagggatcactgggagaagccatggcattatcttcaggcaatttagtctgtcccaaataaaataaatccttgcatgtaaatcattcaaggattatagtaatatttcatatactgaaaagtgtctcataggagtcctcttgcacatctaaaaaggctgaacatttaagtatcccgaattttcttgaattgctttccctatagattaattacaattggatttcatcatttaaaaaccatacttgtatatgtagttataatatgtaaggaatacattgtttataaccagtatgtacttcaaaaatgtgtattgtcaaacatacctaactttcttgcaataaatgcaaaagaaactggaacttgacaattataaatagtaatagtgaagaaaaaatagaaaggttgcaattatataggccatgggtggctcaaaactttgaacatttgagcttaaacaaatgccactctcatgcattctaaattaaaaagttaaaatgattaatagttcaggtggaagaaataagcatactttttgggttttctacacattttgtgtagacaattttaatgtcagtgctgctgtgaactaaagtatgtcatttatgctcaaagtttaattcttcttcttgggatattttaaaaatgctactgagattctgctgtaaatatgactagagaatatattgggtttgctttatttcataggcttaattctttgtaaatctgaatgaccataatagaaatacatttcttgtggcaagtaattcacagttgtaaagtaaataggaaaaattattttatttttattgatgtacattgatagatgccataaatcagtagcaaaaggcacttctaaaggtaagtggtttaagttgcctcaagagagggacaatgtagctttattttacaagaaggcatagttagatttctatgaaatatttattctgtacagttttatatagttttggttcacaaaagtaattattcttgggtgcctttcaagaaaattaaaaatactactcactacaataaaactaaaatgaaaactcaaaaaaaaaaaaaaaaaa

SEQ ID NO: 33 - DNA, 2449 bp, Homo sapiens
gccccctgcattgctgatgctgctgctggcggacatggacgtggtgaatcagctggtggctgggggtcagttccgggtggtcaaggagcccctcggctttgtgaaggtgctgcaatgggtcttcgccatcttcgcctttgccacatgcggcagctacagtggggagctccagctgagcgtggattgtgccaacaagaccgagagtgacctcagcatcgaggtcgagttcgagtaccccttcaggctgcaccaagtgtactttgatgcacccacctgccgagggggcaccaccaaggtcttcttagttggggactactcctcgtcagccgaattctttgtcaccgtggccgtgtttgccttcctctactccatgggggctctggccacctacatcttcctgcagaacaagtaccgagagaataacaaagggcccatgctggactttctggccacggctgtgttcgccttcatgtggctagttagctcatcggcatgggccaaggggctgtcagatgtgaagatggccacagacccagagaacattatcaaggagatgcctgtctgccgccagacagggaacacatgcaaggagctgagagaccctgtgacctcgggactcaacacctcggtggtgttcggcttcctgaacctggtgctctgggtcggcaacctgtggttcgtgtttaaggagacaggctgggccgccccgttcctgcgcgcgcctcccggcgcccccgagaaacaaccggcacccggggacgcctacggcgatgcaggctacgggcagggccccggcgggtacgggccccaggattcctacgggcctcagggcggctaccagcctgactatggtcaaccagccggcagcggtggcagtggctacgggcctcagggcgactatgggcagcaaggctacggcccgcagggtgcacccacctccttctccaatcagatgtagtctggtcagtgaagcccaggaggacctggggggggcaagagctcaggagaaggcctgccccccttcccacccctataccctaggtctccacccctcaagccaggagaccctgtctttgctgtttatatatatatatattatatataaatatctatttatctgtctgagccctgccctcactccactcccctcatccactaggtgcccagtcttgagtgggccccctctcttaccccgtccctttccctgcatcccttggcccctctctgtttaccctccctgtcccctgaggttaaggggatctaaaaggaggacagggagggaacagacctcggctgtgtggggagggtgggcgtgacttcagactctctcctctctctccctccactcctcccaactctggccttggttcctccagcaatgcctgcctgaacaaaggccgttagggaaatccaactccagggttaaagaaaggcagagattgggggggcttggggtagagaggacagtttaggacccaaggtggtcttggagaggaggtgtggagtggaggggtcagcaggggggttgggttccagacagagtggatctggagtctgaaggagaggagtgcgctagagcattctggggtggggcttggaagggcgctgagggcagggttctagaaggggcgaggctttaagcgaggcagaatggtgggctccagagtaggtgggtcttggattggtaccagagcctatggaaagggtgtggcttggaacatttgggagactgagcttgattctaaaggggacagatcttgagcaaggcaagaagtgggattcaggaatgggccaagccagggttccagacagggtggggcttagaatggggcttccatggtggtttcagaaagggcagcccctccccatggtgcagtgaagaaaatgttttacaatggctgggtttgggcagtggagaggggacttggataggagcttccagatgggttttgttaggggtgggggagaatggctctggctacgacttgggacggaagtggcctgagaagagtcgagtgatatggcttgtagggtgaggcgtgggatccagagagaagcaccccaccacacacacccttccccactcccgtgatgaaacagctaggttaataggaggacagaaccaacgggtctgtgggactggcccacccctcttcccccttcccctgcgccctccctccctccacacctccacccgtcctggggtggttggaggcctggtctggagcccctatcctgcaccctctgctatgtctgtgatgtcagtagtgcctgtgatcgtgtgttgccattttgtctggctgtggcccctccttctcccctccagacccctaccctttcccaaacccttcggtattgttcaaagaacccccctccccaaggaagaacaaatatgattctcctctcccaaataaactccttaaccacctagtcaaaaaaaaaaaaaaaa

SEQ ID NO: 34 - DNA, 744 bp, Homo sapiens
aaacgcgggcgggcgggcccgcagtcctgcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatggtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttagacaaaaaaaaaaaaaaaaaaaaaaa

SEQ ID NO: 35 - DNA, 2352 bp, Homo sapiens
agtagcgaccatttccattggcgttagggagtccttggagcgctgtgttgggaccgtggtggtgactggatccaggaggtcgagagtcgttcttctctttgcacagacgtgactctgcagctctttaacggcgcccgctgctctcaacccagcttaccccacgtggtcccatggcggcggccgcgctaaggttccccgttcagggcacagtgacttttgaagacgtggctgtgaaatttacccaggaggaatggaatctccttagtgaggctcagagatgcctgtaccgtgatgtgacgctggagaacctggcacttatgtcctccctgggttgttggtgtggagtggaagatgaggcggcaccttctaagcagagtatttatatacaaagagagactcaggtcaggactcctatggcaggtgtgtctcccaagaaggcccacccctgtgagatgtgtggcccgatcttgggagacattttgcatgtggcagatcatcagggaacacatcacaagcagaaactgcacaggtgtgaggcctgggggaataaattgtatgacagtggaaactttcatcagcaccagaatgagcacattggagagaaaccctacagagggagtgttgaggaggcgttgtttgcgaagaggtgtaagttgcatgtgtcaggggagtcatctgtcttcagtgagagtgggaaagactttttgctcaggtcaggattactccagcaagaggccactcacactgggaagtcaaacagcaaaactgagtgtgtgtctctgtttcatgggggaaaaagccattacagctgtggaggatgcatgaaacattttagcaccaaagatatactcagtcagcacgagagactgctccctacagaagaaccttctgtgtggtgtgaatgtgggaaatcctctagcaaatatgacagcttcagtaatcatcaaggagttcacactagagaaaaaccttatacgtgtgggatatgtgggaaattatttaacagtaagtcccacctccttgtacaccagagaattcacactggagagaagccatatgagtgtgaggtttgtcagaaattttttaggcacaagtaccacctcattgcacaccagagagttcacactggagaaaggccatatgaatgcagtgattgtgggaagtcatttacccacagctctacattccgtgttcataagagagttcacactggtcagaagccttatgagtgcagtgaatgtgggaaatcttttgccgaaagctccagtctcactaaacacaggagagttcacactggagaaaagccttacgggtgcagtgaatgtgaaaaaaaatttaggcaaatctcttcacttcgtcatcatcagagagttcacaaaagaaagggcttatgagtgcagtgagtccagtctcattaaatattggagaatacacacctgagtaagatcctgtgattgcagcaaatgtggaaaatgtttacccaaaggtctgctctccttggatattggagagttcacactagagaagagtccttacaagtaaaataaatttgggcagttttgtagccacacctctgtattccttcaggattatagttaacactgaatgaaggccttactagtgtgacaaatacaggatatttatccagaagtctggctgcataactcacaggagagctgtcatgtgggaggattgctacatgggaggatttgcttgagcctgggaggcagaggttgcagtgagccgagattgcaacactgcagtccagtctggaccacagagtgagaccctgtttcaaaaataaaatatataaatgacatccaataaagtatgcatatttgagctgtaggataagtcttgaattttctttttcttttttttgagacagcctcgctctgtcacccaggctggagtacagtggcataatctcggctcactgcaagccctgcctcctgggttcatgccattctcctgcctcagcctccctaatagctgggactacaggcaaccaccaccactcctaccttttttgtatttttagtagagatggggtttcaccctgttagccaggaagttctcaatctcctgacctcgtgatctgcccgccttggcctctcaaagtgttgggattgcaggcgtgagccactgctcctggcttgaattttcttataatcccatgaaaccatcatcagagtcacaataatgaatttatctaatctatcacctttgtattcacctggagcctttataaagtttttctctatgcagacaaccattgatttactctgctgtataatactttccattttctagaattttctgactgatgtaataaagtgtttcctcttataa

SEQ ID NO: 36 - DNA, 2429 bp, Homo sapiens
gtgccttatagctcccctcccagtggagcaggtgtctgagagtacagtgcagccctgcccttctgtccaccctacagagcccacggccatggcagcccaggcagctggtgtatctaggcagcgggcagccactcaaggtcttggctccaaccaaaacgctttgaagtacttgggccaggatttcaagaccctgaggcaacagtgcttggactcaggggtcctatttaaggaccctgagttcccagcatgtccatcagctttgggctacaaggatcttggaccaggctctccgcaaactcaaggcatcatctggaagcggcccacggagttgtgtcccagccctcagtttatcgttggtggagccacgcgcacagacatttgtcagggtggtctaggtgactgctggcttctggctgccattgcctccctgaccctgaatgaagagctgctttaccgggtggtccccagggaccaggacttccaggagaactatgcgggaatctttcactttcagttctggcagtacggagagtgggtggaggtggtcattgacgacaggctgcccaccaagaatggacagctgctcttcctacactcggaacaaggcaatgaattctggagtgccctgctggagaaagcctatgccaagcttaatggttgttatgaggctctcgctggaggttccacagtggaggggtttgaggatttcacaggtggcatctctgagttttatgacctgaagaaaccaccagccaatctatatcagatcatccggaaggccctctgtgcggggtctctgctgggctgctccattgatgtctccagtgcagccgaagccgaagccatcaccagccagaagctggttaagagtcatgcgtactctgtcactggagtcgaagaggtgaatttccagggccatccagagaagctgatcagactcaggaatccatggggtgaagtggagtggtcgggagcctggagcgatgatgcaccagagtggaatcacatagacccccggcggaaggaagaactggacaagaaagttgaggatggagaattctggatgtcactttcagatttcgtgaggcagttctctcggttggagatctgcaacctgtccccggactctctgagtagcgaggaggtgcacaaatggaacctggtcctgttcaacggccactggacccggggctccacagctgggggctgccagaactacccagccacgtactggaccaatccccagttcaaaatccgtttggatgaagtggatgaggaccaggaggagagcatcggtgaaccctgctgtacagtgctgctgggcctgatgcagaaaaatcgcaggtggcggaagcggataggacaaggcatgcttagcatcggctatgccgtctaccaggttcccaaggagctggagagtcacacggacgcacacttgggccgggatttcttcctggcctaccagccctcagcccgcaccagcacctacgtcaacctgcgggaggtctctggccgggcccggctgccccctggggagtacctggtggtgccatccacatttgaacccttcaaagacggcgagttctgcttgagagtgttctcagagaagaaggcccaggccctagaaattggggatgtggtagctggaaacccatatgagccacatcccagtgaggtggatcaggaagatgaccagttcaggaggctgtttgagaagttggcagggaaggattctgagattactgccaatgcactcaagatacttttgaatgaggcgttttccaagagaacagacataaaattcgatggattcaacatcaacacttgcagggaaatgatcagtctgttggatagcaatggaacgggcactttgggggcggtggaattcaagacgctctggctgaagattcagaagtatctggagatctattgggaaactgattataaccactcgggcaccatcgatgcccacgagatgaggacagccctcaggaaggcaggtttcaccctcaacagccaggtgcagcagaccattgccctgcggtatgcgtgcagcaagcttggcatcaactttgacagcttcgtggcttgtatgatccgcctggagaccctcttcaaactattcagccttctggacgaagacaaggatggcatggttcagctctctctggccgagtggctgtgctgcgtgttggtctgacccggggtttcggacatcagtgacactccctgccccactgcttgcttcttgtcaccccttctctacaattttgtgaacatttatgctccagtggcattcactggttgttcatacctttcttgccctgggtctatttcagcagcactgagctatgagctatgtaagccgacccggtgggcccagtggagggaaagcaatcaattaaagttgtgagccagaatggtttcca

SEQ ID NO: 37 - DNA, 3859 bp, Homo sapiens
aatctatcagggaacggcggtggccggtgcggcgtgttcggtgcgctctggccgctcaggccgtgcggctgggtgagcgcacgcgaggcggcgaggcggcaagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaagcaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcagttcatgaaattatatgaagaaaaaaccgggaacgcttggcactccaaaaatttcacgaagtatcccaaaaagttttaccccctggagattgactatggccaggatgaagaggcagtgaagaagctcacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacagtgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactaccatacgtctcagggagacccaataggcttaatcctgttgggagaagttgcccttggaaacatgtatgaactgaagcacgcttcacatatcagcaggttacccaagggcaagcacagtgtcaaaggtttgggcaaaactacccctgatccttcagctaacattagtctggatggtgtagacgttcctcttgggaccgggatttcatctggtgtgatagacacctctctactatataacgagtacattgtctatgatattgctcaggtaaatctgaagtatctgctgaaactgaaattcaattttaagacctccctgtggtaattgggagaggtagccgagtcacacccggtggctgtggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaaagcttcgttagaatgtctgccttactggtttccccagggaaggaaaaatacacttccacccttttttctaagtgttcgtctttagttttgattttggaaagatgttaagcatttatttttagttaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttg

SEQ ID NO: 38 - DNA, 1283 bp, Homo sapiens
ctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 39 - protein, 103 aa, Homo sapiens [sequence omitted]

SEQ ID NO: 40 - DNA, 2236 bp, Homo sapiens
ataacggcttggcttacctgtaacgggtacgcttcgctttgcgttcctcgcttggccttgcgttcctagcttggcttggcgttagtcgctttgcctggtgtttcttgcttggattggcgtttcctccctcgcattcctttgctgggcttgactttttctctgctgggtttggcattcccttgactgggctgggtgtttccttgggagggggggcttggcctttcctggggtgggcgttgggtccccctggtgggcgtgggctttccccgggtgggtgtgggttttccctgggtggggtggactgggctcccttgctggggttggcaagttttggctgggattgacctttctcttcaaacagattggaaacccagggtttcctgctagttggtgaaactggttggtagacgcgatctgttcgctactaccggcctccccgggctgttaaaagcagatggtgactgaggtttgttcaatgcccgctgcctctgctgtgaagaagccattcgatctcaggagcaagatgggcaagtggtttcaccaccgcttcccctgctgcaaggggagcggcaagagcaacatgggcacttctggagaccacgacgactcctttatgaagatgctcaggagcaagatgggcaagtgttgccaccactgcttcccctgctgcagggggagcggcacgagcaacgtgggcacttctggagaccatgacaactcctttatgaagacgctcaggagcaagatgggcaagtggtgctgtcactgcttcccctgctgcagggggagcggcaagagcaacgtgggcgcttggggagactacgacgacagcgccttcatggagccgaggtaccacgtccgtcgagaagatctggacaagctccacagagctgcctggtggggtaaggtccccagaaaggatctcatcgtcatgctcagggacacggacatgaacaagagggacaagcaaaagaggactgctctacatttggcctctgccaatggaaattcagaagtagtacaactcctgctggacagacgatgtcaacttaatgtccttgacaacaaaaaaaggacagctctgataaaggccgtacaatgccaggaagatgaatgtgtgttaatgttgctggaacatggcgctgatcaaaatattccagatgagtatggaaataccactctacactatgctgtccacaatgaagataaattaatggccaaagcactgctcttatatggtgctgatattgaatcaaaaaacaagtgtggcctcacaccacttttgcttggcgtacatgaacaaaaacagcaagtggtgaaatttttaatcaagaaaaaagctaatttaaatgcacttgatagatatggaagaactgccctcatacttgctgtatgttgtggatcagcaagtatagtcaatcttctccttgagcaaaatgttgatgtatcttctcaagatctatctggacagacggccagagagtatgctgtttctagtcatcatcatgtaatttgtgaattactttctgactataaagaaaaacagatgctaaaaatctcttctgaaaacagcaatccagaacaagacttaaagctaacatcagaggaagagtcacaaaggcttaaagtcagtgaaaatagccagccagagaaaatgtctcaagaaccagaaataaataaggactgtgatagagaggttgaagaagaaataaagaagcatggaagtaatcctgtgggattaccagaaaacctgactaatggtgccagtgctggcaatggtgatgatggattaattccacaaaggaggagcagaaaacctgaaaatcagcaatttcctgacactgagaatgaagagtatcacagtgacgaacaaaatgatacccggaaacaactttctgaagaacagaacactggaatatcacaagatgagattctgactaataaacaaaagcagatagaagtggctgaaaagaaaatgaattctgagctttctcttagtcataagaaagaagaagatctcttgcgtgaaaacagcatgttgcaggaagaaattgccatgctaatttctggagactggaactagatgaaacaaaacatcagaaccagctaagggaaaataaaattttggaggaaattgaaagtgtgaaagaaaagactgataaacttctaagggctatgcaattgaatgaagaagcattaacgaaaaccaatatttaagtacagtggacagcttagg

SEQ ID NO: 41 - DNA, 2813 bp, Homo sapiens
gcgcatctggttgccagggaaacagcggtcccacaggcaggagggatctggaccggcggcttcttccactaccagagaggctgaggtggcgggagagacccctctcctctgggggtaagtggctggcgcggcgagtgtctgcggcgcgcttctgcggccatctgcgccctgactgacggcggccagttcctcgaggaggcccggcgggaaggatttcaatggatattataaagggaaacctagatggaatttcaaaaccagcttcaaattcaagaatacgccctgggagcagaagttcaaatgcttctttggaggtgctctcaacagaaccaggatccttcaaggtcgatactgcaagcaacttgaactctggtaaagaggaccactccgaaagcagtaatacagagaacagaagaactagtaatgatgataagcaggaaagctgctctgagaaaataaaattggctgaagagggatcagatgaagatctggatttggttcaacatcagataatctctgagtgttcagatgaacccaaattaaaagaattagattctcaacttcaagatgctattcagaagatgaaaaaacttgataaaatattggcaaagaaacaacgcagagaaaaagaaattaagaagcaaggtctagaaatgagaataaagctgtgggaagaaattaagtctgcaaaatatagtgaagcttggcaaagtaaagaggagatggaaaatacaaaaaaatttttatctttgactgctgtttctgaagaaactgttggtccttctcatgaggaggaagacaccttttcctcagtgtttcatactcaaatccctccagaagaatatgaaatgcagatgcagaaactcaataaagattttacctgtgatgtggaaagaaatgagtcattgatcaaatcaggaaagaaacctttctcgaatacagaaaagattgagctcaggggtaaacacaaccaggattttattaagagaaacattgagttggccaaggaatcaagaaacccagtggttatggttgacagagagaagaaaaggctggttgagcttttgaaggacttggatgagaaagattccgggctctccagttctgagggtgatcagtctggctgggtggtcccagtaaaaggatatgaacttgcagtcacccagcatcagcagcttgctgaaattgatataaaactccaagaactctctgcagcctcccctacaatttccagtttttctccaagacttgaaaatcggaataatcagaaacctgaccgtgatggtgaaagaaatatggaagtaactccaggagaaaagatacttaggaacaccaaagagcaacgcgatctgcataatcggctgagagagattgatgaaaagctgaaaatgatgaaggaaaatgtgttagagtccacatcatgtctctctgaagaacagttaaagtgtcttctggatgaatgcatacttaaacaaaaatccatcattaaactttcttcagaaagaaaaaaggaagacattgaggacgtaacacctgtgttcccccagctttccaggtccatcatctctaaattgctaaatgaatcagaaacaaaggtccagaaaactgaggtagaagatgcagatatgcttgagagtgaagaatgtgaagcttctaaaggctactatctcactaaagccttgactggacataacatgtcagaagctcttgtcactgaagcagagaatatgaaatgccttcaattttccaaggacgttattattagtgacacaaaagactattttatgtcgaagactcttggcattgggagactgaaaaggccctccttcttagatgatccactgtatggtatcagtgtgagcctttcatcagaagaccaacatctgaaactcagttctccagagaatacaatagcagatgagcaggagactaaagatgcagcagaagaatgtaaagaaccctaatcaaggacttgctgggtgtgcttttcagaaagttggtaatgaagttaaattaaatttcttattgaattatttgaattcaatgaatgcactgcagagactattctttattgattttgacatttggagtcgctctttgtggattatatttccttgatatttttgagacttggggtgtaattgttcagtggtttttgctcattaaaatttctgggaccattgtttataaatttattgctaatcttacagtattaggattcattataaaaaccaacattttaatgtatacgtgttagggtaaaactcgttgaagtgctgtgattgtcctgtattcaattttgtatgtttcacctctactgtgattcagacagatcatggtggtcactgggaatttttgctgtggccctgcttttccttcttcccacttgtctcatgtctgtgaaactggtacaacctgccataagatgaaatgaattgtctcaacaaagcaatattagaagagcctttactatcttattggtgatgacacgtttcttaagtaggagtttgagtgaattatttgatatattactttgttaataatagttaacaatagtttcttattttctttcagagtttgggccttttagattgcatcaaataaagatgagctactttaaaagacagtctgcggtactatttgaagagagatggtttagttacaagtcagtaacttagattcatgagaaaattttgtgttgacattccataagatgagttggcattagaattggaagtccagtttatttctgaatcaaaaaatgagcgggctattgtgctgcctttaataaaatttgagtgatgtatacataa

SEQ ID NO: 42 - DNA, 2919 bp, Homo sapiens
ggccagaagaaatctggcctcggaacacgccattctccgcgccgcttccaataaccactaacatccctaacgagcatccgagccgagggctctgctcggaaatcgtcctggcccaactcggcccttcgagctctcgaagattaccgcatctatttttttttcttttttttcttttcctagcgcagataaagtgagcccggaaagggaaggagggggcggggacaccattgccctgaaagaataaataagtaaataaacaaactggctcctcgccgcagctggacgcggtcggttgagtccaggttgggtcggacctgaacccctaaaagcggaaccgcctcccgccctcgccatcccggagctgagtcgccggcggcggtggctgctgccagacccggagtttcctctttcactggatggagctgaactttgggcggccagagcagcacagctgtccggggatcgctgcacgctgagctccctcggcaagacccagcggcggctcgggatttttttgggggggcggggaccagccccgcgccggcaccatgttcctggcgaccctgtacttcgcgctgccgctcttggacttgctcctgtcggccgaagtgagcggcggagaccgcctggattgcgtgaaagccagtgatcagtgcctgaaggagcagagctgcagcaccaagtaccgcacgctaaggcagtgcgtggcgggcaaggagaccaacttcagcctggcatccggcctggaggccaaggatgagtgccgcagcgccatggaggccctgaagcagaagtcgctctacaactgccgctgcaagcggggtatgaagaaggagaagaactgcctgcgcatttactggagcatgtaccagagcctgcagggaaatgatctgctggaggattccccatatgaaccagttaacagcagattgtcagatatattccgggtggtcccattcatatcagatgtttttcagcaagtggagcacattcccaaagggaacaactgcctggatgcagcgaaggcctgcaacctcgacgacatttgcaagaagtacaggtcggcgtacatcaccccgtgcaccaccagcgtgtccaacgatgtctgcaaccgccgcaagtgccacaaggccctccggcagttctttgacaaggtcccggccaagcacagctacggaatgctcttctgctcctgccgggacatcgcctgcacagagcggaggcgacagaccatcgtgcctgtgtgctcctatgaagagagggagaagcccaactgtttgaatttgcaggactcctgcaagacgaattacatctgcagatctcgccttgcggatttttttaccaactgccagccagagtcaaggtctgtcagcagctgtctaaaggaaaactacgctgactgcctcctcgcctactcggggcttattggcacagtcatgacccccaactacatagactccagtagcctcagtgtggccccatggtgtgactgcagcaacagtgggaacgacctagaagagtgcttgaaatttttgaatttcttcaaggacaatacatgtcttaaaaatgcaattcaagcctttggcaatggctccgatgtgaccgtgtggcagccagccttcccagtacagaccaccactgccactaccaccactgccctccgggttaagaacaagcccctggggccagcagggtctgagaatgaaattcccactcatgttttgccaccgtgtgcaaatttacaggcacagaagctgaaatccaatgtgtcgggcaatacacacctctgtatttccaatggtaattatgaaaaagaaggtctcggtgcttccagccacataaccacaaaatcaatggctgctcctccaagctgtggtctgagcccactgctggtcctggtggtaaccgctctgtccaccctattatctttaacagaaacatcatagctgcattaaaaaaatacaatatggacatgtaaaaagacaaaaaccaagttatctgtttcctgttctcttgtatagctgaaattccagtttaggagctcagttgagaaacagttccattcaactggaacattttttttttttccttttaagaaagcttcttgtgatcctttggggcttctgtgaaaaacctgatgcagtgctccatccaaactcagaaggctttgggatatgctgtattttaaagggacagtttgtaacttgggctgtaaagcaaactggggctgtgttttcgatgatgatgatgatcatgatgatgatcatcatgatcatgatgatgatcatcatgatcatgatgatgattttaacagttttacttctggcctttcctagctagagaaggagttaatatttctaaggtaactcccatatctcctttaatgacattgatttctaatgatataaatttcagcctacattgatgccaagcttttttgccacaaagaagattcttaccaagagtgggctttgtggaaacagctggtactgatgttcacctttatatatgtactagcattttccacgctgatgtttatgtactgtaaacagttctgcactcttgtacaaaagaaaaaacacctgtcacatccaaatatagtatctgtcttttcgtcaaaatagagagtggggaatgagtgtgccgattcaatacctcaatccctgaacgacactctcctaatcctaagccttacctgagtgagaagccctttacctaacaaaagtccaatatagctgaaatgtcgctctaatactctttacacatatgaggttatatgtagaaaaaaattttactactaaatgatttcaactattggctttctatattttgaaagtaatgatattgtctcatttttttactgatggtttaatacaaaatacacagagcttctttcccctcaaaaaaaaaaaaaaaaaa

SEQ ID NO: 43 - DNA, 525 bp, Homo sapiens
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggtaa

SEQ ID NO: 44 - DNA, 4337 bp, Homo sapiens
gcgggagcttctcctgccaggcaggaagacgagtagaagggagcggcatgctggaggctggagcctgagcccctggggctcgccttgctgtgtttggtggtgacgtgggacactgcagctcggccagagtggtagaaatgtcctggtgtaggcttttctggctttgcccgtctagctgctccaagccaggctggaggaggaggagaaggaatcacctgtggtacgctggagcctgcatgtggcgtgactctgcagctcgcctcgtgtgactgatggcagccacggagactgcagctcgacaggagtgattggaaacccggagttacctgctagttggtgaaactggttggtagacgcgatctgttggctactactggcttctcctggctgttaaaagcagatggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcagcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagcaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaattaagaaaaaagcgaatttaaatgcactggatagatatggaagaactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaacaagacttaaagctgacatcagaggaagagtcacaaaggttcaaaggcagtgaaaatagccagccagagaaaatgtctcaagaaccagaaataaataaggatggtgatagagaggttgaagaagaaatgaagaagcatgaaagtaataatgtgggattactagaaaacctgactaatggtgtcactgctggcaatggtgataatggattaattcctcaaaggaagagcagaacacctgaaaatcagcaatttcctgacaacgaaagtgaagagtatcacagaatttgtgaattactttctgactacaaagaaaagcagatgccaaaatactcttctgaaaacagcaacccagaacaagacttaaagctgacatcagaggaagagtcacaaaggcttaaaggcagtgaaaatggccagccagagaaaagatctcaagaaccagaaataaataaggatggtgatagagagctagaaaattttatggctatcgaagaaatgaagaagcacagaagtactcatgtcggattcccagaaaacctgactaatggtgccactgctggcaatggtgatgatggattaattcctccaaggaagagcagaacacctgaaagccagcaatttcctgacactgagaatgaagagtatcacagtgacgaacaaaatgatactcagaagcaattttgtgaagaacagaacactggaatattacacgatgagattctgattcatgaagaaaagcagatagaagtggttgaaaaaatgaattctgagctttctcttagttgtaagaaagaaaaagacatcttgcatgaaaatagtacgttgcgggaagaaattgccatgctaagactggagctagacacaatgaaacatcagagccagctaagagaaaagaaatatttggaggatattgaaagtgtgaaaaaaaggaatgataatcttttaaaggctctacaattgaatgagctcaccatggatgatgataccgctgtgctcgtcattgacaacggctctggcatgtgcaaggccggctttgcgggcgacgatgccccccgggctgtcttcccttccatcgtggggcgccccaggcagcagggcatgatggggggcatgcatcagaaagagtcctatgtgggcaaggaggcccagagcaaaagaggcatcctgaccctgaagtaccccatggaacacggcatcatcaccaactgggatgacatggagaagatctggcaccacaccttctacaacgagctgcgtgtggctcccgaggagcaccccgtcctgctgaccgaggccaccctgaaccctaaggccaaccgcgagaagatgacccagatcatgtttgagaccttcaacaccccagccatgtacgtggccatccaggctgtgctgtccctgtacacctctggccgtactactggcatcgtgatggactctggtgacggggtcacccacactgtgcccatctatgaggggaatgccctcccccatgccaccctgcgcctagacctggctgggcgggaactgcctgactacctcatgaagatcctcaccgagcatggctataggttcaccaccatggccgagcgggaaatcgtgcgtgacatcaaagagaagctgtgctatgttgccctggacttcgagcaggagatggccacggtggcctccagctcctccctagagaagagctacgagctgcccgatggccaggtcatcaccatcggcaacgagcggttccgctgccccgaggcgctcttccagccttgcttcctgggcatggaatcctgtggcatccatgaaactaccttcaactccatcatgaagtctgatgtggacatccgcaaagacctgtacaccaacacagtgctgtctggcggcaccaccatgtaccctggcatggcccacagaatgcagaaggagatcgctgccctggcgcctagcatgatgaagatcaggatcattgctcctcccaagcgcaagtactccgtgtgggtcggtggctccatcctggcctcgctgtccaccttccagcagatgtggatcagcaagcaggagtatgatgagtcaggcccctccattgtccaccgcaaatgcttgtaggtggactctgacttagttgcgttacaccctttcttgacaaaaccaaacttctcagaaaacaacatgagattggcatggctttatttgttttcttgtttcattttttgttttgttttttattggcttgactcaggatttaaaaaccggaatggtgaaggtgacagcagtcggttggaggaagcttcctccaaagttctacaatgtggccaaggactttgattgtaccttgttcttcttttcaatagtcattccaaatattgtgagacgcattgtttcaggaagccccttgccctgctaaaagccaccccacttctctctaaggagaatggcccagtcctctccctagttcacacaggggaggtgatagcattgcttttgtgcaaattacataatgcaaaattttttgaatcttcgccttaatactttttaattttgttttattttgaatgatcagccttcgtggcccccctcttttgtaccccaacttggggtgtatgaaggcttttggtctccctgagagtggctggaggcagccagggcttacctgtactctgacttgaggagagttggataaaagtgcacaccttaaaacaaattgaggaagcacagtatttcagtacagtggacagcttagcatgttgacaactgagaataaaatgctcagttctgaactggacaatgtaagacacaacgaggaaacactggaaat

SEQ ID NO: 45 - DNA, 3440 bp, Homo sapiens
ggtagacgcgatctgttggctactactggcttctcctggctgttaaaagcagatggtggttgaggttgattccatgccggctgcctcttctgtgaagaagccatttggtctcaggagcaagatgggcaagtggtgctgccgttgcttcccctgctacagggagagcggcaagagcaacgtgggcacttctggagaccacgacgactctgctatgaagacactcaggagcaagatgggcaagtggtgccaccactgcttcccctgctgcagggggagtggcaagagcaacgtgggcgcttctggagaccacgacgactctgctatgaagacactcaggaacaagatggggaagtggtgctgccactgcttcccctgctgcagggggagcggcaagagcaaggtgggcgcttggggagactacgatgacagcgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaacaagagttaaagctgacatcagaggaagagtcacaaaggttcaaaggcagtgaaaatagccagccagagaaaatgtctcaagaactagaaataaataaggatggtgatagagaggttgaagaagaaatgaagaagcatgaaagtaataatgtgggattactagaaaacctgactaatggtgtcactgctggcaatggtgataatggattaattcctcaaaggaagagcagaacacctgaaaatcagcaatttcctgacaacgaaagtgaagagtatcacagaatttgtgaattactttctgactacaaagaaaagcagatgccaaaatactcttctgaaaacagcaacccagaacaagacttaaagctgacatcagaggaagagtcacaaaggcttaaaggcagtgaaaatggccagccagagaaaagatctcaagaaccagaaataaataaggatggtgatagagagctagaaaattttatggctatcgaagaaatgaagaagcacggaagtactcatgtcggattcccagaaaacctgactaatggtgccactgctggcaatggtgatgatggattaattcctccaaggaagagcagaacacctgaaagccagcaatttcctgacactgagaatgaagagtatcacagtgacgaacaaaatgatactcagaagcaattttgtgaagaacagaacactggaatattacacgatgagattctgattcatgaagaaaagcagatagaagtggttgaaaaaatgaattctgagctttctcttagttgtaagaaagaaaaagacgtcttgcatgaaaatagtacgttgcgggaagaaattgccatgctaagactggagctagacacaatgaaacatcagagccagctaagagaaaagaaatatttggaggatattgaaagtgtgaaaaaaaagaatgataatcttttaaaggctctacaattgaatgagctcaccatggatgatgataccgccgtgctcgtcattgacaacggctctggcatgtgcaaggccggctttgcgggcgacgatgccccccgggctgtcttcccttccatcgtggggcgccccaggcagcagggcatgatggggggcatgcatcagaaagagtcctatgtgggcaaggaggcccagagcaagagaggcatcctgaccctgaagtaccccatggaacacggcatcatcaccaactgggatgacatggagaagatctggcaccacaccttctacaacgagctgcgtgtggctcccgaggagcaccccatcctgctgaccgaggcccccctgaaccccaaggccaaccgcgagaagatgacccagatcatgtttgagaccttcaacaccccagccatgtacgtggccatccaggccgtgccgtccctgtacacctctggccgtactactggcatcgtgatggactctggtgacggggtcacccacactgtgcccatctatgaggggaatgccctcccccatgccaccctgcgcctagacctggctgggcgggaactgcctgactacctcatgaagatcctcaccgagcgtggctataggttcaccaccatggccgagcgggaaatcgtgcgtgacatcaaagagaagctgtgctatgttgccctggacttcgagcaggagatggccacggcggcctccagctcctccctagagaagagctacgagctgcccgatggccaggtcatcaccatcggcaacgagcggttccgctgccccgaggcgctcttccagccttgcttcctgggcatggaatcctgtggcatccatgaaactaccttcaactccatcatgaagtctgatgtggacatccgcaaagacctgtacaccaacacagtgctgtctggcggcaccaccatgtaccctggcatggcccacagaatgcagaaggagatcgctgccctggcgcctagcatgatgaagatcaggatcattgctcctcccaagcgcaagtactccgtgtgggtcggtggctccatcctggcctcgctgtccaccttccagcagatgtggatcagcaagcaggagtatgatgagtcaggcccctccattgtccaccgcaaatgcttctaggtggactctgacttagttgcgttacaccctttcttgacaaaaccaaacttctcagaaaacaacatgagattggcatggctttatttgttttcttgtttcattttttgttttgttttttattggcttgactcaggatttaaaaaccggaatggtgaaggtg

SEQ ID NO: 46 - DNA, 2933 bp, Homo sapiens
atggtggctgaggttgattcaatgccggctgcctcttctgtgaagaagccatttggtctcaggagcaagatgggcaagtggtgctgccactgctttccctgctgcagggggagcggcaagagcaacgtgggcacttctggagaccgcaacgactcctctgtgaagacgcttgggagcaagaggtgcaagtggtgctgccactgcttcccctgctgcagggggagcggcaagagcaacgtgggcgcttggggagactacgatgacagcgccttcatggatcccaggtaccacgtccatggagaagatctggacaagctccacagagctgcctggtggggtaaagtccccagaaaggatctcatcgtcatgctcagggacactgatgtgaacaagagggacaagcaaaagaggactgctctacatctggcgtctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcactggatagatatggaagaactgctctcatacttgctgtatgttgtggatcagcaaatatagtcagccctctacttgagcaaaatattgatgtatcttctcaagatctggacagacggccagagagtatgctgtttctagtcatcatcatgtaatttgccagttactttctgactacaaagaaaaacagatgttaaaaatctcttctgaaaacagcaatccagaacaagacttaaagctgacatcagaggaagagtcacaaaagcttaaaggaagtgaaaacagccagccagagaaaatgtctcaagaaccagaaataaataaggatggtgatagagagctagaagattttatggctattgaagaagaaatgaagaagcacggaagtactcatgtgggattcccagaaaacctgactaatggtgccgctgctggcaatggtgatgatggattaattcctccaaggaagagcagaacacctgaaagccagcaatttcctgacactgagaatgaagagtatcacagtgatgaacaaaatgatactcagaagcaactttctgaagaacagaacactggaatattacaagatgagattctgttcatgaagaaaagcagatagaagtggctgaaaagaaaatgaattctgggccgggcacgctttctcttagttataagaaagaaaaagacttcttgcatgaaaatagtacgttgcaggaagaagttgccatgctaagactggagctagacataatgaaacatcagagccagctaagagaaaagaaatatttggaggaaattgaaagtgtggaaaaaaagaatgataatcttttaaaggctctacaattgaatgagctcaccatggatgatgataccgctgtgctcgtcattgacaacggctctggcatgtgcaaggccggctttgcgggcgacgatgccccccaggctgtcttcccttctatcgtggggcgccccaggcaccagggcatgatggagggcatgcatcagaaggagtcctatgtgggcaaagaggcccagagcaagagaggcatgctgaccctgaagtaccccatggagcatggcatcatcaccaactgggacgacatggagaagatctggcaccacaccttctacaatgagctgcgtgtggctcctgaggagcaccccatcctgctgactgaggcccccctgaaccccaaggccaaccgtgagaagatgacccagatcatgtttgagaccttcaacaccccagccatgtacgtggccatccaggccgtgctgtccctatacacctctggccgtactactggcatcgtgatggactctggtgatgggttcacccacactgtgcccatctatgaggggaatgccctcccccatgccaccctgcgcctagacctggctgggcgggaactgactgactacctcatgaagatcctcaccgagcgtggctataggttcaccaccacggccgagcaggaaattgtgcgtgacatcaaagagaagctgtgctatgttgccctggactccgagcaggagatggccatggcagcctccagctcctccgtagagaagagctacgagctgcccgatggtcaggtcatcaccatcggcaacgagcggttccgctgccccgaggcgctcttccagccttgcttcctgggcatggaatcctgtggcatccacaaaactaccttcaactccatagtgaagtctgatgtggacatccgcaaagacctgtacaccaacacagtgctgtctggcggcaccaccatgtaccctggcatcgcccacaggatgcagaaggagatcactgccctggcgcctagcattatgaagatcaagatcattgctcctcccaagcgcaagtactccgtgtgggtcggtggctccatcctggcctcgctgtccaccttccagcagatgtggatcagcaagcaggagtatgatgagtcaggcccctccattgtccaccgcaaatgcttctaggtggactctgacttagttgcgttacaccctttcttgacaaaaccaaacttctcagaaaacaacatgagattggcgtggctttatttgttttcttgtttcattttttgttttgttttttattggcttgactcaggatttgaaaaccggaacggcgaaggtg

SEQ ID NO: 47 - DNA, 1222 bp, Homo sapiens
gcttttagagcactgtgatgtaacatgtcaagcagaaatagggagcatgtttacagccattctatgaaaaagtgttcggaatgtacagactagcacagaagctggactaattgaacaagtattgctgaaaatgagtgctgtagatgacatgatagcagagtaccttcaagtttaaatctgagagtgatattcatttggcagaacatcataaacaggttttgtatgatgggaaacttgcaagtagcattacctttacatatactgctaaggccactgatgctcaactctgcctggaatcatcaccaaaagagaatgcatcaatttttgtgcattcccaacatgctctaatgcttcagattcaagtgctttttccactgtttccccaattggataatcggcagctcaatgacagtcaagtggaaacaactgtctgctcctgcttcaggtgcagaaatacagcgatttccagtgccagctgttgagccagtgccagcaccaggggcagattcccctccagggacagcgctggagctagaggaagctccagagccctcctgccgctgccctgggactgcccaggaccagcccagtgaggagctgcctgacttcatggcacctcctgtagagccaccggcctcagccctggagctgaaagtgtggctggagctagaggtggcagagaggggtggccagcacagctccagccagcagctcccacactgctcccagtcctgggcacagtggaagctatggaggcagagaccagggtttgcaatctgggctcctctgcctcactggagagggacttctctcattcagcagagcagcagccctgctgctgaagggcctgctgctactgctgctggggctgtttgcctgcctgcaggaggtgctggagagcaagaaaaggagcctgtgagcaggggttccagcaggtcctcctgctcccagaggcgacctcctcctccaggcatggaggtttgccctcagctgggcatctgggccatttgcccctaacgtgctgcccaggatggcctcctcttgacaggcggacagggggtgagggggccagggggcatctccaaaggaagcttttaaactcagcagctgcaccccagaatctgtatgcctgcacctgcccaaggatttattcatagcttacctaagaatttcaaatttctaccataacactgaataaagtttgactttttgaaacttcaaa

SEQ ID NO: 48 - DNA, 1408 bp, Homo sapiens
atgatcactgaaaataaattaattgatataggagagagtatagctattccagatgaatttaccgaacaagaaaagcagtctggagattggtggaagcgtttggtgtcagcaggaatagctagtgcggttgcacggacatgcacggcacctttagaccgcttgaaagtcatgatgcaggttcatagtttaaagtcaaggaaaatgagattgattagtggccttgagcagttggtgaaagaaggagggattttttccctttggtgaggaaatggtgtaaatgttttaaaaattgcaccagagacagcactcaaggttggggcctatgaacagtataagaaattgcttagttttgatggtgttcatttaggaattcttgaaagatttatatttggctcattggctggtgtaactgcccagacctgtatttaccccatggaggtactaaagaccagactggctataggtaaaactggagagtattcagggattattgattgtggcaagaagcttctaaaacaagaaggtgtcagatcctttttcaaaggttatactcctaacttgctaggcattgtaccttatgccggcatagatcttgctgtttatgagattttgaagaatttttggctagaaaattatgcaggaaactctgtgaatcctgggataatgattttggtgggatgtagtacattgtctaatacttgtggtcagttagccagtttctctgtgaaccttattagaactcgcatgcaggcttcagccccagtggaaaaaggaaaaacaacttctatgattcagctcattcaagaaatatataccaaagaaggaaaattgggattttacaggggtttcacctcaaacatcataaaggtgcttcctgcagtaggcgttggctgtgtggcctatgagaaagtgaagccactttttggattaacctggaagtgatatataaaattgttgtcattgtaatcacttaattgtaagataatacctgtgttataaagagatgttatcttttcctaagaggaaagaaatgtcagataattgttaaaatattttttcattattacaaaatacataaattaaataataatttttataaagttcttgaataagttaaaatatttaatatccaaagaatactatttacattttctactactctcccttgaatcctaatttaaaatttgttcttgatttgatttgaatgtaacataaaacatttggtcagttataggagtttatctttgaaaacgtttttacatttgaaaacttattttaaaaaagctgagcttgaaaatgctttatacactaacagaaaaatcagtgcatttaaaattgatgttatagcaaataatttggagtctgtttcttttacttaagcaaaatggaaaattggtttctaattcct

SEQ ID NO: 49 - DNA, 2633 bp, Homo sapiens
accttctaattctgttattgcaactgcagaccgttacctggcatgctggctgctacctcgctcactcttgtcagagtcggagctacagtcagtgccttcagctctgagctcaggcataccggtccctgtttttgcagttaaggactctaaagtgttgtgacgtgttcatcaagtttttctcaaccataagttaacaattccagtaattgtcatctctcagtcctgattaaacctaattgatttcactagtttttgacccatcatgtgtttgggtttcttctccccagtccctggctctacctcttctgccacaaacgtcagcatggtggtatctgccgaccctttgtccagcgagagggcagagatgaacatcctagaaatcaaccaggaattgcgctcccagctggcagagagcaatcagcagttccgagacctcaaagagaaattccttatatctcaagctactgcctactccctggccaaccagctgaagaaatacaaaaagcaaaatgatcttgaagaggtgaaaggacaagaaacagttgctcccaggctcagcaggggaccactgagagtagacaagtatgaaatcccccaggagtcactggatggatgttgcttgactccttccatccttcctgacctgactccctcctaccacccttattggagcactttgtactcttttgaagacaagcaagtcagcttggctcttgtagacaaaattaaaaaggatcaagaggaggtagaagaccaaagcccaccatgccccaggctcagccaggagctgccagaggtgaaggagcaggaagtcccagaggactccgtgaatgaagtttacttgactccctcagttcaccatgatgtgtctgactgccaccagccttatagcagcaccttgtcctcattggaggatcagcttgcctgctctgctctggatgtagcctcccccaccgaggcggcctgtccccaagggacttggagtggagacttgagccaccaccggtcagaggttcaaatttcacaggcacagctggaaccaagcaccctggtgcccaattgtctgcgactacagctggatcaagggttccactgtgggaacggcttgccccagcggggcctttcctccatgacctgcagcttctcagccaatgctgattctggggatgaagaaccctccccagctggaagatgatgcacttgaaggctcagcaagcaacacacaagggcgtcaagtcactggccggattcatgcctcccttgtcctgatactgaagatcatcaaaagaagactcccgttcagcaagtggagactggcattcagattcgctggcccgcatgcttagagtgcagagataccaaatactgctggaaggatgcaaaggatgataggatgaaagaatgtcacaaaaagcagcttttccacttgataaaaacaactaaaacgcaaagcaagttcaagtcccaacacaatactgcaggggtccttcactgaggattgaatttcagacacagaatactcttgatgacttcaagccactatgctcctttgatttgagaagccacattccatccccctccaattgtgatcaatacctagggagaccaatgcccagatggacaaatagcattgacaggcgttagccctgtttctcaattcccatcatgtagagaacaggagtccgcagctgctggcaggacacagcatgtcagccgggactctgccagggcagagtatgagcaatgccatgttcttgctgaaaacgcttagcctgagtttcataggaggtaaccctcagataactgcagaatgtagaacattgaacaggacaactgacctgtctccttcaaacagtccatgtcaccaccaagaacacaacaaaaaggagaagagacattttgagttcaaaaagagtaaaaagcctatgcagcttatgcttttttagtcattttgaacccaaaacatctcctcatctttttgttgttgtcattgatggtggtgacatggacttgtttgtagaggacaggtcagctgtctggctcaatggtctacattctgaagttatctgaaaatgtcgtcatgattaaatccagcctaaacattttgccaggaactctgcagcgtccatgctgtagcttcctacctcagtccatctgcaggcagagaaggcccagtgtgtccatccccaatgcggtgatactaggatggtcacttggttaaggaggggtctaggagctctgtcccttgtaaagacatcttatttgtaagtaatttggaaagtggtgtgaaatagtataaatatcctgtattctaatgatcttcttcagaacattttatcaccaattaatcaccccgcctgtgtcagttattatatttatgtttgcacaatgaaaattttctatctcaaaatattaccttatacttgcttttgctggcattcttcgtaaaaaagattattccctgcccaaattttaactttcatccaaaattaattttaatttctttttgctggcattctgttgtgaaaaagaatattctctgccccaattatcactttcatccaaaattaattttagtccatcagttaaaattttaagttttaaatctgtttaattaaaacatttcttgcctc

SEQ ID NO: 50 - DNA, 1926 bp, Homo sapiens
ggtagacgcgatctgttcgctactaccggcctcccctggctgttaaaagcagatggtggctgaggctggttcaatgccggctgcctcctctgtgaagaagccatttggtctcagaagcaagatgggcaagtggtgccgccactgcttcccctggtgcagggggagcggcaagagcaacgtgggcacttctggagaccacgacgattctgctatgaagacactcaggagcaagatgggcaagtggtgccgccactgcttcccctggtgcagggggagcagcaagagcaacgtgggcacttctggagaccacgacgactctgctatgaagacactcaggagcaagatgggcaagtggtgctgccactgcttcccctgctgcagggggagcggcaagagcaaagtgggcccttggggagactacgacgacagcgctttcatggagccgaggtaccacgtccgtcgagaagatctggacaagctccacagagctgcctggtggggtaaagtccccagaaaggatctcatcgtcatgctcaaggacactgacatgaacaagaaggacaagcaaaagaggactgctctacatctggcctctgccaatggaaattcagaagtagtaaaactcctgctggacagacgatgtcaacttaatatccttgacaacaaaaagaggacagctctgacaaaggccgtacaatgccgggaagatgaatgtgcgttaatgttgctggaacatggcactgatccgaatattccagatgagtatggaaataccgctctacactatgctatctacaatgaagataaattaatggccaaagcactgctcttatacggtgctgatatcgaatcaaaaaacaagcatggcctcacaccactgttacttggtgtacatgagcaaaaacagcaagtggtgaaatttttaatcaagaaaaaagcaaatttaaatgcactggatagatatggaagaactgctctcatacttgctgtatgttgtggatcggcaagtatagtcagccttctacttgagcaaaacattgatgtatcttctcaagatctatctggacagacggccagagagtatgctgtttctagtcatcataatgtaatttgccagttactttctgactacaaagaaaaacagatgctaaaagtctcttctgaaaacagcaatccagaacaagacttaaagctgacatcagaggaagagtcacaaaggcttaaaggaagtgaaaatagccagccagaggaaatgtctcaagaaccagaaataaataagggtggtgatagaaaggttgaagaagaaatgaagaagcacggaagtactcatatgggattcccagaaaacctgcctaacggtgccactgctgacaatggtgatgatggattaattccaccaaggaaaagcagaacacctgaaagccagcaatttcctgacactgagaatgaacagtatcacagtgatgaacaaaatgatactcagaagcaactttctgaagaacagaacactggaatattacaagatgagattctgattcatgaagaaaagcagatagaagtggctgaaaatgaattctgagctttctcttagttataagaaagaaaaagacctcttgcatgaaaatagtacgttgcaggaagaaattgtcatgctaagactggaactagacgtaatgaaacatcagagccagctaagagaaaagaaatatttggaggaaattgaaagtgtggaaaaaaagaatgataatcttttaaagggtctacaactgaatgagctcaccatggatgatgatactgccgtgctcgtcattgacaacggctctggcatgtgcaaggccggctttgcaggtgacgatgccccccgggctgtcttcccttccatcgtggggtgccccaggcaccagaacatgatggggggcatgcgtc

SEQ ID NO: 51 - DNA, 4174 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaatttaaaaaaaaaaaaaaa

SEQ ID NO: 52 - DNA, 1779 bp, Homo sapiens
attgccgggccagtgcgggagccggagcggagccggggccggagcgggcggaatggagcccctgcgcgcgcccgcgctgcgccgcctgctgccgccgctgctgctcctgctgctgtcactgcccccccgcgcccgggccaagtacgtgcggggcaacctcagttccaaggaggactgggtgttcctgacaagattttgtttcctctcggattacggccgactggacttccgtttccgctaccctgaggccaagtgctgtcagaacatcctcctctattttgatgacccatcccagtggccagccgtgtacaaggcaggggacaaggactgcctggccaaggagtcagtgatccggccggagaacaaccaggtcatcaacctcaccacccagtatgcctggtccggctgtcaggtggtatcagaggagggaacccgctacctgagctgctccagtggccgcagcttccgctcaggtgatggattgcagctggagtatgagatggtcctcaccaatggcaagtccttctggacacgacacttctccgctgatgagtttgggatcctggagacagatgtgaccttcctcctcatcttcatcctcatcttcttcctctcttgttactttggatatttgctgaaaggtcgtcagttgctccacacaacttataaaatgttcatggccgcagcaggagtagaggtcctgagcctcctatttttctgcatctactggggtcaatatgccaccgatggcattggcaacgagagtgtgaagatcttggccaagctgctcttctcctccagcttcctcatcttcctgctgatgcttatcctcctggggaagggattcacggtgacacggggccgcatcagccacgcgggctccgtgaagttgtctgtctacatgaccctgtacacgctcacccatgtggtgctgctcatctacgaggcggaattctttgacccaggccaggtactgtacacgtatgagtcgccggccggctacgggctcattggactgcaggtggcggcctacgtgtggttctgctatgctgtgcttgtctcactgcgacactttcctgagaagcagcctttttatgtgcccttctttgctgcctataccctctggttctttgcggttcctgtcatggccctgattgccaatttcggcatccccaagtgggcccgggagaagattgtcaatggcatccagctggggatccacttgtacgcccatggcgtgtttctgatcatgacccgcccatcagcggccaacaagaacttcccgtaccacgtgcgcacgtcgcagatcgcttcagccggagtccctggacccggagggagccaatccgctgacaaggccttcccgcagcacgtctatgggaacgtgacgtttatcagcgactcggtgcccaacttcacggagctcttctccatccccccgcccgccacctcccccctgccccgagcggcgccggattctgggctcccgctgttccgtgacctccggccccctggcccccttcgagacctctgaccccgctggactccggaacacccgtggtgaccgccgggaccctgcctgtgactctccaggactctgcgaccccgggatggatattgcgatgctggtctcgaccctgaaaccctccctcggatctgtgacctcggacccgtactccatctgccgcatctccattccgggggccttccctcgggtccctggcagaaagacattttaccccttcttgccaaaataaaaaaggattcgtttttatctat

SEQ ID NO: 53 - DNA, 1408 bp, Homo sapiens; identical to SEQ ID NO: 48

SEQ ID NO: 54 - DNA, 1438 bp, Homo sapiens
agcacttcctcatagaccttggatgtgggaggattgcattcagtctagttcctggttgccggctgaaataacctgaattcaagccaggaagaagcagcaatctgtcttctggattaaaactgaagatcaacctactttcaacttactaagaaaggggatcatggacattgaagcatatcttgaaagaattggctataagaagtctaggaacaaattggacttggaaacattaactgacattcttcaacaccagatccgagctgttccctttgagaaccttaacatccattgtggggatgccatggacttaggcttagaggccatttttgatcaagttgtgagaagaaatcggggtggatggtgtctccaggtcaatcatcttctgtactgggctctgaccactattggttttgagaccacgatgttgggagggtatgtttacagcactccagccaaaaaatacagcactggcatgattcaccttctcctgcaggtgaccattgatggcaggaactacattgtcgatgctgggtttggacgctcataccagatgtggcagcctctggagttaatttctgggaaggatcagcctcaggtgccttgtgtcttccgtttgacggaagagaatggattctggtatctagaccaaatcagaagggaacagtacattccaaatgaagaatttcttcattctgatctcctagaagacagcaaataccgaaaaatctactcctttactcttaagcctcgaacaattgaagattttgagtctatgaatacatacctgcagacatctccatcatctgtgtttactagtaaatcattttgttccttgcagaccccagatggggttcactgtttggtgggcttcaccctcacccataggagattcaattataaggacaatacagatctaatagagttcaagactctgagtgaggaagaaatagaaaaagtgctgaaaaatatatttaatatttccttgcagagaaagcttgtgcccaaacatggtgatagattttttactatttagaataaggagtaaaacaatcttgtctatttgtcatccagctcaccagttatcaactgacgacctatcatgtatcttctgtacccttaccttattttgaagaaaatcctagacatcaaatcatttcacctataaaaatgtcatcatatataattaaacagctttttaaagaaacataaccacaaaccttttcaaataataataataataataataaaaaatgtattttaaagatggcctgtggttatcttggaaattggtgatttatgctagaaagcttttaatgttggtttattgttgaattcctagaaaagttttattggtagatgagtaaataaaatattgtaaaaaaacttattgtctataaagtatattaaaacattgttggctaataaaaaaaaaaaaaaa

SEQ ID NO: 55 - DNA, 2931 bp, Homo sapiens
aagtaattgtccgtgtcaggaaggtaggcgtgccaagccgcggctctgcggagaaaccacgaccaccgcggccgccggaaacccaaagcgctccagagcgtccccgggtggccgggcagcaccagggacagcgcccgggactccactggggaccggctcctgggcttcccagcgtcgcgggtagaggtacagctgctccgtgtgccgcaggctccagattctcgccaccccacccctccctcagaaactcggactgctctcgtctgccgtgtggttctcttttcttccgaaaggccagtgtcttatctctccacttcaagtccagaggacttgctcagtctcctccccttaagtcatttccaccatcctcaggcagctgtgggaagccgagagtcctggactgttcgtccgggtgccagcgctggcagtcccagtccgtccggtgcagcagcccggcgcattcccctctctccctccctcttgctctccctccctttctgtcttcctctctttcctcctctactgctccctccctctcttgcctcttaagtttcctgcaccgtgaatccaactgtgccaagccttggctcccgcgaaccaatcctgagcgcgacccgggcactgggacggcgactccgccaaagctggacgaggcagccggacccgtctgcgctcgagcatggagacggagcgcctgggagggcacgtccggggcgctggagacgccaggcccgagtagcttctccatggagcctgcccagagcggtcccttctcgcaggattcgccccaagtcctgtgcggctgctgagagcgctccttgctctgtaaagtggatgtcaggtggatctatgtttctgaaggaacaaagactcaaagaaggcaccgccaaggaagtttgagacgcgggagaatgcaggctgcgtgctggtacgtgcttttcctcctgcagcccaccgtctacttggtcacatgtgccaatttaacgaacggtggaaagtcagaacttctgaaatcaggaagcagcaaatccacactaaagcacatatggacagaaagcagcaaagacttgtctatcagccgactcctgtcacagacttttcgtggcaaagagaatgatacagatttggacctgagatatgacaccccagaaccttattctgagcaagacctctgggactggctgaggaactccacagaccttcaagagcctcggcccagggccaagagaaggcccattgttaaaacgggcaagtttaagaaaatgtttggatggggcgattttcattccaacatcaaaacagtgaagctgaacctgttgataactgggaaaattgtagatcatggcaatgggacatttagtgtttatttcaggcataattcaactggtcaagggaatgtatctgtcagcttggtaccccctacaaaaatcgtggaatttgacttggcacaacaaaccgtgattgatgccaaagattccaagtcttttaattgtcgcattgaatatgaaaaggttgacaaggctaccaagaacacactctgcaactatgacccttcaaaaacctgttaccaggagcaaacccaaagtcatgtatcctggctctgctccaagccctttaaggtgatctgtatttacatttccttttatagtacagattataaactggtacagaaagtgtgccctgactacaactaccacagtgacacaccttactttccctcgggatgaaggtgaacatgggggtgagactgaagcctgaggaattaaaggtcatatgacagggctgttacctcaaagaagaaggtcacatctgttgcctggaatgtgtctacactgctgctcttgtcaactggctgcaaaatacactagtggaaaacactctgatgtaatttctgcccagtcagcttcatccctcagtataattgtaaatcatcacagattttgaattcacacctgaagacatgctctcacatatagaggtacacaaacacaccgtcatgcacatttcagcttgcgtctatcatgattcctgttgagagggctttcattgtctgactcataatggttcaggatcaactatcatcaaacggaaggattaactagacagagaatgtttctaacagttgctgttatggaaatctcttttaaagtcttgagtacatgctaatcaataatctccactcatgcattcctactgcttggagtagctgtactggtaaatactactgtaggagtatctgcttgttaaaatggaaaaatgtgtctttagagctcagtattctttattttacaaacacaacaaaatgtagtaacttttttccagcatacagtaggcacattcaaagtggtccaagatggctcttttttctttgaaaggggcctgttctcagtaaagatgagcaaacatttggaatttacatgtgggcagacattgggataacaactttcatcaccaatcattggacttttgtgaagttgacaccagctaaggctgcttaaaataagttctgatcattatataagaagggaaatgcctggcagacaccatgtaagttataagtgtctgtcttatctttactacacatattgtaacaaattcaatatcctagtcttcatttgtatgaatggtttgtattgtacatagtttaaccaagtgttatttgagctgcttattaatattaacttgtacttgtctctctgcttgttattggttaagaaaaaaggatatgaggaattcattttatcaatgtagctgtgaaggccattaaaaagacaaacttaatgtacagagcatttattcagatcaagtattgttgaaagctatacatatacaacattacagtctgtctgtatttagatattttatttctggaaaaaatgaaatgtacataaaaataaaacacttaaagttgagtttcaataaaaaaaaaaa

SEQ ID NO: 56 - DNA, 1374 bp, Homo sapiens; identical to SEQ ID NO: 15

SEQ ID NO: 57 - DNA, 5497 bp, Homo sapiens
atacacaatcatcatcttactagttctctgtgttgctcgctaaccagtcccccagttcagtagactggagcccagagcctgcttacttgtcaggtgtttattttgtcttgctttttttttttttttaaatgaagtcaaaatgccaataagaccagatctccagcagttggaaaaatgcattgatgatgctttaagaaaaaatgatttcaaacctttgaaaacacttttgcaaattgatatttgtgaagatgtgaagattaaatgcagcaaacagtttttccacaaggtggacaaccttatatgcagggaacttaataaagaggatatccacaatgtttcagccattttggtttctgttggaagatgtggcaaaaatatcagtgtattggggcaagctggacttctaacgatgataaaacaaggactaatacaaaagatggttgcctggtttgaaaaatccaaggacattattcagagtcaaggaaattcaaaagatgaagctgttctaaatatgatagaagacttagttgatcttctgctggtcatacatgatgtcagtgatgaaggtaaaaaacaagtagtggaaagtttcgtacctcgcatttgttccctggttattgactcaagagtgaatatttgtattcagcaagagattataaaaaaaatgaatgctatgcttgacaaaatgcctcaagatgcccggaaaatactctctaaccaagaaatgttaattctcatgagtagtatgggagaaaggattttagatgctggagattatgacttacaggtaggcattgtagaagctttgtgtagaatgaccacagaaaaacaaagacaagaactggcacatcagtggttttcaatggattttattgctaaggcatttaaaagaattaaggactctgaatttgaaacagattgcaggatatttctcaaccttgtaaatggcatgcttggagacaaaagaagggtctttacatttccttgtttatcagcatttcttgataaatatgagctgcaaataccatcagatgaaaaacttgaggaattttggattgattttaatcttgggagtcagactctctcattctacattgctggagataatgatgatcatcaatgggaagcagttactgtgccagaggaaaaagtacaaatatacagcattgaagtgagagaatcaaagaagctactgacaataattctgaaaaatacagtaaaaattagcaaaagagaagggaaagaattgcttttgtattttgacgcatcactagaaatcactaatgtaactcaaaaaatttttggtgcaactaaacatagggaatctatcagaaaacaaggtatttcagttgccaaaacgtcgctgcatatactttttgacgcaagtggatcacagattctagtgccagaaagtcaaatctcaccagtcggagaagagctcgttagtttaaaggaaaaatcaaagtccccaaaggaatttgctaaaccttcaaaatatatcaaaaacagtgacaaagggaatagaaataatagtcagcttgagaaaactactcctagcaaaagaaaaatgtctgaagcatcaatgattgtttctggtgcagatagatacactatgagaagtccagtgcttttcagcaacacatcaataccaccacgaagaagaagaattaaaccaccactgcaaatgacgagctctgcagagaaacctagtgtttctcaaacatcagaaaatagagtggataatgctgcatcactgaaatctagatcatcagaaggaagacatagaagagataatatagacaaacatatcaaaactgctaagtgtgtagaaaacacagaaaataagaatgttgaattcccaaaccaaaattttagtgaactccaggatgttataccagattcacaggcagcggaaaaaagagatcatactatattacctggtgttttagacaacatctgtggaaataaaatacacagcaaatgggcatgttggacacctgtaacaaacattgaactatgtaataaccaaagagcaagtacttcgtcaggagacacattgaatcaagatattgttataaataaaaaacttactaaacaaaaatcatcctcttcaatatctgatcataattctgaaggaacaggaaaagtgaaatataagaaagaacaaaccgaccatatcaaaatagataaagcagaagtagaagtttgcaagaaacacaatcagcaacaaaatcatcctaaatattcagggcagaaaaatactgaaaatgccaagcagagtgattggcctgttgaatctgaaactacttttaaatcggttctcctaaataagacaattgaagaatcgctgatatataggaagaaatacatattgtcaaaagatgtgaatactgctacttgcgataaaaatccatctgctagcaaaaatgtgcaaagtcatagaaaagcagagaaagaattgacttctgagcttaattcctgggattcgaaacaaaaaaaaatgagagaaaagtcaaaagggaaagaatttaccaatgtagcagaatccttgataagccaaatcaataaaagatacaaaacaaaagatgacatcaagtctacaagaaaattaaaggagtctttgattaacagtggtttttcaaacaaacctgttgtacaactcagtaaggaaaaagttcagaaaaaaagctacagaaaactgaagactacctttgttaatgttacttctgaatgcccagtgaatgatgtttacaattttaatttgaatggagctgatgaccctatcataaaacttggaatccaagagtttcaagctacagctaaagaagcttgtgcggataggtcaattagattggtaggtccaaggaatcatgatgaacttaaatcttctgtcaaaacaaaagataaaaaaattataacaaatcatcaaaagaaaaatctgtttagtgatactgaaacagagtacagatgtgatgacagcaagactgatattagctggctaagagaaccgaaatcaaaaccacagctaatagactatagcagaaataaaaatgtgaagaatcataaaagtggaaaatcaagatcatccttggaaaagggacagccaagctctaaaatgacacccagtaaaaatatcacaaaaaagatggacaagacaattccggaaggaagaatcagacttccacgaaaagcaaccaaaacaaaaaaaaactataaagatctctcaaattcagaatcagagtgtgaacaagaattttcacattcatttaaagagaacataccagtaaaggaggagaatatccattccagaatgaaaacggtaaagctaccaaagaaacaacagaaagtcttctgtgctgaaacagaaaaggaactatcaaaacaatggaaaaactcatctctactaaaagatgctatacgagataattgccttgacttatctcccagatctttatctggcagtccatcatctatagaagtaacgagatgtatagagaaaataacagaaaaggattttactcaggattatgactgcataacaaaatctatatcaccttatccaaaaacttcatcacttgaatccttaaatagtaacagtggagttggaggtacaataaagtcacccaaaaacaatgagaaaaacttcctgtgtgcaagtgaaagttgttcaccaattccacgaccactgtttttgcccagacatactccaactaagagtaatactattgtaaatagaaaaaaaataagttctctggtacttacacaagaaacacaaaacagtaacagctattcagatgtaagcagttatagttcagaagaacggtttatggaaattgaatctccacatatcaatgaaaattatatacaaagcaaaagagaggaaagtcatttagcatcttcattatccaagtctagtgaaggaagagagaaaacgtggtttgacatgccctgtgatgctactcatgtatcaggccccacccaacatcttagtcgcaaaagaatatatatagaagataatctaagtaattccaatgaagtagaaatggaagagaaaggagaaaggagagcaaacttgcttcccaaaaaactgtgtaaaattgaagatgcagatcatcatatccacaaaatgtctgaaagtgtatcttcattatcaacaaatgacttttctattccttgggagacctggcaaaatgaatttgcagggatagagatgacttatgagacttacgagaggctcaattcagaatttaagagaaggaataatatccgacataaaatgttgagttattttactacgcagtcttggaaaacagctcagcaacatctgagaacaatgaatcatcaaagtcaggactctaggattaaaaaacttgataaattccaattcattatcatagaggagctggagaattttgaaaaagattcacagtctttaaaagatttggaaaaggaatttgtggacttttgggaaaagatatttcagaagttcagtgcatatcaaaaaagcgaacaacagaggcttcatcttttgaaaacttcattggctaaaagtgtcttctgtaatactgatagtgaagaaactgtttttacatccgagatgtgtttgatgaaagaagatatgaaagtgctgcaagacaggcttcttaaggacatgctagaagaggagcttcttaatgtacgcagagaactgatgtcagtattcatgtctcatgaaagaaatgctaatgtgtgaaatctagtttttatcaccatactttatctaattattattctctgtatataactgaggaaataagaatagtcctacaaagagaaaaatatacatgtcaccgaagcaagtgtaccctttataggaaccctcaaattaaaaaaaaatgtcttttaatggatgagagggaaccactataacatgagtccaagcccagaagacttctgtctatacaatatttttttttaattttggagataaaagctttaagaaactttttgagttaattatactcataaaatgagtttctttaataaattaaattttattgtgtaaaatgtattattacataaaatgtgtttttgaatcaatgcagtttggggatgaatataattaaaatatgtttaataacttagaattcaactaataaaaatttagccacacttacaagggggaggaagtccctagtttaaaatgtataactgagtggtagatcagtactttcagcacactgttggaaacatttattcagatatggctctaatgtattaggaagcactaaatggcctaaaaaagctactacattgcctaaatatgttaattcaatatagaagtcctatttcataaccaggctgtttgacaaatacttttaatctagtagtcattgtaatatcttgctagattaatttataaaaatgagtatacatttgatttgcttttaatgaagttgaaataaatgcttatgtcacttgaataaatataaatcattataaaaaaaaaaaaaaaaaa

SEQ ID NO: 58 - DNA, 440 bp, Homo sapiens
battktagtggtagagtggaatgtatttaaattctctactgtccttgttacatrggtcttkcctccaccctacaaggtgtgtgcttgtaactcaaatttccatttgagtaattagcaattattatttaaaactaacctgaaaataaaaattgtattcaattcattcatagggagcatctacctatttattattaccacataggtgatgtgactctagaaacatcttggtattcaaatagccaattaaaatataaaatgtaatgattttctaaagctactcgttttccttctctcatctctatctactaattggaaagtctattctccaaacacagcaaagatgattgacagaattctaaaaatacacaaatttccctattaaagrgggtgaatggatgttagcactgtatcagacacataatattaaggggatacctbst

SEQ ID NO: 59 - DNA, 1907 bp, Homo sapiens; identical to SEQ ID NO: 9

SEQ ID NO: 60 - DNA, 2939 bp, Homo sapiens
atgcatgctttggacaggtacgcgctcaaacttgacaacgccattattgacgagatcactcccaagcggattggagattgtcccaatacttagacctgtagcaaggccttgggagaaatggtggtgcagcaggagagcaggaacctaaccattgccatcctaaggccctgcattgtgcggagcaacgtggcaccagcttttcctgggttgggttgataatctaaatggatgtagccgactcattattgcggctgggaaagggtttcttctgtccataaaagctactccaatggctgtgggagacttaattccaattccaggtgatacagccgtcagtctcccactagctgtaggatggtgtgctgcagttcacagacctaagtcaatgttagtctaccactgtacgtctggtaacctcaatccctgcaaccggagcaaaatgtgattccaggtcttggcaacctttgaaattccaattccatttgcaagagctttgaggaggccatatgctgatttcaccacaagcaacttcacaacccagtactggaatgccatcagccagcaggcccctgccattatctgtgacttctatctgtggctcattggaaggaaacccaggtgagaagctgagtcaatggctttgagaatgtcactgcatatgggagattgaggccccaaagtctttagggcttccttcagccaaagattaaaggagacaactgaatctgacccatatatacagattgaacacacctactctgaaaatccaaaatctgaaattctccaaaatccaaaaagttttgagtgctgacatgatgccacaagtggaaaattccacacatcttacctcatgtgatgggtcacagtcaaaacacattcaaaactttgtttcatgcataaaattatttaaaatattgtataagattaccttcaggttatgtgtatgtggctaagtgtgtatgaaatgtaagtgaattttgtgtttagatatgggtcccattcccaagatacctcattgcattgaagcaaatattccaaaatctgaaaacagttgaaacccaacacacttctgacccaagcatttcagataaaggatactcaatctgtgtaagttttgaacaaacaaagcagtcatagtgagaagccacagaagcctcctacattaaaaatactccagcataataaaggaaggtaaatgttaaagcgcctgcttgatgaattcagcaagtgatcattcacacaaaaagaaaaccaactgagacgctgtcactagggtttttcaaataggtagagaatcttaaatatccagtaatgatgacaacctcacttactgggagcttactcgtggggctaaggaggatgcgtgatgatcccatttttattgtcacagctaccgaaggaagataccctcatcaacccaattttacaaatggagaaatagaagctcagggaagaatctgaagtagtctcaaaggaagtgacaggaaggatgtggagaaagctgagtgtcaaagtcagtattcaggaccggctttactgctacttagagatgaatgaagaaatcagagggaacgcagtgtgctgatgctaaagcggctgtcaccacccagctgtgtgacataggacatcttctttctctgtctcacttgaataatatgatgtgtcagaggagacatgattgtaattgcctaaagcaattcttgtgatcaagaatcagaagcatgaacagtattgccctctgtgttagcccctttataagggaggaagtcatcttcagcatgctgaattgtcatctttcttagcagtgcaaatgactaaaacttagccaatgtagagtttgtccaaatttggagctcataactcagttcttgagcaaagtgaaaagaaaacattgtgattatggggaaaatatttgtacgggacttatcaaataaagataggaaaagaagaaaactcaaatattataggcagaaatgctaaaggttttaaaatatgtcaggattggaagaaggcatggataaagaacaaagttcagttaggaaagagaaacacagaaggaagagacacaataaaagtcattatgtattctgtgagaagtcagacagtaagatttgtgggaaatgggttggtttgttgtatggtatgtattttagcaataatctttatggcagagaaagctaaaatcctttagcttgcgtgaatgatcacttgctgaattcctcaaggtaggcatgatgaaggagggtttagaggagacacagacacaatgaactgacctagatagaaagccttagtatactcagctaggaatagtgattctgagggcacactgtgacatgattatgtcattacatgtatggtagtgatggggatgatagaaggaagaacttatggcatattttcacccccacaaaagtcagttaaatattgggacactaaccatccaggtcaagaaaagtcacatgccatagccatggtattgcacatcattcatcttgcattctttgagaataagaagatcagtaaatagttcagaagtgggaagctttgtccaggcctgtgtgtgaacccaatgttttgtttagaaatagaacaagtaagttcattgctatagcataacacaaaatttgcataagtggtggtcagcaaatccttgaatgctgcttaatgtgagaggttggtaaaatcctttgtgcaacactctaactccctgaatgttttgctgtgctgggacctgtgcatgccagacaaggccaagctggctgaaagagcaaccagccacctctgcaacctgccacctcctgctggcaggatttgtttttgcatcctgtgaagagccaaggaggcaccagggcataagtctactcacttatatctgtttgtctggaacataacccatgtttgtttttacaacaaataaaattgatcttgaataaaaa

SEQ ID NO: 61 - DNA, 2891 bp, Homo sapiens
ttcgtcccgggcggtgcgttccactgctctggggccggcgccgcgcccagtcccgcttcgggccgcaagccccaccgctcccctccccgggcaggggcgccgcgcagcccgctcccgccgccacctcctcccctgccgccctcctagccggcaggaattgcgcgaccacagcgccgctcgcgtcgcccgcatcagctcagcccgctgccgctcggccctcggcaccgctccgggtccggccgccgcgcggccagggctccccctgcccagcgctcccaggccccgccacgcgtcgccgcgcccagctccagtctcccctccccggggtctcgccagccccttcctgcagccgccgcctccgaaggagcgggtccgccgcgggtaaccatgcctagcaaaaccaagtacaaccttgtggacgatgggcacgacctgcggatccccttgcacaacgaggacgccttccagcacggcatctgctttgaggccaagtacgtaggaagcctggacgtgccaaggcccaacagcagggtggagatcgtggctgccatgcgccggatacggtatgagtttaaagccaagaacatcaagaagaagaaagtgagcattatggtttcagtggatggagtgaaagtgattctgaagaagaagaaaaagcttcttttattgcagaaaaaggaatggacgtgggatgagagcaagatgctggtgatgcaggaccccatctacaggatcttctatgtctctcatgattcccaagacttgaagatcttcagctatatcgctcgagatggtgccagcaatatcttcaggtgtaacgtctttaaatccaagaagaagagccaagctatgagaatcgttcggacggtggggcaggcctttgaggtctgccacaagctgagcctgcagcacacgcagcagaatgcagatggccaggaagatggagagagcgagaggaacagcaacagctcaggagacccaggccgccagctcactggagccgagagggcctccacggccactgcagaggagactgacatcgatgcggtggaggtcccacttccagggaatgatgtcctggaattcagccgaggtgtgactgatctagatgctgtagggaaggaaggaggctctcacacaggctccaaggtttcgcacccccaggagcccatgctgacagcctcacccaggatgctgctcccttcttcttcctcgaagcctccaggcctgggcacagagacaccgctgtccactcaccaccagatgcagctcctccagcagctcctccagcagcagcagcagcagacacaagtggctgtggcccaggtacacttgctgaaggaccagttggctgctgaggctgcggcgcggctggaggcccaggctcgcgtgcatcagcttttgctgcagaacaaggacatgctccagcacatctccctgctggtcaagcaggtgcaagagctggaactgaagctgtcaggacagaacgccatgggctcccaggacagcttgctggagatcaccttccgctccggagccctgcccgtgctctgtgaccccacgacccctaagccagaggacctgcattcgccgccgctgggcgcgggcttggctgactttgcccaccctgcgggcagccccttaggtaggcgcgactgcttggtgaagctggagtgctttcgctttcttccgcccgaggacaccccgcccccagcgcagggcgaggcgctcctgggcggtctggagctcatcaagttccgagagtcaggcatcgcctcggagtacgagtccaacacggacgagagcgaggagcgcgactcgtggtcccaggaggagctgccgcgcctgctgaatgtcctgcagaggcaggaactgggcgacggcctggatgatgagatcgccgtgtaggtgccgagggcgaggagatggaggcggcggcgtggctggaggggccgtgtctggctgctgcccgggtaggggatgcccagtgaatgtgcactgccgaggagaatgccagccagggcccgggagagtgtgaggtttcaggaaagtattgagattctgctttggagggtaaagtggggaagaaatcggattcccagaggtgaatcagctcctctcctacttgtgactagagggtggtggaggtaaggccttccagagcccatggcttcaggagagggtctctctccaggactgccaggctgctggaggacctgcccctacctgctgcatcgtcaggctcccacgctttgtccgtgatgcccccctaccccctcactctccccgtctccatggtcccgaccaggaagggaagccatcggtaccttctcaggtactttgtttctggatatcacgatgctgcgagttgcctaaccctccccctacctttatgagaggaattccttctccaggcccttgctgagattgtagagattgagtgctctggaccgcaaaagccaggctagtccttgtagggtgagcatggaattggaatgtgtcacagtggataagcttttagaggaactgaatccaaacattttctccagccggacattgaatgttgctacaaagggagccttgaagctttaacatggttcaggcccttggtgtgagagcccagggggaggacagcttgtctgctgctccaaatcacttagatctgattcctgttttgaaagtcctgccctgccttcctcctgcctgtagcccagcccatctaaatggaagctgggaattgcccctcacctcccctgtgtcctgtccagctgaagcttttgcagcactttacctctctgaaagccccagaggaccagagcccccagccttacctctcaacctgtcccctccactgggcagtggtggtcagtttttactgcaaaaaaaaaaaa

SEQ ID NO: 62 - DNA, 1851 bp, Homo sapiens
ggatggctctgaagtggacttcagttcttctgctgatacatctcggttgttactttagctctgggagttgtggaaaggtgctggtgtggaccggtgaatacagccattggatgaatatgaagacaatcctgaaagagcttgttcagagaggtcatgaggtgactgtactggcatcttcagcttccattctttttgatcccaatgacgcattcactcttaaactcgaagtttatcctacatctttaactaaaactgaatttgagaatatcatcatgcaacaggttaagagatggtcagacattcaaaaagatagcttttggttatatttttcacaagaacaagaaatcctgtgggaatttcatgacatatttagaaacttctgtaaagatgtagtttcaaataagaaagttatgaaaaaactacaagagtcaagatttgacatcatttttgcagatgctttttttccttgtggtgagctgctggctgcgctacttaacataccgtttgtgtacagtctctgcttcactcctggctacacaattgaaaggcacagtggaggactgattttccctccttcctacatacctgttgttatgtcaaaattaagtgatcaaatgactttcatggagagggtaaaaaacatgatctatgtgctttattttgacttttggttccaaatgtgtgatatgaagaagtgggatcagttttacagtgaagttttaggaagacccactaccttatttgagacaatggggaaagctgacatatggcttatgcgaaactcctggagttttcaatttcctcatccattcttaccaaacattgattttgttggaggactccactgcaaacctgccaaacccctacctaaggaaatggaggaatttgtacagagctctggtgaaaatggtgttgtggtgttttctctggggtcagtgataagtaacatgacagcagaaagggccaacgtaattgcaacagcccttgccaagatcccacaaaaggttctgtggagatttgatgggaataaaccagatgccttaggtctcaatactcggctgtataagtggataccccagaatgaccttctaggtcttccaaaaaccagagcttttataactcatggtggagccaatggcatctatgaggcaatctaccatgggatccctatggtaggcattccattgttttgggatcaacctgataacattgctcacatgaaggccaagggagcagctgttagactggacttccacacaatgtcgagtacagacctgctgaatgcactgaagacagtaattaatgatccttcatataaagagaatgttatgaaattatcaataattcaacatgatcaaccagtaaagcccctgcatcgagcagtcttctggattgaatttgtgatgtgccacaaaggagccaaacaccttcgagttgcagcccgtgacctcacctggttccagtaccactctttggatgtgattgggtttctgctggcctgtgtggcaactgtgatatttgtcgtcacaaagttttgtctgttttgtttctggaagtttgctagaaaagggaagaagggaaaaagagattagttatgtctgacatttgaagctggaaaaccagatagatgggttgacatcagtttattccagcaagaaagaaaagattgttatgcaagatttctttcttcctgtgacaaaaaaaaaaacttttcaaaatctaccttgtcaagtaaaaatttgtttttcagagatttaccacccaggtaatggttagaaatattctgtggcaatgaagaaaacactagggaaaataaaaaataatataaagccaaaaaaaaaaaaaaaaaaa

SEQ ID NO: 63 - DNA, 3884 bp, Homo sapiens
ccgagtgacaaggaggtgggagagggtagcagcatgggctacgcggttggctgccctcagtccccctgctgctgaagctgccctgcccatgcccacccaggccgtggggccaggggcctgccagggctaggagtgggcctgccgttcatgggtctctagggatttccgagatgcctgggaagagaggcttgggctggtggtgggcccggctgcccctttgcctgctcctcagcctttacggcccctggatgccttcctccctgggaaagcccaaaggccaccctcacatgaattccatccgcatagatggggacatcacactgggaggcctgttcccggtgcatggccggggctcagagggcaagccctgtggagaacttaagaaggaaaagggcatccaccggctggaggccatgctgttcgccctggatcgcatcaacaacgacccggacctgctgcctaacatcacgctgggcgcccgcattctggacacctgctccagggacacccatgccctcgagcagtcgctgacctttgtgcaggcgctcatcgagaaggatggcacagaggtccgctgtggcagtggcggcccacccatcatcaccaagcctgaacgtgtggtgggtgtcatcggtgcttcagggagctcggtctccatcatggtggccaacatccttcgcctcttcaagataccccagatcagctacgcctccacagcgccagacctgagtgacaacagccgctacgacttcttctcccgcgtggtgccctcggacacgtaccaggcccaggccatggtggacatcgtccgtgccctcaagtggaactatgtgtccacagtggcctcggagggcagctatggtgagagcggtgtggaggccttcatccagaagtcccgtgaggacgggggcgtgtgcatcgcccagtcggtgaagataccacgggagcccaaggcaggcgagttcgacaagatcatccgccgcctcctggagacttcgaacgccagggcagtcatcatctttgccaacgaggatgacatcaggcgtgtgctggaggcagcacgaagggccaaccagacaggccatttcttctggatgggctctgacagctggggctccaagattgcacctgtgctgcacctggaggaggtggctgagggtgctgtcacgatcctccccaagaggatgtccgtacgaggcttcgaccgctacttctccagccgcacgctggacaacaaccggcgcaacatctggtttgccgagttctgggaggacaacttccactgcaagctgagccgccacgccctcaagaagggcagccacgtcaagaagtgcaccaaccgtgagcgaattgggcaggattcagcttatgagcaggaggggaaggtgcagtttgtgatcgatgccgtgtacgccatgggccacgcgctgcacgccatgcaccgtgacctgtgtcccggccgcgtggggctctgcccgcgcatggaccctgtagatggcacccagctgcttaagtacatccgaaacgtcaacttctcaggcatcgcagggaaccctgtgaccttcaatgagaatggagatgcgcctgggcgctatgacatctaccaataccagctgcgcaacgattctgccgagtacaaggtcattggctcctggactgaccacctgcaccttagaatagagcggatgcactggccggggagcgggcagcagctgccccgctccatctgcagcctgccctgccaaccgggtgagcggaagaagacagtgaagggcatgccttgctgctggcactgcgagccttgcacagggtaccagtaccaggtggaccgctacacctgtaagacgtgtccctatgacatgcggcccacagagaaccgcacgggctgccggcccatccccatcatcaagcttgagtggggctcgccctgggccgtgctgcccctcttcctggccgtggtgggcatcgctgccacgttgttcgtggtgatcacctttgtgcgctacaacgacacgcccatcgtcaaggcctcgggccgtgaactgagctacgtgctgctggcaggcatcttcctgtgctatgccaccaccttcctcatgatcgctgagcccgaccttggcacctgctcgctgcgccgaatcttcctgggactagggatgagcatcagctatgcagccctgctcaccaagaccaaccgcatctaccgcatcttcgagcagggcaagcgctcggtcagtgccccacgcttcatcagccccgcctcacagctggccatcaccttcagcctcatctcgctgcagctgctgggcatctgtgtgtggtttgtggtggacccctcccactcggtggtggacttccaggaccagcggacactcgacccccgcttcgccaggggtgtgctcaagtgtgacatctcggacctgtcgctcatctgcctgctgggctacagcatgctgctcatggtcacgtgcaccgtgtatgccatcaagacacgcggcgtgcccgagaccttcaatgaggccaagcccattggcttcaccatgtacaccacttgcatcgtctggctggccttcatccccatcttctttggcacctcgcagtcggccgacaagctgtacatccagacgacgacgctgacggtctcggtgagtctgagcgcctcggtgtccctgggaatgctctacatgcccaaagtctacatcatcctcttccacccggagcagaacgtgcccaagcgcaagcgcagcctcaaagccgtcgttacggcggccaccatgtccaacaagttcacgcagaagggcaacttccggcccaacggagaggccaagtctgagctctgcgagaaccttgaggccccagcgctggccaccaaacagacttacgtcacttacaccaaccatgcaatctagcgagtccatggagctgagcagcaggaggaggagccgtgaccctgtggaaggtgcgtcgggccagggccacacccaagggcccagctgtcttgcctgcccgtgggcacccacggacgtggcttggtgctgaggatagcagagcccccagccatcactgctggcagcctgggcaaaccgggtgagcaacaggaggacgaggggccggggcggtgccaggctaccacaagaacctgcgtcttggaccattgcccctcccggccccaaaccacaggggctcaggtcgtgtgggccccagtgctagatctctccctcccttcgtctctgtctgtgctgttggcgacccctctgtctgtctccagccctgtctttctgttctcttatctctttgtttcaccttttccctctctggcgtccccggctgcttgtactcttggccttttctgtgtctcctttctggctcttgcctccgcctctctctctcatcctctttgtcctcagctcctcctgctttcttgggtcccaccagtgtcacttttctgccgttttctttcctgttctcctctgcttcattctcgtccagccattgctcccctctccctgccacccttccccagttcaccaaaccttacatgttgcaaaagagaaaaaaggaaaaaaaatcaaaacacaaaaaagccaaaacgaaaacaaatctcgagtgtgttgccaagtgctgcgtcctcctggtggcctctgtgtgtgtccctgtggcccgcagcctgcccgcctgccccgcccatctgccgtgtgtcttgcccgcctgccccgcccgtctgccgtctgtcttgcccgcctgcccgcctgcccctcctgccgaccacacggagttcagtgcctgggtgtttggtgatggttattgacgacaatgtgtagcgcatgattgtttttataccaagaacatttctaataaaaataaacacatggttttgcaaaaaa

SEQ ID NO: 64 - DNA, 2277 bp, Homo sapiens
aattagccgggtgcggtggcgggcgcctgtagtcccagctactccggaggctgaggcaggagaatggcgtgaacccaggagatggagcttgcagtgagccgagatcgcgccactgcactctagcctgggcaacccaaggagactccatctcaaaacaacaacaacaacaacaacaacaacaacaacaaccagctgagcagcgcgggccatctggcagcgggtccagctccagggcgccgtagggcagcggagtgcagtgttcgggtaataggacgcagacggcggggtcgccgggggcttcggggtggcctcagccccaggccatccagccctgtggaccgaatggagtcccacacgctgttgaggtagttgtgggttcccctggcctcgggctgggcgcggggtcagcgcgcctgcaggcggcgcttgcggtacgggctggtgaaagtggagacggacggcaggatggattcacttggccacatggcacgaagctgggaagacggacaccgacctaagtcaatgttagtctactactgtacatctggtaacctcaatccctgcaaccgaggcaaaatgggattccaggtcttggcaacctttgaaattccaattccatttgcgagagctttgaggaggccatatgctgattttaccaccagcaacttcacaacccagtactggaatgccatcagccagcaggcccctgccatcatctgtgacttctatctgtggctcactggaaggaaacccagctaccgaaggaagataccctcatcaacccaattttacaaatggagaaatagaagctaagggaagaatctgaagtagtctcaaaggcagtgacaggaaggatgtggagaaagctgagtgtcaaagtcagtattcatgactagatttactgctacttagagatgaatgaagaaatcagagggaacgcagtgtgctgatgctaaagcagctgtcaccactcagctgtgtgacataggacatcttcttcctctgtctcacttgaataatatgatgtgtcagaggagacatgattgtaattgcctaaagcaatttttgtgatcaagaatcagaaacatgaacagtattgctgtctgtgttagcccctttataagggaggaagtcatcttcagcatgttgaattgtcatctttcttagcagtgcaaatgactaaaacttagccaatgtagagtttatccaaatttggagcaaagtgaaaagaaaccattgtgattatggggaaaatatttgtatgggactattaaataaagacacgaaaagaagaaaacccaaatattataggcagaaatgctaaaggttataaaatatatcaggattggaagaaggcatggataaagaacaaagttcagttaggaaagagaaacacagaaggaagagacacaataaaagtcatgtattctgtgagaagtcagacagatttgtggggaatggattggtttgttgtatggtatgtattttagcaataatctttatggcagagaaagctaaaatcccttagcttgcatgaatgatcacttgctaaattcctcaaggtaggcatgatgaaggagggtttagaggagacacagacacaatgaactgacctagatagaaagccttagtatactcagctaggaatagtgattctgaggacacactgtgacatgattatgtcattacatgtatggtagtgatggggatgatagaaggaagaacttatggcatattttcacccccacaaaaatcagttaaatattgggacactaaccatccaggtcaagaaaagtcacatgccatagccatggtattgcacatcattcatcttgcattctttgagaataagaagatcagtaaatagttcagaagtgggaagctttgtccaggcctgtgtgtgaacccaatgttttgtttagaaatagaacaagtaagttcattgctatagcataacacaaaatttgcataagtggtggtcagcaaatccttgaatgctgcttaatgtgacgaggttggtaaaatcctttgtgcaacactcttactccctgaatgttttgctgtgctggaacttgtgcgtgccagacaaggccaagctgggtgaaagagcaatcagccacctctgcaacctgccacctcctgctggcaggatttgtttttgcatcctgtgaagagccaaggaggcaccagggcataagtctactcacttatatctgtttgtctgaaacataacccatgtttgtttttacaacaaataaaattgatcttgaataaaaa

SEQ ID NO: 65 - DNA, 3680 bp, Homo sapiens
gcggttgtggctcaggggtcagctcctgctagtgccaggacactactgggaggctgggacccgaccaaagcccatggtgtctctggcctgagaacaaggtgtcttgggaccataaggccaggccaccaatggccattgggtcataggggctcagccccaatctttgtctttccctggctccttctgattcagtcccatcagggccctggatcccaagactcagcatccaaggtcccctccaggaatcctggcagctcagcatactttatcctgtttcatctgagagcaaaaatgtaaaattggatgcacagaaaagtgactcaaagtgcttaatgactagaagaaatctaggagcagcaagaaggtaatgtggagggagggacctccatgaccggtgtctgcagagccaggggtacaggcacccagtgctgtggcctggcaccacctgcctctcagagggtgggtggcacactccttaaccagaggacagcaggcctggtcaccagcttttctacctgtccctgtaagcatcacattgctggaggaaaatctcatgccagagcttggaccatccctagctcaggggttaggggttgtcccttggtgacctaaatgaaaaaacaggtccagaacagagttcctgatgctggacactcattcagtctttgaatcgtgggaggggaggcctggtactaggtagacctaacctctttgaggaaccacagagcccaaggctggaaatctccagaatcctccaccccctgatcctccctggggacccctgtggcctgtctcactgagaactcttccatctgtagatgtctgggctgctgtacaagggagtcccctttcaggtgtggtgctagacatggtcactcctgctggatgtctaggtggtagaaaccaaggacctagggaaataccaggtacagcctttccccgctcatccagagcaggacaaacaggccaggcggtgtcaggagcccaggtctccagctggagggaacgtcaaccctgcggtgggagcaggggccctttgcacatcctaggcacagatggtaatgtagacaccacaggtaagctgggcttggtacctacccctccccggattcagaaagaaaccaaacaaggagctttgtgcggaatgaaacctcctttcctcccagaagcactgctgactgtttggtggttgccatttgtggcagtgagcctttgtttgttctgaggttgggctggtttctcctcttggtcctgccctacagatcataaaggagaacagcaagaggtccccagcaaacatccacagatggccttggaacctcaccctgcaggaatgccagtgaacatactgctgacatcttggagctcagtaccctcatagtgtaacggcgtcagtagatctgcctgtgcttggacttcctgtactacccattcctgaggggcgatgcttctgcagggcctgtgacttggtgcacaacttcagacaccatcatcttgcagcagcaccgcaccctcactagccagggtgttgatgacttcctcaaggccaaggccacattcaaggcttcggactttattgatgcgcttgtgctgagcaaggtggcttctccaggatcttaattcaggaggtagaatggagcttgagatcaagtgtctgatcaagcctcagtgtatgggcgctgttcatcctctggtgctgaagcagccaagagacccaagtctgcctggctgcctcttaggatatgacagcagagccagtggcctctactagatcctgtacaacctcacaaaacacccagacatcgggagtgctgccagcctgtgatgcaagagtcctaatcctgaagacattgaatgacctgtcattctgctgtttttaccaaaaaggatcatgaggatcagagaggaaaagtcacttgcccaaaatcacacagctgaacagtggtggagttcaactttgaccatgggctgtctggccccaaggtgtatgcttgcttctctcccaagagactcctttcttatcaggctcaaatgaatgaaaggaggatgttaaagttcctagaagctttaattgaatgaaagttcctagtagatctgtacctactaaaaaccacacttctgaagctacgtggccaccagaagacacagctagtctgccatgtaaaaaaggaaaggtggcgtgtgccctgaaggcgcaggggtgagaggcagggaaatggagacccccacagccagcatcagtggccctcatcacagccctccaggagatatcaaaggagacaacgccattattgacgagatcactcccaagcggattggagattgtcccaatacttagacctatagcaaggccttgggagaaatggtggtgcagcaggagagcaggaacctaaccattgccatcctaaggccctccattgtgcggagcaacgtcgcaccagcttttcctgggttgggttgataatctaaatggatgtagccgactcattattgcggctgggaaagggtttcttctgtccataaaagctactccaatggctgtgggagacttaattccaattccaggtgatacagccgtcaatctcccactagctgtaggatggtgtgtgtgctgcagttcacagggcaaaggagatagaagtggatgaaatgagagaattttctttaattgaactctggttaatgccaaaagtgttcaatcactatgtgtggggaagtttcctggtacaaaggaaaaaaaaacaacctaagtcagtgttagtctaccactgtacatctggtaacctcaatccctgcaaccggggcaaaatgggtttccaggtcttggcaacctttgaaattccaattccatttgagagagctttgacgaggccatatgctgatttcaccaccagcaacttcagaacccagtactggaatgccatcagccagcaggcccctgccatcatctatgacttctatctgtggctcactggaaggaaacccagctaccgaaggaagataccctcatcaacccaattttacaaatggagaaatagaagttaagggaagaatctgaagtagtctcaaaggcagtgacaggaaggatgtggagaaagctgagtgtcaaagtcagtattcaggaccggctttactgctactttgagatgaatgaagaaatcagagggaacgcagtgtgctgatgctaaagcagctgtcaccacccagctgtgtgacataggacatattctttctctgtctcacttgactaatatgatatgtcagaggagacatgattgtaattgcctaaagcaattcttgtgatcaagactcagaagcacgaacagtattgccctctgtgttagcccctttataagggaggatatcatcttcagcatgctgaattgtcatctttcttagcagtgcaaatgactaaaacttagccaatgtagagtttgtccaaatttggagctcataactcagttcttgagcaaagtgaaaagaaaacattgtgattatggggaaaatatttgatgggacttatcaaataaagataggaaaagaagaaaacccaaatattataggcagaaatgctaaaggttttaaaatatgtcaggattggaagaaggcatggataaagaacaaagttcagttaggaaagagaaacacagaaggaagagacacaataaaagtcattatgt

SEQ ID NO: 66 - DNA, 2250 bp, Homo sapiens
atgtccaggcctctcaagcagcacagaaatgagcctacctgtggaaggaaagtttctcttccaaataaagccttagaattaaaggacagagaaacactcaaagcagagtctcctgataaagatggtcttctgaagcctacctgtggaaggaaagtttctcttccaaataaagccttagaattaaaggacagagaaacattcaaagcagctcagatgttccaatcagaatccaagcaataggacgatgaagaacattcttgggattttgagagtttccttgagactctcttacagaatgatgtgtgtttacccaaggctacacatcaaaaagaatttgataccttaagtggaaaattagaagactctcctgataaagatggtcttctgaagcctacctgtggaatgaaaatttctcttccaaataaagccttagaattgaaggacagagaaacattcaaagcagtggatgtgagttctgtagagtccacattcagtctttttggcaaaccgactactgaaaattcacagtctacaaaagttgaggaagactttaatcgtactaccaaggaggcagcaacaaagacagtaactggacaacgagaacgtgatattggcattattgaacgagctccacaagatcaaacaaataagatgcccacatcagaattaggaagaaaagaagatacaaaatcaacttcagattctgagattatctctgtgagtgatacacagaattatgagtgtttacttaggctacatatcaaaaagaaataaagacaacaaatggcaaaatagaagagtctcctgaaaagccttctcactttgagcctgccactgaaatgcaaaactctgttccaaataaaggcttagaatggaagaataaacaaacattgagagcagattcaactaccctatcaaaaatcttggatgcagttccttcttgtgaaagaggaagggaacttaaaaaagatcactgtgaacaaattacagcaaaaatggaacaaacaaaaaataagttttgtgtactacaaaaggaactgtcagaagcaaaagaaataaaatcacagttagagaaccaaaaagctaaatgggaacaagagctctgcagtgtgagattgactttaaatcaagaagaagagaagagatgtcgatatattaaaagaaaaaattagacctgaagagcaacttaggaaaaagttagaagtgaaacaacaacttgaacaggctctcagaatacaagatatagaattgaaaagtgtaacaagtaatttgaatcaggtttctcacactcatgaaagtgaaaatgatctctttcatgaaaattgcatgttgaaaaaggaaattgccatgctaaaactggaagtagccacactgaaacgtcaacaccaggtgaaggaaaataaatactttgaggacattaagattttacaagaaaagaatgctgaacttcaaatgaccctaaaactgaaacagaaaacattaacaaaaagggcatctcagtatagagagcagcttaaagttctgacagcagagaacacgatgctgacttctaaattgaaggaaaaacaagacaaagaaatactggagacagaaattgaatcacaccatcctagactggcttctgctttacaagaccatgatcaaagtgtcacatcaagaaaaaaccaagaacttgctttccacagtgcaggagatgctcatttgcaaggaataatgaatgttgatgtgagtaatacaatatataacaatgaggtgctccatcaaccactttatgaagctcaaaggaaatccaaaagcccaaaaattaatctcaattatgcaggagatgatctaagagaaaatgcattggtttcggaacatgcacaaagagaccgatgtgaaacacagtgtcaaatgaagaaagctgaacacatgtatcaaaatgaacaagataatgtggacaaacacactgaacagcaggagtctctggagcagaaattatttaaactagaaagcaaaaataggtggcttcgacagcaattagtttatgcacataagaaagttaacaaaagcaaggtaacaattaatattcagtttcctgagacgaaaatgcaacgtcatctaaaagagaaaaatgaggaggtattcaattatggtaaccatttaaaagaatgtatagatcaatatgaaaaagagaaagcagaaagagaagtaagtatcaaaaaatataaatacttttcaaccttcctgaaagaaaatggccttggctaa

SEQ ID NO: 67 - DNA, 2535 bp, Homo sapiens
acccttcccccttctccaaccgccaacggccgcctgcgcctgaggcccggcccacgccccatcccgctcgctccacgctgcggcagccccggcggccccaggtgcgcggcccccgccatcccgccccgccgccctgcgccatggaggagggccctctgccgggcgggctgcccagccccgaggatgcgatggtgacggagctcttaagccccgagggtccgttcgcttcggagaacatcggcctgaaggcccccgtgaagtacgaggaggacgagttccacgtcttcaaagaagcgtacctgggcccggcggaccccaaggaacccgtcctgcacgcgttcaaccccgcgctgggcgccgactgcaagggccaggtcaaggcgaagctcgcggggggcgacagcgacggcggggagctcctcggggagtaccccgggatcccagagctcagcgcgctggaggacgtcgcgctcctgcaggccccgcagccgcccgcctgcaacgtgcacttcctgtcctcgctgctacccgcgcaccgcagcccggcggtgttgcccctgggcgcctgggtcctggaaggagcctcccacccgggcgtccgcatgatcccagttgaaatcaaggaagcaggtggtactactacaagtaataatccggaagaagcaactttgcagaatcttcttgctcaggaatcctgttgcaagttcccatcgtcccaggaactagaggatgcctcctgctgttctcttaagaaagattccaacccaatggtgatatgccaattgaaagggggcacacaaatgctatgtatagacaattctagaacaagagaactaaaagcactccatttggttcctcagtatcaagatcaaaataattatctacagtcagatgtccctaaaccaatgactgctttagtagggagatttttgccagcatcaacaaaattaaatctcattacacaacaacttgagggagccttaccatcggtagtcaacgggtctgctttcccctcgggatcaactcttccaggaccaccaaaaataactttggctgggtactgtgactgctttgccagtggggacttttgcaacaactgcaattgtaataattgttgcaacaacttgcatcatgatattgaacggtttaaagccattaaggcatgtcttggtagaaatccagaagctttccagccaaaaattgggaagggccaattgggcaatgtcaagccccagcacaacaaagggtgcaactgcaggaggtcaggctgcctgaagaattactgcgagtgctatgaggcccaaattatgtgttcttctatttgcaaatgcattggttgcaaaaattatgaagaaagcccagaacgaaagacactaatgagcatgccaaactacatgcagactggaggtttggaaggcagccattacctgccaccaacgaaattttcaggacttccaagattcagtcacgataggcggccttcctcatgcatctcctgggaggtggtggaggccacatgcgcctgcctgcttgctcagggagaagaggccgagaaagaacactgctccaagtgcctggcagagcagatgatcctggaggaatttggaaggtgcttatcacagattctccacactgagtttaaatctaagggattgaaaatggagtagagtataaagtgtgaatgcatgttgattttgtcttagtctagaaatctctagtttagaaaggatgtttaggggaacatgaggctggctctgcagcaacaaccaggctcccctgcatccctgggcccagggagtttactcagagctctctgaagatgtggcaacccatgcccccttttctgaggaggtgcatggcctgagcattgtttgtctggcccagaggagagagcttgggttcccatagtcctgggagagtgtctgcagggcggcggagggcagagcagggcaggggagggcacagcaggccctgcgaagggcagagcggggcaggggagggcagagcaggccctgcggagagctcactctggtcgactcttcctctcagagaatgttgctctggaggctgctctgcatgaaaaccctaatggtttcttgtttgtttttcaaattatttagaaataagttctccggatgggctgttgtgataccacttaaaatctctagagaactactgaacacctaaagattttctgtagcgtagatatttccccagaggcacgcgaactgtcagtctttcctaaggcccccgggagacgcaggcaatggggcctcgcaggccaggcttgcaccagcatgtcttgagttagaggacttaaaattatccagtttcttctgtgtttctacttgaattgtggaaaagctctattatccaattaacttctccataattattgttgtaatattattattgtttgtaaaacatggttcacataactagcttgtggaaaccagcaggtaaaatgaattcttaagttgacgcttttggttctgttgtaaagcaaagatgaataaaaatttccaatgtcttcaa

SEQ ID NO: 68 - DNA, 2681 bp, Homo sapiens
aaaggtatctgttaagctaggtaggaactgcagtcggctggttgcttctcatctggagaaagcaggcaactgggcagtgattgaagtgtccagcagggggctggcattctctgtctataagtaacactggttcctcttcagagcctcagctcagcggagctgccgtttgctggtgaagcccgtgacgtgcaaagcatcctgcctataggatttgaggatttctcagtgcagtttttttctacccactttaaacctccagattctaaatatcaggaaagacgctgtgggaaaatagcaggccaaaagttcttagtaaactgcagccagggagactcagactagaatggaggtagaaagaactgatgcagagtgggtttaattctaagcctttttgtggctaagttttgttgttgttaacttattgaatttagagttgtattgcactggtcatgtgaaagccagagcagcaccagtgtcaaaatagtgacagagagttttgaataccatagttagtatatatgtactcagagtatttttattaaagaaggcaaagagcccggcatagatcttatcttcatcttcactcggttgcaaaatcaatagttaagaaatagcatctaagggaacttttaggtgggaaaaaaaatctagagatggctctaaatgactgtttccttctgaacttggaggtggaccatttcatgcactgcaacatctccagtcacagtgcggatctccccgtgaacgatgactggtcccacccggggatcctctatgtcatccctgcagtttatggggttatcattctgataggcctcattggcaacatcactttgatcaagatcttctgtacagtcaagtccatgcgaaacgttccaaacctgttcatttccagtctggctttgggagacctgctcctcctaataacgtgtgctccagtggatgccagcaggtacctggctgacagatggctatttggcaggattggctgcaaactgatcccctttatacagcttacctctgttggggtgtctgtcttcacactcacggcgctctcggcagacagatacaaagccattgtccggccaatggatatccaggcctctcatgccctgatgaagatctgcctcaaagccgcctttatctggatcatctccatgctgctggccattccagaggccgtgttttctgacctccatcccttccatgaggaaagcaccaaccagaccttcattagctgtgccccatacccacactctaatgagcttcaccccaaaatccattctatggcttcctttctggtcttctacgtcattccactgtcgatcatctctgtttactactacttcattgctaaaaatctgatccagagtgcttacaatcttcccgtggaagggaatatacatgtcaagaagcagattgaatcccggaagcgacttgccaagacagtgctggtgtttgtgggcctgttcgccttctgctggctccccaatcatgtcatctacctgtaccgctcctaccactactctgaggtggacacctccatgctccactttgtcaccagcatctgtgcccgcctcctggccttcaccaactcctgcgtgaacccctttgccctctacctgctgagcaagagtttcaggaaacagttcaacactcagctgctctgttgccagcctggcctgatcatccggtctcacagcactggaaggagtacaacctgcatgacctccctcaagagtaccaacccctccgtggccacctttagcctcatcaatggaaacatctgtcacgagcggtatgtctagattgacccttgattttgccccctgagggacggttttgctttatggctagacaggaacccttgcatccattgttgtgtctgtgccctccaaagagccttcagaatgctcctgagtggtgtaggtgggggtggggaggcccaaatgatggatcaccattatattttgaaagaagccatcaagtcttaagtttttcatttcaacttgtgaacgtttcttctgatgtgaagcaaaccttcccttttcagaaaagggaacaagtagaaaattattttttaagcctcaagccctgttaaatggtcgtggccaattatgtcatagaaactgtatgaacaaccagatttacatagcagagaaatcatacattgaatgcttactttgtgaaagacttcaccttgtcatttctttaagcagacgctagtactttagaaatataacttgactctgttttcaggaatatctgtaatacacaaaccaaggaacaacttttatttacactcctaatatgaaaagtcaatcctgtgagagagctccatgtatgagggacactctccaagttgataacaatggaagcgagtttaatataaaacaattccctaagcatttattttttttttaaaaagatgttactgaggacctagaagaaatgctcaatacatactttgaaagcaaaaatacaatcaaacacattgacacgtatataaagatccacgcgtggctgtgcgtgatatctcacactctgaattcttacttgatggaggttttgtttgctgctacggttttaatcatccagggtgccattccaccatagaagagcaatccttttaggaaaaaaaaaatcatgctattaattaatcaaatatctataaatgcata

SEQ ID NO: 69 - DNA, 3307 bp, Homo sapiens; identical to SEQ ID NO: 7

SEQ ID NO: 70 - protein, 581 aa, Homo sapiens
MDIIKGNLDGISKPASNSRIRPGSRSSNASLEVLSTEPGSFKVDTASNLNSGKEDHSESSNTENRRTSNDDKQESCSEKIKLAEEGSDEDLDLVQHQIISECSDEPKLKELDSQLQDAIQKMKKLDKILAKKQRREKEIKKQGLEMRIKLWEEIKSAKYSEAWQSKEEMENTKKFLSLTAVSEETVGPSHEEEDTFSSVFHTQIPPEEYEMQMQKLNKDFTCDVERNESLIKSGKKPFSNTEKIELRGKHNQDFIKRNIELAKESRNPVVMVDREKKRLVELLKDLDEKDSGLSSSEGDQSGWVVPVKGYELAVTQHQQLAEIDIKLQELSAASPTISSFSPRLENRNNQKPDRDGERNMEVTPGEKILRNTKEQRDLHNRLREIDEKLKMMKENVLESTSCLSEEQLKCLLDECILKQKSIIKLSSERKKEDIEDVTPVFPQLSRSIISKLLNESETKVQKTEVEDADMLESEECEASKGYYLTKALTGHNMSEALVTEAENMKCLQFSKDVIISDTKDYFMSKTLGIGRLKRPSFLDDPLYGISVSLSSEDQHLKLSSPENTIADEQETKDAAEECKEP

SEQ ID NO: 71 - protein, 9 aa, Homo sapiens
SLLKFLAKV

SEQ ID NO: 72 - protein, 9 aa, Homo sapiens
MLLVFGIDV

SEQ ID NO: 73 - protein, 9 aa, Homo sapiens
KVTDLVQFL

SEQ ID NO: 74 - protein, 10 aa, Homo sapiens
GLYDGMMEHL

SEQ ID NO: 75 - protein, 9 aa, Homo sapiens
FLWGPRAHA

SEQ ID NO: 76 - protein, 9 aa, Homo sapiens
VIWEALNMM

SEQ ID NO: 77 - protein, 9 aa, Homo sapiens
KMSILKFLA

SEQ ID NO: 78 - protein, 9 aa, Homo sapiens
KNYEDHFPL

SEQ ID NO: 79 - protein, 9 aa, Homo sapiens
FVLVTSLGL

SEQ ID NO: 80 - protein, 9 aa, Homo sapiens
ILFSEASEC

SEQ ID NO: 81 - protein, 9 aa, Homo sapiens
GMLSDVQSM

SEQ ID NO: 82 - protein, 9 aa, Homo sapiens
ILILILSII

SEQ ID NO: 83 - protein, 9 aa, Homo sapiens
GILILILSI

SEQ ID NO: 84 - protein, 9 aa, Homo sapiens
NMMGLYDGM

SEQ ID NO: 85 - protein, 9 aa, Homo sapiens
QIACSSPSV

SEQ ID NO: 86 - protein, 9 aa, Homo sapiens
LIPSTPEEV

SEQ ID NO: 87 - protein, 9 aa, Homo sapiens
IIFIEGYCT

SEQ ID NO: 88 - protein, 8 aa, Homo sapiens
WEALNMGL

SEQ ID NO: 89 - DNA, 50 bp, Homo sapiens
gatccgctaaggggcatctgaaacatccgtcgagtggcagaggcaggata

SEQ ID NO: 90 - DNA, 50 bp, Homo sapiens
gcgccatcttgccctgtagatcattttggggacacctccagtatttcatg

SEQ ID NO: 91 - DNA, 50 bp, Homo sapiens
cgcactctttacggcttcggtggctaaggctcctgcttgctgcactctta

SEQ ID NO: 92 - DNA, 50 bp, Homo sapiens
gtgttcctggagaatgtgattcgggacgcagtcacctacaccgagcacgc

SEQ ID NO: 93 - DNA, 50 bp, Homo sapiens
tgccgtattcttggtgtctggagcagtgcctgacctgtggcgggtgctta

SEQ ID NO: 94 - DNA, 50 bp, Homo sapiens
tagggagtagaaccgtctctcttcttagttggtgactgtttggggcctgg

SEQ ID NO: 95 - DNA, 50 bp, Homo sapiens
ctgcctggtggggtaaggtccccagaaaggatctcatcgtcatgctcagg

SEQ ID NO: 96 - DNA, 50 bp, Homo sapiens
ggtggtcactgggaatttttgctgtggccctgcttttccttcttcccact

SEQ ID NO: 97 - DNA, 50 bp, Homo sapiens
tccctgaacgacactctcctaatcctaagccttacctgagtgagaagccc

SEQ ID NO: 98 - DNA, 50 bp, Homo sapiens
atggtggttgaggttgattccatgccggctgcctcttctgtgaagaagcc

SEQ ID NO: 99 - DNA, 50 bp, Homo sapiens
ccaacatgctctaatgcttcagattcaagtgctttttccactgtttcccc

SEQ ID NO: 100 - DNA, 50 bp, Homo sapiens
ggggtttcacctcaaacatcataaaggtgcttcctgcagtaggcgttggc

SEQ ID NO: 101 - DNA, 50 bp, Homo sapiens
gcaggcagagaaggcccagtgtgtccatccccaatgcggtgatactagga

SEQ ID NO: 102 - DNA, 50 bp, Homo sapiens
agaggacagctctgacaaaggccgtacaatgccgggaagatgaatgtgcg

SEQ ID NO: 103 - DNA, 50 bp, Homo sapiens
gggaggacgcacccccactgctgttttcacatcctttcccttacccacct

SEQ ID NO: 104 - DNA, 50 bp, Homo sapiens
cgggggccttccctcgggtccctggcagaaagacattttaccccttcttg

SEQ ID NO: 105 - DNA, 50 bp, Homo sapiens
gtgaaccttattagaactcgcatgcaggcttcagccccagtggaaaaagg

SEQ ID NO: 106 - DNA, 50 bp, Homo sapiens
gccggctgaaataacctgaattcaagccaggaagaagcagcaatctgtct

SEQ ID NO: 107 - DNA, 50 bp, Homo sapiens
caaagtggtccaagatggctcttttttctttgaaaggggcctgttctcag

SEQ ID NO: 108 - DNA, 50 bp, Homo sapiens
catgatagacacgatgaaatccaccctcaaagagcagttccagtttgtgg

SEQ ID NO: 109 - DNA, 50 bp, Homo sapiens
ggatgagagggaaccactataacatgagtccaagcccagaagacttctgt

SEQ ID NO: 110 - DNA, 50 bp, Homo sapiens
caccacgatgtgcatcaaggaatgcctccgcctctacgcaccggtagtaa

SEQ ID NO: 111 - DNA, 50 bp, Homo sapiens
gctgagtcaatggctttgagaatgtcactgcatatgggagattgaggccc

SEQ ID NO: 112 - DNA, 49 bp, Homo sapiens
cttttgcagcactttacctctctgaaagccccagaggaccagagccccc

SEQ ID NO: 113 - DNA, 50 bp, Homo sapiens
gtgatgtgccacaaaggagccaaacaccttcgagttgcagcccgtgacct

SEQ ID NO: 114 - DNA, 50 bp, Homo sapiens
tcgagtgtgttgccaagtgctgcgtcctcctggtggcctctgtgtgtgtc

SEQ ID NO: 115 - DNA, 50 bp, Homo sapiens
ttgggacactaaccatccaggtcaagaaaagtcacatgccatagccatgg

SEQ ID NO: 116 - DNA, 50 bp, Homo sapiens
tgtgatcaagactcagaagcacgaacagtattgccctctgtgttagcccc

SEQ ID NO: 117 - DNA, 50 bp, Homo sapiens; identical to SEQ ID NO: 116

SEQ ID NO: 118 - DNA, 50 bp, Homo sapiens
agatatttccccagaggcacgcgaactgtcagtctttcctaaggcccccg

SEQ ID NO: 119 - DNA, 50 bp, Homo sapiens
ggaggttttgtttgctgctacggttttaatcatccagggtgccattccac

SEQ ID NO: 120 - DNA, 50 bp, Homo sapiens
cccctaaaatatttctgatggtgcactactctgaggcctgtatggcccct

SEQ ID NO: 121 - DNA, 25 bp, Homo sapiens
aatggactttggaagcagggtgatc

SEQ ID NO: 122 - DNA, 31 bp, Homo sapiens
gaaataatttttccctctcaagctctaagtc

SEQ ID NO: 123 - DNA, 21 bp, Homo sapiens
agaccagctccggtgggaagc

SEQ ID NO: 124 - DNA, 20 bp, Homo sapiens
gggcctcaatggacccaccg

SEQ ID NO: 125 - DNA, 22 bp, Homo sapiens
atccagacacctggagatgctg

SEQ ID NO: 126 - DNA, 25 bp, Homo sapiens
tcacgttgactacgactgagaagcc

SEQ ID NO: 127 - DNA, 22 bp, Homo sapiens
tgctctacatctggcctctgcc

SEQ ID NO: 128 - DNA, 26 bp, Homo sapiens
tctttgctgcctataccctctggttc

SEQ ID NO: 129 - DNA, 24 bp, Homo sapiens
aagaggatgtccgtacgaggcttc

SEQ ID NO: 130 - DNA, 25 bp, Homo sapiens
cagatgagcaggagactaaagatgc

SEQ ID NO: 131 - DNA, 24 bp, Homo sapiens
tagttggcgatggggttggttgac

SEQ ID NO: 132 - DNA, 24 bp, Homo sapiens
tgccgtattcttggtgtctggagc

SEQ ID NO: 133 - DNA, 25 bp, Homo sapiens
cccacagaacacgatgtaagtcttc

SEQ ID NO: 134 - DNA, 20 bp, Homo sapiens
ctgggcctttggcctgcctt

SEQ ID NO: 135 - DNA, 22 bp, Homo sapiens
actccgcaggtattcttgacgc

SEQ ID NO: 136 - DNA, 20 bp, Homo sapiens
gtcttcctgcgctcctcggc

SEQ ID NO: 137 - DNA, 28 bp, Homo sapiens
agcaacattaacgcacattcatcttccc

SEQ ID NO: 138 - DNA, 21 bp, Homo sapiens
gtgctgcgggaaggccttgtc

SEQ ID NO: 139 - DNA, 22 bp, Homo sapiens
ccaattcgctcacggttggtgc

SEQ ID NO: 140 - DNA, 26 bp, Homo sapiens
cactgaacaattacaccccaagtctc

SEQ ID NO: 141 - protein, 9 aa, Homo sapiens
ILILSIIFI

SEQ ID NO: 142 - protein, 9 aa, Homo sapiens
SMPKTGILI